SEQUENCE LISTING (885 sequences)

SEQ ID NO: 1 - protein, 17 aa, synthetic construct
IPESSELTLQELLGEER

SEQ ID NO: 2 - protein, 21 aa, synthetic construct
  SITE 1: note = N-term Fluorene
  SITE 21: note = C-term 1-Anthracene
  MOD_RES 21: note = Lysine(1-Anthracene)
IPESSELTLQELLGEERRAAX

SEQ ID NO: 3 - protein, 21 aa, synthetic construct
  SITE 21: note = C-term 1-Anthracene
  SITE 1: note = N-term Fluorene
  MOD_RES 21: note = Lysine(1-Anthracene)
IPESAELTLQELLGEERRAAX

SEQ ID NO: 4 - protein, 19 aa, synthetic construct
  MOD_RES 14: note = Dehydroalanine
  SITE 19: note = C-term Anthracene
  SITE 1: note = N-term Fluorene
  MOD_RES 19: note = Lysine(Anthracene)
IPQSAELTLQELLXRRKKX

SEQ ID NO: 5 - protein, 12 aa, synthetic construct
ELTLQELLGEER

SEQ ID NO: 6 - protein, 12 aa, synthetic construct
  VARIANT 1: note = X can be Nva, L, Octanonic, Beta-cyclopropyl-L-Ala-OH, Beta-cyclobutyl-L-Ala-OH, Beta-cyclohexyl-L-Ala-OH, Phenylglycine, Homo phenylalanine or Napthylalanine
  MOD_RES 1: note = Nva,Octanonic,Beta-cyclopropyl-L-Ala-OH,Beta-cyclob utyl-L-Ala-OH,Beta-cyclohexyl-L-Ala-OH,Phenylglycine,Homo phenylalanine or Napthylalanine
  VARIANT 2: note = X can be Aad, E or Cysteic acid
  MOD_RES 2: note = Aad or Cysteic acid
  VARIANT 3: note = X can be T, N, Hyp, Cyclopropylglycine, Cyclobutylglycine or Cyclopentylglycine
  MOD_RES 3: note = Hyp, Cyclopropylglycine, Cyclobutylglycine or Cyclopentylglycine
  VARIANT 4: note = X can be Aad, E or Cysteic acid
  MOD_RES 4: note = Aad or Cysteic acid
  VARIANT 6: note = X can be Nva, L, Octanonic, Beta-cyclopropyl-L-Ala-OH, Beta-cyclobutyl-L-Ala-OH, Beta-cyclohexyl-L-Ala-OH, Phenylglycine, Homo phenylalanine or Napthylalanine
  MOD_RES 6: note = Nva,Octanonic,Beta-cyclopropyl-L-Ala-OH,Beta-cyclob utyl-L-Ala-OH,Beta-cyclohexyl-L-Ala-OH,Phenylglycine,Homo phenylalanine or Napthylalanine
  VARIANT 7..8: note = X can be Aad, E or Cysteic acid
  MOD_RES 7..8: note = Aad or Cysteic acid
XXXXQXXXCEER

SEQ ID NO: 7 - protein, 12 aa, synthetic construct
  MOD_RES 9: note = Dehydroalanine
ELTLQELLXEER

SEQ ID NO: 8 - protein, 12 aa, synthetic construct
  MOD_RES 9: note = Dehydroalanine
  MOD_RES 1: note = Aad
  MOD_RES 2: note = Nva
  MOD_RES 3: note = Hyp
  MOD_RES 7: note = Beta-cyclobutyl-L-Ala-OH
  MOD_RES 8: note = Nva
XXXXGEXXXEER

SEQ ID NO: 9 - protein, 12 aa, synthetic construct
  MOD_RES 9: note = Dehydroalanine
  MOD_RES 1: note = Aad
  MOD_RES 2: note = Nva
  MOD_RES 4: note = Beta-cyclopropyl-L-Ala-OH
  MOD_RES 8: note = Nva
XXNXQELXXEER

SEQ ID NO: 10 - protein, 12 aa, synthetic construct
  MOD_RES 9: note = Dehydroalanine
  MOD_RES 1: note = Aad
  MOD_RES 3: note = Hyp
  MOD_RES 4: note = Nva
  MOD_RES 7..8: note = Nva
XLXXQEXXXEER

SEQ ID NO: 11 - protein, 12 aa, synthetic construct
  MOD_RES 9: note = Dehydroalanine
  MOD_RES 4: note = Beta-cyclopropyl-L-Ala-OH
  MOD_RES 7: note = Nva
  MOD_RES 8: note = Phenylglycine
ELNXGEXXXEER

SEQ ID NO: 12 - protein, 12 aa, synthetic construct
  MOD_RES 9: note = Dehydroalanine
  MOD_RES 1: note = Aad
  MOD_RES 2: note = Nva
  MOD_RES 4: note = Nva
  MOD_RES 8: note = Nva
XXNXQELXXEER

SEQ ID NO: 13 - protein, 12 aa, synthetic construct
  MOD_RES 9: note = Dehydroalanine
  MOD_RES 1: note = Aad
  MOD_RES 2: note = Phenylglycine
  MOD_RES 3: note = Hyp
  MOD_RES 4: note = Phenylglycine
  MOD_RES 8: note = Nva
XXXXQELXXEER

SEQ ID NO: 14 - protein, 12 aa, synthetic construct
  MOD_RES 9: note = Dehydroalanine
  MOD_RES 1: note = Aad
  MOD_RES 2: note = Beta-cyclopropyl-L-Ala-OH
  MOD_RES 4: note = Beta-cyclopropyl-L-Ala-OH
  MOD_RES 3: note = Cyclopropylglycine
  MOD_RES 7..8: note = Nva
XXXXAEXXXEER

SEQ ID NO: 15 - protein, 12 aa, synthetic construct
  MOD_RES 9: note = Dehydroalanine
  MOD_RES 1: note = Aad
  MOD_RES 2: note = Homo phenylalanine
  MOD_RES 4: note = Napthylalanine
  MOD_RES 8: note = Beta-cyclobutyl-L-Ala-OH
XXTXAELXXEER

SEQ ID NO: 16 - protein, 12 aa, synthetic construct
  MOD_RES 9: note = Dehydroalanine
  MOD_RES 1: note = Aad
  MOD_RES 2: note = Napthylalanine
  MOD_RES 3: note = Cyclobutylglycine
  MOD_RES 4: note = Beta-cyclopropyl-L-Ala-OH
  MOD_RES 5: note = Aib
  MOD_RES 7: note = Homo phenylalanine
  MOD_RES 8: note = Beta-cyclopropyl-L-Ala-OH
XXXXXEXXXEER

SEQ ID NO: 17 - protein, 12 aa, synthetic construct
  MOD_RES 9: note = Dehydroalanine
  MOD_RES 1: note = Aad
  MOD_RES 2: note = Napthylalanine
  MOD_RES 3: note = Cyclopentylglycine
  MOD_RES 5: note = Aib
  MOD_RES 7: note = Beta-cyclobutyl-L-Ala-OH
XXXLXEXLXEER

SEQ ID NO: 18 - protein, 12 aa, synthetic construct
  MOD_RES 9: note = Dehydroalanine
  MOD_RES 1: note = Aad
  MOD_RES 2: note = Beta-cyclohexyl-L-Ala-OH
  MOD_RES 3: note = Hyp
  MOD_RES 4: note = Napthylalanine
  MOD_RES 7: note = Beta-cyclohexyl-L-Ala-OH
  MOD_RES 8: note = Phenylglycine
XXXXAEXXXEER

SEQ ID NO: 19 - protein, 12 aa, synthetic construct
  MOD_RES 9: note = Dehydroalanine
  MOD_RES 1: note = Aad
  MOD_RES 2: note = Napthylalanine
  MOD_RES 3: note = Cyclopentylglycine
  MOD_RES 4: note = Cyclopropylglycine
  MOD_RES 5: note = Aib
  MOD_RES 6: note = Aad
XXXXXXLLXEER

SEQ ID NO: 20 - protein, 12 aa, synthetic construct
  MOD_RES 1: note = Aad
  VARIANT 2: note = W can be replaced by 4-phenyl phenylalanine, Napthylalanine, QuinolylAlanine or 6-Cl tryptophan
  MOD_RES 2: note = 4-phenyl phenylalanine, Napthylalanine, QuinolylAlanine or 6-Cl tryptophan
  MOD_RES 5: note = Aib
  MOD_RES 6: note = Aad
  MOD_RES 7: note = Beta-cyclobutyl-L-Ala-OH
  VARIANT 3: note = X can be Cyclohexylglycine or Cyclopentylglycine
  MOD_RES 3: note = Cyclohexylglycine or Cyclopentylglycine
  VARIANT 10..12: note = D-Alanine can be replaced by Aib, G,S, Aad, E, Q, Homocysteic acid , V,L,M,P, L-2,3-diaminopropionic acid, K,R,H,W,F,Y and abesent
  SITE 10..12: note = D-Alanine
  MOD_RES 10..12: note = Aib, Aad, Homocysteic acid or L-2,3-diaminopropionic acid
XWXLXXXLCAAA

SEQ ID NO: 21-24 - skipped (4 entries)

SEQ ID NO: 25 - protein, 5 aa, synthetic construct
IPESA

SEQ ID NO: 26 - protein, 18 aa, synthetic construct
IPESSELTLQELLGEERR

SEQ ID NO: 27 - protein, 18 aa, synthetic construct
  SITE 1: note = N-term Biotin-(polyethylene glycol 4)
IPESSELTLQELLGEERR

SEQ ID NO: 28 - protein, 12 aa, synthetic construct
  SITE 1: note = N-term acetylated
  SITE 12: note = C-term CONH2
  DISULFID 9..10: note = Disulfide bond
ELTLQELLCEER

SEQ ID NO: 29 - protein, 12 aa, synthetic construct
  DISULFID 10..11: note = Disulfide bond
  SITE 1: note = N-term acetylated
  SITE 12: note = C-term CONH2
ELTLQELLGCER

SEQ ID NO: 30 - protein, 12 aa, synthetic construct
  MOD_RES 9: note = Dehydroalanine
  SITE 1: note = N-term acetylated
  SITE 12: note = C-term CONH2
ELTLQELLXEER

SEQ ID NO: 31 - protein, 12 aa, synthetic construct
  SITE 1: note = N-term acetylated
  SITE 12: note = C-term CONH2
  MOD_RES 10: note = Dehydroalanine
ELTLQELLGXER

SEQ ID NO: 32 - protein, 12 aa, synthetic construct
  MOD_RES 11: note = Dehydroalanine
  SITE 1: note = N-term acetylated
  SITE 12: note = C-term CONH2
ELTLQELLGEXR

SEQ ID NO: 33 - protein, 14 aa, synthetic construct
  MOD_RES 14: note = Dehydroalanine
  SITE 14: note = C-term NH2
  SITE 1: note = N-term 9-Fluorene carboxyl
IPESAELTLQELLX

SEQ ID NO: 34 - protein, 14 aa, synthetic construct
  SITE 14: note = C-term NH2
  MOD_RES 14: note = Dehydroalanine
  SITE 1: note = N-term 9-Fluorene carboxyl
IPESAELTAQEAAX

SEQ ID NO: 35 - protein, 17 aa, synthetic construct
  SITE 17: note = C-term NH2
  SITE 1: note = N-term 9-Fluorene carboxyl
  MOD_RES 14: note = Dehydroalanine
IPESAELTLQELLXEER

SEQ ID NO: 36 - protein, 6 aa, synthetic construct
HHHHHH

SEQ ID NO: 37 - protein, 5 aa, synthetic construct
  SITE 1: note = N-term 9-fluorenyl acetamido
IPQSA

SEQ ID NO: 38 - protein, 9 aa, synthetic construct
ELTLQEELL

SEQ ID NO: 39 - protein, 6 aa, synthetic construct
  MOD_RES 1: note = Dehydroalanine
  SITE 6: note = C-term 1-anthracenyl acetamido
  MOD_RES 6: note = Lysine(1-anthracenyl acetamido)
XRRKKX

SEQ ID NO: 40 - protein, 5 aa, synthetic construct
  SITE 1: note = N-term Fluorescein isothiocyanate
IPESS

SEQ ID NO: 41 - protein, 12 aa, synthetic construct
  SITE 1: note = N-term Biotin-(polyethylene glycol 4)
ELTLQELLGEER

SEQ ID NO: 42 - protein, 12 aa, synthetic construct
  SITE 1: note = N-term Fluorescein isothiocyanate
ELTLQELLGEER

SEQ ID NO: 43 - protein, 17 aa, synthetic construct
  SITE 1: note = N-term Biotin-(polyethylene glycol 4)
IPESSELTLQELLGEER

SEQ ID NO: 44 - protein, 17 aa, synthetic construct
  SITE 1: note = N-term Fluorescein isothiocyanate
IPESSELTLQELLGEER

SEQ ID NO: 45 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 9: note = Dehydroalanine
XXXAXEAAXEER

SEQ ID NO: 46 - protein, 12 aa, synthetic construct
  SITE 1: note = N-term acetylated
  SITE 12: note = C-term NH2
  MOD_RES 9: note = Dehydroalanine
ELTLQELLXEER

SEQ ID NO: 47 - protein, 11 aa, synthetic construct
  MOD_RES 1: note = Aad
  MOD_RES 3: note = CycloHexylglycine
  MOD_RES 5: note = Aib
  MOD_RES 7: note = Cyclobutylalanine
XWXLXEXLCRQ

SEQ ID NO: 48 - protein, 12 aa, synthetic construct
  SITE 1: note = N-term acetylated
  SITE 12: note = C-term CONH2
  MOD_RES 9: note = Phenylacrylamide
ELTLQELLXEER

SEQ ID NO: 49 - protein, 12 aa, synthetic construct
  SITE 1: note = N-term acetylated
  SITE 12: note = C-term CONH2
  MOD_RES 10: note = Phenylacrylamide
ELTLQELLGXER

SEQ ID NO: 50 - protein, 12 aa, synthetic construct
  MOD_RES 9: note = L-2,3-diaminopropionic acid-acrylamide
  SITE 1: note = N-term acetylated
  SITE 12: note = C-term CONH2
ELTLQELLXEER

SEQ ID NO: 51 - protein, 12 aa, synthetic construct
  MOD_RES 10: note = L-2,3-diaminopropionic acid-acrylamide
  SITE 1: note = N-term acetylated
  SITE 12: note = C-term CONH2
ELTLQELLGXER

SEQ ID NO: 52 - protein, 12 aa, synthetic construct
  MOD_RES 11: note = L-2,3-diaminopropionic acid-acrylamide
  SITE 1: note = N-term acetylated
  SITE 12: note = C-term CONH2
ELTLQELLGEXR

SEQ ID NO: 53 - protein, 12 aa, synthetic construct
  SITE 1: note = N-term acetylated
  SITE 12: note = C-term CONH2
  MOD_RES 10: note = L-2,3-diaminopropionic acid-acrylamide
ELTLQELLGXER

SEQ ID NO: 54 - protein, 12 aa, synthetic construct
  SITE 1: note = N-term acetylated
  SITE 12: note = C-term CONH2
  MOD_RES 11: note = L-2,3-diaminopropionic acid-acrylamide
ELTLQELLGEXR

SEQ ID NO: 55 - protein, 12 aa, synthetic construct
  SITE 1: note = N-term acetylated
  SITE 12: note = C-term CONH2
  MOD_RES 11: note = L-2,3-diaminopropionic acid-propiolamide
ELTLQELLGEXR

SEQ ID NO: 56 - protein, 895 aa, synthetic construct
MEKLHQCYWKSGEPQSDDIEASRMKRAAAKHLIERYYHQLTEGCGNETNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKASAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVKLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNKDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGMLENLYFQGHHHHHHGLNDIFEAQKIEWHE

SEQ ID NO: 57 - protein, 550 aa, synthetic construct
MAHHHHHHPMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIXGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNPMSENLYFQGAMFQDPQERPRKLPQLCTELQTTIHDIILECVYCKQQLLRREVYDFAFRDLCIVYRDGNPYAVCDKCLKFYSKISEYRHYSYSLYGTTLEQQYNKPLSDLLIRCINCQKPLSPEEKQRHLDKKQRFHNIRGRWTGRCMSCSRSSRTRRETQL

SEQ ID NO: 58 - protein, 18 aa, synthetic construct
  SITE 1: note = N-term Fluorescein-5-Isothiocyanate
IPESSELTLQELLGEERR

SEQ ID NO: 59 - protein, 18 aa, synthetic construct
  SITE 1: note = N-term 9-fluorene acetamido
IPESSELTLQELLGEERR

SEQ ID NO: 60 - protein, 18 aa, synthetic construct
  SITE 1: note = N-term 1-fluorene acetamido
IPESSELTLQELLGEERR

SEQ ID NO: 61 - protein, 18 aa, synthetic construct
  SITE 1: note = N-term 1-Indane acetamido
IPESSELTLQELLGEERR

SEQ ID NO: 62 - protein, 18 aa, synthetic construct
  SITE 1: note = N-term 9-fluorenone-2-carboxamido
IPESSELTLQELLGEERR

SEQ ID NO: 63 - protein, 18 aa, synthetic construct
  SITE 1: note = N-term 9-fluorenone-1-carboxamido
IPESSELTLQELLGEERR

SEQ ID NO: 64 - protein, 18 aa, synthetic construct
  SITE 1: note = N-term 9-fluorenone-4-carboxamido
IPESSELTLQELLGEERR

SEQ ID NO: 65 - protein, 18 aa, synthetic construct
  SITE 1: note = N-term anthraquinone-2-carboxamido
IPESSELTLQELLGEERR

SEQ ID NO: 66 - protein, 18 aa, synthetic construct
  SITE 1: note = N-term xanthene-9-carboxamido
IPESSELTLQELLGEERR

SEQ ID NO: 67 - protein, 18 aa, synthetic construct
  SITE 1: note = N-term 1-anthracene carboxamido
IPESSELTLQELLGEERR

SEQ ID NO: 68 - protein, 18 aa, synthetic construct
  SITE 1: note = N-term 2-anthracene carboxamido
IPESSELTLQELLGEERR

SEQ ID NO: 69 - protein, 18 aa, synthetic construct
  SITE 1: note = N-term 1-adamantane carboxamido
IPESSELTLQELLGEERR

SEQ ID NO: 70 - protein, 18 aa, synthetic construct
  SITE 1: note = N-term Triphenyl acetamido
IPESSELTLQELLGEERR

SEQ ID NO: 71 - protein, 18 aa, synthetic construct
  SITE 1: note = N-term Diphenyl acetamido
IPESSELTLQELLGEERR

SEQ ID NO: 72 - protein, 18 aa, synthetic construct
  SITE 1: note = N-term 1-naphthyl carboxamido
IPESSELTLQELLGEERR

SEQ ID NO: 73 - protein, 18 aa, synthetic construct
  SITE 1: note = N-term 1,6-dihydrophenyl carboxamido
IPESSELTLQELLGEERR

SEQ ID NO: 74 - protein, 18 aa, synthetic construct
  SITE 1: note = N-term Pentafluorophenyl carboxamido
IPESSELTLQELLGEERR

SEQ ID NO: 75 - protein, 18 aa, synthetic construct
  SITE 1: note = N-term 6-hydroxy-2-naphthyl carboxamido
IPESSELTLQELLGEERR

SEQ ID NO: 76 - protein, 18 aa, synthetic construct
  SITE 1: note = N-term 1-pyrenebutyl carboxamido
IPESSELTLQELLGEERR

SEQ ID NO: 77 - protein, 18 aa, synthetic construct
  SITE 1: note = N-term 5-Acenaphthene carboxamide
IPESSELTLQELLGEERR

SEQ ID NO: 78 - protein, 18 aa, synthetic construct
  SITE 1: note = N-term 7-Methoxycoumarin-4-acetamido
IPESSELTLQELLGEERR

SEQ ID NO: 79 - protein, 18 aa, synthetic construct
  SITE 1: note = N-term 4-phenyl-phenylalanine
IPESSELTLQELLGEERR

SEQ ID NO: 80 - protein, 18 aa, synthetic construct
  SITE 1: note = N-term Cyclohexyl-alanine
IPESSELTLQELLGEERR

SEQ ID NO: 81 - protein, 21 aa, synthetic construct
  SITE 1: note = N-term acetylated
  SITE 21: note = C-term Fluorescein-5-Isothiocyanate
  MOD_RES 21: note = Lysine (Fluorescein-5-Isothiocyanate)
IPESSELTLQELLGEERRAAX

SEQ ID NO: 82 - protein, 21 aa, synthetic construct
  SITE 1: note = N-term acetylated
  SITE 21: note = C-term fluorene-9-acetamido
  MOD_RES 21: note = Lysine(fluorene-9-acetamido)
IPESSELTLQELLGEERRAAX

SEQ ID NO: 83 - protein, 21 aa, synthetic construct
  SITE 1: note = N-term acetylated
  SITE 21: note = C-term fluorene-1-acetamido
  MOD_RES 21: note = Lysine(fluorene-1-acetamido)
IPESSELTLQELLGEERRAAX

SEQ ID NO: 84 - protein, 21 aa, synthetic construct
  SITE 1: note = N-term acetylated
  SITE 21: note = C-term 1-Indanecarboxamido
  MOD_RES 21: note = Lysine(1-Indanecarboxamido)
IPESSELTLQELLGEERRAAX

SEQ ID NO: 85 - protein, 21 aa, synthetic construct
  SITE 1: note = N-term acetylated
  SITE 21: note = C-term 9-fluorenone-2-carboxamido
  MOD_RES 21: note = Lysine(9-fluorenone-2-carboxamido)
IPESSELTLQELLGEERRAAX

SEQ ID NO: 86 - protein, 21 aa, synthetic construct
  SITE 1: note = N-term acetylated
  SITE 21: note = C-term 9-fluorenone-1-carboxamido
  MOD_RES 21: note = Lysine(9-fluorenone-1-carboxamido)
IPESSELTLQELLGEERRAAX

SEQ ID NO: 87 - protein, 21 aa, synthetic construct
  SITE 1: note = N-term acetylated
  SITE 21: note = C-term 9-fluorenone-4-carboxamido
  MOD_RES 21: note = Lysine(9-fluorenone-4-carboxamido)
IPESSELTLQELLGEERRAAX

SEQ ID NO: 88 - protein, 21 aa, synthetic construct
  SITE 1: note = N-term acetylated
  SITE 21: note = C-term anthraquinone-2-carboxamido
  MOD_RES 21: note = Lysine(anthraquinone-2-carboxamido)
IPESSELTLQELLGEERRAAX

SEQ ID NO: 89 - protein, 21 aa, synthetic construct
  SITE 1: note = N-term acetylated
  SITE 21: note = C-term xanthene-9-carboxamido
  MOD_RES 21: note = Lysine(xanthene-9-carboxamido)
IPESSELTLQELLGEERRAAX

SEQ ID NO: 90 - protein, 21 aa, synthetic construct
  SITE 1: note = N-term acetylated
  SITE 21: note = C-term 1-anthracene carboxamido
  MOD_RES 21: note = Lysine(1-anthracene carboxamido)
IPESSELTLQELLGEERRAAX

SEQ ID NO: 91 - protein, 21 aa, synthetic construct
  SITE 1: note = N-term acetylated
  SITE 21: note = C-term 2-anthracene carboxamido
  MOD_RES 21: note = Lysine(2-anthracene carboxamido)
IPESSELTLQELLGEERRAAX

SEQ ID NO: 92 - protein, 21 aa, synthetic construct
  SITE 1: note = N-term acetylated
  SITE 21: note = C-term 1-adamantane carboxamido
  MOD_RES 21: note = Lysine(1-adamantane carboxamido)
IPESSELTLQELLGEERRAAX

SEQ ID NO: 93 - protein, 21 aa, synthetic construct
  SITE 1: note = N-term acetylated
  SITE 21: note = C-term triphenylacetamido
  MOD_RES 21: note = Lysine(triphenylacetamido)
IPESSELTLQELLGEERRAAX

SEQ ID NO: 94 - protein, 21 aa, synthetic construct
  SITE 1: note = N-term acetylated
  SITE 21: note = C-term diphenylacetamido
  MOD_RES 21: note = Lysine(diphenylacetamido)
IPESSELTLQELLGEERRAAX

SEQ ID NO: 95 - protein, 21 aa, synthetic construct
  SITE 1: note = N-term acetylated
  SITE 21: note = C-term 1-naphthyl carboxamido
  MOD_RES 21: note = Lysine(1-naphthyl carboxamido)
IPESSELTLQELLGEERRAAX

SEQ ID NO: 96 - protein, 21 aa, synthetic construct
  SITE 1: note = N-term acetylated
  SITE 21: note = C-term Exo-norbornene-carboxamido
  SITE 21: note = Lysine(Exo-norbornene-carboxamido)
IPESSELTLQELLGEERRAAX

SEQ ID NO: 97 - protein, 21 aa, synthetic construct
  SITE 1: note = N-term Fluorene
  SITE 21: note = C-term 9-fluorene
  MOD_RES 21: note = Lysine(9-fluorene)
IPESSELTLQELLGEERRAAX

SEQ ID NO: 98 - protein, 21 aa, synthetic construct
  SITE 1: note = N-term Fluorene
  SITE 21: note = C-term 5-Acenaphthene
  MOD_RES 21: note = Lysine(5-Acenaphthene)
IPESSELTLQELLGEERRAAX

SEQ ID NO: 99 - protein, 21 aa, synthetic construct
  SITE 1: note = N-term Fluorene
  SITE 21: note = C-term 1-naphthanlene
  MOD_RES 21: note = Lysine(1-naphthanlene)
IPESSELTLQELLGEERRAAX

SEQ ID NO: 100 - protein, 21 aa, synthetic construct
  SITE 1: note = N-term Anthracene
  SITE 21: note = C-term 2-anthraquinone
  MOD_RES 21: note = Lysine(2-anthraquinone)
IPESSELTLQELLGEERRAAX

SEQ ID NO: 101 - protein, 11 aa, synthetic construct
XXXLXEXLCQX

SEQ ID NO: 102 - protein, 11 aa, synthetic construct
XXXLXEXLCXR

SEQ ID NO: 103 - protein, 22 aa, synthetic construct
  SITE 1: note = N-term Fluorene-(Biotin-polyethylene glycol 4)
  SITE 22: note = C-term 1-Anthracene
KIPESAELTLQELLGEERRAAK

SEQ ID NO: 104 - protein, 21 aa, synthetic construct
  SITE 1: note = N-term Fluorene
  SITE 21: note = C-term 1-Anthracene
IPESAELTAQEAAGEERRAAK

SEQ ID NO: 105 - protein, 5 aa, synthetic construct
RRNKK

SEQ ID NO: 106 - protein, 21 aa, synthetic construct
  SITE 1: note = N-term Fluorene
  SITE 21: note = C-term Anthracene
  MOD_RES 21: note = Lysine(Anthracene)
IPESAELTLQELLGEERRAAX

SEQ ID NO: 107 - protein, 21 aa, synthetic construct
  SITE 21: note = C-term Anthracene
  SITE 1: note = N-term Fluorene
  MOD_RES 14: note = Dehydroalanine
  MOD_RES 21: note = Lysine(Anthracene)
IPESAELTLQELLXEERRAAX

SEQ ID NO: 108 - protein, 22 aa, synthetic construct
  SITE 1: note = N-term Fluorene
  SITE 22: note = C-term Anthracene
  MOD_RES 14: note = Dehydroalanine
  MOD_RES 22: note = Lysine(Anthracene)
IPESAELTLQELLXEERRNKKX

SEQ ID NO: 109 - protein, 19 aa, synthetic construct
  SITE 1: note = N-term Fluorene
  MOD_RES 14: note = Dehydroalanine
  SITE 19: note = C-term Anthracene
  MOD_RES 19: note = Lysine(Anthracene)
IPQSAELTAQEAAXRRKKX

SEQ ID NO: 110 - protein, 12 aa, synthetic construct
  SITE 1: note = N-term acetylated
ELTLQELLGEER

SEQ ID NO: 111 - protein, 12 aa, synthetic construct
  MOD_RES 9: note = Phenylacrylamide
  SITE 1: note = N-term acetylated
ELTLQELLCEER

SEQ ID NO: 112 - protein, 12 aa, synthetic construct
  SITE 1: note = N-term acetylated
  MOD_RES 10: note = Phenylacrylamide
ELTLQELLGCER

SEQ ID NO: 113 - protein, 12 aa, synthetic construct
  SITE 1: note = N-term acetylated
  MOD_RES 9: note = Dehydroalanine
ELTLQELLXEER

SEQ ID NO: 114 - protein, 12 aa, synthetic construct
  SITE 1: note = N-term acetylated
  MOD_RES 10: note = Dehydroalanine
ELTLQELLGXER

SEQ ID NO: 115 - protein, 12 aa, synthetic construct
  MOD_RES 11: note = Dehydroalanine
  SITE 1: note = N-term acetylated
ELTLQELLGEXR

SEQ ID NO: 116 - protein, 12 aa, synthetic construct
  SITE 1: note = N-term acetylated
  MOD_RES 9: note = L-2,3-diaminopropionic acid-acrylamide
ELTLQELLXEER

SEQ ID NO: 117 - protein, 12 aa, synthetic construct
  SITE 1: note = N-term acetylated
  MOD_RES 10: note = L-2,3-diaminopropionic acid-acrylamide
ELTLQELLGXER

SEQ ID NO: 118 - protein, 12 aa, synthetic construct
  MOD_RES 11: note = L-2,3-diaminopropionic acid-acrylamide
  SITE 1: note = N-term acetylated
ELTLQELLGEXR

SEQ ID NO: 119 - protein, 12 aa, synthetic construct
  SITE 1: note = N-term acetylated
  MOD_RES 10: note = 2-(Boc)aminobutyric acid-acrylamide
ELTLQELLGXER

SEQ ID NO: 120 - protein, 12 aa, synthetic construct
  MOD_RES 11: note = 2-(Boc)aminobutyric acid-acrylamide
  SITE 1: note = N-term acetylated
ELTLQELLGEXR

SEQ ID NO: 121 - protein, 12 aa, synthetic construct
  MOD_RES 11: note = 2-(Boc)aminobutyric acid-propiolamide
  SITE 1: note = N-term acetylated
ELTLQELLGEXR

SEQ ID NO: 122 - protein, 21 aa, synthetic construct
  SITE 1: note = N-term 9-fluorenyl acetamido
  SITE 21: note = C-term 1-anthracenyl acetamido
  MOD_RES 14: note = Dehydroalanine
  MOD_RES 21: note = Lysine(1-anthracenyl acetamido)
IPESAELTLQELLXEERRAAX

SEQ ID NO: 123 - protein, 22 aa, synthetic construct
  MOD_RES 14: note = Dehydroalanine
  SITE 1: note = N-term 9-fluorenyl acetamido
  SITE 22: note = C-term 1-anthracenyl acetamido
  MOD_RES 22: note = Lysine(1-anthracenyl acetamido)
IPESAELTLQELLXEERRNKKX

SEQ ID NO: 124 - protein, 22 aa, synthetic construct
  SITE 22: note = C-term 1-anthracenyl acetamido
  SITE 1: note = N-term 9-fluorenyl acetamido
  MOD_RES 14: note = Dehydroalanine
  MOD_RES 22: note = Lysine(1-anthracenyl acetamido)
IPQSAELTLQELLXEARRNKKX

SEQ ID NO: 125 - protein, 22 aa, synthetic construct
  SITE 1: note = N-term 9-fluorenyl acetamido
  SITE 22: note = C-term 1-anthracenyl acetamido
  MOD_RES 14: note = Dehydroalanine
  MOD_RES 22: note = Lysine(1-anthracenyl acetamido)
IPQSAELTLQELLXQARRNKKX

SEQ ID NO: 126 - protein, 21 aa, synthetic construct
  MOD_RES 14: note = Dehydroalanine
  SITE 1: note = N-term 9-fluorenyl acetamido
  SITE 21: note = C-term 1-anthracenyl acetamido
  MOD_RES 21: note = Lysine(1-anthracenyl acetamido)
IPQSAELTLQELLXQARRKKX

SEQ ID NO: 127 - protein, 20 aa, synthetic construct
  SITE 20: note = C-term 1-anthracenyl acetamido
  SITE 1: note = N-term 9-fluorenyl acetamido
  MOD_RES 14: note = Dehydroalanine
  MOD_RES 20: note = Lysine(1-anthracenyl acetamido)
IPQSAELTLQELLXQRRKKX

SEQ ID NO: 128 - protein, 19 aa, synthetic construct
  MOD_RES 14: note = Dehydroalanine
  SITE 1: note = N-term 9-fluorenyl acetamido
  SITE 19: note = C-term 1-anthracenyl acetamido
  MOD_RES 19: note = Lysine(1-anthracenyl acetamido)
IPQSAELTLQELLXRRKKX

SEQ ID NO: 129 - protein, 19 aa, synthetic construct
  SITE 1: note = N-term 9-fluorenyl acetamido
  SITE 19: note = C-term 1-anthracenyl acetamido
  MOD_RES 14: note = Dehydroalanine
  MOD_RES 19: note = Lysine(1-anthracenyl acetamido)
IPQSAELTAQEAAXRRKKX

SEQ ID NO: 130 - protein, 12 aa, synthetic construct
  MOD_RES 1: note = Aad
  MOD_RES 3: note = Hyp
  MOD_RES 7: note = Cyclopropylalanine
  SITE 12: note = C-term CONH2
XVXVAEXVCEER

SEQ ID NO: 131 - protein, 12 aa, synthetic construct
  MOD_RES 1: note = Aad
  MOD_RES 2: note = Cyclobutylalanine
  MOD_RES 3: note = Hyp
  MOD_RES 7: note = Cyclopropylalanine
  SITE 12: note = C-term CONH2
XXXVQEXVCEER

SEQ ID NO: 132 - protein, 12 aa, synthetic construct
  MOD_RES 1: note = Aad
  MOD_RES 3: note = Hyp
  MOD_RES 7: note = Cyclobutylalanine
  SITE 12: note = C-term CONH2
XVXVGEXVCEER

SEQ ID NO: 133 - protein, 12 aa, synthetic construct
  MOD_RES 1: note = Aad
  MOD_RES 4: note = Cyclopropylalanine
  SITE 12: note = C-term CONH2
XVNXQELVCEER

SEQ ID NO: 134 - protein, 12 aa, synthetic construct
  MOD_RES 1: note = Aad
  MOD_RES 3: note = Hyp
  SITE 12: note = C-term CONH2
XLXVQEVVCEER

SEQ ID NO: 135 - protein, 12 aa, synthetic construct
  MOD_RES 4: note = Cyclopropylalanine
  MOD_RES 8: note = Phenyl glycine
  SITE 12: note = C-term CONH2
ELNXGEVXCEER

SEQ ID NO: 136 - protein, 12 aa, synthetic construct
  SITE 12: note = C-term CONH2
  MOD_RES 1: note = Aad
  MOD_RES 2: note = Cyclopropylalanine
  MOD_RES 4: note = Cyclopropylalanine
  MOD_RES 3: note = Cyclobutylglycine
  MOD_RES 7: note = Octanonic
  MOD_RES 8: note = Phenyl glycine
XXXXAEXXCEER

SEQ ID NO: 137 - protein, 12 aa, synthetic construct
  MOD_RES 1: note = Aad
  SITE 12: note = C-term CONH2
XVNVQELVCEER

SEQ ID NO: 138 - protein, 12 aa, synthetic construct
  SITE 12: note = C-term CONH2
  MOD_RES 1: note = Aad
  MOD_RES 2: note = Phenyl glycine
  MOD_RES 4: note = Phenyl glycine
  MOD_RES 3: note = Hyp
XXXXQELVCEER

SEQ ID NO: 139 - protein, 12 aa, synthetic construct
  SITE 12: note = C-term CONH2
  MOD_RES 1: note = Aad
  MOD_RES 2: note = Cyclopropylalanine
  MOD_RES 4: note = Cyclopropylalanine
  MOD_RES 3: note = Cyclopropylglycine
XXXXAEVVCEER

SEQ ID NO: 140 - protein, 12 aa, synthetic construct
  MOD_RES 1: note = Aad
  MOD_RES 2: note = Homo phenylalanine
  MOD_RES 4: note = Napthyl alanine
  MOD_RES 5: note = Aib
  MOD_RES 7: note = Cyclopropylalanine
  SITE 12: note = C-term CONH2
XXTXXEXLCEER

SEQ ID NO: 141 - protein, 12 aa, synthetic construct
  SITE 12: note = C-term CONH2
  MOD_RES 1: note = Aad
  MOD_RES 2: note = Homo phenylalanine
  MOD_RES 4: note = Napthyl alanine
  MOD_RES 8: note = Cyclobutylalanine
XXTXAELXCEER

SEQ ID NO: 142 - protein, 12 aa, synthetic construct
  SITE 12: note = C-term CONH2
  MOD_RES 1: note = Aad
  MOD_RES 2: note = Homo phenylalanine
  MOD_RES 3: note = Cyclobutylglycine
  MOD_RES 4: note = Cyclobutylalanine
  MOD_RES 5: note = Aib
  MOD_RES 7: note = Homo phenylalanine
  MOD_RES 8: note = Cyclobutylalanine
XXXXXEXXCEER

SEQ ID NO: 143 - protein, 12 aa, synthetic construct
  MOD_RES 1: note = Aad
  MOD_RES 2: note = Napthyl alanine
  MOD_RES 3: note = Cyclobutylglycine
  MOD_RES 4: note = Cyclopropylalanine
  MOD_RES 8: note = Cyclopropylalanine
  MOD_RES 5: note = Aib
  MOD_RES 7: note = Homo phenylalanine
  SITE 12: note = C-term CONH2
XXXXXEXXCEER

SEQ ID NO: 144 - protein, 12 aa, synthetic construct
  MOD_RES 1: note = Aad
  MOD_RES 3: note = Cyclopropylglycine
  MOD_RES 4: note = Napthyl alanine
  MOD_RES 5: note = Aib
  MOD_RES 7: note = Cyclobutylalanine
  SITE 12: note = C-term CONH2
XLXXXEXLCEER

SEQ ID NO: 145 - protein, 12 aa, synthetic construct
  MOD_RES 1: note = Aad
  MOD_RES 2: note = Cyclopropylalanine
  MOD_RES 3: note = Hyp
  MOD_RES 4: note = Napthyl alanine
  MOD_RES 7: note = Phenyl glycine
  MOD_RES 8: note = Octanonic
  SITE 12: note = C-term CONH2
XXXXAEXXCEER

SEQ ID NO: 146 - protein, 12 aa, synthetic construct
  SITE 12: note = C-term CONH2
  MOD_RES 1: note = Aad
  MOD_RES 2: note = Homo phenylalanine
  MOD_RES 3: note = Cyclobutylglycine
  MOD_RES 4: note = Cyclobutylalanine
  MOD_RES 7: note = Napthyl alanine
XXXXAEXLCEER

SEQ ID NO: 147 - protein, 12 aa, synthetic construct
  SITE 12: note = C-term CONH2
  MOD_RES 1: note = Aad
  MOD_RES 2: note = Napthyl alanine
  MOD_RES 3: note = Cyclopentylglycine
  MOD_RES 5: note = Aib
  MOD_RES 7: note = Cyclobutylalanine
XXXLXEXLCEER

SEQ ID NO: 148 - protein, 12 aa, synthetic construct
  MOD_RES 1: note = Aad
  MOD_RES 2: note = Cyclohexylalanine
  MOD_RES 3: note = Hyp
  MOD_RES 4: note = Napthyl alanine
  MOD_RES 7: note = Cyclohexylalanine
  MOD_RES 8: note = Phenyl glycine
  SITE 12: note = C-term CONH2
XXXXAEXXCEER

SEQ ID NO: 149 - protein, 12 aa, synthetic construct
  SITE 12: note = C-term CONH2
  MOD_RES 1: note = Aad
  MOD_RES 2: note = Cyclohexylalanine
  MOD_RES 4: note = Cyclohexylalanine
  MOD_RES 5: note = Aib
  MOD_RES 7: note = Cyclopropylalanine
XXNXXEXLCEER

SEQ ID NO: 150 - protein, 12 aa, synthetic construct
  SITE 12: note = C-term CONH2
  MOD_RES 1: note = Aad
  MOD_RES 2: note = Octanonic
  MOD_RES 4: note = Homo phenylalanine
  MOD_RES 5: note = Aib
  MOD_RES 7: note = Cyclobutylalanine
XXTXXEXLCEER

SEQ ID NO: 151 - protein, 12 aa, synthetic construct
  SITE 12: note = C-term CONH2
  MOD_RES 1: note = Aad
  MOD_RES 2: note = Cyclopropylalanine
  MOD_RES 3: note = Cyclopropylglycine
  MOD_RES 4: note = Napthyl alanine
  MOD_RES 5: note = Aib
  MOD_RES 7: note = Cyclobutylalanine
XXXXXEXLCEER

SEQ ID NO: 152 - protein, 12 aa, synthetic construct
  SITE 12: note = C-term CONH2
  MOD_RES 1: note = Aad
  MOD_RES 2: note = Napthyl alanine
  MOD_RES 3: note = Cyclopentylglycine
  MOD_RES 4: note = Cyclopropylalanine
  MOD_RES 5: note = Aib
  MOD_RES 6: note = Aad
XXXXXXLLCEER

SEQ ID NO: 153 - protein, 12 aa, synthetic construct
  SITE 12: note = C-term CONH2
  MOD_RES 1: note = Aad
  MOD_RES 2: note = Homo phenylalanine
  MOD_RES 3: note = Cyclopropylglycine
  MOD_RES 4: note = Cyclobutylalanine
  MOD_RES 7: note = Napthyl alanine
XXXXAEXLCEER

SEQ ID NO: 154 - protein, 12 aa, synthetic construct
  SITE 12: note = C-term CONH2
  MOD_RES 1: note = Aad
  MOD_RES 4: note = Napthyl alanine
  MOD_RES 5: note = Aib
XLTXXELLCEER

SEQ ID NO: 155 - protein, 12 aa, synthetic construct
  SITE 12: note = C-term CONH2
  MOD_RES 1: note = Aad
  MOD_RES 3: note = Hyp
  MOD_RES 4: note = Cyclohexylalanine
  MOD_RES 6: note = Homo cysteic acid
  MOD_RES 7: note = Octanonic
  MOD_RES 8: note = Phenyl glycine
XLXXAXXYCEER

SEQ ID NO: 156 - protein, 12 aa, synthetic construct
  SITE 12: note = C-term CONH2
  MOD_RES 1: note = Aad
  MOD_RES 2: note = Napthyl alanine
  MOD_RES 5: note = Aib
  MOD_RES 7: note = Octanonic
  MOD_RES 8: note = Phenyl glycine
XXNVXEXXCEER

SEQ ID NO: 157 - protein, 12 aa, synthetic construct
  SITE 12: note = C-term CONH2
  MOD_RES 1: note = Homo cysteic acid
  MOD_RES 2: note = Cyclopropylalanine
  MOD_RES 3: note = Hyp
  MOD_RES 8: note = Phenyl glycine
XXXVQELXCEER

SEQ ID NO: 158 - protein, 12 aa, synthetic construct
  SITE 12: note = C-term CONH2
  MOD_RES 1: note = Aad
  MOD_RES 2: note = Napthyl alanine
  MOD_RES 4: note = Homo phenylalanine
  MOD_RES 7: note = Homo phenylalanine
XXTXGEXLCEER

SEQ ID NO: 159 - protein, 12 aa, synthetic construct
  SITE 12: note = C-term CONH2
  MOD_RES 1: note = Aad
  MOD_RES 2: note = Cyclobutylalanine
  MOD_RES 4: note = Cyclohexylalanine
  MOD_RES 7: note = Cyclobutylalanine
XXGXQEXVCEER

SEQ ID NO: 160 - protein, 12 aa, synthetic construct
  SITE 12: note = C-term CONH2
  MOD_RES 1: note = Aad
  MOD_RES 3: note = Cyclobutylglycine
  MOD_RES 4: note = Cyclobutylglycine
  MOD_RES 7: note = Cyclobutylglycine
XVXXAEXVCEER

SEQ ID NO: 161 - protein, 12 aa, synthetic construct
  MOD_RES 1: note = Aad
  MOD_RES 2: note = Homo phenylalanine
  MOD_RES 4: note = Homo phenylalanine
  MOD_RES 7: note = Homo phenylalanine
  SITE 12: note = C-term CONH2
XXTXQEXLCEER

SEQ ID NO: 162 - protein, 12 aa, synthetic construct
  SITE 12: note = C-term CONH2
  MOD_RES 1: note = Aad
  MOD_RES 4: note = Octanonic
  MOD_RES 7: note = Homo phenylalanine
  MOD_RES 8: note = Octanonic
XLNXAEXXCEER

SEQ ID NO: 163 - protein, 12 aa, synthetic construct
  MOD_RES 1: note = Aad
  MOD_RES 2: note = Napthyl alanine
  MOD_RES 7: note = Napthyl alanine
  MOD_RES 4: note = Octanonic
  MOD_RES 8: note = Octanonic
  SITE 12: note = C-term CONH2
XXTXAEXXCEER

SEQ ID NO: 164 - protein, 12 aa, synthetic construct
  SITE 12: note = C-term CONH2
  MOD_RES 1: note = Aad
  MOD_RES 2: note = Octanonic
  MOD_RES 4: note = Cyclohexylalanine
  MOD_RES 8: note = Napthyl alanine
XXNXQEVXCEER

SEQ ID NO: 165 - protein, 12 aa, synthetic construct
  SITE 12: note = C-term CONH2
  MOD_RES 1: note = Aad
  MOD_RES 2: note = Octanonic
  MOD_RES 3: note = Cyclobutylglycine
  MOD_RES 4: note = Cyclohexylalanine
  MOD_RES 8: note = Cyclohexylalanine
XXXXAELXCEER

SEQ ID NO: 166 - protein, 12 aa, synthetic construct
  SITE 12: note = C-term CONH2
  MOD_RES 1: note = Aad
  MOD_RES 2: note = Napthyl alanine
  MOD_RES 3: note = Cyclopropylglycine
  MOD_RES 4: note = Cyclohexylalanine
  MOD_RES 8: note = Cyclohexylalanine
  MOD_RES 7: note = Napthyl alanine
XXXXGEXXCEER

SEQ ID NO: 167 - protein, 12 aa, synthetic construct
  SITE 12: note = C-term CONH2
  MOD_RES 1: note = Aad
  MOD_RES 7..8: note = Cyclobutylalanine
XLGVAEXXCEER

SEQ ID NO: 168 - protein, 12 aa, synthetic construct
  SITE 12: note = C-term CONH2
  MOD_RES 1: note = Aad
  MOD_RES 2: note = Octanonic
  MOD_RES 4: note = QuinolylAlanine
  MOD_RES 8: note = Cyclobutylalanine
XXTXQEVXCEER

SEQ ID NO: 169 - protein, 12 aa, synthetic construct
  SITE 12: note = C-term CONH2
  MOD_RES 2: note = Cyclohexylalanine
  MOD_RES 4: note = Cyclobutylalanine
  MOD_RES 8: note = Napthyl alanine
EXGXQELXCEER

SEQ ID NO: 170 - protein, 12 aa, synthetic construct
  SITE 12: note = C-term CONH2
  MOD_RES 1: note = Aad
  MOD_RES 2: note = Octanonic
  MOD_RES 3: note = Cyclopropylglycine
  MOD_RES 4: note = Napthyl alanine
  MOD_RES 8: note = QuinolylAlanine
XXXXGEVXCEER

SEQ ID NO: 171 - protein, 12 aa, synthetic construct
  SITE 12: note = C-term CONH2
ELTLQELLCEER

SEQ ID NO: 172 - protein, 12 aa, synthetic construct
  SITE 12: note = C-term CONH2
  MOD_RES 3: note = Hyp
  MOD_RES 7: note = Phenyl glycine
ELXVGEXVCEER

SEQ ID NO: 173 - protein, 12 aa, synthetic construct
  SITE 12: note = C-term CONH2
  MOD_RES 3: note = Hyp
  MOD_RES 4: note = Phenyl glycine
  MOD_RES 7: note = Cyclopropylalanine
ELXXQEXLCEER

SEQ ID NO: 174 - protein, 12 aa, synthetic construct
  SITE 12: note = C-term CONH2
  MOD_RES 3: note = Hyp
  MOD_RES 7: note = Cyclobutylalanine
ELXVAEXVCEER

SEQ ID NO: 175 - protein, 12 aa, synthetic construct
  SITE 12: note = C-term CONH2
  MOD_RES 4: note = Phenyl glycine
ELNXQELVCEER

SEQ ID NO: 176 - protein, 12 aa, synthetic construct
  SITE 12: note = C-term CONH2
  MOD_RES 3: note = Cyclopropylglycine
  MOD_RES 4: note = Cyclopropylalanine
EVXXQEVVCEER

SEQ ID NO: 177 - protein, 12 aa, synthetic construct
  SITE 12: note = C-term CONH2
  MOD_RES 4: note = Cyclopropylalanine
EVNXGELVCEER

SEQ ID NO: 178 - protein, 12 aa, synthetic construct
  SITE 12: note = C-term CONH2
  MOD_RES 2: note = Cyclopropylalanine
  MOD_RES 3: note = Hyp
  MOD_RES 4: note = Cyclobutylalanine
EXXXAEVVCEER

SEQ ID NO: 179 - protein, 12 aa, synthetic construct
  SITE 12: note = C-term CONH2
  MOD_RES 3: note = Hyp
  MOD_RES 7: note = Cyclopropylalanine
ELXVAEXVCEER

SEQ ID NO: 180 - protein, 12 aa, synthetic construct
  SITE 12: note = C-term CONH2
  MOD_RES 1: note = Aad
  MOD_RES 3: note = Cyclopropylglycine
  MOD_RES 4: note = Cyclobutylalanine
  MOD_RES 5: note = Aib
  MOD_RES 6: note = Aad
  MOD_RES 7: note = Cyclopropylalanine
XLXXXXXLCERE

SEQ ID NO: 181 - protein, 12 aa, synthetic construct
  SITE 12: note = C-term CONH2
  MOD_RES 1: note = Aad
  MOD_RES 3: note = Cyclobutylglycine
  MOD_RES 4: note = Phenyl glycine
  MOD_RES 8: note = Phenyl glycine
XVXXQEVXCEER

SEQ ID NO: 182 - protein, 12 aa, synthetic construct
  SITE 12: note = C-term CONH2
  MOD_RES 1: note = Aad
  MOD_RES 3: note = Hyp
  MOD_RES 4: note = Cyclobutylalanine
  MOD_RES 8: note = Phenyl glycine
XVXXQELXCEER

SEQ ID NO: 183 - protein, 12 aa, synthetic construct
  SITE 12: note = C-term CONH2
  MOD_RES 1: note = Aad
  MOD_RES 4: note = Phenyl glycine
  MOD_RES 7: note = Cyclopropylalanine
XVTXAEXVCEER

SEQ ID NO: 184 - protein, 12 aa, synthetic construct
  SITE 12: note = C-term CONH2
  MOD_RES 3: note = Cyclopentylglycine
  MOD_RES 7: note = Cyclohexylalanine
ELXLQEXVCEER

SEQ ID NO: 185 - protein, 12 aa, synthetic construct
  SITE 12: note = C-term CONH2
  MOD_RES 1: note = Aad
  MOD_RES 2: note = Cyclopropylalanine
  MOD_RES 3: note = Cyclobutylglycine
  MOD_RES 7: note = Cyclopropylalanine
  MOD_RES 8: note = Phenyl glycine
XXXVAEXXCEER

SEQ ID NO: 186 - protein, 12 aa, synthetic construct
  SITE 12: note = C-term CONH2
  MOD_RES 2: note = Cyclobutylalanine
  MOD_RES 3: note = Cyclobutylglycine
  MOD_RES 4: note = Cyclopropylalanine
  MOD_RES 8: note = Phenyl glycine
EXXXAELXCEER

SEQ ID NO: 187 - protein, 12 aa, synthetic construct
  SITE 12: note = C-term CONH2
  MOD_RES 4: note = Cyclobutylalanine
  MOD_RES 8: note = Phenyl glycine
ELNXAEVYCEER

SEQ ID NO: 188 - protein, 12 aa, synthetic construct
  SITE 12: note = C-term CONH2
  MOD_RES 1: note = Aad
  MOD_RES 2: note = Cyclopropylalanine
  MOD_RES 3: note = Cyclobutylglycine
XXXVQELVCEER

SEQ ID NO: 189 - protein, 12 aa, synthetic construct
  SITE 12: note = C-term CONH2
  MOD_RES 2: note = Cyclobutylalanine
  MOD_RES 3: note = Cyclobutylglycine
  MOD_RES 4: note = Cyclopropylalanine
EXXXQEVVCEER

SEQ ID NO: 190 - protein, 12 aa, synthetic construct
  SITE 12: note = C-term CONH2
  MOD_RES 2: note = Cyclobutylalanine
  MOD_RES 3: note = Cyclobutylalanine
  MOD_RES 4: note = Napthyl alanine
EXXXAELVCEER

SEQ ID NO: 191 - protein, 12 aa, synthetic construct
  SITE 12: note = C-term CONH2
  MOD_RES 3: note = Cyclobutylglycine
ELXLAELVCEER

SEQ ID NO: 192 - protein, 12 aa, synthetic construct
  SITE 12: note = C-term CONH2
  MOD_RES 1: note = Aad
  MOD_RES 3: note = Cyclobutylalanine
  MOD_RES 4: note = Cyclohexylalanine
  MOD_RES 8: note = Phenyl glycine
XVXXGELXCERE

SEQ ID NO: 193 - protein, 12 aa, synthetic construct
  SITE 12: note = C-term CONH2
  MOD_RES 1: note = Aad
  MOD_RES 3: note = Hyp
  MOD_RES 7: note = Cyclobutylalanine
  MOD_RES 8: note = Phenyl glycine
XLXLAEXXCEER

SEQ ID NO: 194 - protein, 12 aa, synthetic construct
  SITE 12: note = C-term CONH2
  MOD_RES 1: note = Aad
  MOD_RES 2: note = Phenyl glycine
  MOD_RES 7: note = Phenyl glycine
  MOD_RES 3: note = Hyp
  MOD_RES 4: note = Cyclopropylalanine
XXXXQEXVCEER

SEQ ID NO: 195 - protein, 12 aa, synthetic construct
  MOD_RES 1: note = Aad
  MOD_RES 3: note = Hyp
  MOD_RES 8: note = Phenyl glycine
  SITE 12: note = C-term CONH2
XVXVQELXCEER

SEQ ID NO: 196 - protein, 12 aa, synthetic construct
  SITE 12: note = C-term CONH2
  MOD_RES 4: note = Cyclobutylalanine
ELNXAEVLCEER

SEQ ID NO: 197 - protein, 12 aa, synthetic construct
  SITE 12: note = C-term CONH2
  MOD_RES 7: note = Cyclobutylalanine
ELNVQEXLCEER

SEQ ID NO: 198 - protein, 12 aa, synthetic construct
  SITE 12: note = C-term CONH2
  MOD_RES 1: note = Aad
  MOD_RES 2: note = Cyclobutylalanine
  MOD_RES 3: note = Cyclopropylglycine
  MOD_RES 5: note = Aib
  MOD_RES 8: note = Phenyl glycine
  MOD_RES 7: note = Cyclobutylalanine
XXXLXEXXCEER

SEQ ID NO: 199 - protein, 12 aa, synthetic construct
  SITE 12: note = C-term CONH2
  MOD_RES 1: note = Aad
  MOD_RES 3: note = Cyclopentylglycine
  MOD_RES 4: note = Cyclopropylalanine
  MOD_RES 7: note = Cyclobutylalanine
XLXXAEXLCERE

SEQ ID NO: 200 - protein, 12 aa, synthetic construct
  SITE 12: note = C-term CONH2
  MOD_RES 1: note = Aad
  MOD_RES 2: note = Cyclohexylalanine
  MOD_RES 3: note = Hyp
  MOD_RES 4: note = Napthyl alanine
  MOD_RES 5: note = Aib
  MOD_RES 8: note = Phenyl glycine
XXXXXEVXCEER

SEQ ID NO: 201 - protein, 12 aa, synthetic construct
  SITE 12: note = C-term CONH2
  MOD_RES 1: note = Aad
  MOD_RES 3: note = Hyp
  MOD_RES 7: note = Phenyl glycine
  MOD_RES 8: note = Cyclopropylalanine
XLXVQEXXCEER

SEQ ID NO: 202 - protein, 12 aa, synthetic construct
  SITE 12: note = C-term CONH2
  MOD_RES 1: note = Aad
  MOD_RES 2: note = Cyclopropylalanine
  MOD_RES 3: note = Cyclobutylglycine
  MOD_RES 7: note = Homo phenylalanine
XXXVAEXVCEER

SEQ ID NO: 203 - protein, 12 aa, synthetic construct
  SITE 12: note = C-term CONH2
  MOD_RES 1: note = Aad
  MOD_RES 7: note = Cyclopropylalanine
XVNLQEXVCEER

SEQ ID NO: 204 - protein, 12 aa, synthetic construct
  SITE 12: note = C-term CONH2
  MOD_RES 1: note = Aad
  MOD_RES 3: note = Cyclobutylglycine
  MOD_RES 7: note = Cyclopropylalanine
XLXVAEXLCERE

SEQ ID NO: 205 - protein, 12 aa, synthetic construct
  SITE 12: note = C-term CONH2
  MOD_RES 1: note = Aad
  MOD_RES 2: note = Homo phenylalanine
  MOD_RES 4: note = Cyclopropylalanine
  MOD_RES 7: note = Cyclobutylalanine
XXNXAEXVCEER

SEQ ID NO: 206 - protein, 12 aa, synthetic construct
  SITE 12: note = C-term CONH2
  MOD_RES 3: note = Hyp
  MOD_RES 4: note = Cyclobutylalanine
  MOD_RES 8: note = Phenyl glycine
ELXXAEVXCEER

SEQ ID NO: 207 - protein, 12 aa, synthetic construct
  SITE 12: note = C-term CONH2
  MOD_RES 1: note = Aad
  MOD_RES 2: note = Cyclopropylalanine
  MOD_RES 3: note = Cyclobutylglycine
  MOD_RES 7: note = Octanonic
  MOD_RES 8: note = Phenyl glycine
XXXVAEXXCEER

SEQ ID NO: 208 - protein, 12 aa, synthetic construct
  SITE 12: note = C-term CONH2
  MOD_RES 2: note = Cyclobutylalanine
  MOD_RES 8: note = Phenyl glycine
EXNVGELXCEER

SEQ ID NO: 209 - protein, 12 aa, synthetic construct
  SITE 12: note = C-term CONH2
  MOD_RES 3: note = Cyclopropylglycine
  MOD_RES 4: note = Cyclopropylalanine
  MOD_RES 7: note = Homo phenylalanine
  MOD_RES 8: note = Cyclopropylalanine
XLXXAEXXCERE

SEQ ID NO: 210 - protein, 12 aa, synthetic construct
  SITE 12: note = C-term CONH2
  MOD_RES 8: note = Homo phenylalanine
ELTLQELXCEER

SEQ ID NO: 211 - protein, 12 aa, synthetic construct
  SITE 12: note = C-term CONH2
  MOD_RES 1: note = Aad
XVTVQEVVCERE

SEQ ID NO: 212 - protein, 12 aa, synthetic construct
  SITE 12: note = C-term CONH2
  MOD_RES 2: note = Cyclopropylalanine
  MOD_RES 3: note = Cyclopropylglycine
EXXLQEVVCEER

SEQ ID NO: 213 - protein, 12 aa, synthetic construct
  SITE 12: note = C-term CONH2
  MOD_RES 1: note = Aad
  MOD_RES 4: note = Cyclopropylalanine
  MOD_RES 8: note = Phenyl glycine
XVNXGELXCEER

SEQ ID NO: 214 - protein, 12 aa, synthetic construct
  SITE 12: note = C-term CONH2
  MOD_RES 1: note = Aad
  MOD_RES 3: note = Hyp
  MOD_RES 8: note = Cyclopropylalanine
XVXLAELXCEER

SEQ ID NO: 215 - protein, 12 aa, synthetic construct
  SITE 12: note = C-term CONH2
  MOD_RES 1: note = Aad
  MOD_RES 2: note = Cyclohexylalanine
  MOD_RES 3: note = Cyclopropylglycine
  MOD_RES 7: note = Cyclobutylalanine
XXXLAEXLCEER

SEQ ID NO: 216 - protein, 12 aa, synthetic construct
  SITE 12: note = C-term CONH2
  MOD_RES 3: note = Cyclobutylglycine
  MOD_RES 5: note = Aib
ELXVXEVLCEER

SEQ ID NO: 217 - protein, 12 aa, synthetic construct
  SITE 12: note = C-term CONH2
  MOD_RES 1: note = Aad
  MOD_RES 3: note = Cyclopentylglycine
  MOD_RES 7: note = Cyclobutylalanine
XVXVQEXVCEER

SEQ ID NO: 218 - protein, 12 aa, synthetic construct
  SITE 12: note = C-term CONH2
  MOD_RES 1: note = Aad
  MOD_RES 3: note = Cyclobutylglycine
  MOD_RES 5: note = Aib
  MOD_RES 7: note = Cyclobutylalanine
  MOD_RES 8: note = Phenyl glycine
XLXVXEXXCEER

SEQ ID NO: 219 - protein, 12 aa, synthetic construct
  SITE 12: note = C-term CONH2
  MOD_RES 1: note = Aad
  MOD_RES 2: note = Cyclopropylalanine
  MOD_RES 4: note = Phenyl glycine
  MOD_RES 7: note = Cyclobutylalanine
XXNYQEXVCEER

SEQ ID NO: 220 - protein, 12 aa, synthetic construct
  SITE 12: note = C-term CONH2
  MOD_RES 1: note = Aad
  MOD_RES 3: note = Hyp
  MOD_RES 7: note = Cyclobutylalanine
  MOD_RES 8: note = Phenyl glycine
XVXVAEXXCEER

SEQ ID NO: 221 - protein, 12 aa, synthetic construct
  MOD_RES 1: note = Aad
  MOD_RES 3: note = Cyclopropylglycine
  MOD_RES 4: note = Cyclopropylalanine
  MOD_RES 7: note = Cyclobutylalanine
  SITE 12: note = C-term CONH2
XVXXGEXVCERE

SEQ ID NO: 222 - protein, 12 aa, synthetic construct
  SITE 12: note = C-term CONH2
  MOD_RES 1: note = Aad
  MOD_RES 3: note = Cyclopropylglycine
  MOD_RES 8: note = Phenyl glycine
XLXLQEVXCERE

SEQ ID NO: 223 - protein, 12 aa, synthetic construct
  SITE 12: note = C-term CONH2
  MOD_RES 1: note = Aad
  MOD_RES 2: note = Cyclohexylalanine
  MOD_RES 4: note = Cyclobutylalanine
  MOD_RES 7: note = Cyclohexylalanine
XXNXAEXVCEER

SEQ ID NO: 224 - protein, 12 aa, synthetic construct
  SITE 12: note = C-term CONH2
  MOD_RES 3: note = Hyp
  MOD_RES 4: note = Phenyl glycine
  MOD_RES 8: note = Cyclopropylalanine
ELXXQELXCEER

SEQ ID NO: 225 - protein, 12 aa, synthetic construct
  SITE 12: note = C-term CONH2
  MOD_RES 1: note = Aad
  MOD_RES 3: note = Hyp
  MOD_RES 4: note = Cyclopropylalanine
XLXXGELVCEER

SEQ ID NO: 226 - protein, 12 aa, synthetic construct
  SITE 12: note = C-term CONH2
  MOD_RES 3: note = Hyp
ELXVAELLCEER

SEQ ID NO: 227 - protein, 12 aa, synthetic construct
  SITE 12: note = C-term CONH2
  MOD_RES 8: note = Phenyl glycine
ELNLQELXCEER

SEQ ID NO: 228 - protein, 12 aa, synthetic construct
  SITE 12: note = C-term CONH2
ELNVAELLCEER

SEQ ID NO: 229 - protein, 12 aa, synthetic construct
  MOD_RES 1: note = Aad
  SITE 12: note = C-term CONH2
  MOD_RES 3: note = Hyp
  MOD_RES 4: note = Phenyl glycine
XLXXQELVCEER

SEQ ID NO: 230 - protein, 12 aa, synthetic construct
  SITE 12: note = C-term CONH2
  MOD_RES 1: note = Aad
  MOD_RES 4: note = Cyclobutylalanine
XLNXQELVCEER

SEQ ID NO: 231 - protein, 12 aa, synthetic construct
  SITE 12: note = C-term CONH2
  MOD_RES 4: note = Cyclopropylalanine
  MOD_RES 7: note = Phenyl glycine
ELNXAEXLCEER

SEQ ID NO: 232 - protein, 12 aa, synthetic construct
  SITE 12: note = C-term CONH2
  MOD_RES 1: note = Aad
XVTVAEVLCERE

SEQ ID NO: 233 - protein, 12 aa, synthetic construct
  SITE 12: note = C-term CONH2
  MOD_RES 1: note = Aad
  MOD_RES 3: note = Hyp
  MOD_RES 4: note = Cyclopropylalanine
  MOD_RES 8: note = Phenyl glycine
XVXXAEVXCEER

SEQ ID NO: 234 - protein, 12 aa, synthetic construct
  SITE 12: note = C-term CONH2
ELNLAEVVCEER

SEQ ID NO: 235 - protein, 12 aa, synthetic construct
  SITE 12: note = C-term CONH2
  MOD_RES 1: note = Aad
  MOD_RES 3: note = Hyp
  MOD_RES 7: note = Homo phenylalanine
XVXLAEXLCEER

SEQ ID NO: 236 - protein, 12 aa, synthetic construct
  SITE 12: note = C-term CONH2
  MOD_RES 1: note = Aad
  MOD_RES 2: note = Phenyl glycine
  MOD_RES 3: note = Hyp
  MOD_RES 4: note = Cyclopropylalanine
XXXXAELVCEER

SEQ ID NO: 237 - protein, 12 aa, synthetic construct
  SITE 12: note = C-term CONH2
  MOD_RES 3: note = Hyp
  MOD_RES 4: note = Cyclobutylalanine
  MOD_RES 7: note = Cyclohexylalanine
ELXXAEXLCEER

SEQ ID NO: 238 - protein, 12 aa, synthetic construct
  SITE 12: note = C-term CONH2
  MOD_RES 1: note = Aad
  MOD_RES 2: note = Cyclobutylalanine
  MOD_RES 7: note = Phenyl glycine
  MOD_RES 4: note = Cyclopropylalanine
XXTXQEXLCEER

SEQ ID NO: 239 - protein, 12 aa, synthetic construct
  SITE 12: note = C-term CONH2
  MOD_RES 3: note = Cyclobutylglycine
  MOD_RES 5: note = Aib
  MOD_RES 7: note = Phenyl glycine
ELXVXEXVCEER

SEQ ID NO: 240 - protein, 12 aa, synthetic construct
  SITE 12: note = C-term CONH2
  MOD_RES 1: note = Aad
  MOD_RES 3: note = Hyp
  MOD_RES 4: note = Octanonic
  MOD_RES 7: note = Cyclopropylalanine
  MOD_RES 8: note = Octanonic
XLXXAEXXCEER

SEQ ID NO: 241 - protein, 12 aa, synthetic construct
  SITE 12: note = C-term CONH2
  MOD_RES 2: note = Cyclopropylalanine
  MOD_RES 4: note = Cyclopropylalanine
  MOD_RES 3: note = Hyp
  MOD_RES 7: note = Cyclopropylalanine
  MOD_RES 8: note = Phenyl glycine
EXXXAEXXCEER

SEQ ID NO: 242 - protein, 12 aa, synthetic construct
  SITE 12: note = C-term CONH2
  MOD_RES 1: note = Aad
  MOD_RES 2: note = Cyclopropylalanine
  MOD_RES 8: note = Phenyl glycine
  MOD_RES 4: note = Cyclobutylalanine
XXTXGEVXCEER

SEQ ID NO: 243 - protein, 12 aa, synthetic construct
  SITE 12: note = C-term CONH2
  MOD_RES 1: note = Aad
  MOD_RES 2: note = Cyclopropylalanine
  MOD_RES 3: note = Hyp
  MOD_RES 8: note = Phenyl glycine
XXXVAEVXCEER

SEQ ID NO: 244 - protein, 12 aa, synthetic construct
  SITE 12: note = C-term CONH2
  MOD_RES 1: note = Aad
  MOD_RES 3: note = Hyp
  MOD_RES 4: note = Cyclopropylalanine
  MOD_RES 8: note = Phenyl glycine
XLXXQEVXCEER

SEQ ID NO: 245 - protein, 12 aa, synthetic construct
  SITE 12: note = C-term CONH2
  MOD_RES 1: note = Aad
  MOD_RES 3: note = Cyclobutylglycine
  MOD_RES 5: note = Aib
XVXVXEVLCEER

SEQ ID NO: 246 - protein, 12 aa, synthetic construct
  SITE 12: note = C-term CONH2
  MOD_RES 1: note = Aad
  MOD_RES 2: note = Cyclopropylalanine
  MOD_RES 3: note = Cyclopropylglycine
  MOD_RES 4: note = Homo phenylalanine
XXXXAEVLCEER

SEQ ID NO: 247 - protein, 12 aa, synthetic construct
  SITE 12: note = C-term CONH2
  MOD_RES 1: note = Aad
  MOD_RES 3: note = Cyclobutylglycine
  MOD_RES 4: note = Phenyl glycine
  MOD_RES 5: note = Aib
XLXXXELLCEER

SEQ ID NO: 248 - protein, 12 aa, synthetic construct
  SITE 12: note = C-term CONH2
  MOD_RES 1: note = Aad
  MOD_RES 3: note = Hyp
  MOD_RES 4: note = Cyclopropylalanine
XLXXAELLCEER

SEQ ID NO: 249 - protein, 12 aa, synthetic construct
  SITE 12: note = C-term CONH2
  MOD_RES 1: note = Aad
  MOD_RES 7: note = Cyclobutylalanine
XLNVGEXLCERE

SEQ ID NO: 250 - protein, 12 aa, synthetic construct
  SITE 12: note = C-term CONH2
  MOD_RES 1: note = Aad
  MOD_RES 3: note = Hyp
  MOD_RES 4: note = Octanonic
  MOD_RES 6: note = Aad
  MOD_RES 7: note = Cyclobutylalanine
  MOD_RES 8: note = Phenyl glycine
XVXXAXXXCEER

SEQ ID NO: 251 - protein, 12 aa, synthetic construct
  SITE 12: note = C-term CONH2
  MOD_RES 1: note = Aad
  MOD_RES 4: note = Phenyl glycine
  MOD_RES 7: note = Cyclopropylalanine
  MOD_RES 8: note = Phenyl glycine
XVTXAEXXCEER

SEQ ID NO: 252 - protein, 12 aa, synthetic construct
  SITE 12: note = C-term CONH2
  MOD_RES 3: note = Cyclopentylglycine
  MOD_RES 4: note = Homo phenylalanine
  MOD_RES 6: note = Aad
  MOD_RES 7: note = Cyclopropylalanine
ELXXAXXVCEER

SEQ ID NO: 253 - protein, 12 aa, synthetic construct
  SITE 12: note = C-term CONH2
  MOD_RES 2: note = Cyclopropylalanine
EXNVGELVCEER

SEQ ID NO: 254 - protein, 12 aa, synthetic construct
  SITE 12: note = C-term CONH2
  MOD_RES 1: note = Aad
  MOD_RES 3: note = Cyclopropylglycine
  MOD_RES 4: note = Napthyl alanine
  MOD_RES 5: note = Aib
XVXXXELLCEER

SEQ ID NO: 255 - protein, 12 aa, synthetic construct
  SITE 12: note = C-term CONH2
  MOD_RES 1: note = Aad
  MOD_RES 2: note = Napthyl alanine
  MOD_RES 3: note = Cyclobutylglycine
  MOD_RES 4: note = Cyclopropylalanine
  MOD_RES 5: note = Aib
  MOD_RES 7: note = Cyclohexylalanine
  MOD_RES 8: note = Phenyl glycine
XXXXXEXXCEER

SEQ ID NO: 256 - protein, 12 aa, synthetic construct
  MOD_RES 1: note = Aad
  MOD_RES 2: note = Cyclohexylalanine
  MOD_RES 3: note = Cyclopropylglycine
  MOD_RES 4: note = Napthyl alanine
  SITE 12: note = C-term CONH2
XXXXAELVCEER

SEQ ID NO: 257 - protein, 12 aa, synthetic construct
  SITE 12: note = C-term CONH2
  MOD_RES 1: note = Aad
  MOD_RES 2: note = Phenyl glycine
  MOD_RES 3: note = Hyp
  MOD_RES 8: note = Phenyl glycine
XXXVQELXCEER

SEQ ID NO: 258 - protein, 12 aa, synthetic construct
  SITE 12: note = C-term CONH2
  MOD_RES 1: note = Aad
  MOD_RES 2: note = Cyclobutylalanine
  MOD_RES 3: note = Cyclobutylglycine
  MOD_RES 5: note = Aib
  MOD_RES 7: note = Cyclobutylalanine
  MOD_RES 8: note = Phenyl glycine
XXXVXEXXCEER

SEQ ID NO: 259 - protein, 12 aa, synthetic construct
  SITE 12: note = C-term CONH2
  MOD_RES 1: note = Aad
  MOD_RES 4: note = Cyclobutylalanine
  MOD_RES 8: note = Phenyl glycine
XLTXQEVXCEER

SEQ ID NO: 260 - protein, 12 aa, synthetic construct
  SITE 12: note = C-term CONH2
  MOD_RES 1: note = Aad
  MOD_RES 2: note = Cyclopropylalanine
  MOD_RES 3: note = Cyclopentylglycine
  MOD_RES 7: note = Cyclobutylalanine
  MOD_RES 8: note = Phenyl glycine
XXXLAEXXCEER

SEQ ID NO: 261 - protein, 12 aa, synthetic construct
  SITE 12: note = C-term CONH2
  MOD_RES 1: note = Aad
  MOD_RES 2: note = Napthyl alanine
  MOD_RES 5: note = Aib
  MOD_RES 8: note = Cyclohexylalanine
XXTVXEVXCEER

SEQ ID NO: 262 - protein, 12 aa, synthetic construct
  SITE 12: note = C-term CONH2
  MOD_RES 1: note = Aad
  MOD_RES 3: note = Hyp
  MOD_RES 4: note = Cyclobutylalanine
  MOD_RES 8: note = Phenyl glycine
XLXXAEVXCEER

SEQ ID NO: 263 - protein, 12 aa, synthetic construct
  SITE 12: note = C-term CONH2
ELXLQELLCEER

SEQ ID NO: 264 - protein, 12 aa, synthetic construct
  SITE 12: note = C-term CONH2
  MOD_RES 1: note = Aad
  MOD_RES 2: note = Napthyl alanine
  MOD_RES 4: note = Cyclopropylalanine
  MOD_RES 7: note = Cyclohexylalanine
  MOD_RES 8: note = Phenyl glycine
XXNXAEXXCEER

SEQ ID NO: 265 - protein, 12 aa, synthetic construct
  SITE 12: note = C-term CONH2
  MOD_RES 1: note = Aad
  MOD_RES 2: note = Cyclopropylalanine
  MOD_RES 3: note = Cyclobutylglycine
XXXVGELVCERE

SEQ ID NO: 266 - protein, 12 aa, synthetic construct
  SITE 12: note = C-term CONH2
  MOD_RES 3: note = Hyp
  MOD_RES 4: note = Phenyl glycine
  MOD_RES 7: note = Cyclopropylalanine
ELXXAEXVCEER

SEQ ID NO: 267 - protein, 12 aa, synthetic construct
  SITE 12: note = C-term CONH2
  MOD_RES 3: note = Cyclobutylglycine
  MOD_RES 4: note = Cyclohexylalanine
ELXXAEVLCEER

SEQ ID NO: 268 - protein, 12 aa, synthetic construct
  SITE 12: note = C-term CONH2
  MOD_RES 3: note = Hyp
  MOD_RES 7: note = Phenyl glycine
ELXVQEXLCEER

SEQ ID NO: 269 - protein, 12 aa, synthetic construct
  SITE 12: note = C-term CONH2
  MOD_RES 3: note = Cyclobutylglycine
  MOD_RES 4: note = Cyclohexylalanine
  MOD_RES 5: note = Aib
  MOD_RES 8: note = Phenyl glycine
ELXXXEVXCEER

SEQ ID NO: 270 - protein, 12 aa, synthetic construct
  SITE 12: note = C-term CONH2
  MOD_RES 1: note = Aad
  MOD_RES 2: note = Cyclopropylalanine
  MOD_RES 3: note = Hyp
  MOD_RES 7: note = Cyclopropylalanine
XXXVQEXVCEER

SEQ ID NO: 271 - protein, 12 aa, synthetic construct
  SITE 12: note = C-term CONH2
  MOD_RES 4: note = Cyclohexylalanine
  MOD_RES 5: note = Aib
  MOD_RES 7: note = Octanonic
ELTXXEXLCERE

SEQ ID NO: 272 - protein, 12 aa, synthetic construct
  SITE 12: note = C-term CONH2
  MOD_RES 1: note = Aad
  MOD_RES 3: note = Cyclopropylglycine
  MOD_RES 4: note = Cyclopropylalanine
  MOD_RES 6: note = Aad
XLXXAXVLCEER

SEQ ID NO: 273 - protein, 12 aa, synthetic construct
  SITE 12: note = C-term CONH2
  MOD_RES 1: note = Aad
  MOD_RES 2: note = Cyclopropylalanine
  MOD_RES 3: note = Hyp
  MOD_RES 7: note = Phenyl glycine
XXXVAEXVCEER

SEQ ID NO: 274 - protein, 12 aa, synthetic construct
  SITE 12: note = C-term CONH2
  MOD_RES 1: note = Aad
  MOD_RES 8: note = Homo phenylalanine
XVTLQELXCEER

SEQ ID NO: 275 - protein, 12 aa, synthetic construct
  SITE 12: note = C-term CONH2
  MOD_RES 1: note = Aad
  MOD_RES 4: note = Cyclopropylalanine
  MOD_RES 5: note = Aib
  MOD_RES 8: note = Phenyl glycine
XVNXXEVXCEER

SEQ ID NO: 276 - protein, 12 aa, synthetic construct
  SITE 12: note = C-term CONH2
  MOD_RES 3: note = Hyp
  MOD_RES 6: note = Aad
  MOD_RES 8: note = Cyclopropylalanine
ELXVGXVXCEER

SEQ ID NO: 277 - protein, 12 aa, synthetic construct
  SITE 12: note = C-term CONH2
  MOD_RES 1: note = Aad
  MOD_RES 2: note = Octanonic
  MOD_RES 4: note = Cyclopropylalanine
  MOD_RES 7: note = Homo phenylalanine
  MOD_RES 8: note = Cyclopropylalanine
XXTXAEXXCEER

SEQ ID NO: 278 - protein, 12 aa, synthetic construct
  SITE 12: note = C-term CONH2
  MOD_RES 1: note = Aad
  MOD_RES 2: note = Cyclopropylalanine
  MOD_RES 4: note = Cyclopropylalanine
  MOD_RES 8: note = Phenyl glycine
XXNXAEVXCEER

SEQ ID NO: 279 - protein, 12 aa, synthetic construct
  SITE 12: note = C-term CONH2
  MOD_RES 1: note = Aad
  MOD_RES 2: note = Octanonic
  MOD_RES 3: note = Cyclopropylglycine
  MOD_RES 4: note = Cyclopropylalanine
  MOD_RES 5: note = Aib
  MOD_RES 7: note = Cyclopropylalanine
XXXXXEXLCEER

SEQ ID NO: 280 - protein, 12 aa, synthetic construct
  SITE 12: note = C-term CONH2
  MOD_RES 1: note = Aad
  MOD_RES 3: note = Cyclopropylglycine
  MOD_RES 4: note = Homo phenylalanine
  MOD_RES 6: note = Aad
  MOD_RES 7: note = Cyclopropylalanine
XVXXAXXVCEER

SEQ ID NO: 281 - protein, 12 aa, synthetic construct
  SITE 12: note = C-term CONH2
  MOD_RES 1: note = Aad
  MOD_RES 3: note = Cyclopropylglycine
  MOD_RES 4: note = Cyclopropylalanine
  MOD_RES 5: note = Aib
  MOD_RES 6: note = Aad
  MOD_RES 7: note = Cyclobutylalanine
  MOD_RES 8: note = Phenyl glycine
XLXXXXXXCEER

SEQ ID NO: 282 - protein, 12 aa, synthetic construct
  SITE 12: note = C-term CONH2
  MOD_RES 1: note = Aad
  MOD_RES 2: note = Napthyl alanine
  MOD_RES 3: note = Cyclopentylglycine
  MOD_RES 4: note = Cyclobutylalanine
  MOD_RES 8: note = Cyclopropylalanine
XXXXGELXCEER

SEQ ID NO: 283 - protein, 12 aa, synthetic construct
  SITE 12: note = C-term CONH2
  MOD_RES 3: note = Hyp
  MOD_RES 4: note = Phenyl glycine
  MOD_RES 8: note = Cyclopropylalanine
  MOD_RES 1: note = Aad
XVXXQELXCEER

SEQ ID NO: 284 - protein, 12 aa, synthetic construct
  SITE 12: note = C-term CONH2
  MOD_RES 3: note = Hyp
  MOD_RES 4: note = Cyclopropylalanine
  MOD_RES 7: note = Cyclobutylalanine
  MOD_RES 8: note = Cyclopropylalanine
  MOD_RES 1: note = Aad
XVXXQEXXCEER

SEQ ID NO: 285 - protein, 12 aa, synthetic construct
  SITE 12: note = C-term CONH2
  MOD_RES 1: note = Aad
  MOD_RES 4: note = Cyclopropylalanine
XVTXAEVLCEER

SEQ ID NO: 286 - protein, 12 aa, synthetic construct
  SITE 12: note = C-term CONH2
  MOD_RES 1: note = Aad
XLNVAEVLCEER

SEQ ID NO: 287 - protein, 12 aa, synthetic construct
  SITE 12: note = C-term CONH2
  MOD_RES 7: note = Phenyl glycine
EVNVAEXVCEER

SEQ ID NO: 288 - protein, 12 aa, synthetic construct
  SITE 12: note = C-term CONH2
  MOD_RES 3: note = Hyp
  MOD_RES 8: note = Cyclopropylalanine
ELXLQELXCEER

SEQ ID NO: 289 - protein, 12 aa, synthetic construct
  SITE 12: note = C-term CONH2
  MOD_RES 1: note = Aad
  MOD_RES 3: note = Hyp
  MOD_RES 4: note = Cyclopropylalanine
  MOD_RES 7..8: note = Phenyl glycine
XLXXQEXXCEER

SEQ ID NO: 290 - protein, 12 aa, synthetic construct
  SITE 12: note = C-term CONH2
  MOD_RES 3: note = Hyp
  MOD_RES 4: note = Cyclopropylalanine
  MOD_RES 8: note = Phenyl glycine
ELXXGEVXCEER

SEQ ID NO: 291 - protein, 12 aa, synthetic construct
  SITE 12: note = C-term CONH2
  MOD_RES 1: note = Aad
  MOD_RES 7: note = Cyclopropylalanine
XVTVGEXLCEER

SEQ ID NO: 292 - protein, 12 aa, synthetic construct
  SITE 12: note = C-term CONH2
  MOD_RES 1: note = Aad
  MOD_RES 3: note = Cyclopropylglycine
  MOD_RES 4: note = Cyclopropylalanine
  MOD_RES 7: note = Cyclopropylalanine
XVXXQEXLCERE

SEQ ID NO: 293 - protein, 12 aa, synthetic construct
  SITE 12: note = C-term CONH2
  MOD_RES 1: note = Aad
  MOD_RES 2: note = Homo phenylalanine
  MOD_RES 3: note = Cyclopropylglycine
  MOD_RES 4: note = Cyclopropylalanine
XXXXAEVVCEER

SEQ ID NO: 294 - protein, 12 aa, synthetic construct
  SITE 12: note = C-term CONH2
  MOD_RES 1: note = Aad
  MOD_RES 5: note = Aib
  MOD_RES 7: note = Cyclopropylalanine
  MOD_RES 8: note = Phenyl glycine
XVNVXEXXCEER

SEQ ID NO: 295 - protein, 12 aa, synthetic construct
  SITE 12: note = C-term CONH2
  MOD_RES 3: note = Cyclobutylglycine
ELXLAEVLCEER

SEQ ID NO: 296 - protein, 12 aa, synthetic construct
  SITE 12: note = C-term CONH2
  MOD_RES 2: note = Phenyl glycine
  MOD_RES 4: note = Phenyl glycine
EXNXGEVVCEER

SEQ ID NO: 297 - protein, 12 aa, synthetic construct
  SITE 12: note = C-term CONH2
  MOD_RES 1: note = Aad
  MOD_RES 2: note = Cyclopropylalanine
  MOD_RES 3: note = Hyp
  MOD_RES 4: note = Phenyl glycine
  MOD_RES 7: note = Cyclopropylalanine
  MOD_RES 8: note = Cyclobutylalanine
XXXXQEXXCEER

SEQ ID NO: 298 - protein, 12 aa, synthetic construct
  SITE 12: note = C-term CONH2
  MOD_RES 1: note = Aad
  MOD_RES 3: note = Hyp
  MOD_RES 7: note = Phenyl glycine
XVXVGEXVCEER

SEQ ID NO: 299 - protein, 12 aa, synthetic construct
  SITE 12: note = C-term CONH2
  MOD_RES 2: note = Cyclobutylalanine
  MOD_RES 4: note = Phenyl glycine
EXNXQEVXCEER

SEQ ID NO: 300 - protein, 12 aa, synthetic construct
  SITE 12: note = C-term CONH2
  MOD_RES 2: note = Cyclopropylalanine
  MOD_RES 7..8: note = Cyclopropylalanine
  MOD_RES 3: note = Hyp
EXXVGEXXCEER

SEQ ID NO: 301 - protein, 12 aa, synthetic construct
  SITE 12: note = C-term CONH2
  MOD_RES 3: note = Hyp
  MOD_RES 7: note = Cyclopropylalanine
ELXVGEXLCEER

SEQ ID NO: 302 - protein, 12 aa, synthetic construct
  SITE 12: note = C-term CONH2
  MOD_RES 2: note = Phenyl glycine
  MOD_RES 4: note = Cyclopropylalanine
EXNXQEVVCEER

SEQ ID NO: 303 - protein, 12 aa, synthetic construct
  SITE 12: note = C-term CONH2
  MOD_RES 2: note = Cyclopropylalanine
  MOD_RES 3: note = Hyp
  MOD_RES 7: note = Phenyl glycine
EXXVQEXVCEER

SEQ ID NO: 304 - protein, 12 aa, synthetic construct
  SITE 12: note = C-term CONH2
  MOD_RES 1: note = Aad
  MOD_RES 2: note = Phenyl glycine
  MOD_RES 3: note = Hyp
  MOD_RES 7: note = Cyclobutylalanine
  MOD_RES 8: note = Cyclopropylalanine
XXXVQEXXCEER

SEQ ID NO: 305 - protein, 12 aa, synthetic construct
  SITE 12: note = C-term CONH2
  MOD_RES 1: note = Aad
  MOD_RES 3: note = Hyp
  MOD_RES 4: note = Cyclopropylalanine
  MOD_RES 5: note = Aib
  MOD_RES 8: note = Phenyl glycine
XLXXXEVXCEER

SEQ ID NO: 306 - protein, 12 aa, synthetic construct
  SITE 12: note = C-term CONH2
  MOD_RES 2: note = Cyclobutylalanine
  MOD_RES 3: note = Cyclobutylglycine
  MOD_RES 7: note = Cyclopropylalanine
  MOD_RES 8: note = Homo phenylalanine
EXXVAEXXCERE

SEQ ID NO: 307 - protein, 12 aa, synthetic construct
  SITE 12: note = C-term CONH2
  MOD_RES 2: note = Cyclopropylalanine
  MOD_RES 3: note = Hyp
  MOD_RES 5: note = Aib
  MOD_RES 7: note = Phenyl glycine
EXXVXEXVCEER

SEQ ID NO: 308 - protein, 12 aa, synthetic construct
  SITE 12: note = C-term CONH2
ELTLQELLCERE

SEQ ID NO: 309 - protein, 12 aa, synthetic construct
  SITE 12: note = C-term CONH2
  MOD_RES 1: note = Aad
  MOD_RES 3: note = Cyclobutylglycine
  MOD_RES 7: note = Cyclopropylalanine
  MOD_RES 8: note = Cyclobutylalanine
XVXVQEXXCEER

SEQ ID NO: 310 - protein, 12 aa, synthetic construct
  SITE 12: note = C-term CONH2
  MOD_RES 1: note = Aad
  MOD_RES 3: note = Cyclopropylglycine
XLXVQEVVCEER

SEQ ID NO: 311 - protein, 12 aa, synthetic construct
  SITE 12: note = C-term CONH2
  MOD_RES 1: note = Aad
  MOD_RES 3: note = Cyclobutylglycine
  MOD_RES 7: note = Cyclopropylalanine
XLXVQEXLCEER

SEQ ID NO: 312 - protein, 12 aa, synthetic construct
  SITE 12: note = C-term CONH2
  MOD_RES 1: note = Aad
  MOD_RES 3: note = Cyclopropylglycine
  MOD_RES 7: note = Cyclopropylalanine
XVXVAEXVCEER

SEQ ID NO: 313 - protein, 12 aa, synthetic construct
  SITE 12: note = C-term CONH2
  MOD_RES 1: note = Aad
  MOD_RES 4: note = Cyclopropylalanine
XVNXAEVVCEER

SEQ ID NO: 314 - protein, 12 aa, synthetic construct
  SITE 12: note = C-term CONH2
  MOD_RES 1: note = Aad
XLTVGEVLCEER

SEQ ID NO: 315 - protein, 12 aa, synthetic construct
  SITE 12: note = C-term CONH2
  MOD_RES 3: note = Hyp
  MOD_RES 4: note = Cyclopropylalanine
  MOD_RES 7: note = Phenyl glycine
ELXXQEXVCEER

SEQ ID NO: 316 - protein, 12 aa, synthetic construct
  SITE 12: note = C-term CONH2
  MOD_RES 1: note = Aad
  MOD_RES 3: note = Hyp
XVXVQELVCEER

SEQ ID NO: 317 - protein, 12 aa, synthetic construct
  SITE 12: note = C-term CONH2
  MOD_RES 1: note = Aad
  MOD_RES 3: note = Hyp
  MOD_RES 8: note = Cyclopropylalanine
XVXVQELXCEER

SEQ ID NO: 318 - protein, 12 aa, synthetic construct
  SITE 12: note = C-term CONH2
  MOD_RES 1: note = Aad
  MOD_RES 2: note = Cyclopropylalanine
  MOD_RES 7: note = Cyclopropylalanine
XXNVQEXLCEER

SEQ ID NO: 319 - protein, 12 aa, synthetic construct
  SITE 12: note = C-term CONH2
  MOD_RES 1: note = Aad
  MOD_RES 3: note = Cyclopropylglycine
  MOD_RES 8: note = Cyclopropylalanine
XVXVAELXCEER

SEQ ID NO: 320 - protein, 12 aa, synthetic construct
  SITE 12: note = C-term CONH2
  MOD_RES 1: note = Aad
  MOD_RES 2: note = Cyclopropylalanine
  MOD_RES 3: note = Cyclobutylglycine
XXXVGELVCEER

SEQ ID NO: 321 - protein, 12 aa, synthetic construct
  SITE 12: note = C-term CONH2
  MOD_RES 1: note = Aad
  MOD_RES 7: note = Cyclobutylalanine
XVNLQEXVCEER

SEQ ID NO: 322 - protein, 12 aa, synthetic construct
  SITE 12: note = C-term CONH2
  MOD_RES 1: note = Aad
  MOD_RES 3: note = Cyclopentylglycine
  MOD_RES 8: note = Cyclopropylalanine
XVXLQELXCEER

SEQ ID NO: 323 - protein, 12 aa, synthetic construct
  SITE 12: note = C-term CONH2
  MOD_RES 1: note = Aad
  MOD_RES 2: note = Phenyl glycine
  MOD_RES 3: note = Hyp
  MOD_RES 8: note = Cyclopropylalanine
XXXVGEVXCEER

SEQ ID NO: 324 - protein, 12 aa, synthetic construct
  SITE 12: note = C-term CONH2
  MOD_RES 1: note = Aad
  MOD_RES 2: note = Cyclopropylalanine
  MOD_RES 3: note = Hyp
  MOD_RES 4: note = Phenyl glycine
  MOD_RES 7: note = Cyclopropylalanine
XXXXQEXVCEER

SEQ ID NO: 325 - protein, 12 aa, synthetic construct
  SITE 12: note = C-term CONH2
  MOD_RES 1: note = Aad
  MOD_RES 2: note = Phenyl glycine
  MOD_RES 3: note = Hyp
  MOD_RES 4: note = Cyclopropylalanine
  MOD_RES 7..8: note = Phenyl glycine
XXXXQEXXCEER

SEQ ID NO: 326 - protein, 12 aa, synthetic construct
  SITE 12: note = C-term CONH2
  MOD_RES 1: note = Aad
  MOD_RES 2: note = Phenyl glycine
  MOD_RES 3: note = Hyp
  MOD_RES 7: note = Phenyl glycine
XYXLQEXVCEER

SEQ ID NO: 327 - protein, 12 aa, synthetic construct
  SITE 12: note = C-term CONH2
  MOD_RES 1: note = Aad
  MOD_RES 3: note = Cyclopropylglycine
  MOD_RES 4: note = Cyclopropylalanine
  MOD_RES 8: note = Phenyl glycine
XVXXQEVXCEER

SEQ ID NO: 328 - protein, 12 aa, synthetic construct
  SITE 12: note = C-term CONH2
  MOD_RES 1: note = Aad
XVTLQELLCEER

SEQ ID NO: 329 - protein, 12 aa, synthetic construct
  SITE 12: note = C-term CONH2
  MOD_RES 1: note = Aad
  MOD_RES 3: note = Cyclobutylglycine
  MOD_RES 8: note = Cyclopropylalanine
XLXVAEVXCEER

SEQ ID NO: 330 - protein, 12 aa, synthetic construct
  SITE 12: note = C-term CONH2
  MOD_RES 1: note = Aad
  MOD_RES 2: note = Cyclopropylalanine
  MOD_RES 4: note = Cyclopropylalanine
  MOD_RES 7: note = Cyclopropylalanine
  MOD_RES 3: note = Cyclopentylglycine
XXXXAEXVCEER

SEQ ID NO: 331 - protein, 12 aa, synthetic construct
  SITE 12: note = C-term CONH2
  MOD_RES 1: note = Aad
  MOD_RES 3: note = Cyclopropylglycine
  MOD_RES 4: note = Cyclobutylalanine
  MOD_RES 8: note = Phenyl glycine
XLXXQEVXCEER

SEQ ID NO: 332 - protein, 12 aa, synthetic construct
  SITE 12: note = C-term CONH2
  MOD_RES 1: note = Aad
  MOD_RES 3: note = Cyclobutylglycine
  MOD_RES 7: note = Cyclopropylalanine
XVXVQEXLCEER

SEQ ID NO: 333 - protein, 12 aa, synthetic construct
  SITE 12: note = C-term CONH2
  MOD_RES 1: note = Aad
  MOD_RES 2: note = Cyclohexylalanine
  MOD_RES 3: note = Hyp
  MOD_RES 7: note = Cyclopropylalanine
  MOD_RES 8: note = Cyclobutylalanine
XXXVQEXXCEER

SEQ ID NO: 334 - protein, 12 aa, synthetic construct
  SITE 12: note = C-term CONH2
  MOD_RES 2: note = Phenyl glycine
  MOD_RES 3: note = Cyclopropylglycine
  MOD_RES 1: note = Aad
XXXVAELLCEER

SEQ ID NO: 335 - protein, 12 aa, synthetic construct
  SITE 12: note = C-term CONH2
  MOD_RES 3: note = Hyp
  MOD_RES 4: note = Phenyl glycine
EVXXQEVVCEER

SEQ ID NO: 336 - protein, 12 aa, synthetic construct
  SITE 12: note = C-term CONH2
  MOD_RES 1: note = Aad
  MOD_RES 2: note = Phenyl glycine
  MOD_RES 3: note = Hyp
  MOD_RES 6: note = Aad
  MOD_RES 7: note = Cyclopropylalanine
XXXVQXXVCEER

SEQ ID NO: 337 - protein, 12 aa, synthetic construct
  SITE 12: note = C-term CONH2
  MOD_RES 1: note = Aad
  MOD_RES 3: note = Cyclopropylglycine
  MOD_RES 4: note = Cyclopropylalanine
  MOD_RES 8: note = Cyclopropylalanine
XLXXQEVXCERE

SEQ ID NO: 338 - protein, 12 aa, synthetic construct
  SITE 12: note = C-term CONH2
  MOD_RES 1: note = Aad
XVNVAELLCEER

SEQ ID NO: 339 - protein, 12 aa, synthetic construct
  SITE 12: note = C-term CONH2
  MOD_RES 1: note = Aad
  MOD_RES 3: note = Cyclopropylglycine
  MOD_RES 4: note = Cyclopropylalanine
  MOD_RES 8: note = Cyclopropylalanine
XLXXQELXCEER

SEQ ID NO: 340 - protein, 12 aa, synthetic construct
  SITE 12: note = C-term CONH2
  MOD_RES 1: note = Aad
  MOD_RES 3: note = Cyclopropylglycine
  MOD_RES 4: note = Cyclobutylalanine
  MOD_RES 7: note = Cyclopropylalanine
XVXXQEXLCEER

SEQ ID NO: 341 - protein, 12 aa, synthetic construct
  SITE 12: note = C-term CONH2
  MOD_RES 1: note = Aad
  MOD_RES 3: note = Cyclobutylalanine
  MOD_RES 4: note = Aib
XVTXXEVVCEER

SEQ ID NO: 342 - protein, 12 aa, synthetic construct
  SITE 12: note = C-term CONH2
  MOD_RES 1: note = Aad
  MOD_RES 2: note = Cyclobutylglycine
  MOD_RES 5: note = Aib
  MOD_RES 8: note = Cyclopropylalanine
XLXVXEVXCEER

SEQ ID NO: 343 - protein, 12 aa, synthetic construct
  SITE 12: note = C-term CONH2
  MOD_RES 1: note = Aad
  MOD_RES 3: note = Cyclobutylglycine
  MOD_RES 4: note = Cyclopropylalanine
XVXXAEVLCEER

SEQ ID NO: 344 - protein, 12 aa, synthetic construct
  SITE 12: note = C-term CONH2
  MOD_RES 1: note = Aad
  MOD_RES 3: note = Cyclopropylglycine
XVXLGELLCEER

SEQ ID NO: 345 - protein, 12 aa, synthetic construct
  SITE 12: note = C-term CONH2
  MOD_RES 1: note = Aad
XVTLAELLCEER

SEQ ID NO: 346 - protein, 12 aa, synthetic construct
  SITE 12: note = C-term CONH2
  MOD_RES 1: note = Aad
  MOD_RES 3: note = Cyclobutylglycine
  MOD_RES 7: note = Cyclopropylalanine
XVXLAEXLCEER

SEQ ID NO: 347 - protein, 12 aa, synthetic construct
  SITE 12: note = C-term CONH2
  MOD_RES 1: note = Aad
  MOD_RES 7: note = Cyclopropylalanine
XLTVQEXLCEER

SEQ ID NO: 348 - protein, 12 aa, synthetic construct
  SITE 12: note = C-term CONH2
  MOD_RES 1: note = Aad
  MOD_RES 3: note = Hyp
  MOD_RES 7: note = Cyclobutylalanine
XVXVAEXLCEER

SEQ ID NO: 349 - protein, 12 aa, synthetic construct
  SITE 12: note = C-term CONH2
  MOD_RES 1: note = Aad
  MOD_RES 2: note = Homo phenylalanine
  MOD_RES 3: note = Hyp
  MOD_RES 8: note = Phenyl glycine
XXXLQEVXCEER

SEQ ID NO: 350 - protein, 12 aa, synthetic construct
  SITE 12: note = C-term CONH2
  MOD_RES 1: note = Aad
  MOD_RES 3: note = Cyclobutylglycine
XVXLAEVVCEER

SEQ ID NO: 351 - protein, 12 aa, synthetic construct
  SITE 12: note = C-term CONH2
  MOD_RES 1: note = Aad
  MOD_RES 3: note = Cyclobutylglycine
  MOD_RES 4: note = Cyclopropylalanine
  MOD_RES 8: note = Phenyl glycine
XLXXQEVXCEER

SEQ ID NO: 352 - protein, 12 aa, synthetic construct
  SITE 12: note = C-term CONH2
  MOD_RES 1: note = Aad
  MOD_RES 2: note = Homo phenylalanine
  MOD_RES 3: note = Hyp
  MOD_RES 7: note = Cyclopropylalanine
XXXVQEXLCEER

SEQ ID NO: 353 - protein, 12 aa, synthetic construct
  SITE 12: note = C-term CONH2
  MOD_RES 1: note = Aad
  MOD_RES 2: note = Cyclopropylalanine
  MOD_RES 3: note = Cyclopentylglycine
XXXLAEVLCEER

SEQ ID NO: 354 - protein, 12 aa, synthetic construct
  SITE 12: note = C-term CONH2
  MOD_RES 1: note = Aad
  MOD_RES 2: note = Phenyl glycine
  MOD_RES 3: note = Hyp
  MOD_RES 4: note = Cyclopropylalanine
  MOD_RES 8: note = Cyclopropylalanine
XXXXAEVXCEER

SEQ ID NO: 355 - protein, 12 aa, synthetic construct
  SITE 12: note = C-term CONH2
  MOD_RES 1: note = Aad
  MOD_RES 2: note = Cyclopropylalanine
  MOD_RES 4: note = Phenyl glycine
  MOD_RES 8: note = Phenyl glycine
XXTXQEVXCEER

SEQ ID NO: 356 - protein, 12 aa, synthetic construct
  SITE 12: note = C-term CONH2
  MOD_RES 1: note = Aad
  MOD_RES 2: note = Phenyl glycine
  MOD_RES 4: note = Phenyl glycine
XXNXQEVVCEER

SEQ ID NO: 357 - protein, 12 aa, synthetic construct
  SITE 12: note = C-term CONH2
  MOD_RES 1: note = Aad
  MOD_RES 3: note = Hyp
  MOD_RES 4: note = Phenyl glycine
  MOD_RES 8: note = Phenyl glycine
XLXXQELXCEER

SEQ ID NO: 358 - protein, 12 aa, synthetic construct
  SITE 12: note = C-term CONH2
  MOD_RES 1: note = Aad
  MOD_RES 3: note = Cyclopropylglycine
  MOD_RES 4: note = Cyclobutylalanine
  MOD_RES 7: note = Cyclopropylalanine
XVXXAEXLCEER

SEQ ID NO: 359 - protein, 12 aa, synthetic construct
  SITE 12: note = C-term CONH2
  MOD_RES 1: note = Aad
  MOD_RES 3: note = Hyp
XLXVAELLCEER

SEQ ID NO: 360 - protein, 12 aa, synthetic construct
  SITE 12: note = C-term CONH2
  MOD_RES 1: note = Aad
  MOD_RES 3: note = Hyp
XVXLAELLCEER

SEQ ID NO: 361 - protein, 12 aa, synthetic construct
  SITE 12: note = C-term CONH2
  MOD_RES 1: note = Aad
  MOD_RES 3: note = Cyclobutylglycine
XVXVAEVLCEER

SEQ ID NO: 362 - protein, 12 aa, synthetic construct
  SITE 12: note = C-term CONH2
  MOD_RES 1: note = Aad
  MOD_RES 2: note = Cyclopropylalanine
  MOD_RES 7: note = Cyclopropylalanine
  MOD_RES 3: note = Hyp
  MOD_RES 4: note = Phenyl glycine
XXXXAEXVCEER

SEQ ID NO: 363 - protein, 12 aa, synthetic construct
  SITE 12: note = C-term CONH2
  MOD_RES 1: note = Aad
  MOD_RES 3: note = Hyp
  MOD_RES 8: note = Cyclopropylalanine
XVXVAEVXCEER

SEQ ID NO: 364 - protein, 12 aa, synthetic construct
  SITE 12: note = C-term CONH2
  MOD_RES 1: note = Aad
  MOD_RES 2: note = Phenyl glycine
  MOD_RES 8: note = Phenyl glycine
  MOD_RES 3: note = Hyp
  MOD_RES 4: note = Cyclopropylalanine
XXXXAEVXCEER

SEQ ID NO: 365 - protein, 12 aa, synthetic construct
  SITE 12: note = C-term CONH2
  MOD_RES 1: note = Aad
  MOD_RES 2: note = Cyclopropylalanine
  MOD_RES 3: note = Cyclopropylglycine
  MOD_RES 8: note = Phenyl glycine
XXXVAELXCEER

SEQ ID NO: 366 - protein, 12 aa, synthetic construct
  SITE 12: note = C-term CONH2
  MOD_RES 1: note = Aad
  MOD_RES 3: note = Cyclopentylglycine
  MOD_RES 7: note = Cyclopropylalanine
XVXLAEXVCERE

SEQ ID NO: 367 - protein, 12 aa, synthetic construct
  SITE 12: note = C-term CONH2
  MOD_RES 1: note = Aad
  MOD_RES 2: note = Cyclopropylalanine
  MOD_RES 3: note = Cyclopropylglycine
  MOD_RES 5: note = Aib
  MOD_RES 8: note = Phenyl glycine
XXXLXEVXCEER

SEQ ID NO: 368 - protein, 12 aa, synthetic construct
  SITE 12: note = C-term CONH2
  MOD_RES 1: note = Aad
  MOD_RES 3: note = Cyclopropylglycine
  MOD_RES 7: note = Octanonic
  MOD_RES 8: note = Phenyl glycine
XVXVAEXXCEER

SEQ ID NO: 369 - protein, 12 aa, synthetic construct
  SITE 12: note = C-term CONH2
  MOD_RES 1: note = Aad
  MOD_RES 4: note = Cyclopropylalanine
  MOD_RES 7: note = Phenyl glycine
XVTXAEXVCEER

SEQ ID NO: 370 - protein, 12 aa, synthetic construct
  SITE 12: note = C-term CONH2
  MOD_RES 1: note = Aad
  MOD_RES 3: note = Hyp
  MOD_RES 7: note = Cyclobutylalanine
XVXVQEXVCERE

SEQ ID NO: 371 - protein, 12 aa, synthetic construct
  SITE 12: note = C-term CONH2
  MOD_RES 1: note = Aad
  MOD_RES 3: note = Hyp
  MOD_RES 7: note = Cyclopropylalanine
XVXVAEXLCEER

SEQ ID NO: 372 - protein, 12 aa, synthetic construct
  SITE 12: note = C-term CONH2
  MOD_RES 1: note = Aad
  MOD_RES 3: note = Hyp
  MOD_RES 4: note = Cyclopropylalanine
  MOD_RES 8: note = Cyclobutylalanine
XVXXAEVXCEER

SEQ ID NO: 373 - protein, 12 aa, synthetic construct
  SITE 12: note = C-term CONH2
  MOD_RES 1: note = Aad
  MOD_RES 4: note = Cyclopropylalanine
  MOD_RES 7: note = Phenyl glycine
XVNXAEXVCEER

SEQ ID NO: 374 - protein, 12 aa, synthetic construct
  SITE 12: note = C-term CONH2
  MOD_RES 1: note = Aad
  MOD_RES 3: note = Hyp
  MOD_RES 4: note = Phenyl glycine
  MOD_RES 5: note = Aib
  MOD_RES 8: note = Phenyl glycine
XVXXXEVXCEER

SEQ ID NO: 375 - protein, 12 aa, synthetic construct
  SITE 12: note = C-term CONH2
  MOD_RES 1: note = Aad
  MOD_RES 3: note = Hyp
  MOD_RES 4: note = Cyclobutylalanine
XLXXGEVLCEER

SEQ ID NO: 376 - protein, 12 aa, synthetic construct
  SITE 12: note = C-term CONH2
  MOD_RES 3: note = Cyclobutylglycine
  MOD_RES 5: note = Aib
ELXVXEVVCEER

SEQ ID NO: 377 - protein, 12 aa, synthetic construct
  SITE 12: note = C-term CONH2
  MOD_RES 3: note = Hyp
  MOD_RES 7: note = Cyclobutylalanine
ELXVQEXVCEER

SEQ ID NO: 378 - protein, 12 aa, synthetic construct
  SITE 12: note = C-term CONH2
  MOD_RES 1: note = Aad
  MOD_RES 3: note = Cyclopropylglycine
  MOD_RES 4: note = Cyclopropylalanine
  MOD_RES 7: note = Cyclobutylalanine
XVXXGEXVCEER

SEQ ID NO: 379 - protein, 12 aa, synthetic construct
  SITE 12: note = C-term CONH2
  MOD_RES 1: note = Aad
  MOD_RES 8: note = Phenyl glycine
XVNVAEVXCEER

SEQ ID NO: 380 - protein, 12 aa, synthetic construct
  SITE 12: note = C-term CONH2
  MOD_RES 1: note = Aad
  MOD_RES 3: note = Hyp
  MOD_RES 8: note = Phenyl glycine
XVXLQELXCEER

SEQ ID NO: 381 - protein, 12 aa, synthetic construct
  SITE 12: note = C-term CONH2
  MOD_RES 1: note = Aad
XLTVGEVVCEER

SEQ ID NO: 382 - protein, 12 aa, synthetic construct
  SITE 12: note = C-term CONH2
  MOD_RES 1: note = Aad
  MOD_RES 2: note = Phenyl glycine
  MOD_RES 7: note = Cyclopropylalanine
XXNVQEXLCEER

SEQ ID NO: 383 - protein, 12 aa, synthetic construct
  SITE 12: note = C-term CONH2
  MOD_RES 1: note = Aad
  MOD_RES 3: note = Cyclopropylglycine
XLXVAELLCEER

SEQ ID NO: 384 - protein, 12 aa, synthetic construct
  SITE 12: note = C-term CONH2
  MOD_RES 3: note = Hyp
  MOD_RES 4: note = Cyclopropylalanine
  MOD_RES 7: note = Cyclopropylalanine
  MOD_RES 8: note = Phenyl glycine
ELXXAEXXCEER

SEQ ID NO: 385 - protein, 12 aa, synthetic construct
  SITE 12: note = C-term CONH2
  MOD_RES 1: note = Aad
  MOD_RES 3: note = Cyclopropylglycine
  MOD_RES 4: note = Cyclopropylalanine
  MOD_RES 8: note = Cyclopropylalanine
XVXXGEVXCEER

SEQ ID NO: 386 - protein, 12 aa, synthetic construct
  SITE 12: note = C-term CONH2
  MOD_RES 1: note = Aad
  MOD_RES 4: note = Phenyl glycine
XVNXAEVVCEER

SEQ ID NO: 387 - protein, 12 aa, synthetic construct
  SITE 12: note = C-term CONH2
  MOD_RES 1: note = Aad
  MOD_RES 3: note = Hyp
  MOD_RES 4: note = Cyclopropylalanine
  MOD_RES 8: note = Cyclopropylalanine
XVXXQELXCEER

SEQ ID NO: 388 - protein, 12 aa, synthetic construct
  SITE 12: note = C-term CONH2
  MOD_RES 1: note = Aad
  MOD_RES 3: note = Cyclopropylglycine
  MOD_RES 4: note = Cyclopropylalanine
  MOD_RES 7: note = Phenyl glycine
XVXXGEXLCEER

SEQ ID NO: 389 - protein, 12 aa, synthetic construct
  SITE 12: note = C-term CONH2
  MOD_RES 1: note = Aad
  MOD_RES 3: note = Hyp
  MOD_RES 4: note = Cyclopropylalanine
XLXXAEVLCEER

SEQ ID NO: 390 - protein, 12 aa, synthetic construct
  SITE 12: note = C-term CONH2
  MOD_RES 1: note = Aad
  MOD_RES 3: note = Cyclobutylglycine
  MOD_RES 7: note = Cyclopropylalanine
  MOD_RES 8: note = Phenyl glycine
XLXVQEXXCEER

SEQ ID NO: 391 - protein, 12 aa, synthetic construct
  SITE 12: note = C-term CONH2
  MOD_RES 1: note = Aad
  MOD_RES 3: note = Hyp
  MOD_RES 7: note = Cyclohexylalanine
XLXVQEXVCEER

SEQ ID NO: 392 - protein, 12 aa, synthetic construct
  SITE 12: note = C-term CONH2
  MOD_RES 3: note = Hyp
  MOD_RES 4: note = Phenyl glycine
  MOD_RES 7: note = Cyclopropylalanine
ELXXQEXVCEER

SEQ ID NO: 393 - protein, 12 aa, synthetic construct
  SITE 12: note = C-term CONH2
  MOD_RES 4: note = Phenyl glycine
EVNXAEVVCEER

SEQ ID NO: 394 - protein, 12 aa, synthetic construct
  SITE 12: note = C-term CONH2
  MOD_RES 3: note = Hyp
ELXVAEVVCEER

SEQ ID NO: 395 - protein, 12 aa, synthetic construct
  SITE 12: note = C-term CONH2
  MOD_RES 2: note = Cyclopropylalanine
  MOD_RES 3: note = Hyp
  MOD_RES 4: note = Phenyl glycine
  MOD_RES 7: note = Phenyl glycine
  MOD_RES 8: note = Cyclopropylalanine
EXXXQEXXCEER

SEQ ID NO: 396 - protein, 12 aa, synthetic construct
  SITE 12: note = C-term CONH2
  MOD_RES 3: note = Cyclobutylglycine
  MOD_RES 7: note = Cyclopropylalanine
  MOD_RES 8: note = Phenyl glycine
ELXVQEXXCEER

SEQ ID NO: 397 - protein, 12 aa, synthetic construct
  SITE 12: note = C-term CONH2
  MOD_RES 3: note = Hyp
  MOD_RES 6: note = Aad
  MOD_RES 8: note = Cyclopropylalanine
ELXVQXVXCEER

SEQ ID NO: 398 - protein, 12 aa, synthetic construct
  SITE 12: note = C-term CONH2
  MOD_RES 2: note = Cyclobutylalanine
  MOD_RES 8: note = Phenyl glycine
EXTVAEVXCEER

SEQ ID NO: 399 - protein, 12 aa, synthetic construct
  SITE 12: note = C-term CONH2
  MOD_RES 3: note = Hyp
ELXVQELVCEER

SEQ ID NO: 400 - protein, 12 aa, synthetic construct
  SITE 12: note = C-term CONH2
  MOD_RES 3: note = Cyclobutylglycine
  MOD_RES 8: note = Phenyl glycine
ELXVGEVXCEER

SEQ ID NO: 401 - protein, 12 aa, synthetic construct
  SITE 12: note = C-term CONH2
  MOD_RES 1: note = Aad
  MOD_RES 8: note = Phenyl glycine
XLTVGELXCEER

SEQ ID NO: 402 - protein, 12 aa, synthetic construct
  SITE 12: note = C-term CONH2
  MOD_RES 1: note = Aad
  MOD_RES 2: note = Cyclobutylalanine
  MOD_RES 4: note = Cyclopropylalanine
  MOD_RES 8: note = Phenyl glycine
XXTXAELXCEER

SEQ ID NO: 403 - protein, 12 aa, synthetic construct
  SITE 12: note = C-term CONH2
  MOD_RES 1: note = Aad
  MOD_RES 3: note = Hyp
  MOD_RES 7: note = Cyclobutylalanine
  MOD_RES 8: note = Phenyl glycine
XLXVAEXXCEER

SEQ ID NO: 404 - protein, 12 aa, synthetic construct
  SITE 12: note = C-term CONH2
  MOD_RES 1: note = Aad
  MOD_RES 3: note = Hyp
  MOD_RES 4: note = Cyclobutylalanine
  MOD_RES 7: note = Cyclobutylalanine
  MOD_RES 8: note = Phenyl glycine
XVXXAEXXCEER

SEQ ID NO: 405 - protein, 12 aa, synthetic construct
  SITE 12: note = C-term CONH2
  MOD_RES 1: note = Aad
  MOD_RES 2: note = Phenyl glycine
  MOD_RES 3: note = Cyclopropylglycine
XXXVAEVLCEER

SEQ ID NO: 406 - protein, 12 aa, synthetic construct
  SITE 12: note = C-term CONH2
  MOD_RES 1: note = Aad
  MOD_RES 3: note = Hyp
  MOD_RES 7: note = Octanonic
  MOD_RES 8: note = Cyclopropylalanine
XLXVQEXXCEER

SEQ ID NO: 407 - protein, 12 aa, synthetic construct
  SITE 12: note = C-term CONH2
  MOD_RES 3: note = Hyp
  MOD_RES 4: note = Cyclopropylalanine
  MOD_RES 8: note = Phenyl glycine
EVXXAEVXCEER

SEQ ID NO: 408 - protein, 12 aa, synthetic construct
  SITE 12: note = C-term CONH2
  MOD_RES 1: note = Aad
  MOD_RES 2: note = Phenyl glycine
  MOD_RES 3: note = Hyp
  MOD_RES 8: note = Phenyl glycine
XXXLQEVXCEER

SEQ ID NO: 409 - protein, 12 aa, synthetic construct
  SITE 12: note = C-term CONH2
  MOD_RES 1: note = Aad
  MOD_RES 3: note = Hyp
  MOD_RES 7: note = Cyclopropylalanine
  MOD_RES 8: note = Phenyl glycine
XVXLAEXXCEER

SEQ ID NO: 410 - protein, 12 aa, synthetic construct
  SITE 12: note = C-term CONH2
  MOD_RES 2: note = Cyclobutylalanine
  MOD_RES 3: note = Cyclopropylglycine
  MOD_RES 4: note = Cyclopropylalanine
EXXXQEVVCEER

SEQ ID NO: 411 - protein, 12 aa, synthetic construct
  SITE 12: note = C-term CONH2
  MOD_RES 1: note = Aad
  MOD_RES 2: note = Cyclopropylalanine
  MOD_RES 3: note = Cyclobutylglycine
  MOD_RES 4: note = Cyclobutylalanine
  MOD_RES 8: note = Phenyl glycine
XXXXQEVXCEER

SEQ ID NO: 412 - protein, 12 aa, synthetic construct
  SITE 12: note = C-term CONH2
  MOD_RES 1: note = Aad
  MOD_RES 3: note = Cyclobutylglycine
  MOD_RES 4: note = Cyclopropylalanine
  MOD_RES 5: note = Aib
XVXXXEVVCEER

SEQ ID NO: 413 - protein, 12 aa, synthetic construct
  SITE 12: note = C-term CONH2
  MOD_RES 1: note = Aad
  MOD_RES 3: note = Cyclopentylglycine
  MOD_RES 8: note = Phenyl glycine
XVXLGELXCEER

SEQ ID NO: 414 - protein, 12 aa, synthetic construct
  SITE 12: note = C-term CONH2
  MOD_RES 1: note = Aad
  MOD_RES 3: note = Hyp
  MOD_RES 4: note = Phenyl glycine
  MOD_RES 7: note = Cyclopropylalanine
  MOD_RES 8: note = Phenyl glycine
XVXXQEXXCEER

SEQ ID NO: 415 - protein, 12 aa, synthetic construct
  SITE 12: note = C-term CONH2
  MOD_RES 3: note = Cyclopropylglycine
  MOD_RES 4: note = Cyclopropylalanine
  MOD_RES 8: note = Phenyl glycine
EVXXQEVXCEER

SEQ ID NO: 416 - protein, 12 aa, synthetic construct
  SITE 12: note = C-term CONH2
  MOD_RES 1: note = Aad
  MOD_RES 2: note = Phenyl glycine
  MOD_RES 3: note = Hyp
XXXVAEVVCEER

SEQ ID NO: 417 - protein, 12 aa, synthetic construct
  SITE 12: note = C-term CONH2
  MOD_RES 1: note = Aad
  MOD_RES 8: note = Phenyl glycine
XVTLGELXCERE

SEQ ID NO: 418 - protein, 12 aa, synthetic construct
  SITE 12: note = C-term CONH2
  MOD_RES 3: note = Cyclopropylglycine
  MOD_RES 6: note = Aad
ELXVQXVVCEER

SEQ ID NO: 419 - protein, 12 aa, synthetic construct
  SITE 12: note = C-term CONH2
  MOD_RES 1: note = Aad
  MOD_RES 3: note = Cyclobutylglycine
  MOD_RES 4: note = Cyclopropylalanine
  MOD_RES 7: note = Cyclopropylalanine
XLXXAEXVCERE

SEQ ID NO: 420 - protein, 12 aa, synthetic construct
  SITE 12: note = C-term CONH2
  MOD_RES 2: note = Cyclobutylalanine
  MOD_RES 3: note = Hyp
  MOD_RES 8: note = Cyclopropylalanine
EXXVQELXCEER

SEQ ID NO: 421 - protein, 12 aa, synthetic construct
  SITE 12: note = C-term CONH2
  MOD_RES 1: note = Aad
  MOD_RES 3: note = Hyp
  MOD_RES 7: note = Cyclopropylalanine
XLXLQEXLCEER

SEQ ID NO: 422 - protein, 12 aa, synthetic construct
  SITE 12: note = C-term CONH2
  MOD_RES 1: note = Aad
XVTVAEVLCEER

SEQ ID NO: 423 - protein, 12 aa, synthetic construct
  SITE 12: note = Aad
  MOD_RES 7: note = Cyclobutylalanine
XVTLAEXVCEER

SEQ ID NO: 424 - protein, 12 aa, synthetic construct
  SITE 12: note = C-term CONH2
  MOD_RES 1: note = Aad
  MOD_RES 5: note = Aib
  MOD_RES 6: note = Cyclobutylalanine
XLTVXEXLCEER

SEQ ID NO: 425 - protein, 12 aa, synthetic construct
  SITE 12: note = C-term CONH2
  MOD_RES 1: note = Aad
  MOD_RES 4: note = Cyclopropylalanine
  MOD_RES 7: note = Cyclopropylalanine
  MOD_RES 5: note = Aib
XVTXXEXLCEER

SEQ ID NO: 426 - protein, 12 aa, synthetic construct
  SITE 12: note = C-term CONH2
  MOD_RES 1: note = Aad
  MOD_RES 2: note = Cyclopropylalanine
  MOD_RES 3: note = Cyclopropylglycine
XXXLQELVCEER

SEQ ID NO: 427 - protein, 12 aa, synthetic construct
  SITE 12: note = C-term CONH2
  MOD_RES 1: note = Aad
  MOD_RES 4: note = Cyclopropylalanine
  MOD_RES 5: note = Aib
XLTXXEVLCEER

SEQ ID NO: 428 - protein, 12 aa, synthetic construct
  SITE 12: note = C-term CONH2
  MOD_RES 1: note = Aad
  MOD_RES 3: note = Cyclobutylglycine
  MOD_RES 4: note = Cyclobutylalanine
  MOD_RES 7: note = Octanonic
XLXXAEXLCEER

SEQ ID NO: 429 - protein, 12 aa, synthetic construct
  SITE 12: note = C-term CONH2
  MOD_RES 1: note = Aad
  MOD_RES 3: note = Cyclopropylglycine
  MOD_RES 4: note = Cyclobutylalanine
XVXXGEVVCEER

SEQ ID NO: 430 - protein, 12 aa, synthetic construct
  SITE 12: note = C-term CONH2
  MOD_RES 1: note = Aad
  MOD_RES 2: note = Phenyl glycine
  MOD_RES 7: note = Cyclobutylalanine
XXNVAEXVCEER

SEQ ID NO: 431 - protein, 12 aa, synthetic construct
  SITE 12: note = C-term CONH2
  MOD_RES 1: note = Aad
  MOD_RES 4: note = Cyclobutylalanine
  MOD_RES 8: note = Phenyl glycine
XVTXAELXCEER

SEQ ID NO: 432 - protein, 12 aa, synthetic construct
  SITE 12: note = C-term CONH2
  MOD_RES 1: note = Aad
  MOD_RES 3: note = Cyclobutylglycine
  MOD_RES 7: note = Cyclopropylalanine
XLXVAEXLCEER

SEQ ID NO: 433 - protein, 12 aa, synthetic construct
  SITE 12: note = C-term CONH2
  MOD_RES 1: note = Aad
  MOD_RES 3: note = Cyclobutylglycine
  MOD_RES 7: note = Cyclobutylalanine
XLXVQEXVCEER

SEQ ID NO: 434 - protein, 12 aa, synthetic construct
  SITE 12: note = C-term CONH2
  MOD_RES 1: note = Aad
  MOD_RES 5: note = Aib
  MOD_RES 8: note = Phenyl glycine
XLNVXELXCEER

SEQ ID NO: 435 - protein, 12 aa, synthetic construct
  SITE 12: note = C-term CONH2
  MOD_RES 1: note = Aad
  MOD_RES 3: note = Cyclobutylglycine
XLXVAELLCEER

SEQ ID NO: 436 - protein, 12 aa, synthetic construct
  SITE 12: note = C-term CONH2
  MOD_RES 1: note = Aad
  MOD_RES 2: note = Homo phenylalanine
  MOD_RES 3: note = Cyclobutylglycine
  MOD_RES 7: note = Cyclobutylalanine
XXXVAEXLCEER

SEQ ID NO: 437 - protein, 12 aa, synthetic construct
  SITE 12: note = C-term CONH2
  MOD_RES 1: note = Aad
  MOD_RES 3: note = Cyclobutylglycine
  MOD_RES 4: note = Cyclopropylalanine
  MOD_RES 8: note = Cyclobutylalanine
XLXXXELXCEER

SEQ ID NO: 438 - protein, 12 aa, synthetic construct
  SITE 12: note = C-term CONH2
  MOD_RES 1: note = Aad
  MOD_RES 2: note = Homo phenylalanine
  MOD_RES 3: note = Cyclopropylglycine
  MOD_RES 5: note = Aib
  MOD_RES 7: note = Cyclobutylalanine
XXXLXEXVCEER

SEQ ID NO: 439 - protein, 12 aa, synthetic construct
  SITE 12: note = C-term CONH2
  MOD_RES 1: note = Aad
  MOD_RES 4: note = Cyclopropylalanine
XVNXAEVLCEER

SEQ ID NO: 440 - protein, 12 aa, synthetic construct
  SITE 12: note = C-term CONH2
  MOD_RES 1: note = Aad
  MOD_RES 3: note = Cyclopropylglycine
  MOD_RES 4: note = Cyclopropylalanine
  MOD_RES 8: note = Phenyl glycine
XVXXAELXCEER

SEQ ID NO: 441 - protein, 12 aa, synthetic construct
  MOD_RES 1: note = Aad
  MOD_RES 3: note = Cyclobutylglycine
  MOD_RES 4: note = Cyclopropylalanine
  MOD_RES 5: note = Aib
  MOD_RES 8: note = Phenyl glycine
  SITE 12: note = C-term CONH2
XVXXXEVXCEER

SEQ ID NO: 442 - protein, 12 aa, synthetic construct
  MOD_RES 1: note = Aad
  MOD_RES 3: note = Cyclopentylglycine
  MOD_RES 4: note = Cyclohexylalanine
  SITE 12: note = C-term CONH2
XVXXAELVCEER

SEQ ID NO: 443 - protein, 12 aa, synthetic construct
  MOD_RES 1: note = Aad
  MOD_RES 4: note = Cyclobutylalanine
  MOD_RES 8: note = Cyclobutylalanine
  SITE 12: note = C-term CONH2
XVNXAEVXCEER

SEQ ID NO: 444 - protein, 12 aa, synthetic construct
  MOD_RES 1: note = Aad
  MOD_RES 2: note = Cyclopropylalanine
  MOD_RES 3: note = Cyclobutylglycine
  MOD_RES 4: note = Cyclobutylalanine
  MOD_RES 8: note = Phenyl glycine
  SITE 12: note = C-term CONH2
XXXXQELXCEER

SEQ ID NO: 445 - protein, 12 aa, synthetic construct
  MOD_RES 1: note = Aad
  MOD_RES 2: note = Cyclopropylalanine
  MOD_RES 3: note = Cyclobutylglycine
  MOD_RES 7: note = Homo phenylalanine
  MOD_RES 8: note = Cyclopropylalanine
  SITE 12: note = C-term CONH2
XXXVQEXXCEER

SEQ ID NO: 446 - protein, 12 aa, synthetic construct
  MOD_RES 1: note = Aad
  MOD_RES 3: note = Cyclopropylglycine
  MOD_RES 4: note = Phenyl glycine
  SITE 12: note = C-term CONH2
XVXXAELLCEER

SEQ ID NO: 447 - protein, 12 aa, synthetic construct
  MOD_RES 1: note = Aad
  MOD_RES 3: note = Cyclobutylglycine
  MOD_RES 7: note = Cyclobutylalanine
  SITE 12: note = C-term CONH2
XLXLGEXLCEER

SEQ ID NO: 448 - protein, 12 aa, synthetic construct
  MOD_RES 1: note = Aad
  MOD_RES 3: note = Cyclobutylglycine
  MOD_RES 4: note = Cyclopropylalanine
  MOD_RES 5: note = Aib
  MOD_RES 7: note = Cyclobutylalanine
  SITE 12: note = C-term CONH2
XVXXXEXLCEER

SEQ ID NO: 449 - protein, 12 aa, synthetic construct
  MOD_RES 1: note = Aad
  MOD_RES 3: note = Cyclopropylglycine
  MOD_RES 4: note = Cyclopropylalanine
  MOD_RES 5: note = Aib
  MOD_RES 7: note = Octanonic
  SITE 12: note = C-term CONH2
XVXXXEXLCEER

SEQ ID NO: 450 - protein, 12 aa, synthetic construct
  MOD_RES 1: note = Aad
  MOD_RES 3: note = Cyclopropylglycine
  MOD_RES 4: note = Cyclopropylalanine
  MOD_RES 8: note = Phenyl glycine
  SITE 12: note = C-term CONH2
XLXXAELXCEER

SEQ ID NO: 451 - protein, 12 aa, synthetic construct
  MOD_RES 1: note = Aad
  MOD_RES 2: note = Cyclobutylalanine
  SITE 12: note = C-term CONH2
XXTVGEVVCEER

SEQ ID NO: 452 - protein, 12 aa, synthetic construct
  MOD_RES 1: note = Aad
  MOD_RES 3: note = Cyclopropylglycine
  SITE 12: note = C-term CONH2
XVXVAEVLCEER

SEQ ID NO: 453 - protein, 12 aa, synthetic construct
  MOD_RES 1: note = Aad
  MOD_RES 4: note = Cyclobutylalanine
  MOD_RES 8: note = Phenyl glycine
  SITE 12: note = C-term CONH2
XVTXAEVXCEER

SEQ ID NO: 454 - protein, 12 aa, synthetic construct
  MOD_RES 1: note = Aad
  MOD_RES 3: note = Cyclopentylglycine
  MOD_RES 5: note = Aib
  MOD_RES 7: note = Cyclopropylalanine
  SITE 12: note = C-term CONH2
XLXLXEXLCEER

SEQ ID NO: 455 - protein, 12 aa, synthetic construct
  MOD_RES 1: note = Aad
  MOD_RES 7: note = Cyclopropylalanine
  SITE 12: note = C-term CONH2
XVTVGEXVCEER

SEQ ID NO: 456 - protein, 12 aa, synthetic construct
  MOD_RES 1: note = Aad
  MOD_RES 3: note = Cyclopropylglycine
  SITE 12: note = C-term CONH2
XVXVAEVVCEER

SEQ ID NO: 457 - protein, 12 aa, synthetic construct
  MOD_RES 3: note = Cyclopropylglycine
  SITE 12: note = C-term CONH2
ELXVAEVVCEER

SEQ ID NO: 458 - protein, 12 aa, synthetic construct
  MOD_RES 1: note = Aad
  SITE 12: note = C-term CONH2
XLNLQEVLCEER

SEQ ID NO: 459 - protein, 12 aa, synthetic construct
  MOD_RES 3: note = Cyclobutylglycine
  SITE 12: note = C-term CONH2
ELXVAEVLCEER

SEQ ID NO: 460 - protein, 12 aa, synthetic construct
  MOD_RES 1: note = Aad
  MOD_RES 3: note = Cyclobutylglycine
  MOD_RES 4: note = Cyclopropylalanine
  MOD_RES 7: note = Homo phenylalanine
  SITE 12: note = C-term CONH2
XLXXQEXLCEER

SEQ ID NO: 461 - protein, 12 aa, synthetic construct
  MOD_RES 1: note = Aad
  MOD_RES 7: note = Cyclopropylalanine
  SITE 12: note = C-term CONH2
XVNVGEXVCEER

SEQ ID NO: 462 - protein, 12 aa, synthetic construct
  MOD_RES 1: note = Aad
  MOD_RES 3: note = Hyp
  MOD_RES 7: note = Cyclobutylalanine
  SITE 12: note = C-term CONH2
XVXVQEXVCEER

SEQ ID NO: 463 - protein, 12 aa, synthetic construct
  MOD_RES 1: note = Aad
  SITE 12: note = C-term CONH2
XVTVQELVCEER

SEQ ID NO: 464 - protein, 12 aa, synthetic construct
  MOD_RES 1: note = Aad
  MOD_RES 3: note = Hyp
  MOD_RES 4: note = Cyclopropylalanine
  MOD_RES 7: note = Cyclobutylalanine
  SITE 12: note = C-term CONH2
XLXXAEXVCEER

SEQ ID NO: 465 - protein, 12 aa, synthetic construct
  MOD_RES 1: note = Aad
  MOD_RES 7: note = Cyclobutylalanine
  SITE 12: note = C-term CONH2
XVNVAEXVCEER

SEQ ID NO: 466 - protein, 12 aa, synthetic construct
  MOD_RES 1: note = Aad
  MOD_RES 4: note = Phenyl glycine
  SITE 12: note = C-term CONH2
XVTXAEVLCEER

SEQ ID NO: 467 - protein, 12 aa, synthetic construct
  MOD_RES 1: note = Aad
  MOD_RES 4: note = Cyclopropylalanine
  MOD_RES 7: note = Homo phenylalanine
  SITE 12: note = C-term CONH2
XVTXQEXVCEER

SEQ ID NO: 468 - protein, 12 aa, synthetic construct
  MOD_RES 1: note = Aad
  MOD_RES 2: note = Octanonic
  MOD_RES 4: note = Cyclopropylalanine
  SITE 12: note = C-term CONH2
XXNXGELLCEER

SEQ ID NO: 469 - protein, 12 aa, synthetic construct
  MOD_RES 1: note = Aad
  MOD_RES 3: note = Cyclobutylglycine
  MOD_RES 4: note = Cyclobutylalanine
  MOD_RES 8: note = Phenyl glycine
  SITE 12: note = C-term CONH2
XLXXQELXCEER

SEQ ID NO: 470 - protein, 12 aa, synthetic construct
  MOD_RES 1: note = Aad
  MOD_RES 3: note = Cyclobutylglycine
  MOD_RES 4: note = Cyclobutylalanine
  MOD_RES 8: note = Cyclohexylalanine
  SITE 12: note = C-term CONH2
XVXXAEVXCEER

SEQ ID NO: 471 - protein, 12 aa, synthetic construct
  MOD_RES 1: note = Aad
  MOD_RES 3: note = Hyp
  MOD_RES 5: note = Aib
  MOD_RES 7: note = Cyclohexylalanine
  MOD_RES 8: note = Cyclobutylalanine
  SITE 12: note = C-term CONH2
XLXVXEXXCEER

SEQ ID NO: 472 - protein, 12 aa, synthetic construct
  MOD_RES 1: note = Aad
  MOD_RES 4: note = Cyclobutylalanine
  SITE 12: note = C-term CONH2
XVTXQELVCEER

SEQ ID NO: 473 - protein, 12 aa, synthetic construct
  MOD_RES 1: note = Aad
  MOD_RES 4: note = Cyclobutylalanine
  MOD_RES 7: note = Phenyl glycine
  SITE 12: note = C-term CONH2
XVTXAEXVCEER

SEQ ID NO: 474 - protein, 12 aa, synthetic construct
  MOD_RES 1: note = Aad
  MOD_RES 3: note = Hyp
  MOD_RES 5: note = Aib
  MOD_RES 8: note = Phenyl glycine
  SITE 12: note = C-term CONH2
XVXVXEVXCEER

SEQ ID NO: 475 - protein, 12 aa, synthetic construct
  MOD_RES 1: note = Aad
  MOD_RES 3: note = Cyclobutylglycine
  SITE 12: note = C-term CONH2
XVXLQEVVCEER

SEQ ID NO: 476 - protein, 12 aa, synthetic construct
  MOD_RES 1: note = Aad
  MOD_RES 2: note = Cyclopropylalanine
  MOD_RES 3: note = Hyp
  MOD_RES 7: note = Homo phenylalanine
  MOD_RES 8: note = Cyclobutylalanine
  SITE 12: note = C-term CONH2
XXXVQEXXCEER

SEQ ID NO: 477 - protein, 12 aa, synthetic construct
  MOD_RES 1: note = Aad
  MOD_RES 4: note = Homo phenylalanine
  SITE 12: note = C-term CONH2
XVNXQELVCEER

SEQ ID NO: 478 - protein, 12 aa, synthetic construct
  MOD_RES 1: note = Aad
  MOD_RES 3: note = Cyclobutylglycine
  MOD_RES 5: note = Aib
  MOD_RES 7: note = Cyclobutylalanine
  SITE 12: note = C-term CONH2
XLXLXEXLCEER

SEQ ID NO: 479 - protein, 12 aa, synthetic construct
  MOD_RES 1: note = Aad
  MOD_RES 2: note = Octanonic
  MOD_RES 3: note = Cyclopropylglycine
  MOD_RES 4: note = Cyclopropylalanine
  MOD_RES 5: note = Aib
  SITE 12: note = C-term CONH2
XXXXXELLCEER

SEQ ID NO: 480 - protein, 12 aa, synthetic construct
  MOD_RES 1: note = Aad
  MOD_RES 4: note = Cyclopropylalanine
  MOD_RES 8: note = Phenyl glycine
  SITE 12: note = C-term CONH2
XLTXQEVXCEER

SEQ ID NO: 481 - protein, 12 aa, synthetic construct
  MOD_RES 1: note = Aad
  MOD_RES 4: note = Cyclopropylalanine
  MOD_RES 7: note = Phenyl glycine
  MOD_RES 8: note = Cyclopropylalanine
  SITE 12: note = C-term CONH2
XVNXQEXXCEER

SEQ ID NO: 482 - protein, 12 aa, synthetic construct
  MOD_RES 1: note = Aad
  MOD_RES 3: note = Hyp
  MOD_RES 4: note = Cyclopropylalanine
  MOD_RES 8: note = Phenyl glycine
  SITE 12: note = C-term CONH2
XVXXQELXCEER

SEQ ID NO: 483 - protein, 12 aa, synthetic construct
  MOD_RES 1: note = Aad
  MOD_RES 2: note = Cyclopropylalanine
  MOD_RES 3: note = Cyclopropylglycine
  MOD_RES 4: note = Cyclopropylalanine
  MOD_RES 8: note = Cyclopropylalanine
  SITE 12: note = C-term CONH2
XXXXAEVXCEER

SEQ ID NO: 484 - protein, 12 aa, synthetic construct
  MOD_RES 1: note = Aad
  MOD_RES 4: note = Cyclopropylalanine
  MOD_RES 7: note = Cyclobutylalanine
  MOD_RES 8: note = Cyclopropylalanine
  SITE 12: note = C-term CONH2
XLTXAEXXCEER

SEQ ID NO: 485 - protein, 12 aa, synthetic construct
  MOD_RES 4: note = Cyclopropylalanine
  MOD_RES 7: note = Homo phenylalanine
  SITE 12: note = C-term CONH2
ELTXQEXVCEER

SEQ ID NO: 486 - protein, 12 aa, synthetic construct
  MOD_RES 1: note = Aad
  MOD_RES 2: note = Cyclopropylalanine
  MOD_RES 3: note = Hyp
  MOD_RES 4: note = Phenyl glycine
  MOD_RES 7: note = Cyclopropylalanine
  SITE 12: note = C-term CONH2
XXXXAEXLCEER

SEQ ID NO: 487 - protein, 12 aa, synthetic construct
  MOD_RES 1: note = Aad
  MOD_RES 3: note = Cyclopropylglycine
  MOD_RES 4: note = Phenyl glycine
  MOD_RES 8: note = Phenyl glycine
  SITE 12: note = C-term CONH2
XLXXQELXCEER

SEQ ID NO: 488 - protein, 12 aa, synthetic construct
  MOD_RES 1: note = Aad
  MOD_RES 3: note = Hyp
  MOD_RES 4: note = Phenyl glycine
  MOD_RES 5: note = Aib
  MOD_RES 8: note = Phenyl glycine
  SITE 12: note = C-term CONH2
XLXXXEVXCEER

SEQ ID NO: 489 - protein, 12 aa, synthetic construct
  MOD_RES 1: note = Aad
  MOD_RES 2: note = Cyclopropylalanine
  MOD_RES 3: note = Cyclopropylglycine
  MOD_RES 7: note = Cyclobutylalanine
  MOD_RES 8: note = Phenyl glycine
  SITE 12: note = C-term CONH2
XXXVQEXXCEER

SEQ ID NO: 490 - protein, 12 aa, synthetic construct
  MOD_RES 3: note = Cyclobutylglycine
  MOD_RES 5: note = Aib
  MOD_RES 8: note = Octanonic
  SITE 12: note = C-term CONH2
ELXVXEVXCEER

SEQ ID NO: 491 - protein, 12 aa, synthetic construct
  MOD_RES 1: note = Aad
  MOD_RES 4: note = Cyclopropylalanine
  MOD_RES 8: note = Phenyl glycine
  SITE 12: note = C-term CONH2
XVTXGEVXCEER

SEQ ID NO: 492 - protein, 12 aa, synthetic construct
  MOD_RES 1: note = Aad
  MOD_RES 3: note = Hyp
  MOD_RES 4: note = Cyclopropylalanine
  MOD_RES 6: note = Aad
  MOD_RES 7: note = Phenyl glycine
  SITE 12: note = C-term CONH2
XVXXAXXVCEER

SEQ ID NO: 493 - protein, 12 aa, synthetic construct
  MOD_RES 1: note = Aad
  MOD_RES 2: note = Phenyl glycine
  MOD_RES 7: note = Cyclopropylalanine
  SITE 12: note = C-term CONH2
XXNVGEXLCEER

SEQ ID NO: 494 - protein, 12 aa, synthetic construct
  MOD_RES 1: note = Aad
  MOD_RES 7: note = Cyclopropylalanine
  SITE 12: note = C-term CONH2
XLNVAEXLCEER

SEQ ID NO: 495 - protein, 12 aa, synthetic construct
  MOD_RES 1: note = Aad
  MOD_RES 3: note = Hyp
  MOD_RES 7: note = Cyclobutylalanine
  SITE 12: note = C-term CONH2
XLXVAEXLCEER

SEQ ID NO: 496 - protein, 12 aa, synthetic construct
  MOD_RES 1: note = Aad
  MOD_RES 3: note = Hyp
  MOD_RES 8: note = Phenyl glycine
  SITE 12: note = C-term CONH2
XVXLAEVXCEER

SEQ ID NO: 497 - protein, 12 aa, synthetic construct
  MOD_RES 1: note = Aad
  MOD_RES 3: note = Hyp
  MOD_RES 4: note = Cyclobutylalanine
  MOD_RES 7: note = Homo phenylalanine
  MOD_RES 8: note = Cyclopropylalanine
  SITE 12: note = C-term CONH2
XVXXAEXXCEER

SEQ ID NO: 498 - protein, 12 aa, synthetic construct
  MOD_RES 4: note = Cyclobutylalanine
  SITE 12: note = C-term CONH2
ELNXAELVCEER

SEQ ID NO: 499 - protein, 12 aa, synthetic construct
  MOD_RES 7: note = Octanonic
  SITE 12: note = C-term CONH2
ELTVAEXVCEER

SEQ ID NO: 500 - protein, 12 aa, synthetic construct
  MOD_RES 1: note = Aad
  MOD_RES 3: note = Cyclopropylglycine
  MOD_RES 4: note = Homo phenylalanine
  SITE 12: note = C-term CONH2
XLXXAELLCEER

SEQ ID NO: 501 - protein, 12 aa, synthetic construct
  MOD_RES 2: note = Cyclobutylalanine
  MOD_RES 4: note = Phenyl glycine
  MOD_RES 7: note = Phenyl glycine
  SITE 12: note = C-term CONH2
EXTXQEXVCEER

SEQ ID NO: 502 - protein, 12 aa, synthetic construct
  MOD_RES 1: note = Aad
  MOD_RES 3: note = Hyp
  MOD_RES 4: note = Phenyl glycine
  MOD_RES 7: note = Cyclopropylalanine
  SITE 12: note = C-term CONH2
XVXXQEXVCEER

SEQ ID NO: 503 - protein, 12 aa, synthetic construct
  MOD_RES 1: note = Aad
  MOD_RES 8: note = Cyclopropylalanine
  SITE 12: note = C-term CONH2
XVTLAEVXCEER

SEQ ID NO: 504 - protein, 12 aa, synthetic construct
  MOD_RES 1: note = Aad
  MOD_RES 2: note = Cyclopropylalanine
  MOD_RES 8: note = Phenyl glycine
  SITE 12: note = C-term CONH2
XXTVAEVXCEER

SEQ ID NO: 505 - protein, 12 aa, synthetic construct
  MOD_RES 1: note = Aad
  MOD_RES 2: note = Homo phenylalanine
  MOD_RES 3: note = Hyp
  MOD_RES 4: note = Cyclopropylalanine
  MOD_RES 7: note = Cyclopropylalanine
  SITE 12: note = C-term CONH2
XXXXGEXVCEER

SEQ ID NO: 506 - protein, 12 aa, synthetic construct
  MOD_RES 1: note = Aad
  MOD_RES 3: note = Cyclobutylglycine
  MOD_RES 7: note = Phenyl glycine
  SITE 12: note = C-term CONH2
XVXVAEXVCEER

SEQ ID NO: 507 - protein, 12 aa, synthetic construct
  MOD_RES 1: note = Aad
  MOD_RES 2: note = Homo phenylalanine
  MOD_RES 7: note = Cyclopropylalanine
  MOD_RES 8: note = Phenyl glycine
  SITE 12: note = C-term CONH2
XXNLQEXXCEER

SEQ ID NO: 508 - protein, 12 aa, synthetic construct
  MOD_RES 1: note = Aad
  MOD_RES 2: note = Octanonic
  MOD_RES 3: note = Cyclopropylglycine
  MOD_RES 4: note = Cyclobutylalanine
  MOD_RES 7: note = Homo phenylalanine
  SITE 12: note = C-term CONH2
XXXXGEXLCEER

SEQ ID NO: 509 - protein, 12 aa, synthetic construct
  MOD_RES 1: note = Aad
  MOD_RES 3: note = Hyp
  MOD_RES 7: note = Phenyl glycine
  MOD_RES 8: note = Phenyl glycine
  SITE 12: note = C-term CONH2
XVXVAEXXCEER

SEQ ID NO: 510 - protein, 12 aa, synthetic construct
  MOD_RES 1: note = Aad
  MOD_RES 7: note = Cyclopropylalanine
  MOD_RES 8: note = Phenyl glycine
  SITE 12: note = C-term CONH2
XVTLGEXXCEER

SEQ ID NO: 511 - protein, 12 aa, synthetic construct
  MOD_RES 1: note = Aad
  MOD_RES 3: note = Hyp
  MOD_RES 4: note = Cyclobutylalanine
  SITE 12: note = C-term CONH2
XVXXAEVLCEER

SEQ ID NO: 512 - protein, 12 aa, synthetic construct
  MOD_RES 1: note = Aad
  MOD_RES 4: note = Cyclopropylalanine
  MOD_RES 8: note = Phenyl glycine
  SITE 12: note = C-term CONH2
XLTXAELXCEER

SEQ ID NO: 513 - protein, 12 aa, synthetic construct
  MOD_RES 1: note = Aad
  MOD_RES 3: note = Hyp
  MOD_RES 4: note = Cyclopropylalanine
  MOD_RES 5: note = Aib
  MOD_RES 8: note = Phenyl glycine
  SITE 12: note = C-term CONH2
XVXXXELXCEER

SEQ ID NO: 514 - protein, 12 aa, synthetic construct
  MOD_RES 3: note = Cyclopropylglycine
  MOD_RES 5: note = Aib
  MOD_RES 8: note = Phenyl glycine
  SITE 12: note = C-term CONH2
ELXLXEVXCEER

SEQ ID NO: 515 - protein, 12 aa, synthetic construct
  MOD_RES 1: note = Aad
  MOD_RES 3: note = Cyclopentylglycine
  MOD_RES 5: note = Aib
  MOD_RES 7: note = Cyclohexylalanine
  MOD_RES 8: note = Phenyl glycine
  SITE 12: note = C-term CONH2
XLXVXEXXCEER

SEQ ID NO: 516 - protein, 12 aa, synthetic construct
  MOD_RES 1: note = Aad
  MOD_RES 3: note = Cyclopentylglycine
  MOD_RES 4: note = Phenyl glycine
  MOD_RES 5: note = Aib
  MOD_RES 6: note = Aad
  MOD_RES 7: note = Cyclobutylalanine
  SITE 12: note = C-term CONH2
XVXXXXXLCEER

SEQ ID NO: 517 - protein, 12 aa, synthetic construct
  MOD_RES 2: note = Cyclobutylalanine
  MOD_RES 3: note = Cyclopropylglycine
  MOD_RES 4: note = Cyclopropylalanine
  MOD_RES 8: note = Cyclopropylalanine
  SITE 12: note = C-term CONH2
EXXXAEVXCEER

SEQ ID NO: 518 - protein, 12 aa, synthetic construct
  MOD_RES 1: note = Aad
  MOD_RES 2: note = Cyclopropylalanine
  MOD_RES 7: note = Cyclopropylalanine
  SITE 12: note = C-term CONH2
XXTLGEXLCEER

SEQ ID NO: 519 - protein, 12 aa, synthetic construct
  MOD_RES 1: note = Aad
  MOD_RES 2: note = Cyclopropylalanine
  MOD_RES 3: note = Hyp
  MOD_RES 4: note = Phenyl glycine
  MOD_RES 5: note = Aib
  MOD_RES 8: note = Cyclopropylalanine
  SITE 12: note = C-term CONH2
XXXXXELXCEER

SEQ ID NO: 520 - protein, 12 aa, synthetic construct
  MOD_RES 1: note = Aad
  MOD_RES 4: note = Cyclobutylalanine
  MOD_RES 7: note = Homo phenylalanine
  SITE 12: note = C-term CONH2
XVNXAEXVCEER

SEQ ID NO: 521 - protein, 12 aa, synthetic construct
  MOD_RES 1: note = Aad
  MOD_RES 3: note = Cyclopropylglycine
  MOD_RES 5: note = Aib
  MOD_RES 7: note = Cyclobutylalanine
  SITE 12: note = C-term CONH2
XVXLXEXVCEER

SEQ ID NO: 522 - protein, 12 aa, synthetic construct
  MOD_RES 1: note = Aad
  MOD_RES 4: note = Phenyl glycine
  SITE 12: note = C-term CONH2
XVNXAELVCEER

SEQ ID NO: 523 - protein, 12 aa, synthetic construct
  MOD_RES 1: note = Aad
  MOD_RES 2: note = Phenyl glycine
  MOD_RES 3: note = Hyp
  MOD_RES 7: note = Homo phenylalanine
  SITE 12: note = C-term CONH2
XXXVAEXVCEER

SEQ ID NO: 524 - protein, 12 aa, synthetic construct
  MOD_RES 1: note = Aad
  MOD_RES 3: note = Hyp
  MOD_RES 4: note = Cyclohexylalanine
  MOD_RES 7: note = Cyclobutylalanine
  SITE 12: note = C-term CONH2
XVXXAEXVCEER

SEQ ID NO: 525 - protein, 12 aa, synthetic construct
  MOD_RES 1: note = Aad
  MOD_RES 2: note = Cyclopropylalanine
  MOD_RES 3: note = Hyp
  MOD_RES 8: note = Cyclobutylalanine
  SITE 12: note = C-term CONH2
XXXVAELXCEER

SEQ ID NO: 526 - protein, 12 aa, synthetic construct
  MOD_RES 3: note = Hyp
  MOD_RES 5: note = Aib
  SITE 12: note = C-term CONH2
ELXVXEVLCEER

SEQ ID NO: 527 - protein, 12 aa, synthetic construct
  MOD_RES 1: note = Aad
  MOD_RES 3: note = Cyclopropylglycine
  MOD_RES 4: note = Cyclopropylalanine
  MOD_RES 8: note = Phenyl glycine
  SITE 12: note = C-term CONH2
XVXXAEVXCEER

SEQ ID NO: 528 - protein, 12 aa, synthetic construct
  MOD_RES 1: note = Aad
  SITE 12: note = C-term CONH2
XVTVQEVLCEER

SEQ ID NO: 529 - protein, 12 aa, synthetic construct
  MOD_RES 1: note = Aad
  MOD_RES 3: note = Hyp
  MOD_RES 4: note = Cyclopropylalanine
  MOD_RES 6: note = Aad
  MOD_RES 8: note = Phenyl glycine
  SITE 12: note = C-term CONH2
XLXXAXLXCEER

SEQ ID NO: 530 - protein, 12 aa, synthetic construct
  MOD_RES 1: note = Aad
  MOD_RES 2: note = Cyclobutylalanine
  MOD_RES 4: note = Homo phenylalanine
  MOD_RES 7: note = Cyclohexylalanine
  SITE 12: note = C-term CONH2
XXTXGEXLCEER

SEQ ID NO: 531 - protein, 12 aa, synthetic construct
  MOD_RES 1: note = Aad
  MOD_RES 2: note = Cyclopropylalanine
  MOD_RES 4: note = Napthyl alanine
  MOD_RES 7: note = Napthyl alanine
  SITE 12: note = C-term CONH2
XXNXAEXVCEER

SEQ ID NO: 532 - protein, 12 aa, synthetic construct
  MOD_RES 1: note = Aad
  MOD_RES 4: note = Napthyl alanine
  MOD_RES 5: note = Aib
  MOD_RES 7: note = Homo phenylalanine
  MOD_RES 8: note = Phenyl glycine
  SITE 12: note = C-term CONH2
XLNXXEXXCEER

SEQ ID NO: 533 - protein, 12 aa, synthetic construct
  MOD_RES 1: note = Aad
  MOD_RES 3: note = Cyclopentylglycine
  MOD_RES 4: note = Napthyl alanine
  MOD_RES 5: note = Aib
  MOD_RES 7: note = Cyclopropylalanine
  MOD_RES 8: note = Homo phenylalanine
  SITE 12: note = C-term CONH2
XVXXXEXXCEER

SEQ ID NO: 534 - protein, 12 aa, synthetic construct
  MOD_RES 1: note = Aad
  MOD_RES 3: note = Hyp
  MOD_RES 4: note = Cyclopropylalanine
  SITE 12: note = C-term CONH2
XVXXAELVCEER

SEQ ID NO: 535 - protein, 12 aa, synthetic construct
  MOD_RES 3: note = Hyp
  MOD_RES 4: note = Cyclopropylalanine
  SITE 12: note = C-term CONH2
ELXXAEVVCEER

SEQ ID NO: 536 - protein, 12 aa, synthetic construct
  MOD_RES 3: note = Cyclobutylglycine
  MOD_RES 8: note = Cyclopropylalanine
  SITE 12: note = C-term CONH2
ELXVQEVXCEER

SEQ ID NO: 537 - protein, 12 aa, synthetic construct
  MOD_RES 1: note = Aad
  MOD_RES 3: note = Cyclopropylglycine
  MOD_RES 4: note = Cyclobutylalanine
  SITE 12: note = C-term CONH2
XVXXQEVVCEER

SEQ ID NO: 538 - protein, 12 aa, synthetic construct
  MOD_RES 1: note = Aad
  MOD_RES 2: note = Cyclopropylalanine
  MOD_RES 4: note = Napthyl alanine
  MOD_RES 5: note = Aib
  MOD_RES 6: note = Aad
  MOD_RES 8: note = Homo phenylalanine
  SITE 12: note = C-term CONH2
XXNXXXVXCEER

SEQ ID NO: 539 - protein, 12 aa, synthetic construct
  MOD_RES 1: note = Aad
  MOD_RES 2: note = Napthyl alanine
  MOD_RES 5: note = Aib
  MOD_RES 7: note = Cyclohexylalanine
  MOD_RES 8: note = Phenyl glycine
  SITE 12: note = C-term CONH2
XXNLXEXXCEER

SEQ ID NO: 540 - protein, 12 aa, synthetic construct
  MOD_RES 3: note = Cyclopentylglycine
  MOD_RES 4: note = Napthyl alanine
  MOD_RES 5: note = Aib
  MOD_RES 6: note = Aad
  MOD_RES 7: note = Cyclobutylalanine
  MOD_RES 8: note = Phenyl glycine
  SITE 12: note = C-term CONH2
ELXXXXXXCEER

SEQ ID NO: 541 - protein, 12 aa, synthetic construct
  MOD_RES 3: note = Hyp
  SITE 12: note = C-term CONH2
ELXVAELVCEER

SEQ ID NO: 542 - protein, 12 aa, synthetic construct
  MOD_RES 1: note = Aad
  MOD_RES 3: note = Hyp
  MOD_RES 4: note = Napthyl alanine
  MOD_RES 7: note = Cyclohexylalanine
  MOD_RES 8: note = Homo phenylalanine
  SITE 12: note = C-term CONH2
XVXXAEXXCEER

SEQ ID NO: 543 - protein, 12 aa, synthetic construct
  MOD_RES 3: note = Hyp
  MOD_RES 7: note = Cyclobutylalanine
  SITE 12: note = C-term CONH2
ELXVGEXVCEER

SEQ ID NO: 544 - protein, 12 aa, synthetic construct
  MOD_RES 3: note = Cyclopropylglycine
  MOD_RES 7: note = Cyclopropylalanine
  SITE 12: note = C-term CONH2
ELXVAEXVCEER

SEQ ID NO: 545 - protein, 12 aa, synthetic construct
  MOD_RES 1: note = Aad
  MOD_RES 4: note = Cyclopropylalanine
  MOD_RES 8: note = Phenyl glycine
  SITE 12: note = C-term CONH2
XLTXQELXCEER

SEQ ID NO: 546 - protein, 12 aa, synthetic construct
  MOD_RES 4: note = Napthyl alanine
  MOD_RES 5: note = Aib
  MOD_RES 7: note = Homo phenylalanine
  SITE 12: note = C-term CONH2
ELTXXEXVCEER

SEQ ID NO: 547 - protein, 12 aa, synthetic construct
  MOD_RES 1: note = Aad
  MOD_RES 3: note = Hyp
  MOD_RES 4: note = Cyclobutylalanine
  SITE 12: note = C-term CONH2
XVXXGEVLCEER

SEQ ID NO: 548 - protein, 12 aa, synthetic construct
  MOD_RES 1: note = Aad
  MOD_RES 2: note = Napthyl alanine
  MOD_RES 4: note = Cyclopropylalanine
  MOD_RES 7: note = Napthyl alanine
  SITE 12: note = C-term CONH2
XXNXGEXLCEER

SEQ ID NO: 549 - protein, 12 aa, synthetic construct
  MOD_RES 4: note = Napthyl alanine
  MOD_RES 5: note = Aib
  MOD_RES 7: note = Homo phenylalanine
  MOD_RES 8: note = Cyclopropylalanine
  SITE 12: note = C-term CONH2
ELTXXEXXCEER

SEQ ID NO: 550 - protein, 12 aa, synthetic construct
  MOD_RES 1: note = Aad
  MOD_RES 3: note = Hyp
  MOD_RES 4: note = Napthyl alanine
  MOD_RES 7: note = Napthyl alanine
  MOD_RES 8: note = Phenyl glycine
  SITE 12: note = C-term CONH2
XVXXQEXXCEER

SEQ ID NO: 551 - protein, 12 aa, synthetic construct
  MOD_RES 1: note = Aad
  MOD_RES 4: note = Napthyl alanine
  MOD_RES 5: note = Aib
  MOD_RES 7: note = Cyclohexylalanine
  SITE 12: note = C-term CONH2
XLTXXEXVCEER

SEQ ID NO: 552 - protein, 12 aa, synthetic construct
  MOD_RES 1: note = Aad
  MOD_RES 2: note = Napthyl alanine
  MOD_RES 3: note = Hyp
  MOD_RES 5: note = Aib
  MOD_RES 7: note = Homo phenylalanine
  MOD_RES 8: note = Phenyl glycine
  SITE 12: note = C-term CONH2
XXXVXEXXCEER

SEQ ID NO: 553 - protein, 12 aa, synthetic construct
  MOD_RES 1: note = Aad
  MOD_RES 4: note = Cyclobutylalanine
  SITE 12: note = C-term CONH2
XVTXGELVCEER

SEQ ID NO: 554 - protein, 12 aa, synthetic construct
  MOD_RES 1: note = Aad
  MOD_RES 2: note = Octanonic
  MOD_RES 3: note = Cyclopropylglycine
  MOD_RES 4: note = Homo phenylalanine
  MOD_RES 5: note = Aib
  MOD_RES 7: note = Napthyl alanine
  SITE 12: note = C-term CONH2
XXXXXEXLCEER

SEQ ID NO: 555 - protein, 12 aa, synthetic construct
  MOD_RES 3: note = Hyp
  MOD_RES 4: note = Phenyl glycine
  SITE 12: note = C-term CONH2
ELXXAEVVCEER

SEQ ID NO: 556 - protein, 12 aa, synthetic construct
  MOD_RES 3: note = Cyclobutylglycine
  MOD_RES 8: note = Phenyl glycine
  SITE 12: note = C-term CONH2
ELXVAELXCEER

SEQ ID NO: 557 - protein, 12 aa, synthetic construct
  MOD_RES 1: note = Aad
  MOD_RES 2: note = Napthyl alanine
  MOD_RES 3: note = Hyp
  MOD_RES 4: note = Homo phenylalanine
  MOD_RES 7: note = Homo phenylalanine
  MOD_RES 8: note = Phenyl glycine
  SITE 12: note = C-term CONH2
XXXXAEXXCEER

SEQ ID NO: 558 - protein, 12 aa, synthetic construct
  MOD_RES 1: note = Aad
  MOD_RES 2: note = Cyclopropylalanine
  MOD_RES 3: note = Hyp
  SITE 12: note = C-term CONH2
XXXVQELLCEER

SEQ ID NO: 559 - protein, 12 aa, synthetic construct
  MOD_RES 3: note = Hyp
  MOD_RES 4: note = Cyclobutylalanine
  SITE 12: note = C-term CONH2
ELXXGELVCEER

SEQ ID NO: 560 - protein, 12 aa, synthetic construct
  MOD_RES 1: note = Aad
  MOD_RES 2: note = Phenyl glycine
  MOD_RES 3: note = Hyp
  SITE 12: note = C-term CONH2
XXXVGELVCEER

SEQ ID NO: 561 - protein, 12 aa, synthetic construct
  MOD_RES 2: note = Cyclobutylalanine
  MOD_RES 8: note = Cyclopropylalanine
  SITE 12: note = C-term CONH2
EXNVAELXCEER

SEQ ID NO: 562 - protein, 12 aa, synthetic construct
  MOD_RES 1: note = Aad
  MOD_RES 2: note = Octanonic
  MOD_RES 4: note = Cyclopropylalanine
  MOD_RES 7: note = Cyclobutylalanine
  MOD_RES 8: note = Napthyl alanine
  SITE 12: note = C-term CONH2
XXNXGEXXCEER

SEQ ID NO: 563 - protein, 12 aa, synthetic construct
  MOD_RES 1: note = Aad
  MOD_RES 3: note = Cyclopentylglycine
  MOD_RES 4: note = Napthyl alanine
  MOD_RES 7: note = Homo phenylalanine
  SITE 12: note = C-term CONH2
XVXXGEXLCEER

SEQ ID NO: 564 - protein, 12 aa, synthetic construct
  MOD_RES 4: note = Cyclopropylalanine
  MOD_RES 7: note = Phenyl glycine
  SITE 12: note = C-term CONH2
ELNXGEXVCEER

SEQ ID NO: 565 - protein, 12 aa, synthetic construct
  MOD_RES 1: note = Aad
  MOD_RES 3: note = Cyclopentylglycine
  MOD_RES 4: note = Cyclohexylalanine
  MOD_RES 5: note = Aib
  MOD_RES 7: note = Homo phenylalanine
  MOD_RES 8: note = Cyclobutylalanine
  SITE 12: note = C-term CONH2
XLXXXEXXCEER

SEQ ID NO: 566 - protein, 12 aa, synthetic construct
  MOD_RES 3: note = Hyp
  MOD_RES 7: note = Cyclopropylalanine
  SITE 12: note = C-term CONH2
ELXVQEXLCEER

SEQ ID NO: 567 - protein, 12 aa, synthetic construct
  MOD_RES 1: note = Aad
  MOD_RES 2: note = Napthyl alanine
  MOD_RES 4: note = Homo phenylalanine
  MOD_RES 5: note = Aib
  MOD_RES 8: note = Phenyl glycine
  SITE 12: note = C-term CONH2
XXTXXEVXCEER

SEQ ID NO: 568 - protein, 12 aa, synthetic construct
  MOD_RES 1: note = Aad
  MOD_RES 2: note = Napthyl alanine
  MOD_RES 4: note = Homo phenylalanine
  MOD_RES 6: note = Aad
  MOD_RES 7: note = Cyclopropylalanine
  SITE 12: note = C-term CONH2
XXNXAXXLCEER

SEQ ID NO: 569 - protein, 12 aa, synthetic construct
  MOD_RES 1: note = Aad
  MOD_RES 2: note = Phenyl glycine
  MOD_RES 3: note = Cyclopropylglycine
  MOD_RES 4: note = Napthyl alanine
  MOD_RES 5: note = Aib
  SITE 12: note = C-term CONH2
XXXXXELLCEER

SEQ ID NO: 570 - protein, 12 aa, synthetic construct
  SITE 12: note = C-term CONH2
ELTVQEVVCEER

SEQ ID NO: 571 - protein, 12 aa, synthetic construct
  MOD_RES 1: note = Aad
  MOD_RES 3: note = Hyp
  MOD_RES 4: note = Cyclopropylalanine
  SITE 12: note = C-term CONH2
XLXXQEVLCEER

SEQ ID NO: 572 - protein, 12 aa, synthetic construct
  MOD_RES 1: note = Aad
  MOD_RES 2: note = Cyclohexylalanine
  MOD_RES 3: note = Hyp
  MOD_RES 4: note = Cyclohexylalanine
  MOD_RES 7: note = Napthyl alanine
  MOD_RES 8: note = Cyclobutylalanine
  SITE 12: note = C-term CONH2
XXXXGEXXCEER

SEQ ID NO: 573 - protein, 12 aa, synthetic construct
  MOD_RES 1: note = Aad
  MOD_RES 2: note = Homo phenylalanine
  MOD_RES 4: note = Homo phenylalanine
  MOD_RES 7: note = Homo phenylalanine
  MOD_RES 8: note = Cyclobutylalanine
  SITE 12: note = C-term CONH2
XXNXAEXXCEER

SEQ ID NO: 574 - protein, 12 aa, synthetic construct
  MOD_RES 1: note = Aad
  MOD_RES 2: note = Cyclopropylalanine
  MOD_RES 3: note = Cyclopropylglycine
  MOD_RES 5: note = Aib
  MOD_RES 8: note = Napthyl alanine
  SITE 12: note = C-term CONH2
XXXLXELXCEER

SEQ ID NO: 575 - protein, 12 aa, synthetic construct
  MOD_RES 1: note = Aad
  MOD_RES 2: note = Napthyl alanine
  MOD_RES 3: note = Cyclobutylglycine
  MOD_RES 4: note = Cyclopropylalanine
  MOD_RES 5: note = Aib
  MOD_RES 7: note = Homo phenylalanine
  SITE 12: note = C-term CONH2
XXXXXEXVCEER

SEQ ID NO: 576 - protein, 12 aa, synthetic construct
  MOD_RES 1: note = Aad
  MOD_RES 2: note = Homo phenylalanine
  MOD_RES 3: note = Hyp
  MOD_RES 4: note = Napthyl alanine
  MOD_RES 6: note = Aad
  MOD_RES 7: note = Cyclobutylalanine
  SITE 12: note = C-term CONH2
XXXXAXXLCEER

SEQ ID NO: 577 - protein, 12 aa, synthetic construct
  MOD_RES 1: note = Aad
  MOD_RES 2: note = Cyclopropylalanine
  MOD_RES 3: note = Cyclobutylglycine
  MOD_RES 4: note = Napthyl alanine
  MOD_RES 7: note = Cyclohexylalanine
  SITE 12: note = C-term CONH2
XXXXAEXLCEER

SEQ ID NO: 578 - protein, 12 aa, synthetic construct
  MOD_RES 1: note = Aad
  MOD_RES 2: note = Napthyl alanine
  MOD_RES 3: note = Cyclopropylglycine
  MOD_RES 4: note = Cyclobutylalanine
  MOD_RES 7: note = Octanonic
  MOD_RES 8: note = Phenyl glycine
  SITE 12: note = C-term CONH2
XXXXAEXXCEER

SEQ ID NO: 579 - protein, 12 aa, synthetic construct
  MOD_RES 1: note = Aad
  MOD_RES 2: note = Cyclobutylalanine
  MOD_RES 4: note = Napthyl alanine
  MOD_RES 7: note = Cyclohexylalanine
  SITE 12: note = C-term CONH2
XXTXGEXLCEER

SEQ ID NO: 580 - protein, 12 aa, synthetic construct
  MOD_RES 2: note = Cyclobutylalanine
  SITE 12: note = C-term CONH2
EXTLQELLCEER

SEQ ID NO: 581 - protein, 12 aa, synthetic construct
  SITE 12: note = C-term CONH2
ELTVAEVLCEER

SEQ ID NO: 582 - protein, 12 aa, synthetic construct
  MOD_RES 3: note = Hyp
  MOD_RES 8: note = Cyclopropylalanine
  SITE 12: note = C-term CONH2
ELXVAEVXCEER

SEQ ID NO: 583 - protein, 12 aa, synthetic construct
  SITE 12: note = C-term CONH2
ELNVAELVCEER

SEQ ID NO: 584 - protein, 12 aa, synthetic construct
  MOD_RES 1: note = Aad
  MOD_RES 3: note = Cyclobutylglycine
  MOD_RES 8: note = Cyclopropylalanine
  SITE 12: note = C-term CONH2
XVXVQEVXCEER

SEQ ID NO: 585 - protein, 12 aa, synthetic construct
  MOD_RES 1: note = Aad
  MOD_RES 4: note = Cyclobutylalanine
  MOD_RES 7: note = Homo phenylalanine
  MOD_RES 8: note = Napthyl alanine
  SITE 12: note = C-term CONH2
XVNXAEXXCEER

SEQ ID NO: 586 - protein, 12 aa, synthetic construct
  MOD_RES 4: note = Cyclohexylalanine
  MOD_RES 7: note = Homo phenylalanine
  SITE 12: note = C-term CONH2
ELNXGEXLCEER

SEQ ID NO: 587 - protein, 12 aa, synthetic construct
  MOD_RES 1: note = Aad
  MOD_RES 3: note = Cyclobutylglycine
  MOD_RES 7: note = Cyclopropylalanine
  SITE 12: note = C-term CONH2
XVXVQEXVCEER

SEQ ID NO: 588 - protein, 12 aa, synthetic construct
  MOD_RES 1: note = Aad
  MOD_RES 3: note = Hyp
  MOD_RES 7: note = Cyclopropylalanine
  SITE 12: note = C-term CONH2
XVXLGEXLCEER

SEQ ID NO: 589 - protein, 12 aa, synthetic construct
  MOD_RES 1: note = Aad
  MOD_RES 2: note = Octanonic
  MOD_RES 4: note = Cyclohexylalanine
  MOD_RES 7: note = Cyclopropylalanine
  MOD_RES 8: note = Octanonic
  SITE 12: note = C-term CONH2
XXNXAEXXCEER

SEQ ID NO: 590 - protein, 12 aa, synthetic construct
  MOD_RES 1: note = Aad
  MOD_RES 2: note = Napthyl alanine
  MOD_RES 3: note = Hyp
  MOD_RES 4: note = Cyclohexylalanine
  MOD_RES 7: note = Cyclopropylalanine
  MOD_RES 8: note = Cyclohexylalanine
  SITE 12: note = C-term CONH2
XXXXAEXXCEER

SEQ ID NO: 591 - protein, 12 aa, synthetic construct
  MOD_RES 1: note = Aad
  MOD_RES 2: note = Cyclohexylalanine
  MOD_RES 3: note = Cyclopropylglycine
  MOD_RES 4: note = Napthyl alanine
  MOD_RES 5: note = Aib
  MOD_RES 7: note = Cyclopropylalanine
  MOD_RES 8: note = Phenyl glycine
  SITE 12: note = C-term CONH2
XXXXXEXXCEER

SEQ ID NO: 592 - protein, 12 aa, synthetic construct
  MOD_RES 1: note = Aad
  MOD_RES 2: note = Napthyl alanine
  MOD_RES 3: note = Cyclopentylglycine
  MOD_RES 4: note = Cyclobutylalanine
  MOD_RES 5: note = Aib
  MOD_RES 7: note = Cyclobutylalanine
  MOD_RES 8: note = Phenyl glycine
  SITE 12: note = C-term CONH2
XXXXXEXXCEER

SEQ ID NO: 593 - protein, 12 aa, synthetic construct
  MOD_RES 3: note = Hyp
  MOD_RES 4: note = Cyclohexylalanine
  MOD_RES 5: note = Aib
  MOD_RES 7: note = Cyclobutylalanine
  MOD_RES 8: note = Octanonic
  SITE 12: note = C-term CONH2
ELXXXEXXCEER

SEQ ID NO: 594 - protein, 12 aa, synthetic construct
  MOD_RES 1: note = Aad
  MOD_RES 3: note = Cyclobutylglycine
  MOD_RES 4: note = Cyclohexylalanine
  MOD_RES 7: note = Cyclohexylalanine
  MOD_RES 8: note = Octanonic
  SITE 12: note = C-term CONH2
XVXXAEXXCEER

SEQ ID NO: 595 - protein, 12 aa, synthetic construct
  MOD_RES 1: note = Aad
  MOD_RES 2: note = Homo phenylalanine
  MOD_RES 4: note = Homo phenylalanine
  MOD_RES 5: note = Aib
  MOD_RES 7: note = Cyclobutylalanine
  SITE 12: note = C-term CONH2
XXTXXEXLCEER

SEQ ID NO: 596 - protein, 12 aa, synthetic construct
  MOD_RES 1: note = Aad
  MOD_RES 2: note = Homo phenylalanine
  MOD_RES 4: note = Cyclohexylalanine
  MOD_RES 8: note = Homo phenylalanine
  SITE 12: note = C-term CONH2
XXNXQELXCEER

SEQ ID NO: 597 - protein, 12 aa, synthetic construct
  MOD_RES 1: note = Aad
  MOD_RES 3: note = Hyp
  MOD_RES 4: note = Napthyl alanine
  MOD_RES 7: note = Homo phenylalanine
  MOD_RES 8: note = Homo phenylalanine
  SITE 12: note = C-term CONH2
XLXXQEXXCEER

SEQ ID NO: 598 - protein, 12 aa, synthetic construct
  MOD_RES 1: note = Aad
  MOD_RES 4: note = Homo phenylalanine
  MOD_RES 5: note = Aib
  MOD_RES 7: note = Cyclobutylalanine
  MOD_RES 8: note = Homo phenylalanine
  SITE 12: note = C-term CONH2
XLTXXEXXCEER

SEQ ID NO: 599 - protein, 12 aa, synthetic construct
  MOD_RES 1: note = Aad
  MOD_RES 2: note = Cyclohexylalanine
  MOD_RES 4: note = Napthyl alanine
  MOD_RES 7: note = Cyclobutylalanine
  SITE 12: note = C-term CONH2
XXNXAEXLCEER

SEQ ID NO: 600 - protein, 12 aa, synthetic construct
  MOD_RES 1: note = Aad
  MOD_RES 3: note = Cyclobutylglycine
  MOD_RES 4: note = Napthyl alanine
  MOD_RES 5: note = Aib
  MOD_RES 7: note = Cyclobutylalanine
  SITE 12: note = C-term CONH2
XLXXXEXLCEER

SEQ ID NO: 601 - protein, 12 aa, synthetic construct
  MOD_RES 1: note = Aad
  MOD_RES 4: note = Napthyl alanine
  MOD_RES 7: note = Octanonic
  SITE 12: note = C-term CONH2
XLNXAEXLCEER

SEQ ID NO: 602 - protein, 12 aa, synthetic construct
  MOD_RES 1: note = Aad
  MOD_RES 2: note = Napthyl alanine
  MOD_RES 4: note = Homo phenylalanine
  MOD_RES 8: note = Phenyl glycine
  SITE 12: note = C-term CONH2
XXTXAELXCEER

SEQ ID NO: 603 - protein, 12 aa, synthetic construct
  MOD_RES 1: note = Aad
  MOD_RES 3: note = Hyp
  MOD_RES 4: note = Napthyl alanine
  MOD_RES 5: note = Aib
  MOD_RES 7: note = Cyclopropylalanine
  MOD_RES 8: note = Homo phenylalanine
  SITE 12: note = C-term CONH2
XLXXXEXXCEER

SEQ ID NO: 604 - protein, 12 aa, synthetic construct
  MOD_RES 1: note = Aad
  MOD_RES 2: note = Homo phenylalanine
  MOD_RES 4: note = Cyclobutylalanine
  MOD_RES 7: note = Napthyl alanine
  MOD_RES 8: note = Cyclobutylalanine
  SITE 12: note = C-term CONH2
XXNXAEXXCEER

SEQ ID NO: 605 - protein, 12 aa, synthetic construct
  MOD_RES 1: note = Aad
  MOD_RES 2: note = Napthyl alanine
  MOD_RES 4: note = Cyclopropylalanine
  MOD_RES 5: note = Aib
  MOD_RES 7: note = Cyclohexylalanine
  MOD_RES 8: note = Phenyl glycine
  SITE 12: note = C-term CONH2
XXTXXEXXCEER

SEQ ID NO: 606 - protein, 12 aa, synthetic construct
  MOD_RES 1: note = Aad
  MOD_RES 2: note = Cyclopropylalanine
  MOD_RES 4: note = Octanonic
  MOD_RES 5: note = Aib
  MOD_RES 8: note = Homo phenylalanine
  SITE 12: note = C-term CONH2
XXTXXELXCEER

SEQ ID NO: 607 - protein, 12 aa, synthetic construct
  MOD_RES 1: note = Aad
  MOD_RES 3: note = Cyclobutylglycine
  MOD_RES 4: note = Napthyl alanine
  MOD_RES 5: note = Aib
  MOD_RES 7..8: note = Cyclobutylalanine
  SITE 12: note = C-term CONH2
XVXXXEXXCEER

SEQ ID NO: 608 - protein, 12 aa, synthetic construct
  MOD_RES 1: note = Aad
  MOD_RES 2: note = Homo phenylalanine
  MOD_RES 3: note = Cyclopropylglycine
  MOD_RES 4: note = Napthyl alanine
  MOD_RES 5: note = Aib
  MOD_RES 7: note = Cyclopropylalanine
  SITE 12: note = C-term CONH2
XXXXXEXLCEER

SEQ ID NO: 609 - protein, 12 aa, synthetic construct
  MOD_RES 1: note = Aad
  MOD_RES 2: note = Cyclopropylalanine
  MOD_RES 3: note = Cyclopropylglycine
  MOD_RES 5: note = Aib
  MOD_RES 7: note = Napthyl alanine
  MOD_RES 8: note = Cyclohexylalanine
  SITE 12: note = C-term CONH2
XXXVXEXXCEER

SEQ ID NO: 610 - protein, 12 aa, synthetic construct
  MOD_RES 1: note = Aad
  MOD_RES 2: note = Octanonic
  MOD_RES 4: note = Napthyl alanine
  MOD_RES 8: note = Phenyl glycine
  SITE 12: note = C-term CONH2
XXTXAELXCEER

SEQ ID NO: 611 - protein, 12 aa, synthetic construct
  MOD_RES 1: note = Aad
  MOD_RES 2: note = Homo phenylalanine
  MOD_RES 4: note = Phenyl glycine
  MOD_RES 5: note = Aib
  MOD_RES 8: note = Cyclohexylalanine
  SITE 12: note = C-term CONH2
XXTXXELXCEER

SEQ ID NO: 612 - protein, 12 aa, synthetic construct
  MOD_RES 1: note = Aad
  MOD_RES 3: note = Hyp
  MOD_RES 4: note = Cyclohexylalanine
  MOD_RES 5: note = Aib
  MOD_RES 7: note = Cyclohexylalanine
  MOD_RES 8: note = Homo phenylalanine
  SITE 12: note = C-term CONH2
XLXXXEXXCEER

SEQ ID NO: 613 - protein, 12 aa, synthetic construct
  MOD_RES 1: note = Aad
  MOD_RES 4: note = Napthyl alanine
  MOD_RES 7: note = Octanonic
  SITE 12: note = C-term CONH2
XLTXAEXLCEER

SEQ ID NO: 614 - protein, 12 aa, synthetic construct
  MOD_RES 1: note = Aad
  MOD_RES 2: note = Homo phenylalanine
  MOD_RES 4: note = Napthyl alanine
  MOD_RES 7: note = Cyclobutylalanine
  SITE 12: note = C-term CONH2
XXTXAEXLCEER

SEQ ID NO: 615 - protein, 12 aa, synthetic construct
  MOD_RES 4: note = Phenyl glycine
  SITE 12: note = C-term CONH2
ELNXGELVCEER

SEQ ID NO: 616 - protein, 12 aa, synthetic construct
  MOD_RES 3: note = Hyp
  SITE 12: note = C-term CONH2
ELXVQEVLCEER

SEQ ID NO: 617 - protein, 12 aa, synthetic construct
  MOD_RES 4: note = Cyclopropylalanine
  MOD_RES 7: note = Phenyl glycine
  SITE 12: note = C-term CONH2
ELNXAEXVCEER

SEQ ID NO: 618 - protein, 12 aa, synthetic construct
  MOD_RES 1: note = Aad
  MOD_RES 3: note = Cyclopropylglycine
  MOD_RES 4: note = Napthyl alanine
  MOD_RES 5: note = Aib
  MOD_RES 8: note = Cyclobutylalanine
  SITE 12: note = C-term CONH2
XVXXXELXCEER

SEQ ID NO: 619 - protein, 12 aa, synthetic construct
  MOD_RES 1: note = Aad
  MOD_RES 3: note = Hyp
  MOD_RES 8: note = Phenyl glycine
  SITE 12: note = C-term CONH2
XVXVGEVXCEER

SEQ ID NO: 620 - protein, 12 aa, synthetic construct
  SITE 12: note = C-term CONH2
ELNVQEVVCEER

SEQ ID NO: 621 - protein, 12 aa, synthetic construct
  MOD_RES 1: note = Aad
  MOD_RES 2: note = Cyclopropylalanine
  MOD_RES 3: note = Hyp
  MOD_RES 8: note = Cyclopropylalanine
  SITE 12: note = C-term CONH2
XXXVQELXCEER

SEQ ID NO: 622 - protein, 12 aa, synthetic construct
  MOD_RES 2: note = Cyclobutylalanine
  MOD_RES 3: note = Cyclopropylglycine
  MOD_RES 4: note = Napthyl alanine
  MOD_RES 5: note = Aib
  MOD_RES 7: note = Cyclobutylalanine
  SITE 12: note = C-term CONH2
EXXXXEXLCEER

SEQ ID NO: 623 - protein, 12 aa, synthetic construct
  MOD_RES 3: note = Hyp
  MOD_RES 4: note = Cyclopropylalanine
  SITE 12: note = C-term CONH2
ELXXQEVLCEER

SEQ ID NO: 624 - protein, 12 aa, synthetic construct
  MOD_RES 1: note = Aad
  MOD_RES 2: note = Napthyl alanine
  MOD_RES 5: note = Aib
  MOD_RES 7: note = Homo phenylalanine
  SITE 12: note = C-term CONH2
XXNVXEXLCEER

SEQ ID NO: 625 - protein, 12 aa, synthetic construct
  MOD_RES 1: note = Aad
  MOD_RES 2: note = Cyclopropylalanine
  MOD_RES 3: note = Hyp
  MOD_RES 4: note = Phenyl glycine
  MOD_RES 8: note = Cyclopropylalanine
  SITE 12: note = C-term CONH2
XXXXQELXCEER

SEQ ID NO: 626 - protein, 12 aa, synthetic construct
  MOD_RES 1: note = Aad
  MOD_RES 2: note = Napthyl alanine
  MOD_RES 3: note = Cyclobutylglycine
  MOD_RES 5: note = Aib
  MOD_RES 6: note = Aad
  MOD_RES 8: note = Cyclobutylalanine
  SITE 12: note = C-term CONH2
XXXLXXVXCEER

SEQ ID NO: 627 - protein, 12 aa, synthetic construct
  MOD_RES 1: note = Aad
  MOD_RES 7: note = Cyclopropylalanine
  MOD_RES 8: note = Phenyl glycine
  SITE 12: note = C-term CONH2
XVNVQEXXCEER

SEQ ID NO: 628 - protein, 12 aa, synthetic construct
  MOD_RES 7: note = Phenyl glycine
  SITE 12: note = C-term CONH2
ELNVAEXVCEER

SEQ ID NO: 629 - protein, 12 aa, synthetic construct
  MOD_RES 3: note = Cyclopropylglycine
  MOD_RES 4: note = Cyclopropylalanine
  MOD_RES 8: note = Phenyl glycine
  SITE 12: note = C-term CONH2
ELXXQEVXCEER

SEQ ID NO: 630 - protein, 12 aa, synthetic construct
  MOD_RES 1: note = Aad
  MOD_RES 3: note = Hyp
  MOD_RES 4: note = Cyclopropylalanine
  MOD_RES 6: note = Aad
  MOD_RES 8: note = Phenyl glycine
  SITE 12: note = C-term CONH2
XVXXAXVXCEER

SEQ ID NO: 631 - protein, 12 aa, synthetic construct
  MOD_RES 1: note = Aad
  MOD_RES 2: note = Cyclopropylalanine
  MOD_RES 3: note = Cyclobutylglycine
  MOD_RES 4: note = Cyclopropylalanine
  MOD_RES 5: note = Aib
  MOD_RES 7: note = Napthyl alanine
  SITE 12: note = C-term CONH2
XXXXXEXLCEER

SEQ ID NO: 632 - protein, 12 aa, synthetic construct
  MOD_RES 1: note = Aad
  MOD_RES 2: note = Octanonic
  MOD_RES 4: note = Cyclobutylalanine
  MOD_RES 7: note = Cyclohexylalanine
  SITE 12: note = C-term CONH2
XXNXGEXLCEER

SEQ ID NO: 633 - protein, 12 aa, synthetic construct
  MOD_RES 3: note = Hyp
  MOD_RES 8: note = Phenyl glycine
  SITE 12: note = C-term CONH2
ELXVQELXCEER

SEQ ID NO: 634 - protein, 12 aa, synthetic construct
  SITE 12: note = C-term CONH2
ELNVQEVLCEER

SEQ ID NO: 635 - protein, 12 aa, synthetic construct
  MOD_RES 1: note = Aad
  MOD_RES 2: note = Napthyl alanine
  MOD_RES 3: note = Cyclobutylglycine
  MOD_RES 4: note = Cyclopropylalanine
  MOD_RES 7: note = Homo phenylalanine
  SITE 12: note = C-term CONH2
XXXXAEXLCEER

SEQ ID NO: 636 - protein, 12 aa, synthetic construct
  MOD_RES 1: note = Aad
  MOD_RES 2: note = Napthyl alanine
  MOD_RES 3: note = Hyp
  MOD_RES 5: note = Aib
  MOD_RES 7: note = Cyclohexylalanine
  SITE 12: note = C-term CONH2
XXXVXEXLCEER

SEQ ID NO: 637 - protein, 12 aa, synthetic construct
  MOD_RES 1: note = Aad
  MOD_RES 2: note = Cyclohexylalanine
  MOD_RES 3: note = Cyclobutylglycine
  MOD_RES 4: note = Phenyl glycine
  MOD_RES 5: note = Aib
  MOD_RES 7: note = Cyclobutylalanine
  SITE 12: note = C-term CONH2
XXXXXEXLCEER

SEQ ID NO: 638 - protein, 12 aa, synthetic construct
  MOD_RES 2: note = Cyclobutylalanine
  MOD_RES 3: note = Hyp
  MOD_RES 4: note = Cyclopropylalanine
  SITE 12: note = C-term CONH2
EXXXQEVVCEER

SEQ ID NO: 639 - protein, 12 aa, synthetic construct
  MOD_RES 8: note = Phenyl glycine
  SITE 12: note = C-term CONH2
ELNVAEVXCEER

SEQ ID NO: 640 - protein, 12 aa, synthetic construct
  MOD_RES 1: note = Aad
  MOD_RES 3: note = Hyp
  MOD_RES 4: note = Cyclopropylalanine
  MOD_RES 7: note = Phenyl glycine
  SITE 12: note = C-term CONH2
XVXXGEXVCEER

SEQ ID NO: 641 - protein, 12 aa, synthetic construct
  MOD_RES 3: note = Hyp
  SITE 12: note = C-term CONH2
ELXVQEVVCEER

SEQ ID NO: 642 - protein, 12 aa, synthetic construct
  MOD_RES 1: note = Aad
  MOD_RES 2: note = Phenyl glycine
  MOD_RES 3: note = Hyp
  MOD_RES 4: note = Cyclopropylalanine
  MOD_RES 8: note = Phenyl glycine
  SITE 12: note = C-term CONH2
XXXXQEVXCEER

SEQ ID NO: 643 - protein, 12 aa, synthetic construct
  MOD_RES 1: note = Aad
  MOD_RES 3: note = Hyp
  MOD_RES 7: note = Cyclopropylalanine
  SITE 12: note = C-term CONH2
XLXVQEXLCEER

SEQ ID NO: 644 - protein, 12 aa, synthetic construct
  MOD_RES 1: note = Aad
  MOD_RES 4: note = Cyclopropylalanine
  MOD_RES 8: note = Phenyl glycine
  SITE 12: note = C-term CONH2
XVNXGEVXCEER

SEQ ID NO: 645 - protein, 12 aa, synthetic construct
  MOD_RES 3: note = Hyp
  MOD_RES 8: note = Cyclopropylalanine
  SITE 12: note = C-term CONH2
ELXVGEVXCEER

SEQ ID NO: 646 - protein, 12 aa, synthetic construct
  MOD_RES 1: note = Aad
  MOD_RES 3: note = Hyp
  MOD_RES 7: note = Cyclobutylalanine
  SITE 12: note = C-term CONH2
XVXVAEXVCEER

SEQ ID NO: 647 - protein, 12 aa, synthetic construct
  MOD_RES 3: note = Hyp
  MOD_RES 7: note = Cyclopropylalanine
  SITE 12: note = C-term CONH2
EVXVQEXVCEER

SEQ ID NO: 648 - protein, 12 aa, synthetic construct
  MOD_RES 1: note = Aad
  MOD_RES 4: note = Cyclopropylalanine
  MOD_RES 8: note = Phenyl glycine
  SITE 12: note = C-term CONH2
XLTXGEVXCEER

SEQ ID NO: 649 - protein, 12 aa, synthetic construct
  MOD_RES 1: note = Aad
  MOD_RES 4: note = Cyclopropylalanine
  MOD_RES 8: note = Phenyl glycine
  SITE 12: note = C-term CONH2
XLTXAEVXCEER

SEQ ID NO: 650 - protein, 12 aa, synthetic construct
  MOD_RES 1: note = Aad
  MOD_RES 3: note = Hyp
  MOD_RES 4: note = Cyclopropylalanine
  MOD_RES 8: note = Phenyl glycine
  SITE 12: note = C-term CONH2
XLXXGELXCEER

SEQ ID NO: 651 - protein, 12 aa, synthetic construct
  MOD_RES 1: note = Aad
  MOD_RES 2: note = Phenyl glycine
  MOD_RES 3: note = Hyp
  MOD_RES 7: note = Cyclopropylalanine
  SITE 12: note = C-term CONH2
XXXVGEXVCEER

SEQ ID NO: 652 - protein, 12 aa, synthetic construct
  MOD_RES 1: note = Aad
  SITE 12: note = C-term CONH2
XLTVQEVVCEER

SEQ ID NO: 653 - protein, 12 aa, synthetic construct
  MOD_RES 1: note = Aad
  MOD_RES 3: note = Cyclopropylglycine
  MOD_RES 4: note = Cyclohexylalanine
  MOD_RES 7: note = Octanonic
  MOD_RES 8: note = Phenyl glycine
  SITE 12: note = C-term CONH2
XLXXGEXXCEER

SEQ ID NO: 654 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 11: note = Aad
  MOD_RES 12: note = Hyp
XXXLXEXLCKXX

SEQ ID NO: 655 - protein, 12 aa, synthetic construct
  MOD_RES 1: note = Aad
  MOD_RES 2: note = Napthylalanine
  MOD_RES 3: note = Cyclohexylglycine
  MOD_RES 7: note = Cyclobutylalanine
  MOD_RES 12: note = Aib
XXXLAEXLCVRX

SEQ ID NO: 656 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
XXXLXEXLCKKQ

SEQ ID NO: 657 - protein, 12 aa, synthetic construct
  MOD_RES 1: note = Aad
  MOD_RES 2: note = 4-phenyl phenylalanine
  MOD_RES 3: note = Cyclopentylglycine
  MOD_RES 5: note = Aib
  MOD_RES 7: note = Cyclobutylalanine
  MOD_RES 12: note = Hyp
XXXLXEXLCKAX

SEQ ID NO: 658 - protein, 12 aa, synthetic construct
  MOD_RES 1: note = Aad
  MOD_RES 2: note = Napthylalanine
  MOD_RES 3: note = Cyclohexylglycine
  MOD_RES 7: note = Cyclobutylalanine
XXXLAEXLCVRG

SEQ ID NO: 659 - protein, 12 aa, synthetic construct
  MOD_RES 1: note = Aad
  MOD_RES 2: note = Napthylalanine
  MOD_RES 3: note = Cyclohexylglycine
  MOD_RES 7: note = Cyclobutylalanine
  MOD_RES 12: note = Hyp
XXXLAEXLCREX

SEQ ID NO: 660 - protein, 12 aa, synthetic construct
  MOD_RES 1: note = Aad
  MOD_RES 2: note = Napthylalanine
  MOD_RES 3: note = Cyclohexylglycine
  MOD_RES 7: note = Cyclohexylglycine
  MOD_RES 12: note = Hyp
XXXLAEXLCKKX

SEQ ID NO: 661 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 12: note = Aib
XXXLXEXLCRRX

SEQ ID NO: 662 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
XXXLXEXLCYQL

SEQ ID NO: 663 - protein, 12 aa, synthetic construct
  MOD_RES 1: note = Aad
  MOD_RES 3: note = Cyclohexylglycine
  MOD_RES 7: note = Cyclobutylalanine
  MOD_RES 11: note = Aad
  MOD_RES 12: note = Hyp
XWXLAEXLCHXX

SEQ ID NO: 664 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 12: note = Aad
XXXLXEXLCRKX

SEQ ID NO: 665 - protein, 12 aa, synthetic construct
  MOD_RES 1: note = Aad
  MOD_RES 2: note = Napthylalanine
  MOD_RES 3: note = Cyclohexylglycine
  MOD_RES 7: note = Cyclobutylalanine
XXXLTEXLCQLE

SEQ ID NO: 666 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
XXXLXEXLCKQK

SEQ ID NO: 667 - protein, 12 aa, synthetic construct
  MOD_RES 1: note = Aad
  MOD_RES 2: note = Napthylalanine
  MOD_RES 3: note = Cyclohexylglycine
  MOD_RES 7: note = Cyclohexylglycine
  MOD_RES 12: note = Hyp
XXXLAEXLCKQX

SEQ ID NO: 668 - protein, 12 aa, synthetic construct
  MOD_RES 1: note = Aad
  MOD_RES 3: note = Napthylalanine
  MOD_RES 7: note = Cyclobutylalanine
XWXLAEXLCALW

SEQ ID NO: 669 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
XXXLXEXLCVER

SEQ ID NO: 670 - protein, 12 aa, synthetic construct
  MOD_RES 1: note = Aad
  MOD_RES 2: note = Napthylalanine
  MOD_RES 3: note = Cyclohexylglycine
  MOD_RES 7: note = Cyclohexylglycine
  MOD_RES 12: note = Aib
XXXLAEXLCRAX

SEQ ID NO: 671 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
XXXLXEXLCKEM

SEQ ID NO: 672 - protein, 12 aa, synthetic construct
  MOD_RES 1: note = Aad
  MOD_RES 2: note = Napthylalanine
  MOD_RES 3: note = Cyclohexylglycine
  MOD_RES 7: note = Cyclobutylalanine
  MOD_RES 10: note = Aib
XXXLTEXLCXER

SEQ ID NO: 673 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
XXXLXEXLCVKG

SEQ ID NO: 674 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 11: note = Aad
  MOD_RES 12: note = Hyp
XXXLXEXLCHXX

SEQ ID NO: 675 - protein, 12 aa, synthetic construct
  MOD_RES 1: note = Aad
  MOD_RES 3: note = Cyclopentylglycine
  MOD_RES 7: note = Cyclobutylalanine
XWXLTEXLCHAQ

SEQ ID NO: 676 - protein, 12 aa, synthetic construct
  MOD_RES 1: note = Aad
  MOD_RES 2: note = Napthylalanine
  MOD_RES 3: note = Cyclohexylglycine
  MOD_RES 7: note = Cyclohexylglycine
  MOD_RES 10: note = Hyp
  MOD_RES 12: note = Hyp
XXXLAEXLCXEX

SEQ ID NO: 677 - protein, 12 aa, synthetic construct
  MOD_RES 1: note = Aad
  MOD_RES 2: note = Napthylalanine
  MOD_RES 3: note = Cyclohexylglycine
  MOD_RES 7: note = Cyclohexylglycine
  MOD_RES 12: note = Hyp
XXXLAEXLCVKX

SEQ ID NO: 678 - protein, 12 aa, synthetic construct
  MOD_RES 1: note = Aad
  MOD_RES 2: note = 4-phenyl phenylalanine
  MOD_RES 3: note = Cyclohexylglycine
  MOD_RES 5: note = Aib
  MOD_RES 7: note = Cyclobutylalanine
  MOD_RES 10: note = Hyp
XXXLXEXLCXKE

SEQ ID NO: 679 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 10: note = Aib
XXXLXEXLCXRR

SEQ ID NO: 680 - protein, 12 aa, synthetic construct
  MOD_RES 1: note = Aad
  MOD_RES 2: note = Napthylalanine
  MOD_RES 3: note = Cyclopentylglycine
  MOD_RES 7: note = Cyclobutylalanine
XXXLTEXLCHGQ

SEQ ID NO: 681 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
XWXLXEXLCHVQ

SEQ ID NO: 682 - protein, 12 aa, synthetic construct
  MOD_RES 1: note = Aad
  MOD_RES 2: note = Napthylalanine
  MOD_RES 3: note = Cyclohexylglycine
  MOD_RES 7: note = Cyclobutylalanine
XXXLTEXLCQAK

SEQ ID NO: 683 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
  MOD_RES 10: note = Hyp
XWXLXEXLCXYT

SEQ ID NO: 684 - protein, 12 aa, synthetic construct
  MOD_RES 1: note = Aad
  MOD_RES 2: note = Napthylalanine
  MOD_RES 3: note = Cyclopentylglycine
  MOD_RES 7: note = Cyclobutylalanine
  MOD_RES 11..12: note = Hyp
XXXLTEXLCYXX

SEQ ID NO: 685 - protein, 12 aa, synthetic construct
  MOD_RES 1: note = Aad
  MOD_RES 2: note = 4-phenyl phenylalanine
  MOD_RES 3: note = Cyclohexylglycine
  MOD_RES 7: note = Cyclobutylalanine
  MOD_RES 12: note = Hyp
XXXLAEXLCVRX

SEQ ID NO: 686 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclohexylglycine
  MOD_RES 12: note = Hyp
XXXLXEXLCKTX

SEQ ID NO: 687 - protein, 12 aa, synthetic construct
  MOD_RES 1: note = Aad
  MOD_RES 2: note = Napthylalanine
  MOD_RES 3: note = Cyclohexylglycine
  MOD_RES 7: note = Cyclobutylalanine
XXXLAEXLCHKV

SEQ ID NO: 688 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
XXXLXEXLCHQQ

SEQ ID NO: 689 - protein, 12 aa, synthetic construct
  MOD_RES 1: note = Aad
  MOD_RES 2: note = Napthylalanine
  MOD_RES 3: note = Cyclohexylglycine
  MOD_RES 7: note = Cyclohexylglycine
  MOD_RES 11: note = Hyp
XXXLAEXLCVXT

SEQ ID NO: 690 - protein, 12 aa, synthetic construct
  MOD_RES 1: note = Aad
  MOD_RES 2: note = Napthylalanine
  MOD_RES 3: note = Cyclohexylglycine
  MOD_RES 7: note = Cyclobutylalanine
  MOD_RES 12: note = Aib
XXXLAEXLCFWX

SEQ ID NO: 691 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
  MOD_RES 10: note = Aad
  MOD_RES 12: note = Hyp
XWXLXEXLCXHX

SEQ ID NO: 692 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
  MOD_RES 10: note = Aib
XWXLXEXLCXRE

SEQ ID NO: 693 - protein, 12 aa, synthetic construct
  MOD_RES 2: note = 4-phenyl phenylalanine
  MOD_RES 3: note = Cyclopentylglycine
  MOD_RES 5: note = Aib
  MOD_RES 7: note = Cyclobutylalanine
WXXLXEXLCAKK

SEQ ID NO: 694 - protein, 11 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
XXXLXEXLCRH

SEQ ID NO: 695 - protein, 11 aa, synthetic construct
  MOD_RES 1: note = Aad
  MOD_RES 2: note = 4-phenyl phenylalanine
  MOD_RES 3: note = Cyclopentylglycine
  MOD_RES 5: note = Aib
  MOD_RES 7: note = Cyclobutylalanine
XXXLXEXLCKR

SEQ ID NO: 696 - protein, 11 aa, synthetic construct
  MOD_RES 2: note = QuinolylAlanine
  MOD_RES 3: note = Cyclopentylglycine
  MOD_RES 5: note = Aib
  MOD_RES 7: note = Cyclobutylalanine
RXXLXEXLCRK

SEQ ID NO: 697 - protein, 11 aa, synthetic construct
  MOD_RES 1: note = Aad
  MOD_RES 2: note = Napthylalanine
  MOD_RES 3: note = Cyclohexylglycine
  MOD_RES 7: note = Cyclohexylglycine
XXXLAEXLCQR

SEQ ID NO: 698 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
  MOD_RES 11: note = Aad
  MOD_RES 12: note = Hyp
XWXLXEXLCKXX

SEQ ID NO: 699 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
XXXLXEXLCVAK

SEQ ID NO: 700 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
XXXLXEXLCVGK

SEQ ID NO: 701 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
  MOD_RES 12: note = Aib
XXXLXEXLCQRX

SEQ ID NO: 702 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
  MOD_RES 12: note = Aib
XWXLXEXLCQRX

SEQ ID NO: 703 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
  MOD_RES 11: note = Aad
  MOD_RES 12: note = Hyp
XXXLXEXLCKXX

SEQ ID NO: 704 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
  MOD_RES 12: note = Aib
XXXLXEXLCGRX

SEQ ID NO: 705 - protein, 12 aa, synthetic construct
  MOD_RES 1: note = Aad
  MOD_RES 3: note = Cyclopentylglycine
  MOD_RES 5: note = Aib
  MOD_RES 7: note = Cyclobutylalanine
XWXLXEXLCKFH

SEQ ID NO: 706 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
  MOD_RES 10: note = Aib
XXXLXEXLCXRG

SEQ ID NO: 707 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
XXXLXEXLCAVK

SEQ ID NO: 708 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
  MOD_RES 12: note = Hyp
XWXLXEXLCRGX

SEQ ID NO: 709 - protein, 12 aa, synthetic construct
  MOD_RES 1: note = Aad
  MOD_RES 3: note = Cyclopentylglycine
  MOD_RES 5: note = Aib
  MOD_RES 7: note = Cyclobutylalanine
  MOD_RES 10: note = Aib
XWXLXEXLCXMY

SEQ ID NO: 710 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 12: note = Hyp
XXXLXEXLCKEX

SEQ ID NO: 711 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
  MOD_RES 12: note = Aad
XWXLXEXLCGKX

SEQ ID NO: 712 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
  MOD_RES 11: note = Aad
XWXLXEXLCAXR

SEQ ID NO: 713 - protein, 12 aa, synthetic construct
  MOD_RES 1: note = Aad
  MOD_RES 3: note = Cyclopentylglycine
  MOD_RES 5: note = Aib
  MOD_RES 7: note = Cyclobutylalanine
XWXLXEXLCKQA

SEQ ID NO: 714 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
  MOD_RES 10: note = Aad
XXXLXEXLCXKQ

SEQ ID NO: 715 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
  MOD_RES 12: note = Aad
XWXLXEXLCKVX

SEQ ID NO: 716 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
  MOD_RES 12: note = Aad
XWXLXEXLCXVR

SEQ ID NO: 717 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
  MOD_RES 10: note = Aad
XXXLXEXLCXQH

SEQ ID NO: 718 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
  MOD_RES 10: note = Aad
XXXLXEXLCXHQ

SEQ ID NO: 719 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
  MOD_RES 10: note = Aad
XXXLXEXLCXGH

SEQ ID NO: 720 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
XXXLXEXLCREE

SEQ ID NO: 721 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 12: note = Aad
XXXLXEXLCVWX

SEQ ID NO: 722 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
XXXLXEXLCLFH

SEQ ID NO: 723 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
XXXLXEXLCKEF

SEQ ID NO: 724 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 11: note = Aad
XXXLXEXLCRXE

SEQ ID NO: 725 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 12: note = Aad
XXXLXEXLCLWX

SEQ ID NO: 726 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
XXXLXEXLCWHH

SEQ ID NO: 727 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
  MOD_RES 12: note = Hyp
XXXLXEXLCMVX

SEQ ID NO: 728 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
  MOD_RES 10: note = Aad
XXXLXEXLCXKG

SEQ ID NO: 729 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
XWXLXEXLCGVE

SEQ ID NO: 730 - protein, 12 aa, synthetic construct
  MOD_RES 1: note = Aad
  MOD_RES 2: note = Napthylalanine
  MOD_RES 3: note = Cyclohexylglycine
  MOD_RES 7: note = Cyclobutylalanine
  MOD_RES 12: note = Hyp
XXXLAEXLCKVX

SEQ ID NO: 731 - protein, 12 aa, synthetic construct
  MOD_RES 1: note = Aad
  MOD_RES 2: note = 4-phenyl phenylalanine
  MOD_RES 3: note = Cyclopentylglycine
  MOD_RES 5: note = Aib
  MOD_RES 7: note = Cyclobutylalanine
  MOD_RES 11: note = Aad
  MOD_RES 12: note = Hyp
XXXLXEXLCKXX

SEQ ID NO: 732 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclohexylglycine
  MOD_RES 10: note = Aib
XXXLXEXLCXKQ

SEQ ID NO: 733 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 10: note = Aib
  MOD_RES 12: note = Hyp
XXXLXEXLCXRX

SEQ ID NO: 734 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
XXXLXEXLCKAG

SEQ ID NO: 735 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 11: note = Hyp
XXXLXEXLCVXQ

SEQ ID NO: 736 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
XXXLXEXLCHHH

SEQ ID NO: 737 - protein, 12 aa, synthetic construct
  MOD_RES 1: note = Aad
  MOD_RES 2: note = Napthylalanine
  MOD_RES 3: note = Cyclopentylglycine
  MOD_RES 7: note = Cyclobutylalanine
XXXLVEXLCTYE

SEQ ID NO: 738 - protein, 11 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
XXXLXEXLCKQ

SEQ ID NO: 739 - protein, 12 aa, synthetic construct
  MOD_RES 1: note = Aad
  MOD_RES 2: note = 4-phenyl phenylalanine
  MOD_RES 3: note = Cyclohexylglycine
  MOD_RES 5: note = Aib
  MOD_RES 7: note = Cyclobutylalanine
  MOD_RES 11: note = Aad
  MOD_RES 12: note = Hyp
XXXLXEXLCKXX

SEQ ID NO: 740 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 10: note = Aad
  MOD_RES 12: note = Hyp
XXXLXEXLCXKX

SEQ ID NO: 741 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
XXXLXEXLCKGV

SEQ ID NO: 742 - protein, 12 aa, synthetic construct
  MOD_RES 1: note = Aad
  MOD_RES 2: note = 4-phenyl phenylalanine
  MOD_RES 3: note = Cyclohexylglycine
  MOD_RES 5: note = Aib
  MOD_RES 7: note = Cyclobutylalanine
XXXLXEXLCVER

SEQ ID NO: 743 - protein, 12 aa, synthetic construct
  MOD_RES 1: note = Aad
  MOD_RES 2: note = 4-phenyl phenylalanine
  MOD_RES 3: note = Cyclopentylglycine
  MOD_RES 5: note = Aib
  MOD_RES 7: note = Cyclobutylalanine
XXXLXEXLCVGK

SEQ ID NO: 744 - protein, 12 aa, synthetic construct
  MOD_RES 1: note = Aad
  MOD_RES 2: note = 4-phenyl phenylalanine
  MOD_RES 3: note = Cyclopentylglycine
  MOD_RES 5: note = Aib
  MOD_RES 7: note = Cyclobutylalanine
XXXLXEXLCYLQ

SEQ ID NO: 745 - protein, 12 aa, synthetic construct
  MOD_RES 1: note = Aad
  MOD_RES 2: note = 4-phenyl phenylalanine
  MOD_RES 3: note = Cyclohexylglycine
  MOD_RES 5: note = Aib
  MOD_RES 7: note = Cyclobutylalanine
  MOD_RES 10: note = Aad
  MOD_RES 11: note = Hyp
XXXLXEXLCXXK

SEQ ID NO: 746 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 12: note = Hyp
XXXLXEXLCFKX

SEQ ID NO: 747 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
XXXLXEXLCVRQ

SEQ ID NO: 748 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 11: note = Aad
XXXLXEXLCQXR

SEQ ID NO: 749 - protein, 12 aa, synthetic construct
  MOD_RES 1: note = Aad
  MOD_RES 2: note = 4-phenyl phenylalanine
  MOD_RES 3: note = Cyclopentylglycine
  MOD_RES 5: note = Aib
  MOD_RES 7: note = Cyclobutylalanine
  MOD_RES 12: note = Aib
XXXLXEXLCGKX

SEQ ID NO: 750 - protein, 12 aa, synthetic construct
  MOD_RES 1: note = Aad
  MOD_RES 2: note = 4-phenyl phenylalanine
  MOD_RES 3: note = Cyclopentylglycine
  MOD_RES 5: note = Aib
  MOD_RES 7: note = Cyclobutylalanine
XXXLXEXLCVAK

SEQ ID NO: 751 - protein, 12 aa, synthetic construct
  MOD_RES 1: note = Aad
  MOD_RES 2: note = 4-phenyl phenylalanine
  MOD_RES 3: note = Cyclohexylglycine
  MOD_RES 5: note = Aib
  MOD_RES 7: note = Cyclobutylalanine
  MOD_RES 10: note = Aad
  MOD_RES 12: note = Hyp
XXXLXEXLCXHX

SEQ ID NO: 752 - protein, 11 aa, synthetic construct
  MOD_RES 1: note = Aad
  MOD_RES 2: note = Napthylalanine
  MOD_RES 3: note = Cyclohexylglycine
  MOD_RES 7: note = Cyclobutylalanine
XXXLAEXLCRR

SEQ ID NO: 753 - protein, 12 aa, synthetic construct
  MOD_RES 1: note = Aad
  MOD_RES 3: note = Cyclopentylglycine
  MOD_RES 5: note = Aib
  MOD_RES 7: note = Cyclobutylalanine
  MOD_RES 12: note = Aib
XWXLXEXLCYRX

SEQ ID NO: 754 - protein, 12 aa, synthetic construct
  MOD_RES 1: note = Aad
  MOD_RES 2: note = 4-phenyl phenylalanine
  MOD_RES 3: note = Cyclopentylglycine
  MOD_RES 5: note = Aib
  MOD_RES 7: note = Cyclobutylalanine
  MOD_RES 12: note = Aib
XXXLXEXLCGRX

SEQ ID NO: 755 - protein, 12 aa, synthetic construct
  MOD_RES 1: note = Aad
  MOD_RES 2: note = 4-phenyl phenylalanine
  MOD_RES 3: note = Cyclopentylglycine
  MOD_RES 5: note = Aib
  MOD_RES 7: note = Cyclobutylalanine
XXXLXEXLCGKA

SEQ ID NO: 756 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
XWXLXEXLCKVG

SEQ ID NO: 757 - protein, 12 aa, synthetic construct
  MOD_RES 1: note = Aad
  MOD_RES 2: note = 4-phenyl phenylalanine
  MOD_RES 3: note = Cyclohexylglycine
  MOD_RES 5: note = Aib
  MOD_RES 7: note = Cyclobutylalanine
XXXLXEXLCFLH

SEQ ID NO: 758 - protein, 12 aa, synthetic construct
  MOD_RES 1: note = Aad
  MOD_RES 2: note = 4-phenyl phenylalanine
  MOD_RES 3: note = Cyclopentylglycine
  MOD_RES 5: note = Aib
  MOD_RES 7: note = Cyclobutylalanine
XXXLXEXLCGKG

SEQ ID NO: 759 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
XXXLXEXLCKFH

SEQ ID NO: 760 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 12: note = Aib
XXXLXEXLCKQX

SEQ ID NO: 761 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
XXXLXEXLCVVK

SEQ ID NO: 762 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
XXXLXEXLCKEM

SEQ ID NO: 763 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
XXXLXEXLCLAK

SEQ ID NO: 764 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
  MOD_RES 12: note = Aad
XXXLXEXLCQHX

SEQ ID NO: 765 - protein, 12 aa, synthetic construct
  MOD_RES 1: note = Aad
  MOD_RES 2: note = 4-phenyl phenylalanine
  MOD_RES 3: note = Cyclohexylglycine
  MOD_RES 5: note = Aib
  MOD_RES 7: note = Cyclobutylalanine
  MOD_RES 10: note = Aib
XXXLXEXLCXRG

SEQ ID NO: 766 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
  MOD_RES 12: note = Hyp
XXXLXEXLCKFX

SEQ ID NO: 767 - protein, 12 aa, synthetic construct
  MOD_RES 1: note = Aad
  MOD_RES 2: note = 4-phenyl phenylalanine
  MOD_RES 3: note = Cyclohexylglycine
  MOD_RES 5: note = Aib
  MOD_RES 7: note = Cyclobutylalanine
  MOD_RES 12: note = Hyp
XXXLXEXLCKVX

SEQ ID NO: 768 - protein, 12 aa, synthetic construct
  MOD_RES 1: note = Aad
  MOD_RES 2: note = 4-phenyl phenylalanine
  MOD_RES 3: note = Cyclohexylglycine
  MOD_RES 5: note = Aib
  MOD_RES 7: note = Cyclobutylalanine
  MOD_RES 12: note = Hyp
XXXLXEXLCEKX

SEQ ID NO: 769 - protein, 12 aa, synthetic construct
  MOD_RES 1: note = Aad
  MOD_RES 2: note = 4-phenyl phenylalanine
  MOD_RES 3: note = Cyclohexylglycine
  MOD_RES 5: note = Aib
  MOD_RES 7: note = Cyclobutylalanine
XXXLXEXLCYLH

SEQ ID NO: 770 - protein, 11 aa, synthetic construct
  MOD_RES 1: note = Aad
  MOD_RES 2: note = 4-phenyl phenylalanine
  MOD_RES 3: note = Cyclopentylglycine
  MOD_RES 5: note = Aib
  MOD_RES 7: note = Cyclobutylalanine
XXXLXEXLCKQ

SEQ ID NO: 771 - protein, 11 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
XXXLXEXLCHR

SEQ ID NO: 772 - protein, 11 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
  MOD_RES 11: note = Aad
XXXLXEXLCLX

SEQ ID NO: 773 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
XWXLXEXLCEQH

SEQ ID NO: 774 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
XXXLXEXLCVAQ

SEQ ID NO: 775 - protein, 12 aa, synthetic construct
  MOD_RES 1: note = Aad
  MOD_RES 2: note = 4-phenyl phenylalanine
  MOD_RES 3: note = Cyclohexylglycine
  MOD_RES 5: note = Aib
  MOD_RES 7: note = Cyclobutylalanine
XXXLXEXLCVAK

SEQ ID NO: 776 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
XXXLXEXLCAEK

SEQ ID NO: 777 - protein, 5 aa, synthetic construct
IPQSA

SEQ ID NO: 778 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
XXXLXEXLCQQL

SEQ ID NO: 779 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 12: note = Aib
XXXLXEXLCKRX

SEQ ID NO: 780 - protein, 12 aa, synthetic construct
  MOD_RES 1: note = Aad
  MOD_RES 2: note = 4-phenyl phenylalanine
  MOD_RES 3: note = Cyclopentylglycine
  MOD_RES 5: note = Aib
  MOD_RES 7: note = Cyclobutylalanine
  MOD_RES 11: note = Aib
XXXLXEXLCEXR

SEQ ID NO: 781 - protein, 12 aa, synthetic construct
  MOD_RES 1: note = Aad
  MOD_RES 3: note = Cyclopentylglycine
  MOD_RES 5: note = Aib
  MOD_RES 7: note = Cyclobutylalanine
  MOD_RES 12: note = Aad
XWXLXEXLCQKX

SEQ ID NO: 782 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 12: note = Hyp
XXXLXEXLCRVX

SEQ ID NO: 783 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
XXXLXEXLCKQA

SEQ ID NO: 784 - protein, 12 aa, synthetic construct
  MOD_RES 1: note = Aad
  MOD_RES 3: note = Cyclopentylglycine
  MOD_RES 5: note = Aib
  MOD_RES 7: note = Cyclobutylalanine
  MOD_RES 10: note = Aad
XXXLXEXLCXVK

SEQ ID NO: 785 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
XWXLXEXLCVVK

SEQ ID NO: 786 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
XWXLXEXLCVKG

SEQ ID NO: 787 - protein, 12 aa, synthetic construct
  MOD_RES 1: note = Aad
  MOD_RES 2: note = 4-phenyl phenylalanine
  MOD_RES 3: note = Cyclopentylglycine
  MOD_RES 5: note = Aib
  MOD_RES 7: note = Cyclobutylalanine
XXXLXEXLCKAQ

SEQ ID NO: 788 - protein, 12 aa, synthetic construct
  MOD_RES 1: note = Aad
  MOD_RES 3: note = Cyclopentylglycine
  MOD_RES 5: note = Aib
  MOD_RES 7: note = Cyclobutylalanine
XWXLXEXLCFKH

SEQ ID NO: 789 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 12: note = Aib
XXXLXEXLCAKX

SEQ ID NO: 790 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
  MOD_RES 12: note = Hyp
XWXLXEXLCVKX

SEQ ID NO: 791 - protein, 12 aa, synthetic construct
  MOD_RES 1: note = Aad
  MOD_RES 3: note = Cyclopentylglycine
  MOD_RES 5: note = Aib
  MOD_RES 7: note = Cyclobutylalanine
XWXLXEXLCLFH

SEQ ID NO: 792 - protein, 12 aa, synthetic construct
  MOD_RES 1: note = Aad
  MOD_RES 2: note = 4-phenyl phenylalanine
  MOD_RES 3: note = Cyclopentylglycine
  MOD_RES 5: note = Aib
  MOD_RES 7: note = Cyclobutylalanine
  MOD_RES 12: note = Hyp
XXXLXEXLCVTX

SEQ ID NO: 793 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
XWXLXEXLCTHF

SEQ ID NO: 794 - protein, 12 aa, synthetic construct
  MOD_RES 1: note = Aad
  MOD_RES 2: note = 4-phenyl phenylalanine
  MOD_RES 3: note = Cyclopentylglycine
  MOD_RES 5: note = Aib
  MOD_RES 7: note = Cyclobutylalanine
  MOD_RES 10: note = Aib
XXXLXEXLCXRG

SEQ ID NO: 795 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
  MOD_RES 12: note = Hyp
XWXLXEXLCKYX

SEQ ID NO: 796 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 11: note = Aib
XXXLXEXLCKXA

SEQ ID NO: 797 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
  MOD_RES 10: note = Aad
  MOD_RES 12: note = Aib
XXXLXEXLCXLX

SEQ ID NO: 798 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 10: note = Hyp
XXXLXEXLCXYK

SEQ ID NO: 799 - protein, 12 aa, synthetic construct
  MOD_RES 1: note = Aad
  MOD_RES 2: note = 4-phenyl phenylalanine
  MOD_RES 3: note = Cyclohexylglycine
  MOD_RES 5: note = Aib
  MOD_RES 7: note = Cyclobutylalanine
  MOD_RES 10: note = Aib
XXXLXEXLCXRH

SEQ ID NO: 800 - protein, 12 aa, synthetic construct
  MOD_RES 1: note = Aad
  MOD_RES 3: note = Cyclohexylglycine
  MOD_RES 5: note = Aib
  MOD_RES 7: note = Cyclobutylalanine
  MOD_RES 12: note = Aad
XWXLXEXLCFQX

SEQ ID NO: 801 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
XXXLXEXLCKQG

SEQ ID NO: 802 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
XXXLXEXLCAKG

SEQ ID NO: 803 - protein, 12 aa, synthetic construct
  MOD_RES 1: note = Aad
  MOD_RES 2: note = Napthylalanine
  MOD_RES 3: note = Cyclohexylglycine
  MOD_RES 5: note = Aib
  MOD_RES 7: note = Cyclobutylalanine
  MOD_RES 12: note = Hyp
XXXLXEXLCKLX

SEQ ID NO: 804 - protein, 12 aa, synthetic construct
  MOD_RES 1: note = Aad
  MOD_RES 2: note = Napthylalanine
  MOD_RES 3: note = Cyclopentylglycine
  MOD_RES 5: note = Aib
  MOD_RES 7: note = Cyclobutylalanine
XXXLXEXLCRKA

SEQ ID NO: 805 - protein, 12 aa, synthetic construct
  MOD_RES 1: note = Aad
  MOD_RES 4: note = Cyclobutylalanine
  MOD_RES 6: note = Aad
  SITE 12: note = C-term CONH2
XVNXAXVVCEER

SEQ ID NO: 806 - protein, 21 aa, synthetic construct
  SITE 1: note = N-term 9-fluorenyl acetamido
  SITE 21: note = C-term 5-acenaphthyl acetamido
  MOD_RES 21: note = Lysine(5-acenaphthyl acetamido)
IPESSELTLQELLGEERRAAX

SEQ ID NO: 807 - protein, 21 aa, synthetic construct
  SITE 1: note = N-term 9-fluorenyl acetamido
  SITE 21: note = C-term 1-naphthyl acetamido
  MOD_RES 21: note = Lysine(1-naphthyl acetamido)
IPESSELTLQELLGEERRAAX

SEQ ID NO: 808 - protein, 21 aa, synthetic construct
  SITE 1: note = N-term 1-anthracene carboxamido
  SITE 21: note = C-term 2-anthraquinone-yl-acetamido
  MOD_RES 21: note = Lysine(2-anthraquinone-yl-acetamido)
IPESSELTLQELLGEERRAAX

SEQ ID NO: 809 - protein, 21 aa, synthetic construct
  SITE 1: note = N-term 9-fluorenyl acetamido
  SITE 21: note = C-term 1-anthracenyl acetamido
  MOD_RES 21: note = Lysine(1-anthracenyl acetamido)
IPESSELTLQELLGEERRAAX

SEQ ID NO: 810 - protein, 21 aa, synthetic construct
  SITE 1: note = N-term 9-fluorenyl acetamido
  SITE 21: note = C-term 1-anthracenyl acetamido
  MOD_RES 21: note = Lysine(1-anthracenyl acetamido)
IPESAELTLQELLGEERRAAX

SEQ ID NO: 811 - protein, 22 aa, synthetic construct
  SITE 1: note = N-term fluorene-(Biotin-(polyethylene glycol 4)
  SITE 22: note = C-term 1-anthracenyl acetamido
  MOD_RES 22: note = Lysine(1-anthracenyl acetamido)
KIPESAELTLQELLGEERRAAX

SEQ ID NO: 812 - protein, 21 aa, synthetic construct
  SITE 1: note = N-term 9-fluorenyl acetamido
  SITE 21: note = C-term 1-anthracenyl acetamido
  MOD_RES 21: note = Lysine(1-anthracenyl acetamido)
IPESAELTAQEAAGEERRAAX

SEQ ID NO: 813 - protein, 18 aa, synthetic construct
  MOD_RES 1: note = bAla
XIPESSELTLQELLGEER

SEQ ID NO: 814 - protein, 18 aa, synthetic construct
  SITE 1: note = N-term fluorescein isothiocyanate
  MOD_RES 1: note = bAla
XIPESSELTLQELLGEER

SEQ ID NO: 815 - protein, 18 aa, synthetic construct
  SITE 1: note = N-term fluorescein isothiocyanate
  MOD_RES 1: note = bAla
XAPESSELTLQELLGEER

SEQ ID NO: 816 - protein, 18 aa, synthetic construct
  SITE 1: note = N-term fluorescein isothiocyanate
  MOD_RES 1: note = bAla
XIAESSELTLQELLGEER

SEQ ID NO: 817 - protein, 18 aa, synthetic construct
  SITE 1: note = N-term fluorescein isothiocyanate
  MOD_RES 1: note = bAla
XIPASSELTLQELLGEER

SEQ ID NO: 818 - protein, 18 aa, synthetic construct
  SITE 1: note = N-term fluorescein isothiocyanate
  MOD_RES 1: note = bAla
XIPEASELTLQELLGEER

SEQ ID NO: 819 - protein, 18 aa, synthetic construct
  SITE 1: note = N-term fluorescein isothiocyanate
  MOD_RES 1: note = bAla
XIPESAELTLQELLGEER

SEQ ID NO: 820 - protein, 18 aa, synthetic construct
  SITE 1: note = N-term fluorescein isothiocyanate
  MOD_RES 1: note = bAla
XIPESSALTLQELLGEER

SEQ ID NO: 821 - protein, 18 aa, synthetic construct
  SITE 1: note = N-term fluorescein isothiocyanate
  MOD_RES 1: note = bAla
XIPESSEATLQELLGEER

SEQ ID NO: 822 - protein, 18 aa, synthetic construct
  SITE 1: note = N-term fluorescein isothiocyanate
  MOD_RES 1: note = bAla
XIPESSELALQELLGEER

SEQ ID NO: 823 - protein, 18 aa, synthetic construct
  SITE 1: note = N-term fluorescein isothiocyanate
  MOD_RES 1: note = bAla
XIPESSELTAQELLGEER

SEQ ID NO: 824 - protein, 18 aa, synthetic construct
  SITE 1: note = N-term fluorescein isothiocyanate
  MOD_RES 1: note = bAla
XIPESSELTLAELLGEER

SEQ ID NO: 825 - protein, 18 aa, synthetic construct
  SITE 1: note = N-term fluorescein isothiocyanate
  MOD_RES 1: note = bAla
XIPESSELTLQALLGEER

SEQ ID NO: 826 - protein, 18 aa, synthetic construct
  SITE 1: note = N-term fluorescein isothiocyanate
  MOD_RES 1: note = bAla
XIPESSELTLQEALGEER

SEQ ID NO: 827 - protein, 18 aa, synthetic construct
  SITE 1: note = N-term fluorescein isothiocyanate
  MOD_RES 1: note = bAla
XIPESSELTLQELAGEER

SEQ ID NO: 828 - protein, 18 aa, synthetic construct
  MOD_RES 1: note = bAla
  SITE 1: note = N-term fluorescein isothiocyanate
XIPESSELTLQELLAEER

SEQ ID NO: 829 - protein, 18 aa, synthetic construct
  SITE 1: note = N-term fluorescein isothiocyanate
  MOD_RES 1: note = bAla
XIPESSELTLQELLGAER

SEQ ID NO: 830 - protein, 18 aa, synthetic construct
  MOD_RES 1: note = bAla
  SITE 1: note = N-term fluorescein isothiocyanate
XIPESSELTLQELLGEAR

SEQ ID NO: 831 - protein, 18 aa, synthetic construct
  SITE 1: note = N-term fluorescein isothiocyanate
  MOD_RES 1: note = bAla
XIPESSELTLQELLGEEA

SEQ ID NO: 832 - protein, 8 aa, synthetic construct
ELTLQELL

SEQ ID NO: 833 - protein, 60 aa, unidentified
  source 1..60: note = Human papillomavirus type 16
RTAMFQDPQERPRKLPQLCTELQTTIHDIILECVYCKQQLLRREVYDFAFRDLCIVYRDG

SEQ ID NO: 834 - protein, 59 aa, unidentified
  source 1..59: note = Human papillomavirus 39
MARFHNPAERPYKLPDLCTTLDTTLQDITIACVYCRRPLQQTEVYEFAFSDLYVVYRDG

SEQ ID NO: 835 - protein, 59 aa, unidentified
  source 1..59: note = Human papillomavirus type 18
MARFEDPTRRPYKLPDLCTELNTSLQDIEITCVYCKTVLELTEVFEFAFKDLFVVYRDS

SEQ ID NO: 836 - protein, 59 aa, unidentified
  source 1..59: note = Human papillomavirus 45
MARFDDPKQRPYKLPDLCTELNTSLQDVSIACVYCKATLERTEVYQFAFKDLCIVYRDC

SEQ ID NO: 837 - protein, 57 aa, unidentified
  source 1..57: note = Human papillomavirus 51
MFEDKRERPRTLHELCEALNVSMHNIQVVCVYCKKELCRADVYNVAFTEIKIVYRDN

SEQ ID NO: 838 - protein, 60 aa, unidentified
  source 1..60: note = Human papillomavirus 56
MEPQFNNPQERPRSLHHLSEVLEIPLIDLRLSCVYCKKELTRAEVYNFACTELKLVYRDD

SEQ ID NO: 839 - protein, 57 aa, unidentified
  source 1..57: note = Human papillomavirus 52
MFEDPATRPRTLHELCEVLEESVHEIRLQCVQCKKELQRREVYKFLFTDLRIVYRDN

SEQ ID NO: 840 - protein, 57 aa, unidentified
  source 1..57: note = Human papillomavirus 33
MFQDTEEKPRTLHDLCQALETTIHNIELQCVECKKPLQRSEVYDFAFADLTVVYREG

SEQ ID NO: 841 - protein, 57 aa, unidentified
  source 1..57: note = Human papillomavirus 58
MFQDAEEKPRTLHDLCQALETSVHEIELKCVECKKTLQRSEVYDFVFADLRIVYRDG

SEQ ID NO: 842 - protein, 56 aa, unidentified
  source 1..56: note = Human papillomavirus 31
MFKNPAERPRKLHELSSALEIPYDELRLNCVYCKGLTETEVLDFAFTDLTIVYRDD

SEQ ID NO: 843 - protein, 57 aa, unidentified
  source 1..57: note = Human papillomavirus 35
MFQDPAERPYKLHDLCNEVEESIHEICLNCVYCKQELQRSEVYDFACYDLCIVYREC

SEQ ID NO: 844 - protein, 59 aa, unidentified
  source 1..59: note = Human papillomavirus 59
MARFEDPTQRPYKLPDLSTTLNIPLHDIRINCVFCKEELQEREVFEFAFNDLFIVYRDC

SEQ ID NO: 845 - protein, 158 aa, unidentified
  source 1..158: note = Human papillomavirus type 16
MHQKRTAMFQDPQERPRKLPQLCTELQTTIHDIILECVYCKQQLLRREVYDFAFRDICIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 846 - protein, 158 aa, unidentified
  source 1..158: note = Human papillomavirus type 18
MARFEDPTRRPYKLPDLCTELNTSLQDIEITCVYCKTVLELTEVFEFAFKDIFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQV

SEQ ID NO: 847 - protein, 18 aa, synthetic construct
  SITE 1: note = N-term fluorescein isothiocyanate
IPESSELTLQELLGEERR

SEQ ID NO: 848 - protein, 17 aa, synthetic construct
  SITE 1: note = N-term 9-fluorenyl acetamido
APESSELTLQELLGEER

SEQ ID NO: 849 - protein, 17 aa, synthetic construct
  SITE 1: note = N-term 9-fluorenyl acetamido
IAESSELTLQELLGEER

SEQ ID NO: 850 - protein, 17 aa, synthetic construct
  SITE 1: note = N-term 9-fluorenyl acetamido
IPASSELTLQELLGEER

SEQ ID NO: 851 - protein, 17 aa, synthetic construct
  SITE 1: note = N-term 9-fluorenyl acetamido
IPEASELTLQELLGEER

SEQ ID NO: 852 - protein, 17 aa, synthetic construct
  SITE 1: note = N-term 9-fluorenyl acetamido
IPESAELTLQELLGEER

SEQ ID NO: 853 - protein, 17 aa, synthetic construct
  SITE 1: note = N-term 9-fluorenyl acetamido
IPESSALTLQELLGEER

SEQ ID NO: 854 - protein, 17 aa, synthetic construct
  SITE 1: note = N-term 9-fluorenyl acetamido
IPESSEATLQELLGEER

SEQ ID NO: 855 - protein, 17 aa, synthetic construct
  SITE 1: note = N-term 9-fluorenyl acetamido
IPESSELALQELLGEER

SEQ ID NO: 856 - protein, 17 aa, synthetic construct
  SITE 1: note = N-term 9-fluorenyl acetamido
IPESSELTAQELLGEER

SEQ ID NO: 857 - protein, 17 aa, synthetic construct
  SITE 1: note = N-term 9-fluorenyl acetamido
IPESSELTLAELLGEER

SEQ ID NO: 858 - protein, 17 aa, synthetic construct
  SITE 1: note = N-term 9-fluorenyl acetamido
IPESSELTLQALLGEER

SEQ ID NO: 859 - protein, 17 aa, synthetic construct
  SITE 1: note = N-term 9-fluorenyl acetamido
IPESSELTLQEALGEER

SEQ ID NO: 860 - protein, 17 aa, synthetic construct
  SITE 1: note = N-term 9-fluorenyl acetamido
IPESSELTLQELAGEER

SEQ ID NO: 861 - protein, 17 aa, synthetic construct
  SITE 1: note = N-term 9-fluorenyl acetamido
IPESSELTLQELLAEER

SEQ ID NO: 862 - protein, 17 aa, synthetic construct
  SITE 1: note = N-term 9-fluorenyl acetamido
IPESSELTLQELLGAER

SEQ ID NO: 863 - protein, 17 aa, synthetic construct
  SITE 1: note = N-term 9-fluorenyl acetamido
IPESSELTLQELLGEAR

SEQ ID NO: 864 - protein, 17 aa, synthetic construct
  SITE 1: note = N-term 9-fluorenyl acetamido
IPESSELTLQELLGEEA

SEQ ID NO: 865 - protein, 12 aa, synthetic construct
ELTLQELLCEER

SEQ ID NO: 866 - protein, 21 aa, synthetic construct
  SITE 1: note = N-term 2-phenyl-4-quinoline carboxylic Acid
  SITE 21: note = C-term allyloxycarbonyl
  MOD_RES 21: note = Lysine(allyloxycarbonyl)
IPESSELTLQELLGEERRAAX

SEQ ID NO: 867 - protein, 21 aa, synthetic construct
  SITE 1: note = N-term 1-(phenylsulfonyl)-1h-indole-2-carboxylic Acid
  SITE 21: note = C-term allyloxycarbonyl
  MOD_RES 21: note = Lysine(allyloxycarbonyl)
IPESSELTLQELLGEERRAAX

SEQ ID NO: 868 - protein, 21 aa, synthetic construct
  SITE 1: note = N-term 4-phthalimido benzoic Acid
  SITE 21: note = C-term allyloxycarbonyl
  MOD_RES 21: note = Lysine(allyloxycarbonyl)
IPESSELTLQELLGEERRAAX

SEQ ID NO: 869 - protein, 21 aa, synthetic construct
  SITE 1: note = N-term 1-phenyl-1h-indole-2-carboxylic Acid
  SITE 21: note = C-term allyloxycarbonyl
  MOD_RES 21: note = Lysine(allyloxycarbonyl)
IPESSELTLQELLGEERRAAX

SEQ ID NO: 870 - protein, 21 aa, synthetic construct
  SITE 1: note = N-term 3-phenyl-1h-indole-2-carboxylic Acid
  SITE 21: note = C-term allyloxycarbonyl
  MOD_RES 21: note = Lysine(allyloxycarbonyl)
IPESSELTLQELLGEERRAAX

SEQ ID NO: 871 - protein, 21 aa, synthetic construct
  SITE 1: note = N-term 9-oxo-10(9h)-acridine acetic Acid
  SITE 21: note = C-term allyloxycarbonyl
  MOD_RES 21: note = Lysine(allyloxycarbonyl)
IPESSELTLQELLGEERRAAX

SEQ ID NO: 872 - protein, 21 aa, synthetic construct
  SITE 1: note = N-term 1h,2h,3h-cyclopenta[b]quinoline-9-carboxylic Acid
  SITE 21: note = C-term allyloxycarbonyl
  MOD_RES 21: note = Lysine(allyloxycarbonyl)
IPESSELTLQELLGEERRAAX

SEQ ID NO: 873 - protein, 21 aa, synthetic construct
  SITE 1: note = N-term tetrahydrocarbazole-8-carboxylic Acid
  SITE 21: note = C-term allyloxycarbonyl
  MOD_RES 21: note = Lysine(allyloxycarbonyl)
IPESSELTLQELLGEERRAAX

SEQ ID NO: 874 - protein, 21 aa, synthetic construct
  SITE 1: note = N-term 2,3,4,9-tetrahydro-1h-carbazole-6-carboxylic acid
  SITE 21: note = C-term allyloxycarbonyl
  MOD_RES 21: note = Lysine(allyloxycarbonyl)
IPESSELTLQELLGEERRAAX

SEQ ID NO: 875 - protein, 21 aa, synthetic construct
  SITE 1: note = N-term 9-methyl-9h-carbazole-3-carboxylic Acid
  SITE 21: note = C-term allyloxycarbonyl
  MOD_RES 21: note = Lysine(allyloxycarbonyl)
IPESSELTLQELLGEERRAAX

SEQ ID NO: 876 - protein, 21 aa, synthetic construct
  SITE 1: note = N-term 9-carbazole acetic Acid
  SITE 21: note = C-term allyloxycarbonyl
  MOD_RES 21: note = Lysine(allyloxycarbonyl)
IPESSELTLQELLGEERRAAX

SEQ ID NO: 877 - protein, 21 aa, synthetic construct
  SITE 1: note = N-term dibenzo[b,d]furan-4-carboxylic Acid
  SITE 21: note = C-term allyloxycarbonyl
  MOD_RES 21: note = Lysine(allyloxycarbonyl)
IPESSELTLQELLGEERRAAX

SEQ ID NO: 878 - protein, 21 aa, synthetic construct
  SITE 1: note = N-term Fluorene-9-acetic Acid
  SITE 21: note = C-term allyloxycarbonyl
  MOD_RES 21: note = Lysine(allyloxycarbonyl)
IPESSELTLQELLGEERRAAX

SEQ ID NO: 879 - protein, 21 aa, synthetic construct
  SITE 1: note = N-term 9-fluorenyl acetamido
  SITE 21: note = C-term 9-fluorenyl acetamido
  MOD_RES 21: note = Lysine(9-fluorenyl acetamido)
IPESSELTLQELLGEERRAAX

SEQ ID NO: 880 - protein, 18 aa, synthetic construct
  SITE 1: note = N-term 9-Fluorene carboxyl
  SITE 18: note = C-term 1-anthracenyl acetamido
  MOD_RES 14: note = Dehydroalanine
  MOD_RES 18: note = Lysine(1-anthracenyl acetamido)
IPQSAELTLQELLXRRKX

SEQ ID NO: 881 - protein, 18 aa, synthetic construct
  SITE 1: note = N-term 9-fluorenyl acetamido
  SITE 18: note = C-term 1-anthracenyl acetamido
  MOD_RES 18: note = Lysine(1-anthracenyl acetamido)
IPQSAEATLQEAAXRRKX

SEQ ID NO: 882 - protein, 4 aa, synthetic construct
  SITE 1: note = N-term fluorescein isothiocyanate
IPES

SEQ ID NO: 883 - protein, 4 aa, synthetic construct
IPES

SEQ ID NO: 884 - protein, 5 aa, synthetic construct
IPESS

SEQ ID NO: 885 - protein, 4 aa, synthetic construct
CCCC

(19) United States
(12) Patent Application Publication
(10) Pub. No.: US 2026/0297131 A1
(43) Pub. Date: Oct. 1, 2026

(54) COMPOSITIONS AND METHODS FOR PEPTIDE-BASED MODULATORS OF HUMAN PAPILLOMAVIRUS ONCOPROTEIN E6

(71) Applicants: Calico Life Sciences LLC, South San Francisco, CA (US); Massachusetts Institute of Technology, Cambridge, MA (US)

(72) Inventors: Xiyun Ye, Sai Ying Pun (HK); Peiyuan Zhang, Boston, MA (US); Jason Tao, Cambridge, MA (US); John C. K. Wang, South San Francisco, CA (US); Amirhossein Mafi, South San Francisco, CA (US); Nathalie M. Grob, Cambridge, MA (US); Anthony J. Quartararo, Boston, MA (US); Hannah T. Baddock, South San Francisco, CA (US); Ian Foe, South San Francisco, CA (US); Andrei Loas, Boston, MA (US); Dan L. Eaton, South San Francisco, CA (US); Qi Hao, Foster City, CA (US); Aaron H. Nile, South San Francisco, CA (US); Bradley L. Pentelute, Revere, MA (US)

(21) Appl. No.: 19/390,189

(22) Filed: Nov. 14, 2025

Related U.S. Application Data
(63) Continuation of application No. PCT/US2024/029542, filed on May 15, 2024.
(60) Provisional application No. 63/502,313, filed on May 15, 2023.

(51) Int. Cl. C07K 7/08; C07K 14/005

(52) U.S. Cl. CPC C07K 7/08; C07K 14/005; C07K 2319/00; C12N 2710/20022

(57) ABSTRACT

This disclosure relates to synthetic peptides of E6AP and complexes of synthetic peptides with HPV E16, methods of making such peptides and complexes, and methods of using such peptides and complexes for blocking, inhibiting, or preventing the interaction of E6AP with HPV E16. The synthetic peptides and complexes of synthetic peptides with HPV E16 can further comprise warheads to introduce covalent linkages between the synthetic peptides with HPV E16.

### CROSS REFERENCE TO RELATED APPLICATIONS

This application is a continuation of International (PCT) Patent Application No. PCT/US2024/029542 filed on May 15, 2024, which claims priority to and the benefit of U.S. Provisional Patent Application No. 63/502,313, filed on May 15, 2023, the entire contents of each of which are incorporated by reference herein for all purposes.

### SEQUENCE LISTING

The instant application contains a Sequence Listing which has been filed electronically in XML format and is hereby incorporated by reference in its entirety. Said XML file copy, created on Sep. 10, 2024, is named CLS-035WO_SL.xml. and is 2,110,761 bytes in size.

### BACKGROUND

Human papillomavirus (HPV) infections account for nearly all cervical cancer cases, the fourth most common cancer in women worldwide. High-risk forms of HPV are causative in multiple cancers, including cervical, vaginal, oropharyngeal, and potentially a subset of prostate cancers. Despite the introduction of HPV-directed vaccines over ten years ago, the lack of their widespread distribution, uptake by the general population, and availability continues to make HPV positive (HPV+) cancers prevalent. Among ~200 identified HPV strains, high-risk HPV16 and HPV18 are responsible for ~75% of HPV-associated cervical cancers. Current treatment strategies for HPV cancers include radiation therapy, surgery, chemotherapy, monoclonal antibody(mAb), and checkpoint blockade. There are no currently approved targeted therapeutics against HPV.

### SUMMARY

In one aspect, the disclosure provides for a synthetic peptide comprising an amino acid sequence IPESSELTLQELLGEER (SEQ ID NO: 1) or ELTLQELLGEER (SEQ ID NO: 5). In some embodiments, the N- or C-terminus of the peptide is modified with a chemical moiety. In some embodiments, the chemical moiety is attached at the N-terminus and is selected from the group of Fluorescein-5-Isothiocyanate, 9-fluorene acetamido, 1-fluorene acetamido, 1-Indane acetamido, 9-fluorenone-2-carboxamido, 9-fluorenone-1-carboxamido, 9-fluorenone-4-carboxamido, anthraquinone-2-carboxamido, xanthene-9-carboxamido, 1-anthracene carboxamido, 2-anthracene carboxamido, 1-adamantane carboxamido, Triphenyl acetamido, Diphenyl acetamido, 1-naphthyl carboxamido, 1,6-dihydrophenyl carboxamido, Pentafluorophenyl carboxamido, 6-hydroxy-2-naphthyl carboxamido, 1-pyrenebutyl carboxamido, 5-Acenaphthene carboxamide, 7-Methoxycoumarin-4-acetamido, 4-phenyl-phenylalanine, and Cyclohexyl-alanine. In some embodiments, the chemical moiety is attached at the C-terminus and is selected from the group of Fluorescein-5-Isothiocyanate, fluorene-9-acetamido, fluorene-1-acetamido, 1-Indanecarboxamido, 9-fluorenone-2-carboxamido, 9-fluorenone-1-carboxamido, 9-fluorenone-4-carboxamido, anthraquinone-2-carboxamido, xanthene-9-carboxamido, 1-anthracene carboxamido, 2-anthracene carboxamido, 1-adamantane carboxamido, triphenylacetamido, diphenylacetamido, 1-naphthyl carboxamido, and Exo-norbomene-carboxamido. In some embodiments, the chemical moiety is selected from the group of 9-fluorenyl, 1-fluorenyl, 1-Indanyl, 9-oxofluoren-3-yl, 9-oxofluoren-4-yl, 9-oxofluoren-1-yl, 2-anthraquinonyl, 9-xanthyl, 1-anthracenyl, 2-anthracenyl, Adamantyl, Triphenylmethyl, Diphenylmethyl, 1-naphthyl, 2,6-dihydroxy-phenyl, Pentafluoro-phenyl, 6-Hydroxynaphthyl, 1-pyrenebutyl, 5-acenaphthyl, Coumarin, Biphenyl, Cyclohexyl, Norborenyl, and Fluorescein. In some embodiments, a first chemical moiety is attached at the N-terminus of the peptide and a second chemical moiety is attached at the C-terminus of the peptide. In some embodiments, a first chemical moiety is selected from the group of Biotin-PEG4, Fluorene, Anthracene, and Fluorene-(Biotin-PEG4-)K, and the second chemical moiety is selected from the group of 9-fluorene, 5-Acenaphthene, 1-naphthanlene, 2-anthraquinone, and 1-Anthracene. In some embodiments, the peptide comprises Fluorene-IPESSELTLQELLGEERRAA-K(1-Anthracene (SEQ ID NO:2) or Fluorene-IPESAELTLQELLGEERRAA-K(1-Anthracene (SEQ ID NO:3).

In another aspect, the disclosure provides for a synthetic peptide modified with a warhead, and the peptide is selected from TABLE 2C, TABLE 2D, or TABLE 2E. In some embodiments, the warhead is at position 9 with respect to SEQ ID NO: 3. In some embodiments, the warhead is selected from the group of phenylacrylamide (Ph-acr), Dap-acrylamide (dap-acr), Dab-acrylamide (dab-acr), Dap-propiolamide (dap-ppa), and dehydroalanine Dha. In some embodiments, the peptide comprises Fluorene-IPQSAELTLQELL(DHA)RRKKK(Anthracene) (SEQ ID NO: 4). In some embodiments, the warhead is selected from the group of phenyl fluorosulfate (FS), phenyl sulfonyl fluoride(SF), phenyl Carbamate (p-PhC), phenyl Carbamate (m-PhC), and disulfide (DS).

In another aspect, the disclosure provides for a synthetic peptide comprising an amino acid sequence X1X2X3X2QX1X2X2CEER (SEQ ID NO: 6), wherein X1 is Nva, Leu, Aoc, Cpa, Cba, Cha, Phg, Hof, or Naf; X2 is Aad, Glu, or Cya; X3 is Thr, Asn, Hyp, Cpg, Cbg, or Ceg; and X4 is d-Ala, Aib, Gly, or Gln. In some embodiments, the peptide is selected from TABLES 3-31. In some embodiments, the peptide is selected from SEQ ID NO: 7-19. In some embodiments, the peptide comprises SEQ ID NO: 17.

In another aspect, the disclosure provides for a synthetic peptide comprising an amino acid sequence Aad-X1-X2-Leu-Aib-Aad-Cba-Leu-Cys-X3-X3-X3 (SEQ ID NO: 20) wherein X1 is PhF, Naf, Qua, Clw, or Trp; X2 is Chg or Ceg; and X3 is d-Ala, Aib, Gly, Ser, Aad, Glu, Gln, Hoc, Val, Leu, Met, Pro, Dap, Lys, Arg, His, Trp, Phe, Tyr, or Null. In some embodiments, the peptide is 11 or 12 amino acids long. In some embodiments, the N-terminus of the peptide is further modified with a modifier selected from 2-Phenyl-4-quinolinecarboxylic acid, 1-(Phenylsulfonyl)-1H-indole-2-carboxylic acid, 6-Fluoro-2-naphthoic acid, 4-phthalimidobenzoic acid, Xanthene 9-carboxylic acid, 1-Pyrenebutyric acid, 5-Acenaphthenecarboxylic acid, 1-Phenyl-1H-indole-2-carboxylic acid, Indole-2-carboxlic acid, quinoline-4-carboxylic acid, and 10-(Carboxymethyl)-9(10H)acridone. In some embodiments, the peptide is selected from TABLES 32-39.

In another aspect, the disclosure provides for a synthetic peptide comprising the formula

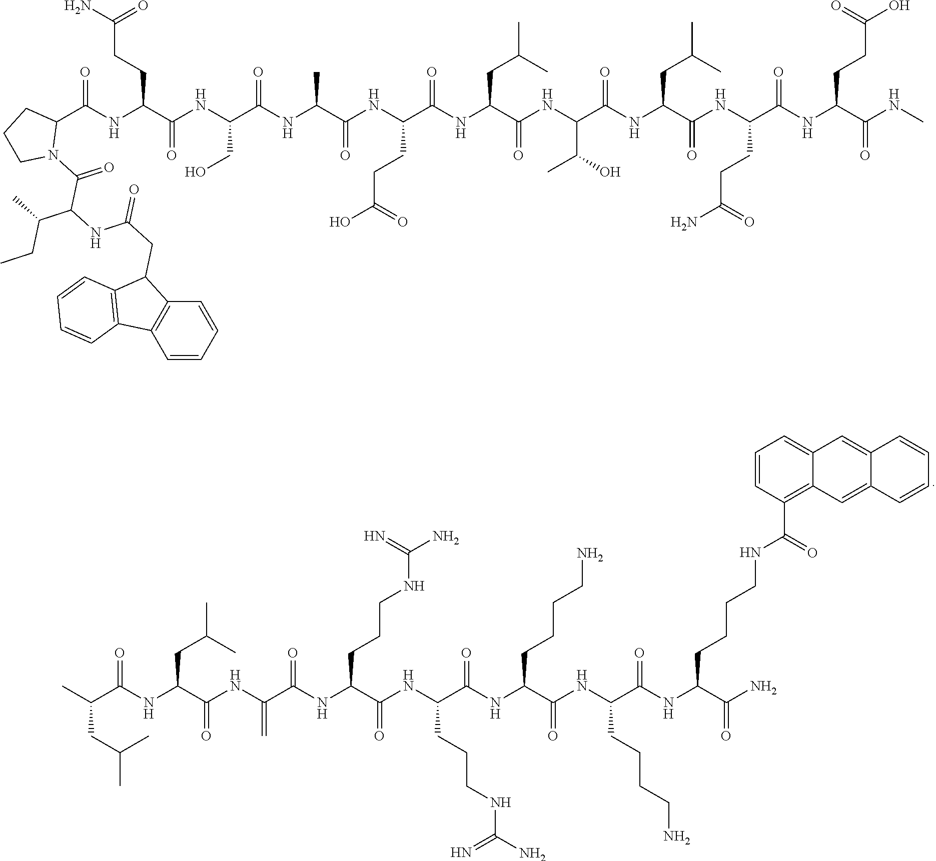

In some embodiments, the peptide further comprises a warhead. In some embodiments, the warhead is selected from the group of phenylacrylamide (Ph-acr), Dap-acrylamide (dap-acr), Dab-acrylamide (dab-acr), Dap-propiolamide (dap-ppa), and dehydroalanine Dha.

In some embodiments, the synthetic peptide is an HPV16E6/E6AP specific inhibitor.

In one aspect, the disclosure provides for a synthetic peptide disclosed herein, comprising one or more additional modifications selected from: acetylated, formylated, propanoylated, hexanoylated, or myristoylated N-terminus; amidated C-terminus; substitution of one or more L-amino acid with a D-amino acid; substitution of one or more amino acid with a methyl-amino acid; and substitution of an α-amino acid with a β-amino acids.

In one aspect, the disclosure provides for a synthetic peptide/HPV16E6 complex, wherein the peptide is selected from any one of the synthetic peptides disclosed herein. In some embodiments, the synthetic peptide and the HPV16E6 in the complex are covalently linked. In some embodiments, the synthetic peptide is covalently linked to amino acid residue Cys-58, Arg-84, or Arg-13 of HPV16E6. In some embodiments, the peptide/HPV16E6 complex disclosed herein the complex is inhibited in binding of human E6AP.

In some embodiments, a pharmaceutical composition comprising the synthetic peptide described herein and a pharmaceutically acceptable salt or carrier is provided.

### BRIEF DESCRIPTION OF THE DRAWINGS

A better understanding of the features and advantages of the present disclosure will be obtained by reference to the following detailed description that sets forth illustrative embodiments, in which the principles of the disclosure are utilized, and the accompanying drawings of which:

FIGS. 1A-1F depict rational design of E6AP-mimicking peptides. (FIG. 1A) Left: X-ray crystal structure of the ternary protein complex formed by 16E6 (“E6”), MBP fused to the E6AP LXXLL peptide (dark grey), and p53. PDB: 4XR8. Right: Interaction interface of 16E6 and the LXXLL motif of E6AP. The E6AP LXXLL peptide binds the 16E6 hydrophobic groove and drives peptide recognition. (FIG. 1B) Rational design of high-affinity 16E6 binding peptide. The N-terminal and C-terminal lysine side chain primary amines were modified with various small molecules listed in FIG. 1C. (FIG. 1C) Chemical library of small molecules used for modifying E6AP-based peptides. 9-Fluorenyl, 1-fluorenyl, 9-oxofluoren-3-yl, 9-oxofluoren-4-yl, 9-oxofluoren-1-yl, 2-anthraquinonyl, 9-xanthyl, 1-anthracenyl, 2-anthracenyl, 1-naphthyl, 6-Hydroxynaphthyl, 1-pyrenebutyl, 5-acenaphthyl, and Fluorescein are the planar aromatic residues; Adamantyl, Biphenyl, Cyclohexyl, and Norborenyl are the hydrophobic residues; and the remaining indicated are other aromatics. (FIG. 1D) (SEQ ID Nos: 26-27,806-812 and 879) Table of N- and C-terminal modified E6AP peptide mimics. The binding dissociation constant K_{D} is measured by bio-layer interferometry (BLI) competition assay, except for *K_{D} measured by direct BLI. (FIG. 1E) BLI competition assay was used to measure the binding of 6′ and 6′-3L3A. Streptavidin tips were immobilized with 1-biotin and dipped into a solution of various concentration of analyte and 16E6. (FIG. 1F) Direct BLI measurement of 6′-biotin, parameters are estimated to be: kₒₙ=1.5E+05 M-1s-1, k_{off}=4.3E-03 s⁻¹, and K_{D}=3.0±1.8 nM.

FIGS. 2A-2C depict the determination of peptide 1 and 16E6 binding constant by direct binding assay in BLI and competition BLI binding assay. (FIG. 2A) Structure of peptide 1 and peptide 1-biotin (FIG. 2B) BLI measurement of 1-biotin and 16E6, parameters are estimated to be: kₒₙ=40200 M⁻¹s⁻¹, k_{off}=0.0851 s⁻¹ and K_{D}=2.1±0.1 μM (N=3). Indicated MBP-16E6 protein concentration was used in each channel on the right. kₒₙ=on rate constant, k_{off}=off rate constant, K_{D}=kinetic apparent dissociation constant. (FIG. 2C) Peptide 1 binding was assessed by competition binding assay. Various concentrations of peptide 1 were mixed with 30 nM MBP-16E6 protein. Peptide 1-biotin was immobilized onto the streptavidin sensor tips to compete for MBP-16E6 protein with various concentrations of unlabeled peptide 1 in the solution (33333 nM, 8333 nM, 2083 nM, 520 nM, 130 nM, 32 nM, 2.0 nM or 0.1 nM). The dissociation binding constant was estimated to be K_{D}=2.3±0.5 μM. Error is the curve fitting standard error of the mean (SEM) reported by the Prism 8 software (N=3).

FIG. 3 (SEQ ID Nos: 813-831 and 883) shows peptides used in alanine scanning of the N-terminal modified E6AP peptide. 17 single alanine mutants of E6AP peptides were synthesized. Binding affinity was measured by competition BLI binding assay (N=3). *NB: no binding. *bAla=beta-Alanine

FIGS. 4A and 4B show structure-activity-relationship(SAR) study of small molecule modifications. (FIG. 4A) N-terminal modifications SAR. α: modification is not sensitive to ring substitution position. β: tricyclic, planar, and aromatic molecules improve the affinity to the low nano-molar range. *: Removing a phenyl ring from the structure or breaking the strain between two phenyl moieties will decrease the binding by 30-50 folds. (FIG. 4B) C-terminal modifications SAR. α: binding improvement of anthracene modification is not sensitive to ring substitution position. α: two phenyl rings are required for a mid-micromolar binding. *: Removing a phenyl ring from the structure, breaking the strain between two phenyl moieties, will decrease the binding by 30-50 folds. K_{D} values are measured by BLI binding assay in a competition mode.

FIG. 5A shows the structure of peptide 6′-biotin (6′-biotin).

FIG. 5B shows a direct BLI binding assay of 6′-biotin against SUMO-MDM2.

FIG. 5C shows a direct BLI binding assay of peptide 6′-biotin against THRA.

FIG. 6A shows the analysis of the main binding interface of 16E6 and E6AP. Cys58 of 16E6. Residues of E6AP were colored based on their measured reactivity towards Cys58 of 16E6: Gly9, Glu10, Glu11, and Arg12.

FIG. 6B shows structures of electrophiles.

FIG. 6C shows a scheme of the bind-and-react strategy. Structure of peptide E3 containing a Dha highlighted in grey.

FIG. 6D shows the apparent kinetic constant kₐₚₚ calculated from the time-course of protein depletion assay, where 50 nM MBP-16E6 was incubated with different concentrations of E3.

FIG. 6E shows a series value of Kₐₚₚ that was plotted against the corresponding E3 concentration to estimate k_{inact} and Kᵢ.

FIG. 7A (SEQ ID Nos: 110-121) shows cross-linking experiments between MBP-E6 and peptides E0-E11. All reactions were performed using 1×PBS, pH=7.4, as the buffer. Conditions of crosslinking experiments were listed. Cross-link yield was interpolated using LC-MS protein deconvolution mass spectrum. The percentage was obtained by dividing the peak area of cross-linked protein by the sum of uncross-linked and cross-linked protein peak area (N=2).

FIG. 7B shows the structure of electrophiles.

FIG. 8 (SEQ ID Nos: 122-129) shows as sequence table and results of a binding experiments of E6AP-mimicking peptides (peptides 7-13). Apparent Kᵢ is determined by BLI. Peptides were in competition with immobilized 1-Biotin following a 30 min incubation with 16E6. Dha: dehydroalanine.

FIGS. 9A-9E show examples of affinity matured peptide for improved reactivity. FIG. 9A shows the main binding interface of 16E6 and E6AP. Cys58 of 16E6 is targeted by an electrophile substituted at Gly14. FIG. 9B (SEQ ID Nos: 128-129) shows sequence table of E6AP-mimicking peptide 13 and peptide 13-3L3A (negative control peptide). Apparent Kᵢ is determined by BLI. n.b. (non-binding). Peptides were in competition with immobilized 1-Biotin following a 30 min. incubation with 16E6. Dha: dehydroalanine. Structure of peptide 13, Dha, tri-leucine. FIG. 9C shows the results of a BLI competition assay measurement of 13 and 13-3L3A estimated apparent Kᵢ in unit of nM. FIG. 9D shows the apparent kinetic constant kₐₚₚ calculated from the time-course of protein depletion assay, where 10 nM MBP-16E6 was incubated with different concentration of 13.

FIG. 9E shows a series value of Kₐₚₚ was plotted against the corresponding peptide 13 concentration to estimate k_{inact} and Kᵢ.

FIGS. 10A-10E show crosslinking of reactive peptides to 16E6. FIG. 10A shows a crosslinking reaction of MBP-16E6 (1 μM) and reactide 13 (3 μM). Quantitative mono-crosslinked MBP-16E6-13 was observed at 2 h. (FIG. 10B) Crosslinking of MBP-16E6 C58S (1 μM) and peptide 13 (3 μM), no appreciable amount of reaction product was observed after 12 h. FIG. 10C shows crosslinking of MBP-16E6 (1 μM) and peptide 13-3L3A (3 μM), no appreciable amount of reaction product was observed after 12 h. FIG. 10D shows protein-selective intermolecular crosslinking of MBP-16E6 in a protein mixture. Only 16E6 was modified, as indicated by the LC-MS analysis. FIG. 10E shows a BLI assay determined 16E6/E6AP complex formation. 1 μM of Biotin-E6AP protein was immobilized onto streptavidin tip and dipped into 1 μM of MBP-16E6 only, MBP-16E6 mixed with 1 μM of 13 or 1 μM of 13-3L3A.

FIG. 11 shows the control peptide 13-3L3A crosslink to THRA. Crosslink reaction of 1 μM THRA (PDB: 1nav) and 10 μM 13-3L3A, at 37° C. for 12h. PDB codes of structures used: 1nav.

FIG. 12A shows the structure of 13-biotin

FIG. 12B shows a protein deconvolution mass spec of MBP-16E6 conjugates with peptide 13-biotin (Molecular Weight: 3231.90).

FIG. 12C shows a 13-biotin direct K_{D} is determined by BLI assay. k_{off}=6.79E-05 s⁻¹, kₒₙ=5.31E+04 M⁻¹s⁻¹, apparent direct K_{D}=1.3±0.6 nM (N=3).

FIG. 12D shows a 13-biotin association and dissociation to THRA as determined by direct BLI assay (N=1).

FIG. 12E shows a 13-biotin association and dissociation to MDM2 as determined by direct BLI assay (N=1).

FIG. 12F shows the structure of 13-3L3A-biotin

FIG. 12G shows no observable crosslink between MBP-16E6 and 13-3L3A-biotin. Protein deconvolution mass spec of 1 μM MBP-16E6 that were treated with 20 μM 13-3L3A-biotin (Molecular Weight: 3103.69) for 12 h in 1×PBS at 37° C. Mass-spec was recorded using a 6550 Agilent Q-TOF coupled to Infinite II 1290 LC system, with an Aeries C4 column (Phenomenex) using 1 to 61 B % linear ramping gradients.

FIG. 12H shows 13-3L3A-biotin showing no observable association to 1 μM and 0.5 μM MBP-16E6 as determined by direct BLI assay, where 13-3L3A-biotin was immobilized onto the streptavidin tips and dipped into MBP-16E6 solutions.

FIGS. 13A-13F show molecular modeling of the 16E6-13 conjugate. (FIG. 13A(SEQ ID NO: 880)) Structural representation of 16E6 (white) bound to peptide 13 (dark grey) featuring the Dha warhead (black), obtained through ~120 ns MD simulation. Structure represents the averaged configurations of 13 within calculated clusters (see methods). (FIG. 13B (SEQ ID NO: 832)) Trajectory analysis of root mean squared differences (r.m.s.d) during MD simulatioms, showing stability and modest conformational changes of the complex. FIG. 13C (SEQ ID NO: 832) shows a comparison of molecular modeling of the 13:16E6 complex with the X-ray structure of 16E6 (coral)-E6AP LXXLL peptide (yellow), p53 (plum) complex (PDB ID: 4XR8). All r.m.s.d. were calculated for the Ca atoms. All structures were compared to the initial struture. FIG. 13D shows intermolecular interactions between 16E6 (white) and 13 (blue), identified and analyzed from the MD simulation. Interactions shown in FIG. 13D are persistent during the ~120 ns MD simulation. Dotted lines represent hydrogen bonding. (FIG. 13E) Covered surfaces by the N term [within 5 Å]: Patch1 in black [V38,Y39,C40,K41, R62,E63], Patch2 in yellow [C73,F76,Y77,I80, Y83,R84,H85,R136]. Left: E6 and 13 N-term detailed interactions. FIG. 13F shows covered surfaces by the Cterm [within 5A]: Patch1 [M8,F9,Q10, D11, P12, Q13, E14, R15, P16, R17, K18,L19, P20,Q21,D25]. Patch2 in yellow [A53,D56,R55,L57, C58*]. Patch3 in purple [Y99, K101, D105,L107, I108, R109, C110, C113,Q114,K115, P116,L117, R136,R138,W139,T140]. Left: E6 and C-term 13 detailed interactions.

FIGS. 14A-14B show an illustration of the native E6AP peptide. (FIG. 14A) E6AP peptide bound to 16E6 protein, surrounded by ~6K water molecules (lines) and ions (spheres). (FIG. 14B) Comparison of X-ray structure of 16E6 (light grey, top) bound to E6AP (light grey, bottom) (PDB ID: 4XR8) with relaxed 16E6 (black/dark grey, top)-E6AP peptide (dark grey, bottom) complex after 1.1 s MD simulation. P53 and MBP were omitted for clarity. The r.m.s.d. was calculated for the Cα atoms.

FIGS. 15A-15D show an illustration of structural modeling of 16E6-bound Peptide-13. (FIG. 15A(SEQ ID NO: 880)) Structure of Peptide-13. (FIG. 15B) E6AP native core adopts an alpha helical conformation in Peptide-13 structural model obtained by computational conformational sampling. (FIG. 15C) molecular docking of Peptide-13 to 16E6 protein. (FIG. 15D) Molecular modeling of 16E6-Peptide-13 complex, featuring the covalent link between Peptide-13 warhead (Dha) and the 16E6 hotspot (Cys58).

FIG. 16 shows an illustration of molecular docking of the native E6AP peptide in the 16E6 (left). The MOE molecular docking procedure accurately describes the interactions between 16E6 and E6AP native peptide (right).

FIG. 17 (SEQ ID NO: 832) shows an illustration of molecular modeling of 16E6-Peptide-13 complexes. The structures are ordered from left to right based on: the largest binding affinity between the peptide-13 core sequence and 16E6 and the lowest r.m.s.d of the peptide-13 core sequence compared to the crystallographic native E6AP peptide.

FIG. 18 shows Reactide 13 stability in media stability assay. Ten μM peptide was incubated in RPMI with 10% FBS at 37° C. for the indicated time. Proteins were removed from the mixture and analyzed by LC-MS. Percentage of the remaining peptide was estimated by dividing the area under extract ion chromatogram (EIC) to the area of time zero (N=3).

FIGS. 19A-B (SEQ ID Nos:833-846) show protein sequence alignment of high-risk type HPV E6 using CLUSTAL O (1.2.4) sequence alignment tool. The alignment includes for the following high-risk subtypes: 18, 31, 33, 35, 39, 45, 51, 52, 56, 58, 59, 66, and 68. For example, FIG. 19B shows the protein sequence alignment of HPV16E6 and HPV18 E6. In the conserved regions, HPV16E6 (48-64) and HPV18E6 (43-59), the Phe53 of HPV18 E6 aligns with the Cys58 of HPV16 E6. This Phe52 residue made HPV18E6 insensitive to cysteine reactive bind-and-react inhibition strategy.

FIG. 20 shows the structures of various peptides as indicated.

FIG. 21 left panel shows direct BLI measurement of Biotin-PEG4-ELTLQELLGEER (SEQ ID NO: 41). Biotin ligand was immobilized onto the sensor tip. BLI was recorded after incubating various concentrations of MBP 16E6. Right panel shows FP binding assay of FITC-ELTLQELLGEER (SEQ ID NO: 42). FP was recorded after incubating various concentrations of MBP 16E6 with 100 nM FITC-ELTLQELLGEER (SEQ ID NO: 42). FP was performed with a BioTek H1 plate reader. Excitation: 485/20, Emission: 528/20. (Green) Filter set. Read Height: 10.5 mm. Gain sets to 50. Temperature 27° C.

FIG. 22 (SEQ ID Nos: 7, 27 and 847) shows the structure of the E3 peptide (ELT-G9DHA) and BLI competition assay, and FP competition assay. The K_{D} was measured by competition BLI binding assay. Biotin-peg4-IPESSELTLQELLGEER (SEQ ID NO: 43) was immobilized to the SA tip. Various concentrations of analyte in the solution compete with the immobilized peptide for 40 nM MBP-16E6. N=2, competition curves are shown. IC₅₀ is measured by competition FP assay. Various concentrations of analyte peptide were mixed with 100 nM FITC-IPESSELTLQELLGEER (SEQ ID NO: 44) and 450 nM MBP-16E6 solution, incubated for 30 min. Polarization was recorded using BioTek H1. N=2 biological replicates, competition curves are shown. FP was performed with a BioTek H1 plate reader. Excitation: 485/20, Emission: 528/20. (Green) Filter set. Read Height: 10.5 mm. Gain sets to 50. Temperature 27° C.

FIG. 23 (SEQ ID Nos: 44, 848-864, and 882) shows the_{D}-alanine scanning of IPESSELTLQELLGEER (SEQ ID NO: 1) peptide. K_{D} was measured by competition BLI binding assay. Biotin-peg4-IPESSELTLQELLGEER (SEQ ID NO: 43) was immobilized to the SA tip. Various concentrations of analyte in the solution compete with the immobilized peptide for 40 nM MBP-16E6. Representative competition curves are shown.

FIG. 24 (SEQ ID Nos: 6 and 865) shows the E6 Library I design with a library size of 1.42 M.

FIG. 25A shows an illustration of ReAct-ASMS platform. (FIG. 25B) Mix disulfide peptides with different R group showcased different crosslink rate. Right: time-course of crosslink reaction between reactides and MBP 16E6 protein, monitored by LC-MS. At 6 h, cysteamine (pβME) showed a crosslink yield of 76%. (FIG. 25C(SEQ ID NO: 865)) A scheme of the bind-and-react reaction and dissociating peptide-protein conjugates. Kᵢ denotes the binding constant. K_{inact} denotes first-order rate. Peptides were dissociated from protein by DTT and subsequently sequenced by an nLC-MSMS. Right: Mass-spec analysis of dissociating peptide-protein conjugates.

FIG. 26A(SEQ ID Nos: 7-19) shows a sequence table of resynthesized 16E6 binding peptides. Apparent Kᵢ is determined by BLI. Peptides were in competition with immobilized 1-Biotin following a 30 min incubation with 16E6. IC₅₀ was determined by FP. Peptides were in competition with 1-FITC following a 30 min incubation with 16E6.

FIG. 26B shows competitive crosslink assay. MBP-16E6 (1 μM) and Dha peptide mixture (12 μM), mono-crosslinked MBP-16E6-18 was observed at 2 h with a yield of 83%.

FIG. 27A illustrates the structure of peptides 18 and 23, dehydroalanine and unnatural amino acids (highlighted). Apparent Kᵢ is determined by BLI. (FIGS. 27A and 27C) Peptides were in competition with immobilized 1-Biotin following a 30 min incubation with 16E6. IC₅₀ was determined by FP. (FIGS. 27B and 27D) Peptides were in competition with 1-FITC following a 30 min incubation with 16E6.

FIG. 27B shows the structure of peptides 14, 18 and 23, dehydroalanine and unnatural amino acids (highlighted). 14: apparent Kᵢ of 1.3±0.4 μM. IC₅₀ of 766±180 nM. 18: apparent Kᵢ of 177±29 nM, IC₅₀ of 330±55 nM. 23: apparent Kᵢ of 179±33 nM, IC₅₀ of 389±109 nM. Apparent Kᵢ is measured by competition BLI binding assay. Biotin-peg4-IPESSELTLQELLGEER (SEQ ID NO: 43) was immobilized to the SA tip. Various concentrations of analyte in the solution compete with the immobilized peptide for 40 nM or 100 nM MBP-16E6. N=2, competition curves are shown. IC₅₀ is measured by competition FP assay. Various concentration of analyte was mixed with 100 nM FITC-IPESSELTLQELLGEER (SEQ ID NO: 44) and 450 nM MBP-16E6 solution, incubated for 30 min. FP was performed with a BioTek H1 plate reader. Excitation: 485/20, Emission: 528/20. (Green) Filter set. Read Height: 10.5 mm. Gain sets to 50. Temperature 27° C.

FIG. 27C (SEQ ID NO: 43) shows graphs depicting BLI competition assays of peptide ELTLQELL(Dha)EER (SEQ ID NO: 7), peptide 14, peptide 23, peptide 18, and peptide 18-3L3A.

FIG. 27D (SEQ ID NO: 847) shows graphs depicting FP competition assays of peptide ELTLQELL(Dha)EER (SEQ ID NO: 7), peptide 14, peptide 23, peptide 18, and peptide 18-3L3A (order from top to bottom).

FIG. 28A shows a crosslinking reaction of MBP-16E6 (1 μM) and reactide 18 (10 μM). Quantitative mono-crosslinked MBP-16E6-18 was observed at 4 h.

FIG. 28B shows crosslinking of MBP-16E6 (1 μM) and peptide 18-3L3A (10 μM).

FIG. 28C shows crosslinking of MBP-16E6 C58S (1 μM) and peptide 18 (10 μM).

FIG. 28D shows crosslinking of MBP-16E6 L57A (1 μM) and peptide 18 (10 μM).

FIG. 28E shows apparent kinetic constant kₐₚₚ was calculated from the time-course of protein depletion assay, where 25 nM MBP-16E6 was incubated with different concentration of 18.

FIG. 28F shows a series value of Kₐₚₚ was plotted against the corresponding reactide 18 concentration to estimate k_{inact} and Kᵢ. k_{inact} of 0.018 s-1, Kᵢ value of 792 nM and k_{inact}/Kᵢ ratio of 22.7 mM⁻¹ s⁻¹.

FIGS. 29A-29B (SEQ ID Nos: 866-878) show a small molecule library of IPESS (SEQ ID NO: 884) peptides. K_{D} is measured by competition BLI binding assay. Biotin-peg4-IPESSELTLQELLGEER (SEQ ID NO: 43) was immobilized to the streptavidin (SA) tips. Various concentrations of analyte in the solution compete with the immobilized peptide for 40 nM MBP-16E6. (N=2).

FIG. 30 (SEQ ID Nos: 25 and 777) shows structures of exemplary peptide 18 analogs. Apparent Kᵢ is determined by competition BLI binding assay. Biotin-PEG4-IPESSELTLQELLGEER (SEQ ID NO: 43) was immobilized to the streptavidin (SA) tips. Various concentrations of analyte compete with the immobilized peptide for 40 nM or 100 nM MBP-16E6 in the solution (n=2). Competition curves are shown.

FIG. 31A (SEQ ID Nos: 25 and 777) shows the structure of reactide 18's analogs.

FIG. 31B shows crosslink assay: MBP-16E6 (1 μM) and Dha peptide (4 μM), mono-crosslinked MBP-16E6-18 was observed at 1 h with a yield of 83% (18-Naf2car), 85% (18-car-IPQSA (SEQ ID NO: 777)) and 76% (18-car-IPQSA (SEQ ID NO: 777)-truncEER).

FIG. 31C (SEQ ID NO: 777) shows the IC₅₀ is measured by competition FP assay. Various concentration of analyte peptide was mixed with 100 nM FITC-IPESSELTLQELLGEER (SEQ ID NO: 44) and 450 nM MBP-16E6 solution, incubated for 30 min. FP was performed with a BioTek H1 plate reader. Excitation: 485/20, Emission: 528/20. (Green) Filter set. Read Height: 10.5 mm. Gain sets to 50. Temperature 27° C.

FIG. 32 shows E6 Library II design. Above: library II structure. Below: monomer structures in randomized position 2, 3, 10, 11, and 12.

FIGS. 33A-33D (SEQ ID NO: 880) show a design of targeting R84 on HPV16 E6 from the position of A6 in the IPQSA (SEQ ID NO: 777) reactide and targeting R138 from the position of R16.

FIGS. 33E-33F show a competition experiment for Pep1 and Pep4.

FIG. 33G shows the structures of Pep1 and Pep4.

FIGS. 34A and 34B show cell toxicity assays towards HPV16 E6 positive cell lines of reactide 13 in SiHa and CaSki cells and control cell lines lacking 16E6 expression: HeLa and c-33a. At 96 hr, cell viability was estimated using MTT assay. Concentration of the compounds was 20 μM, 10 μM, 5 μM, 2.5 μM, 1.25 μM, 0.4 μM, and 0.08 μM.

FIG. 35 (SEQ ID Nos: 128 and 881) shows p53 protein levels in CaSki cells treated with reactide 13.

FIGS. 36A and 36B shows the results from LC-MS and MALS and gel staining for E E6AP, MBP-16E6 (4C4S), and MBP-16E6 (4C4S C58S).

FIGS. 37A-37D show crosslinking of 13-TAMRA to MBP-16E6 (4C4S) and MBP-16E6 C58S (4C4S) in HPV-negative HT1080 lysate. HT1080 lysates (50 μg) were incubated overnight at 4° C. with 2 μM each of MBP-16E6 WT or C58S (each 4C4S), and 13-TAMRA (10 μM). The mixtures were then separated by SDS-PAGE and transferred onto nitrocellulose. Fluorescence scans of the protein gel provided a visualization of both the free 13-TAMRA and 13-TAMRA crosslinked to WT MBP-16E6 with (FIG. 37A) short and (FIG. 37B) long exposure times. Additional minor fluorescent signals from 13-TAMRA crosslinked to unidentified proteins in the lysate at FIG. 37B. (FIG. 37C) Ponceau S staining of the nitrocellulose membrane after transfer displayed the combination of MBP-16E6 (either WT or C58S), 13-TAMRA, and HT1080 lysate. (FIG. 37D) The Western blot for MBP-16E6 and GAPDH, alongside a 488 nm fluorescence scan for 13-TAMRA, showed selective binding of 13-TAMRA to MBP-16E6 WT but not C58S.

FIGS. 38A-38D show crosslinking of 13-biotin to MBP-16E6 (4C4S) and MBP-16E6 C58S (4C4S) in HPV-negative HT1080 lysate. HT1080 lysates (50 μg) were incubated overnight at 4° C. with 2 μM each of MBP-16E6 WT or C58S (each 4C4S), and 13-biotin (10 μM) or 13-3L3A-biotin (10 μM). The mixtures were then separated by SDS-PAGE and transferred onto nitrocellulose. (FIG. 38A) Ponceau staining of the nitrocellulose membrane provides an overview of the distribution of MBP-16E6 (WT or C58S), 13-biotin or 13-3L3A-biotin, and HT1080 lysate. (FIG. 38B) Western blot analysis of MBP-16E6 and GAPDH, detailing antibody interactions. The same blot was subsequently incubated with streptavidin-680, enabling the visualization of 13-biotin and 13-3L3A-biotin under (FIG. 38C) short and (FIG. 38D) long exposures. The interaction between MBP-16E6 and 13-biotin remains observable. Additional bands indicate at 10 μM, binding of 13-biotin and 13-3L3A-biotin to unidentified proteins present in the HT1080 lysate.

FIGS. 39A-39G show streptavidin pull-down analysis of 13-biotin, 13-3L3A-biotin, or control (beads alone with DMSO) using quantitative multiplexed mass spectrometry. HPV-HT1080 or HPV16+ CaSki cell lysates (2 mg) were treated with 13-biotin (5 μM), 13-3L3A-biotin (5 μM), or DMSO (0.5%) overnight at 4° C. and subjected to pull-down using streptavidin beads. The samples were then TMT labeled, digested with trypsin, and analyzed by mass spectrometry. (FIG. 39A) Heat map showing the relative enrichment in CaSki cells treated with 13-biotin relative to those treated with 13-3L3A-biotin. Volcano plot comparisons for HT1080 cells are shown for (FIG. 39B) 13-3L3A-biotin vs. 13-biotin, (FIG. 39C) DMSO vs. 13-biotin, and (FIG. 39D) DMSO vs. 13-3L3A-biotin. Volcano plot comparisons for CaSki cells are presented for (FIG. 39E) 13-3L3A-biotin vs. 13-biotin, (FIG. 39F) DMSO vs. 13-biotin, and (FIG. 39G) DMSO vs. 13-3L3A-biotin. The data represent results from two independent experiments. The top five proteins with the highest −Log 10(q-value) are highlighted.

### DETAILED DESCRIPTION

The present disclosure is based, in part, upon the development of synthetic peptides that bind Human Papilloma Virus 16 E early protein 6 (HPV16E6 or 16E6) in a covalent or non covalent manner to form peptide-16E6 complexes. Additionally, the peptide-16E6 complexes can modulate the function of p53 that promotes ubiquitination. The synthetic peptides and peptidomimetics can be used to modulate or abrogate HPV16E6/p53 binding and signaling in HPV 16 positive cancer cells.

Various components and aspects of the disclosure are described in further detail in the subsections below.

### I. Definitions

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the disclosure, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method provided and described herein can be combined in a variety of ways without departing from the spirit and scope of the present disclosure and invention(s) herein, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of invention(s) provided, described, and depicted herein.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

As used herein, the terms “comprises,” “comprising,” “includes,” “including,” “has,” “having,” “is,” “are” or any other variation thereof, are intended to cover a non-exclusive inclusion. They are to be interpreted synonymously with the phrases “having at least” or “including at least”. The term “consisting of” refers to including, and being limited to, whatever follows the phrase “consisting of”

As used herein, the term “comprising” also specifically includes embodiments “consisting of” and “consisting essentially of” the recited elements, unless specifically indicated otherwise. Similarly, the term “consisting essentially of” is intended to include embodiments encompassed by the term “consisting of”.

It should be understood that the order of steps or order for performing certain actions is immaterial so long as the present invention remain operable. Moreover, two or more steps or actions may be conducted simultaneously.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present disclosure and does not pose a limitation on the scope of any invention(s) unless claimed. No language in the specification should be construed as indicating any non-claimed element as essential to the practice of that provided by the present disclosure.

As used herein, “residue” refers to a position in a protein and its associated amino acid identity.

The term “modulation” refers to an increase or decrease in the level of a target molecule or the function of a target molecule. The term “modulator” as used herein refers to modulation of (e.g., an increase or decrease in) the level of a target molecule or the function of a target molecule.

### Chemical Definitions

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

When a range of values is listed, it is intended to encompass each value and sub-range within the range. For example, “C1-C6 alkyl” is intended to encompass, C1, C2, C3, C4, C5, C6, C1-C6, C1-C5, C1-C4, C1-C3, C1-C2, C2-C6, C2-C5, C2-C4, C2-C3, C3-C6, C3-C5, C3-C4, C4-C6, C4-C5, and C5-C6 alkyl.

“Aryl” refers to a radical of a monocyclic or polycyclic (e.g., bicyclic or tricyclic) 4n+2 aromatic ring system (e.g., having 6, 10, or 14 p electrons shared in a cyclic array) having 6-14 ring carbon atoms and zero heteroatoms provided in the aromatic ring system (“C6-C14 aryl”). In some embodiments, an aryl group has six ring carbon atoms (“C6 aryl”; e.g., phenyl). In some embodiments, an aryl group has ten ring carbon atoms (“C10 aryl”; e.g., naphthyl such as 1-naphthyl and 2-naphthyl). In some embodiments, an aryl group has fourteen ring carbon atoms (“C14 aryl”; e.g., anthracyl). An aryl group may be described as, e.g., a C6-C10-membered aryl, wherein the term “membered” refers to the non-hydrogen ring atoms within the moiety. Aryl groups include, but are not limited to, phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C6-C14 aryl. In certain embodiments, the aryl group is substituted C6-C14 aryl.

“Halo” or “halogen,” independently or as part of another substituent, mean, unless otherwise stated, a fluorine (F), chlorine (Cl), bromine (Br), or iodine (I) atom. The term “halide” by itself or as part of another substituent, refers to a fluoride, chloride, bromide, or iodide atom. In certain embodiments, the halo group is either fluorine or chlorine.

The use of any and all examples, or exemplary language herein, for example, “such as” or “including,” is intended merely to illustrate better the present disclosure and does not pose a limitation on the scope of any invention(s) unless claimed. No language in the specification should be construed as indicating any non-claimed element as essential to the practice of that provided by the present disclosure.

### II. Human Papilloma Virus 16

The high-risk virus variants, including HPV16, drive tumorigenesis in part by promoting the degradation of the tumor suppressor p53. This degradation is mediated by the HPV early protein 6 (16E6), which recruits the E3 ubiquitin ligase E6AP and redirects its activity towards ubiquitinating p53. Targeting the protein interaction interface between HPV 16E6 and E6AP is a promising modality to mitigate HPV-mediated degradation of p53. HPV-encoded early protein 6 (E6) and early protein 7 (E7) are primary transforming viral proteins enhancing cancer cell proliferation and contributing to cancer progression. HPV E7 primarily binds and inactivates retinoblastoma protein (pRB) and related pocket proteins, p107 and p130, inducing their proteasome-dependent degradation and promoting cell cycle entry. Multiple host proteins interact with 16E6 via a leucine-rich LXXLL motif, including E6BP, IRF3, paxillin and tuberin; PDZ proteins such as MAGI-1; and other proteins such as p53, E6AP, MAML1, and p300/CBP. Nevertheless, the interaction of 16E6 with the E3 ubiquitin ligase, E6AP, and p53 is thought to be a central transformative pathway of cell immortalization. E6 and E7 are active in different cell stages. E7 promotes cell entry to the division phase S, in turn, E6 prevents E7-induced apoptosis by degrading the apoptosis-inducing protein p53. HPV+ tumors mostly contain non-mutant p53, as a result, silencing E6 with siRNA can rescue p53 and initiate apoptosis in HPV+ cancer cell lines. Therefore, checkpoint networks are primed in HPV+ cells awaiting E6 disruption, providing support for its suitability as an oncology target.

HPV16 E6 (16E6) hijacks E6AP to form a complex with p53 that promotes ubiquitination of p53, leading to its subsequent proteasome-mediated degradation, while neither E6 nor E6AP interact with p53 alone. p53 mediates stress response, cell proliferation, and apoptosis, and its downregulation or mutation is a hallmark of carcinogenesis directly affecting efficacy of cancer therapy. It was reported that targeting the ubiquitination catalytical domain of ubiquitin ligase E6AP (HECT domain) represses p53 ubiquitination in vitro. However, E6AP is a regulator of the proteostasis signaling network and has wide distribution patterns in humans. Additionally, E6AP have been associated with Angelman syndrome and Prader-Will syndrome, diseases associated with developmental defects. As such, targeting E6AP could result in on-target toxicity although acute inactivation has not been assessed. Given the potential toxicity associated with targeting E6AP and the lack of 16E6 in healthy human cells, targeting the viral protein 16E6 can provide a wide therapeutic index in HPV+ cancers to limit on-target side-effect.

Efforts have been made in the past decades to target the 16E6/E6AP protein-protein interactions (PPIs). Ribozymes and gene-silencing siRNA that reduce or remove the activity of the HPV E6 oncogene and its protein product have been shown to induce apoptosis in cancer cells. Polyhydroxy flavonoid display low micromolar E6 inhibition IC₅₀ values and cytotoxicity in HPV+ cancer cells but have not been successful in clinical trials, possibly due to unclear structure-activity relationships (SAR), poor stability, off-target binding, and low specificity. Biomolecules, including E6-binding antibodies and mini-proteins with dissociation constants (K_{D}) of 10 nM-60 nM, exhibit nanomolar affinity to E6. However, their inhibitory effect is unsurprisingly hindered by poor penetration through the cell membrane.

### III. E6AP Peptides

Disclosed herein are synthetic peptides based on E6AP and libraries thereof.

In one aspect, the disclosure provides for a synthetic peptide comprising an amino acid sequence IPESSELTLQELLGEER (SEQ ID NO: 1).

In one aspect, the disclosure provides for a synthetic peptide comprising an amino acid sequence ELTLQELLGEER (SEQ ID NO: 5).

In some embodiments, the peptide comprises Fluorene-IPESSELTLQELLGEERRAA-K(1-Anthracene (SEQ ID NO:2) or Fluorene-IPESAELTLQELLGEERRAA-K(1-Anthracene (SEQ ID NO:3).

In another aspect, the disclosure provides for a synthetic peptide selected from TABLE 2C, TABLE 2D, or TABLE 2E modified with a warhead. In some embodiments, the peptide comprises Fluorene-IPQSAELTLQELL(DHA)RRKKK(Anthracene) (SEQ ID NO: 4).

In another aspect, the disclosure provides for a synthetic peptide comprising an amino acid sequence X₁X₂X₃X₂QX₁X₂X₂CEER (SEQ ID NO: 6), wherein X₁ is Nva, Leu, Aoc, Cpa, Cba, Cha, Phg, Hof, or Naf; X₂ is Aad, Glu, or Cya; X₃ is Thr, Asn, Hyp, Cpg, Cbg, or Ceg; and X₄ is d-Ala, Aib, Gly, or Gln.

In some embodiments, the peptide is selected from TABLES 3-31. In some embodiments, the peptide is selected from SEQ ID NO: 7-19. In some embodiments, the peptide comprises SEQ ID NO: 17. In some embodiments, the peptide is selected from TABLES 32-39.

In another aspect, the disclosure provides for a synthetic peptide comprising an amino acid sequence Aad-X₁-X₂-Leu-Aib-Aad-Cba-Leu-Cys-X₃-X₃-X₃ (SEQ ID NO: 20) wherein X₁ is PhF, Naf, Qua, Clw, or Trp; X₂ is Chg or Ceg; and X₃ is d-Ala, Aib, Gly, Ser, Aad, Glu, Gln, Hoc, Val, Leu, Met, Pro, Dap, Lys, Arg, His, Trp, Phe, Tyr, or Null.

In some embodiments, the peptide is 11 or 12 amino acids long.

In another aspect, the disclosure provides for a synthetic peptide comprising the formula

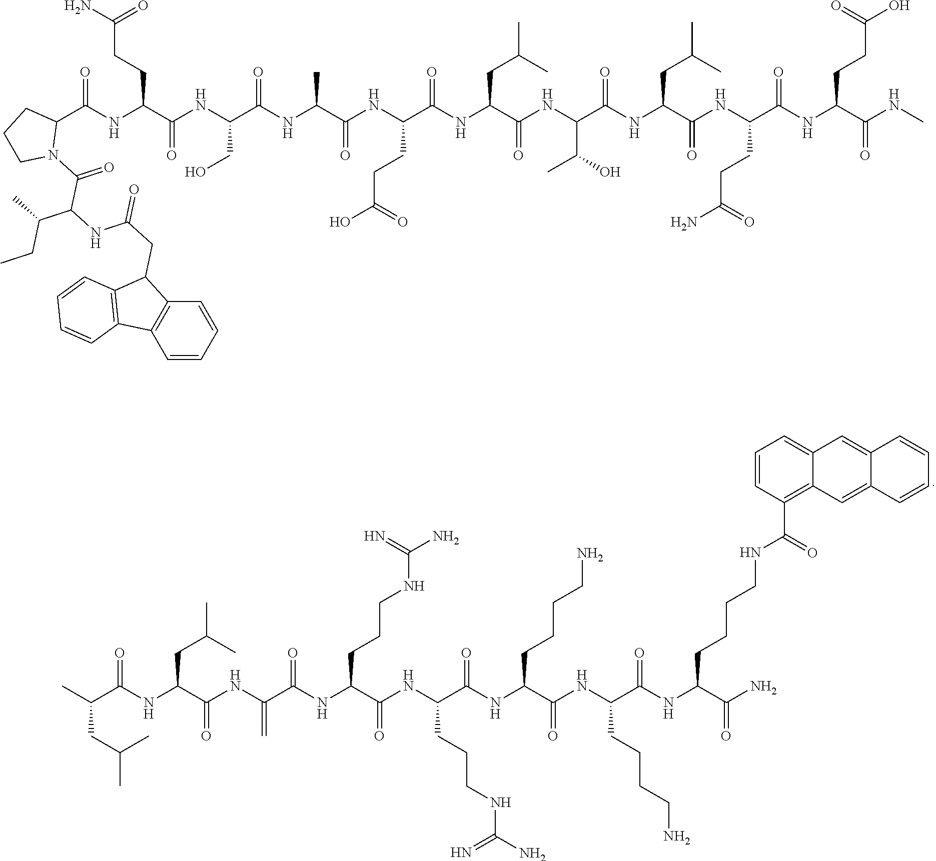

In another aspect, the disclosure provides for a synthetic peptide comprising the formula

Name: e6apep
Sequence:
(SEQ ID NO: 26)
IPESSELTLQELLGEERR

In another aspect, the disclosure provides for a synthetic peptide comprising the formula

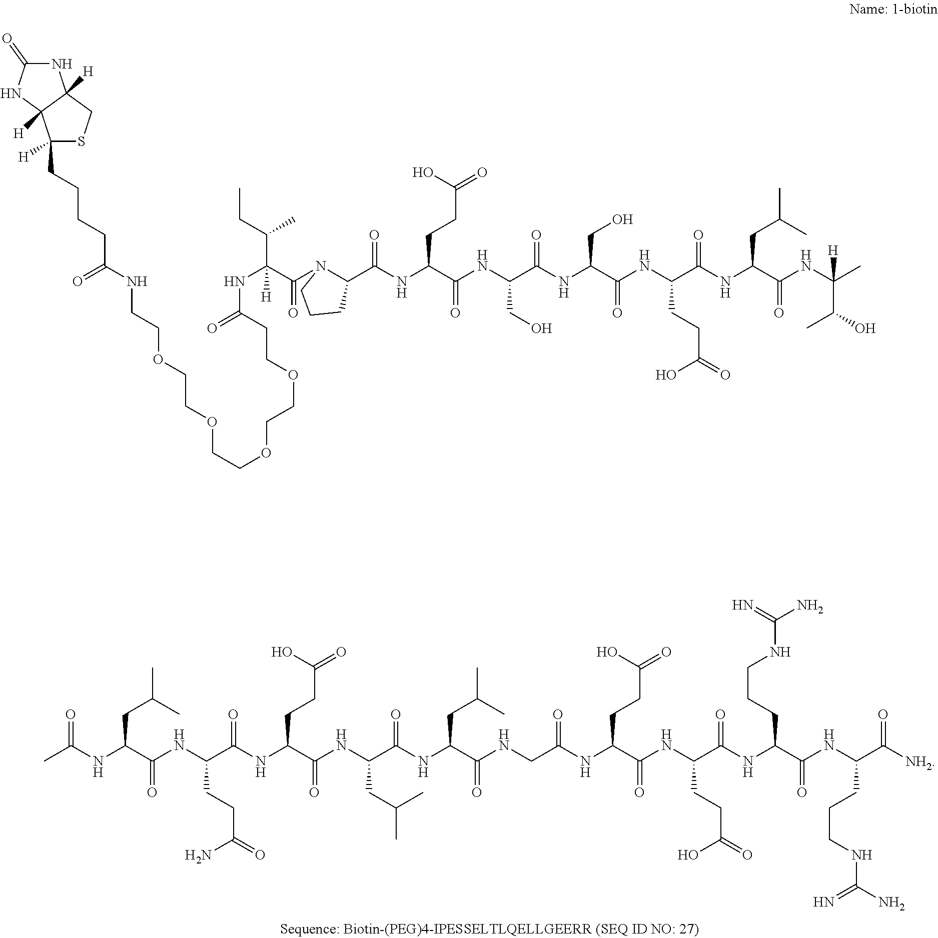

In another aspect, the disclosure provides for a synthetic peptide comprising the formula

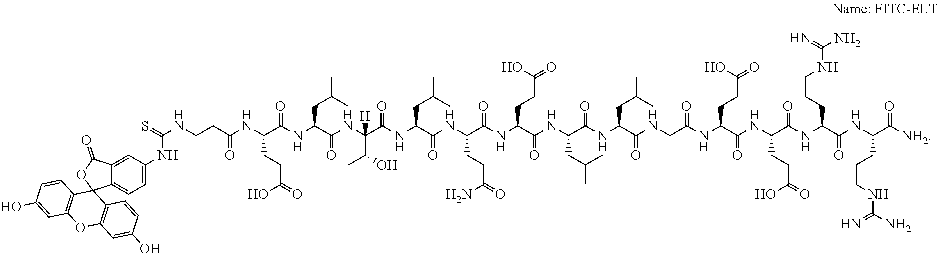 [SMILES: CC(C)C[C@H](NC(=O)[C@H](CC(C)C)NC(=O)[C@H](CCC(=O)O)NC(=O)[C@H](CCC(N)=O)NC(=O)[C@H](CC(C)C)NC(=O)[C@@H](NC(=O)[C@H](CC(C)C)NC(=O)[C@H](CCC(=O)O)NC(=O)CCNC(=S)Nc1ccc2c(c1)C(=O)OC21c2ccc(O)cc2Oc2cc(O)ccc21)[C@@H](C)O)C(=O)NCC(=O)N[C@@H](CCC(=O)O)C(=O)N[C@@H](CCC(=O)O)C(=O)N[C@@H](CCCNC(=N)N)C(=O)N[C@@H](CCCNC(=N)N)C(N)=O]

In another aspect, the disclosure provides for a synthetic peptide comprising the formula

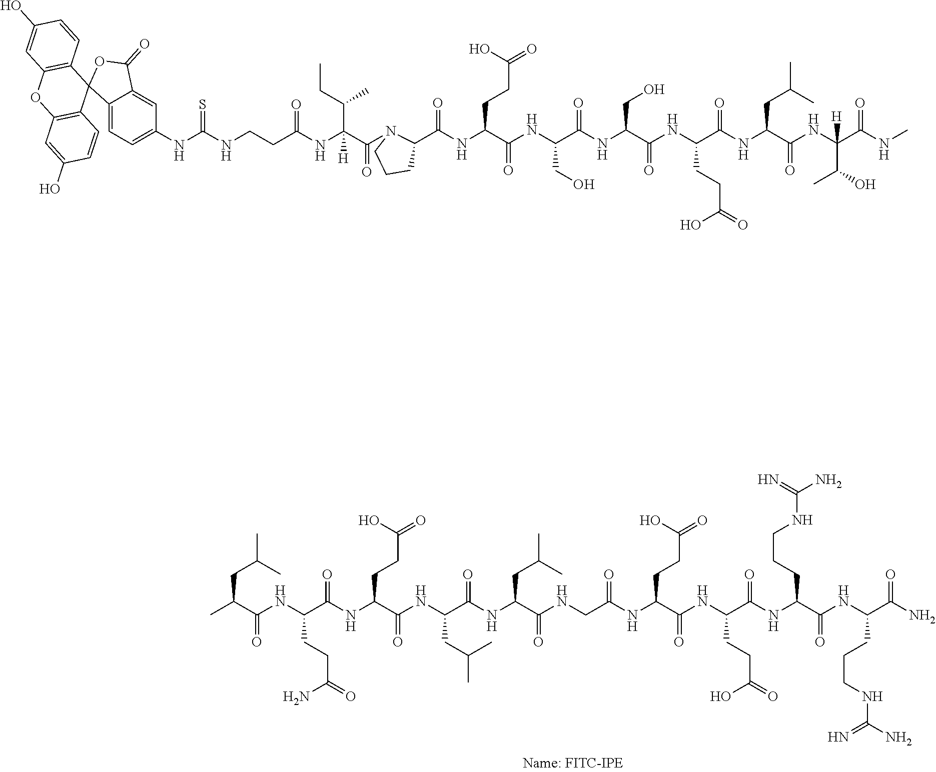

In another aspect, the disclosure provides for a synthetic peptide comprising the formula

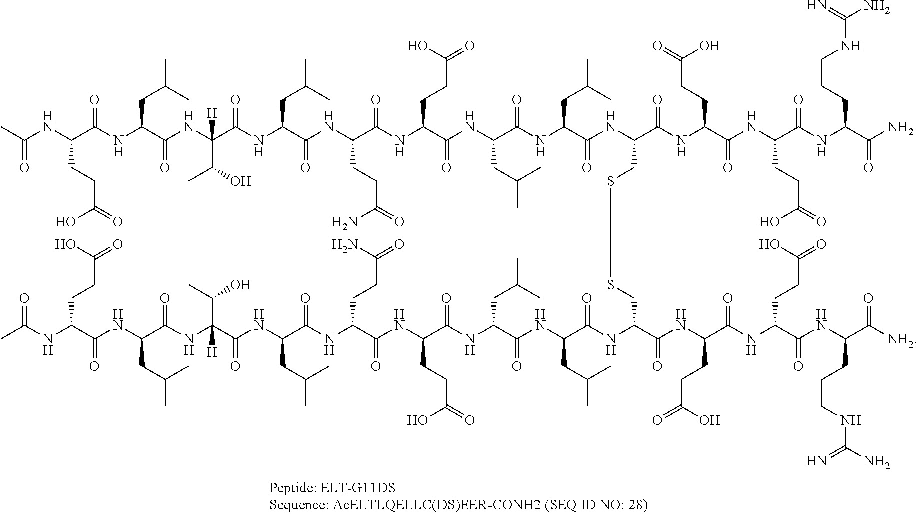

In another aspect, the disclosure provides for a synthetic peptide comprising the formula

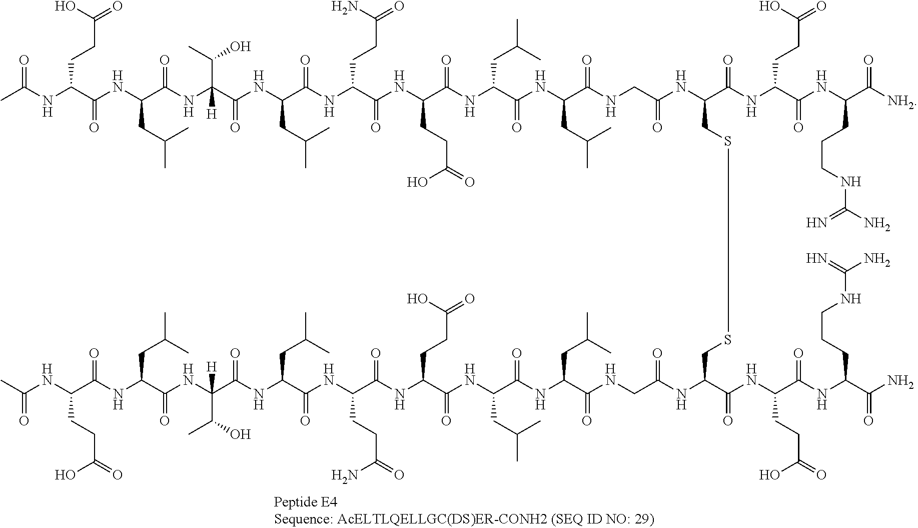 [SMILES: CC(=O)N[C@@H](CCC(=O)O)C(=O)N[C@@H](CC(C)C)C(=O)N[C@H](C(=O)N[C@@H](CC(C)C)C(=O)N[C@@H](CCC(N)=O)C(=O)N[C@@H](CCC(=O)O)C(=O)N[C@@H](CC(C)C)C(=O)N[C@@H](CC(C)C)C(=O)NCC(=O)N[C@@H](CSSC[C@@H](NC(=O)CNC(=O)[C@@H](CC(C)C)NC(=O)[C@@H](CC(C)C)NC(=O)[C@@H](CCC(=O)O)NC(=O)[C@@H](CCC(N)=O)NC(=O)[C@@H](CC(C)C)NC(=O)[C@H](NC(=O)[C@@H](CC(C)C)NC(=O)[C@@H](CCC(=O)O)NC(C)=O)[C@H](C)O)C(=O)N[C@H](CCC(=O)O)C(=O)N[C@H](CCCNC(=N)N)C(N)=O)C(=O)N[C@@H](CCC(=O)O)C(=O)N[C@@H](CCCNC(=N)N)C(N)=O)[C@@H](C)O]

In another aspect, the disclosure provides for a synthetic peptide comprising the formula

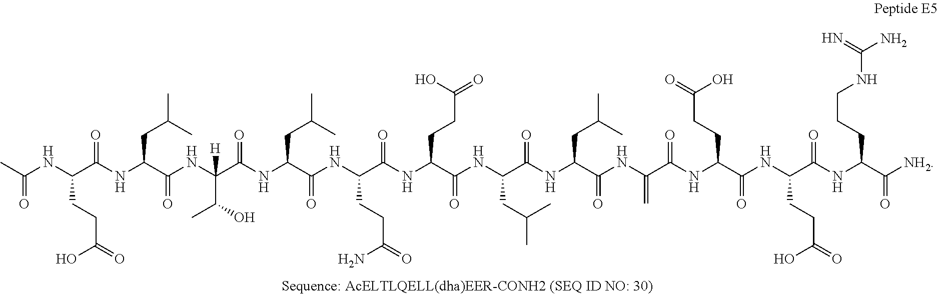 [SMILES: C=C(NC(=O)[C@H](CC(C)C)NC(=O)[C@H](CC(C)C)NC(=O)[C@H](CCC(=O)O)NC(=O)[C@H](CCC(N)=O)NC(=O)[C@H](CC(C)C)NC(=O)[C@@H](NC(=O)[C@H](CC(C)C)NC(=O)[C@H](CCC(=O)O)NC(C)=O)[C@@H](C)O)C(=O)N[C@@H](CCC(=O)O)C(=O)N[C@@H](CCC(=O)O)C(=O)N[C@@H](CCCNC(=N)N)C(N)=O]

In another aspect, the disclosure provides for a synthetic peptide comprising the formula

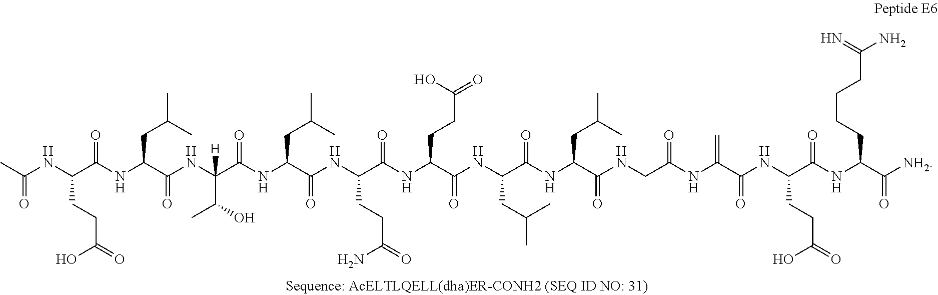 [SMILES: C=C(NC(=O)CNC(=O)[C@H](CC(C)C)NC(=O)[C@H](CC(C)C)NC(=O)[C@H](CCC(=O)O)NC(=O)[C@H](CCC(N)=O)NC(=O)[C@H](CC(C)C)NC(=O)[C@@H](NC(=O)[C@H](CC(C)C)NC(=O)[C@H](CCC(=O)O)NC(C)=O)[C@@H](C)O)C(=O)N[C@@H](CCC(=O)O)C(=O)N[C@@H](CCCCC(=N)N)C(N)=O]

In another aspect, the disclosure provides for a synthetic peptide comprising the formula

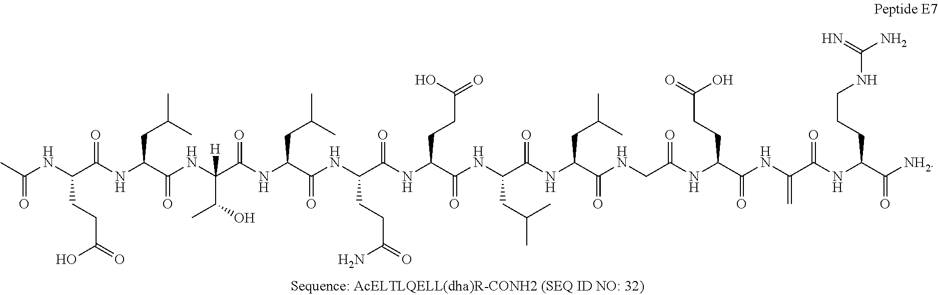 [SMILES: C=C(NC(=O)[C@H](CCC(=O)O)NC(=O)CNC(=O)[C@H](CC(C)C)NC(=O)[C@H](CC(C)C)NC(=O)[C@H](CCC(=O)O)NC(=O)[C@H](CCC(N)=O)NC(=O)[C@H](CC(C)C)NC(=O)[C@@H](NC(=O)[C@H](CC(C)C)NC(=O)[C@H](CCC(=O)O)NC(C)=O)[C@@H](C)O)C(=O)N[C@@H](CCCNC(=N)N)C(N)=O]

In another aspect, the disclosure provides for a synthetic peptide comprising the formula

Name: 294-201-001-DHA
(SEQ ID NO: 33)
Sequence: 9-Fluorene carboxyl-IPESAELTLQELL(DHA)-NH2

In another aspect, the disclosure provides for a synthetic peptide comprising the formula

Name: 294-201-002-DHA
(SEQ ID NO: 34)
Sequence: 9-Fluorene carboxyl-IPESAELTAQEAA(DHA)-NH2

In another aspect, the disclosure provides for a synthetic peptide comprising the formula

Name: 294-201-03-DHA
(SEQ ID NO: 35)
Sequence: 9-Fluorene carboxyl-IPESAELTLQELL(DHA)EER-
NH2

In another aspect, the disclosure provides for a synthetic peptide comprising the formula

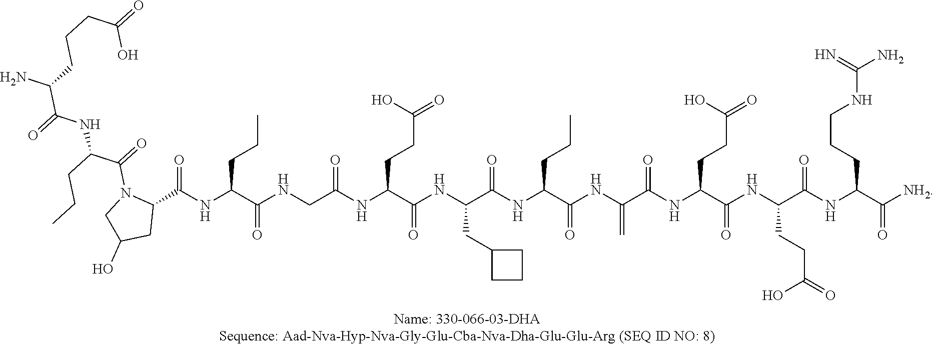 [SMILES: C=C(NC(=O)[C@H](CCC)NC(=O)[C@H](CC1CCC1)NC(=O)[C@H](CCC(=O)O)NC(=O)CNC(=O)[C@H](CCC)NC(=O)[C@@H]1CC(O)CN1C(=O)[C@H](CCC)NC(=O)[C@H](N)CCCC(=O)O)C(=O)N[C@@H](CCC(=O)O)C(=O)N[C@@H](CCC(=O)O)C(=O)N[C@@H](CCCNC(=N)N)C(N)=O]

In another aspect, the disclosure provides for a synthetic peptide comprising the formula

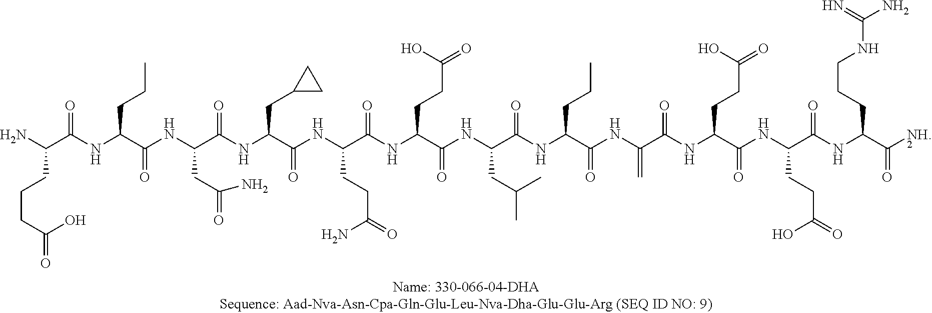

In another aspect, the disclosure provides for a synthetic peptide comprising the formula

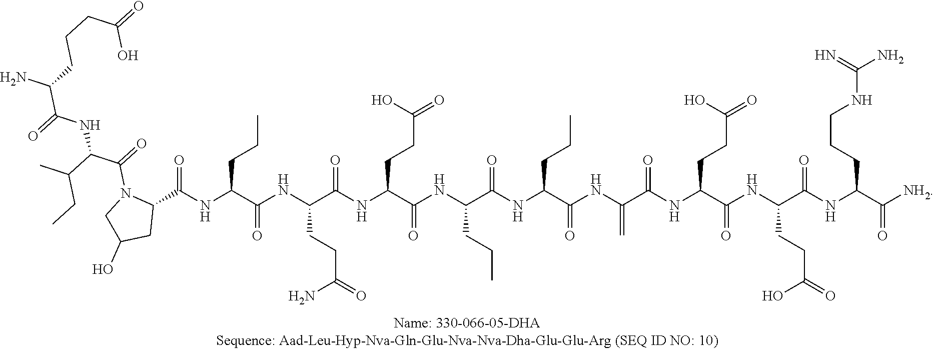 [SMILES: C=C(NC(=O)[C@H](CCC)NC(=O)[C@H](CCC)NC(=O)[C@H](CCC(=O)O)NC(=O)[C@H](CCC(N)=O)NC(=O)[C@H](CCC)NC(=O)[C@@H]1CC(O)CN1C(=O)[C@@H](NC(=O)[C@H](N)CCCC(=O)O)C(C)CC)C(=O)N[C@@H](CCC(=O)O)C(=O)N[C@@H](CCC(=O)O)C(=O)N[C@@H](CCCNC(=N)N)C(N)=O]

In another aspect, the disclosure provides for a synthetic peptide comprising the formula

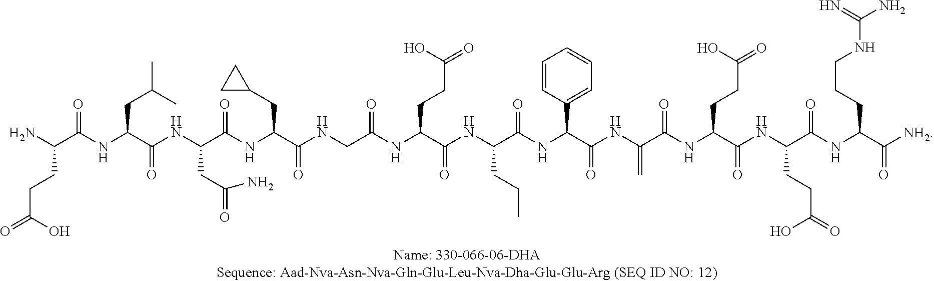 [SMILES: C=C(NC(=O)[C@@H](NC(=O)[C@H](CCC)NC(=O)[C@H](CCC(=O)O)NC(=O)CNC(=O)[C@H](CC1CC1)NC(=O)[C@H](CC(N)=O)NC(=O)[C@H](CC(C)C)NC(=O)[C@@H](N)CCC(=O)O)c1ccccc1)C(=O)N[C@@H](CCC(=O)O)C(=O)N[C@@H](CCC(=O)O)C(=O)N[C@@H](CCCNC(=N)N)C(N)=O]

In another aspect, the disclosure provides for a synthetic peptide comprising the formula

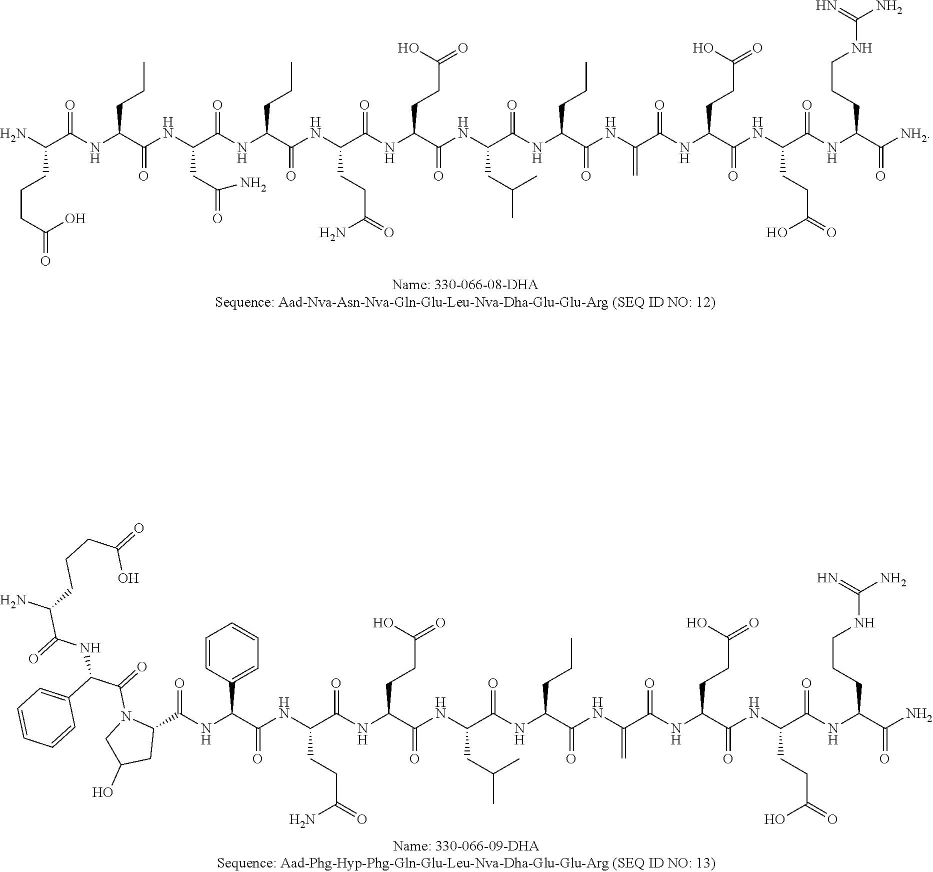 [SMILES: C=C(NC(=O)[C@H](CCC)NC(=O)[C@H](CC(C)C)NC(=O)[C@H](CCC(=O)O)NC(=O)[C@H](CCC(N)=O)NC(=O)[C@H](CCC)NC(=O)[C@H](CC(N)=O)NC(=O)[C@H](CCC)NC(=O)[C@@H](N)CCCC(=O)O)C(=O)N[C@@H](CCC(=O)O)C(=O)N[C@@H](CCC(=O)O)C(=O)N[C@@H](CCCNC(=N)N)C(N)=O ; C=C(NC(=O)[C@H](CCC)NC(=O)[C@H](CC(C)C)NC(=O)[C@H](CCC(=O)O)NC(=O)[C@H](CCC(N)=O)NC(=O)[C@@H](NC(=O)[C@@H]1CC(O)CN1C(=O)[C@@H](NC(=O)[C@H](N)CCCC(=O)O)c1ccccc1)c1ccccc1)C(=O)N[C@@H](CCC(=O)O)C(=O)N[C@@H](CCC(=O)O)C(=O)N[C@@H](CCCNC(=N)N)C(N)=O]

In another aspect, the disclosure provides for a synthetic peptide comprising the formula

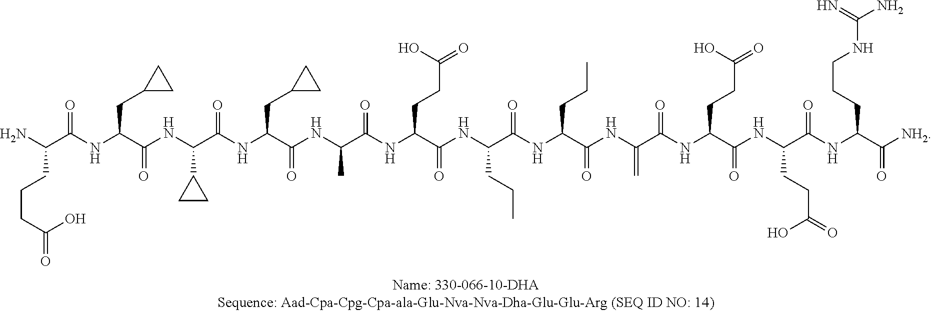 [SMILES: C=C(NC(=O)[C@H](CCC)NC(=O)[C@H](CCC)NC(=O)[C@H](CCC(=O)O)NC(=O)[C@@H](C)NC(=O)[C@H](CC1CC1)NC(=O)[C@@H](NC(=O)[C@H](CC1CC1)NC(=O)[C@@H](N)CCCC(=O)O)C1CC1)C(=O)N[C@@H](CCC(=O)O)C(=O)N[C@@H](CCC(=O)O)C(=O)N[C@@H](CCCNC(=N)N)C(N)=O]

In another aspect, the disclosure provides for a synthetic peptide comprising the formula

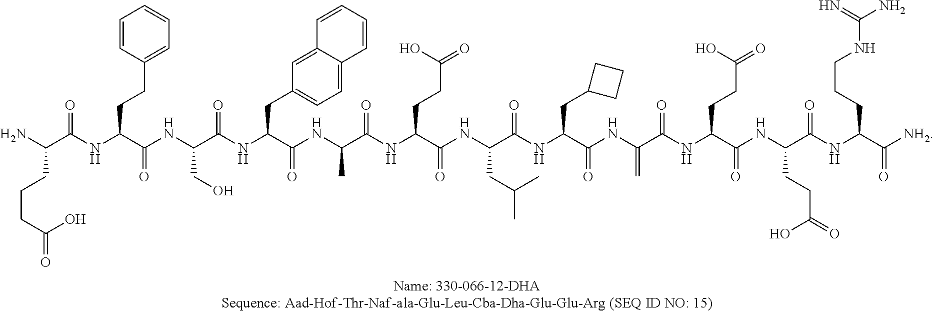 [SMILES: C=C(NC(=O)[C@H](CC1CCC1)NC(=O)[C@H](CC(C)C)NC(=O)[C@H](CCC(=O)O)NC(=O)[C@@H](C)NC(=O)[C@H](Cc1ccc2ccccc2c1)NC(=O)[C@H](CO)NC(=O)[C@H](CCc1ccccc1)NC(=O)[C@@H](N)CCCC(=O)O)C(=O)N[C@@H](CCC(=O)O)C(=O)N[C@@H](CCC(=O)O)C(=O)N[C@@H](CCCNC(=N)N)C(N)=O]

In another aspect, the disclosure provides for a synthetic peptide comprising the formula

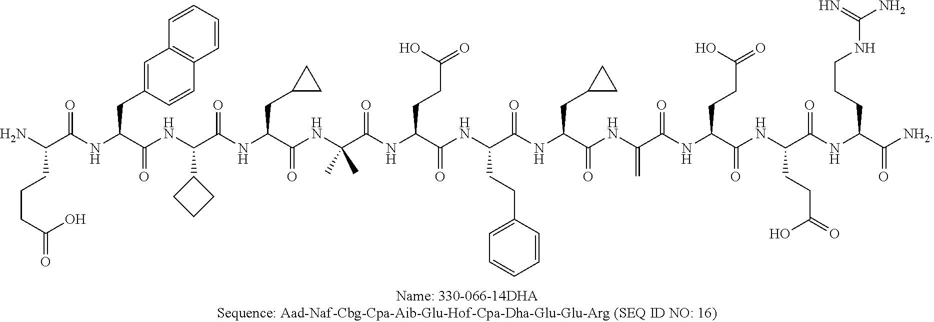

In another aspect, the disclosure provides for a synthetic peptide comprising the formula

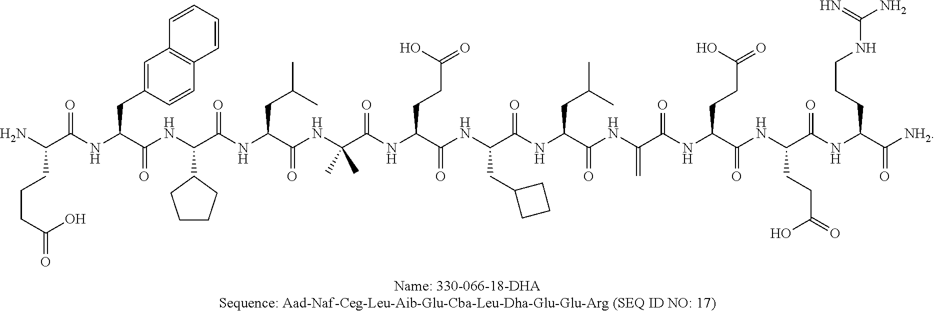

In another aspect, the disclosure provides for a synthetic peptide comprising the formula

In another aspect, the disclosure provides for a synthetic peptide comprising the formula

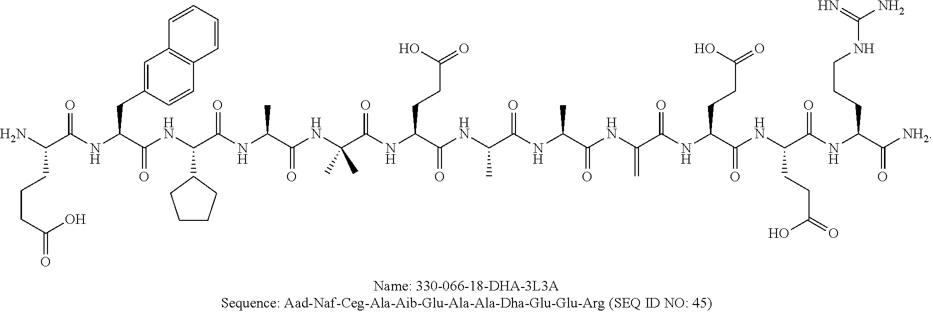

In another aspect, the disclosure provides for a synthetic peptide comprising the formula

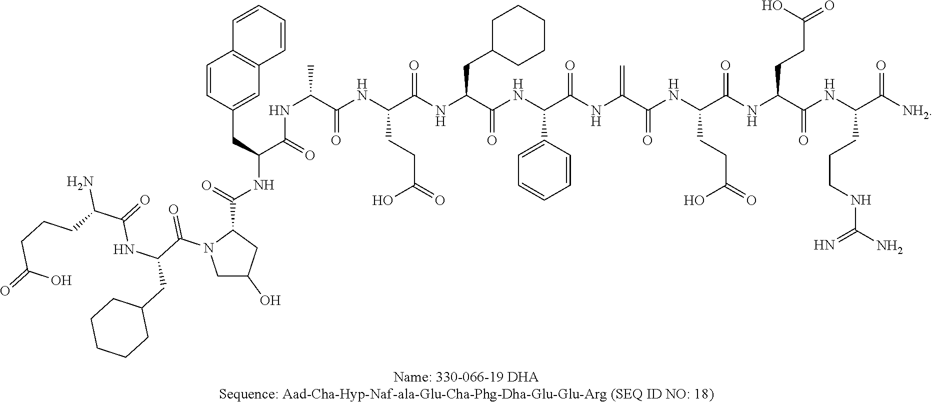 [SMILES: C=C(NC(=O)[C@@H](NC(=O)[C@H](CC1CCCCC1)NC(=O)[C@H](CCC(=O)O)NC(=O)[C@@H](C)NC(=O)[C@H](Cc1ccc2ccccc2c1)NC(=O)[C@@H]1CC(O)CN1C(=O)[C@H](CC1CCCCC1)NC(=O)[C@@H](N)CCCC(=O)O)c1ccccc1)C(=O)N[C@@H](CCC(=O)O)C(=O)N[C@@H](CCC(=O)O)C(=O)N[C@@H](CCCNC(=N)N)C(N)=O]

In another aspect, the disclosure provides for a synthetic peptide comprising the formula

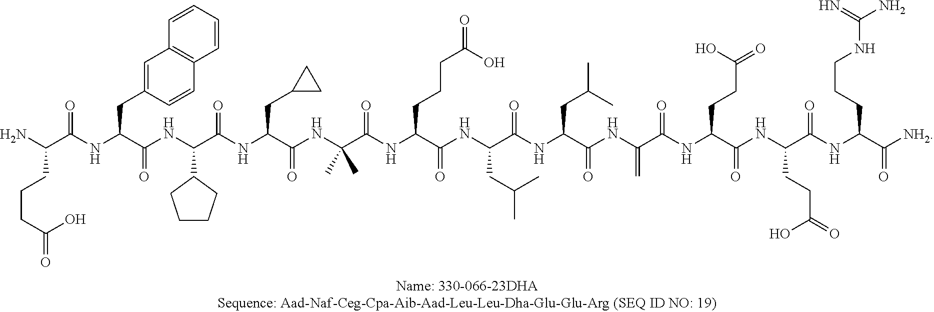

In another aspect, the disclosure provides for a synthetic peptide comprising the formula

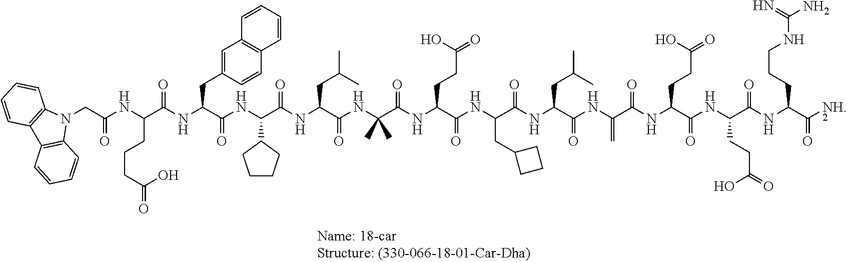

In another aspect, the disclosure provides for a synthetic peptide comprising the formula

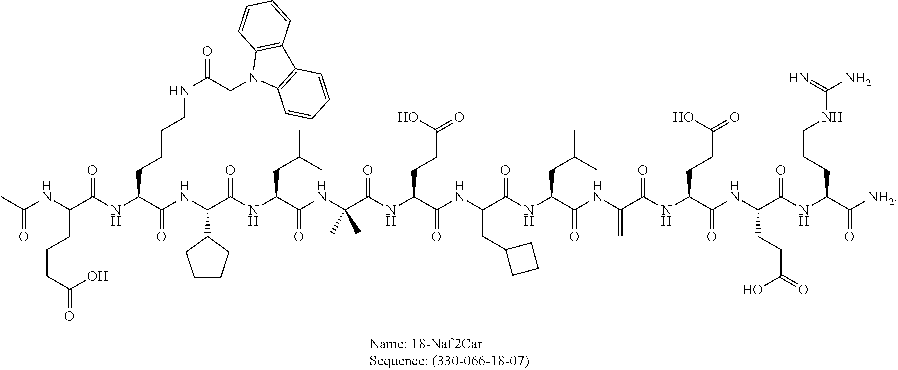

In another aspect, the disclosure provides for a synthetic peptide comprising the formula

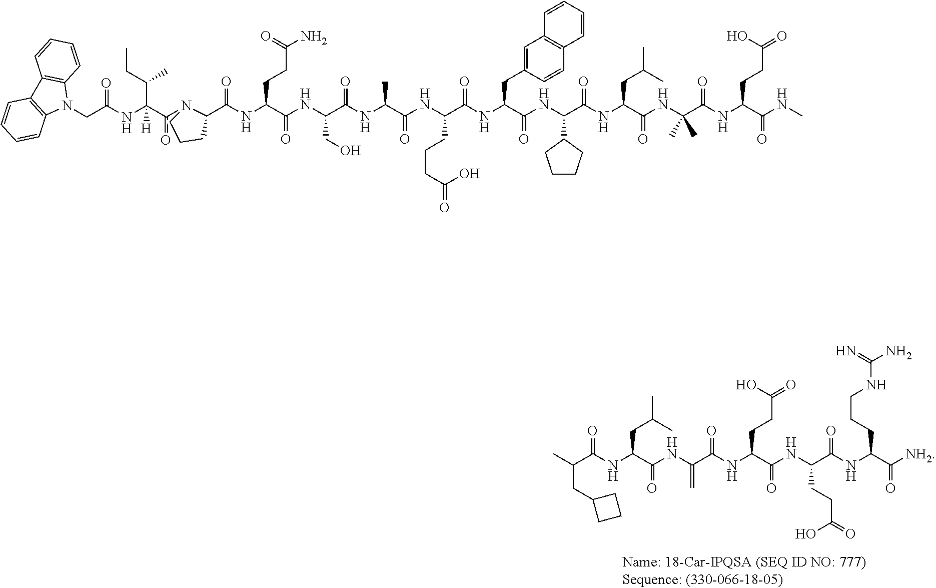

In another aspect, the disclosure provides for a synthetic peptide comprising the formula

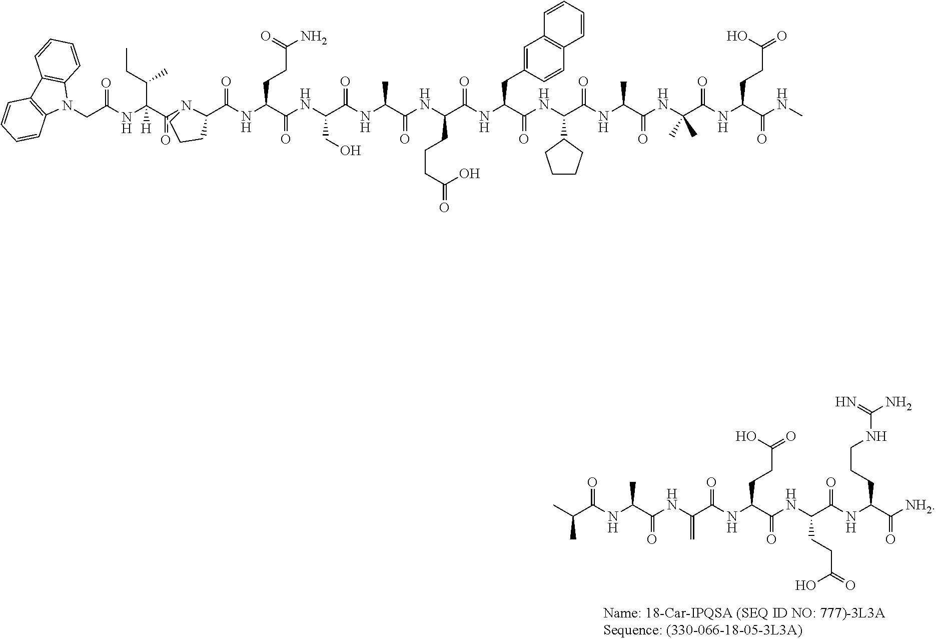

In another aspect, the disclosure provides for a synthetic peptide comprising the formula

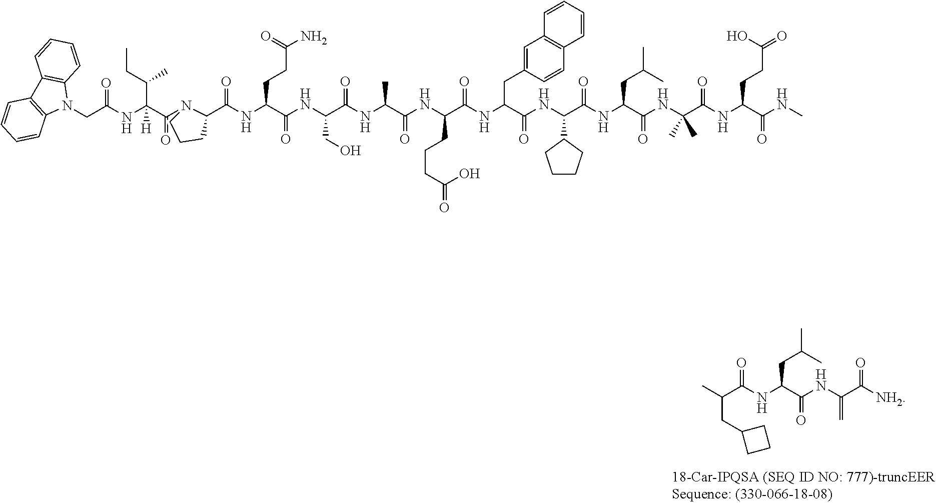

### N-Terminal Modified Peptide

In another aspect, the disclosure provides for a synthetic peptide comprising the formula

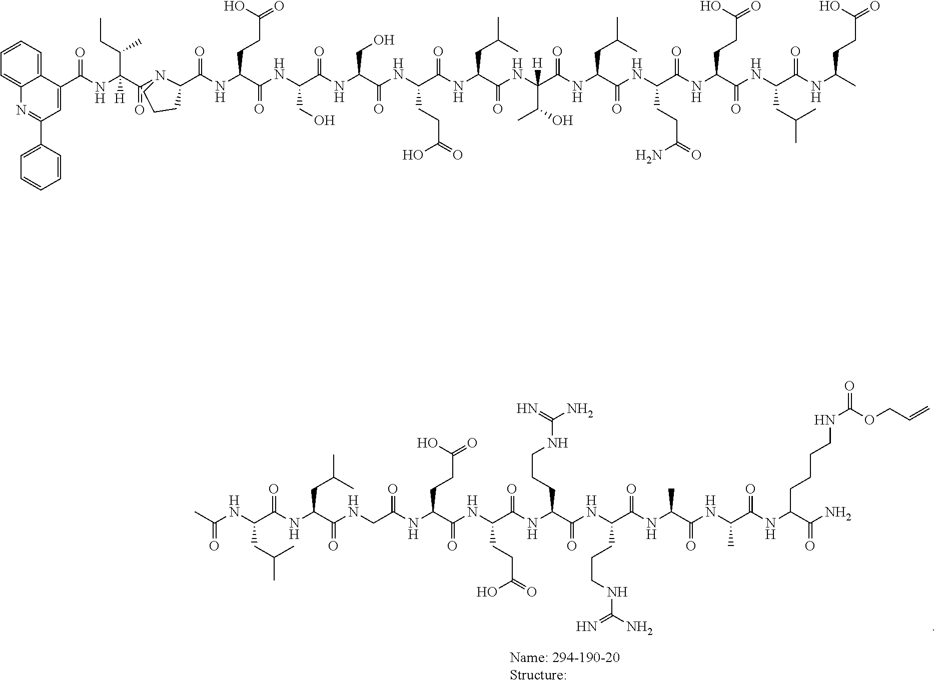 [SMILES: CC[C@H](C)[C@H](NC(=O)c1cc(-c2ccccc2)nc2ccccc12)C(=O)N1CCC[C@H]1C(=O)N[C@@H](CCC(=O)O)C(=O)N[C@@H](CO)C(=O)N[C@@H](CO)C(=O)N[C@@H](CCC(=O)O)C(=O)N[C@@H](CC(C)C)C(=O)N[C@H](C(=O)N[C@@H](CC(C)C)C(=O)N[C@@H](CCC(N)=O)C(=O)N[C@@H](CCC(=O)O)C(=O)N[C@@H](CC(C)C)C(=O)N[C@H](C)CCC(=O)O)[C@@H](C)O ; C=CCOC(=O)NCCCCC(NC(=O)[C@H](C)NC(=O)[C@H](C)NC(=O)[C@H](CCCNC(=N)N)NC(=O)[C@H](CCCNC(=N)N)NC(=O)[C@H](CCC(=O)O)NC(=O)[C@H](CCC(=O)O)NC(=O)CNC(=O)[C@H](CC(C)C)NC(=O)[C@H](CC(C)C)NC(C)=O)C(N)=O]

In another aspect, the disclosure provides for a synthetic peptide comprising the formula

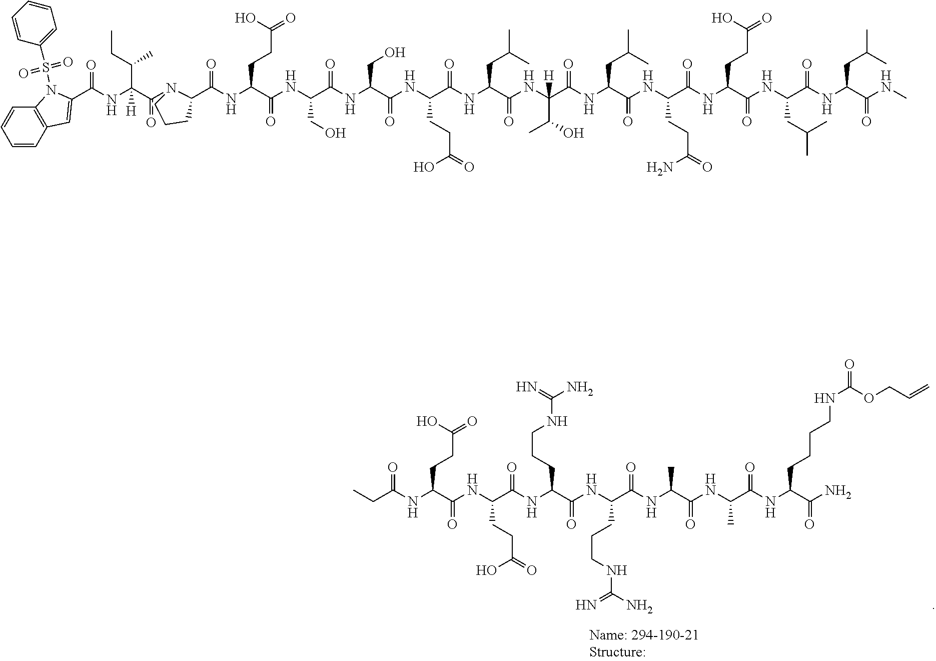

In another aspect, the disclosure provides for a synthetic peptide comprising the formula

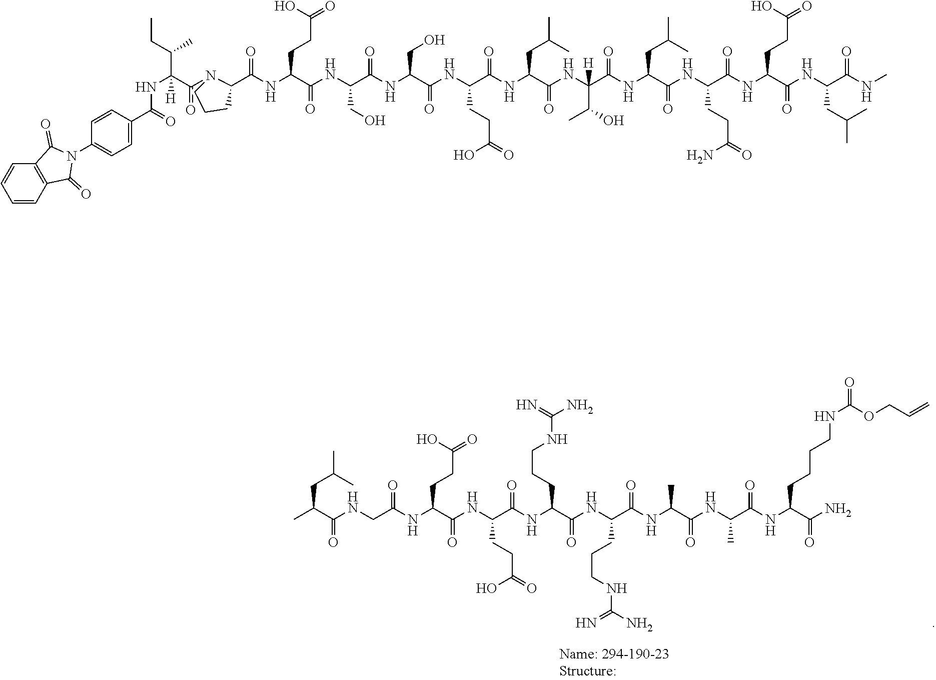

In another aspect, the disclosure provides for a synthetic peptide comprising the formula

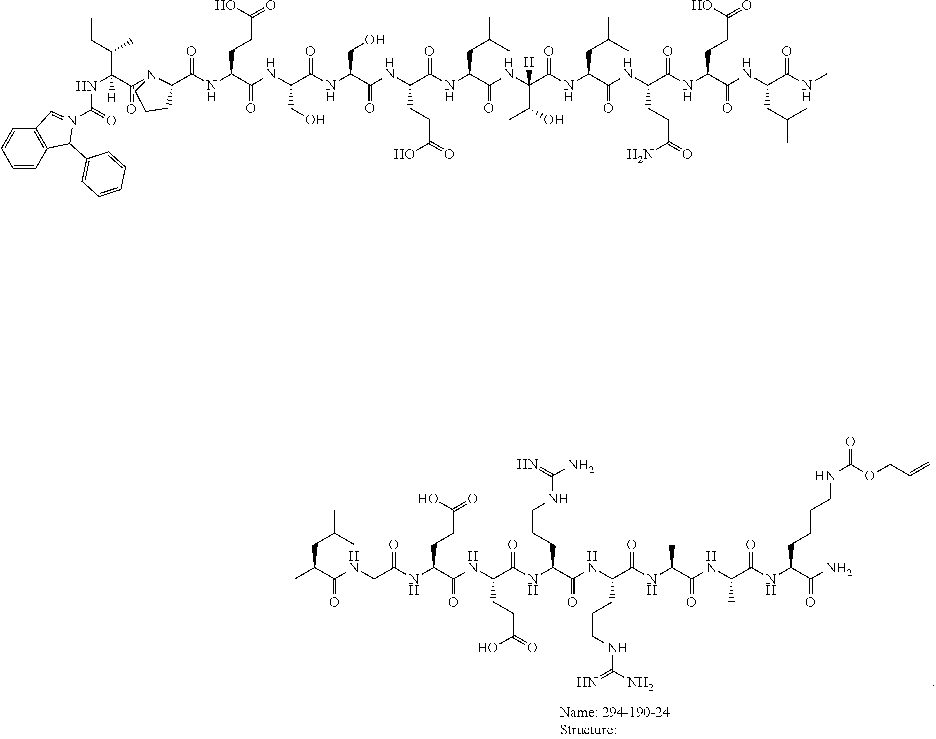

In another aspect, the disclosure provides for a synthetic peptide comprising the formula

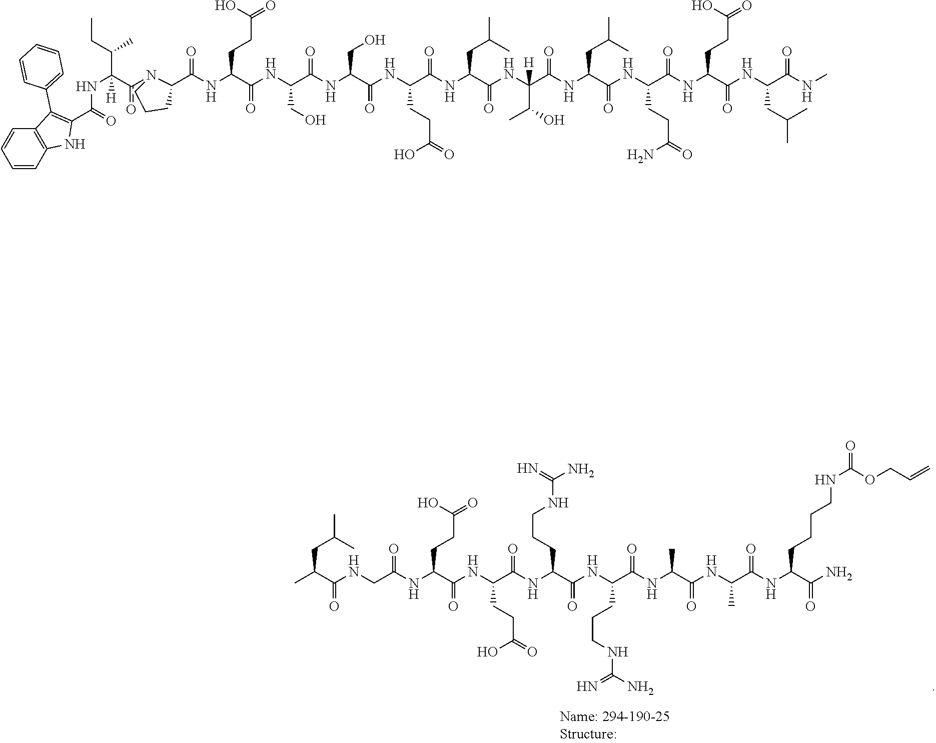

In another aspect, the disclosure provides for a synthetic peptide comprising the formula

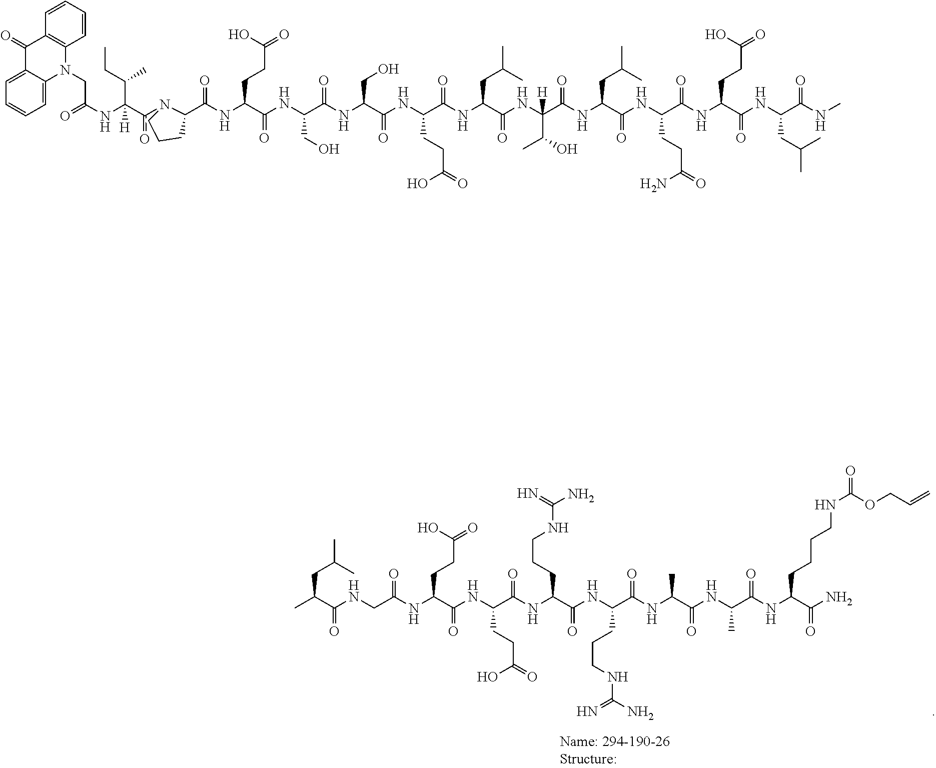

In another aspect, the disclosure provides for a synthetic peptide comprising the formula

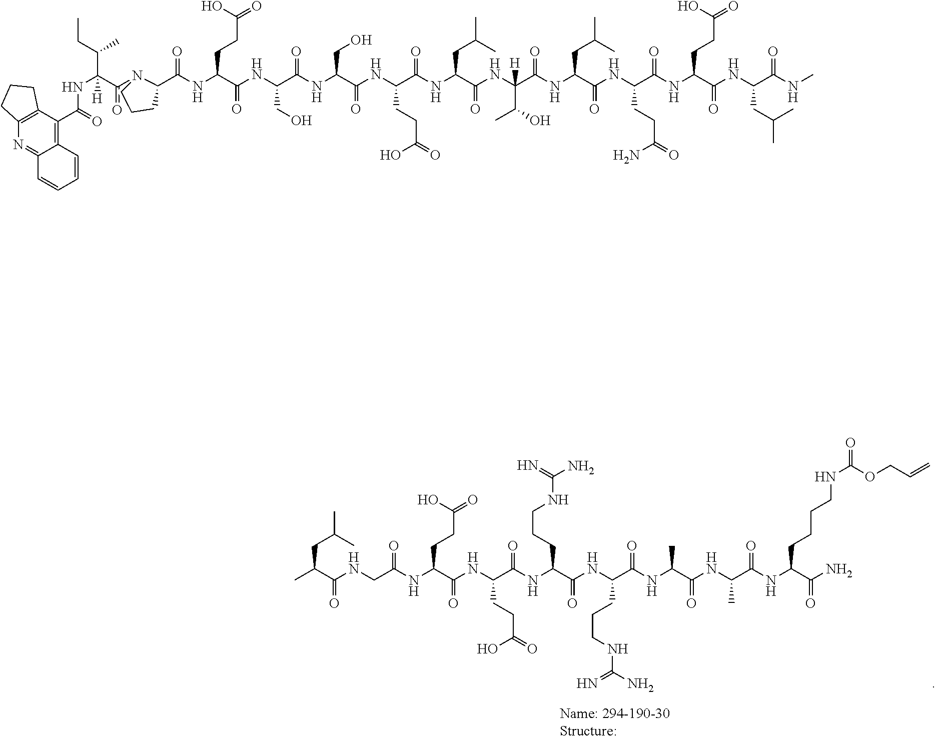

In another aspect, the disclosure provides for a synthetic peptide comprising the formula

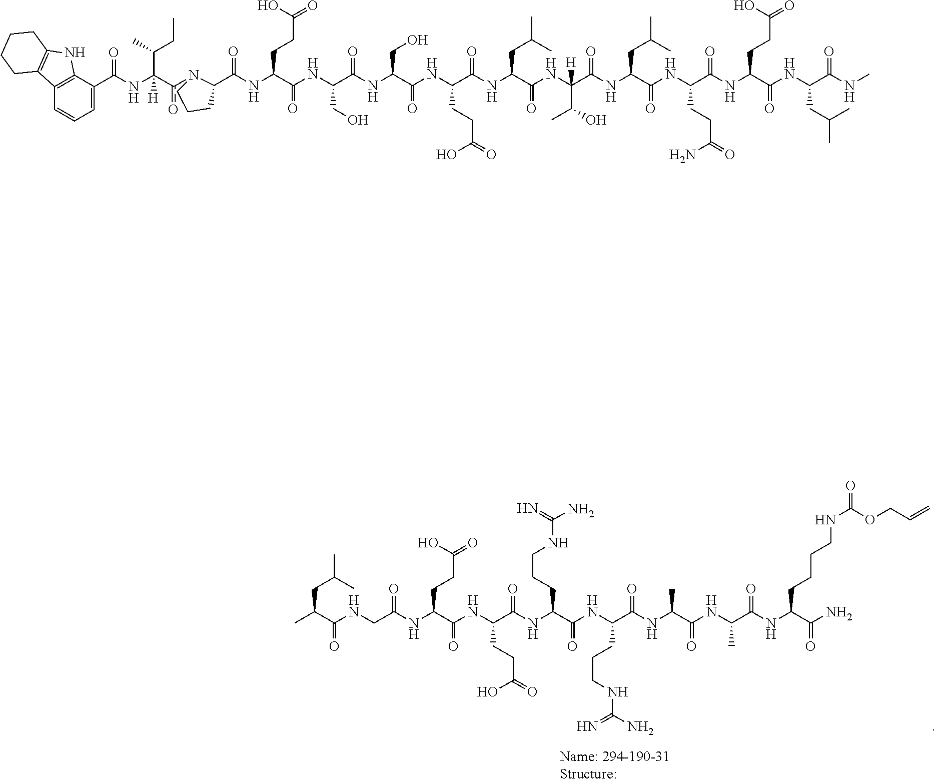

In another aspect, the disclosure provides for a synthetic peptide comprising the formula

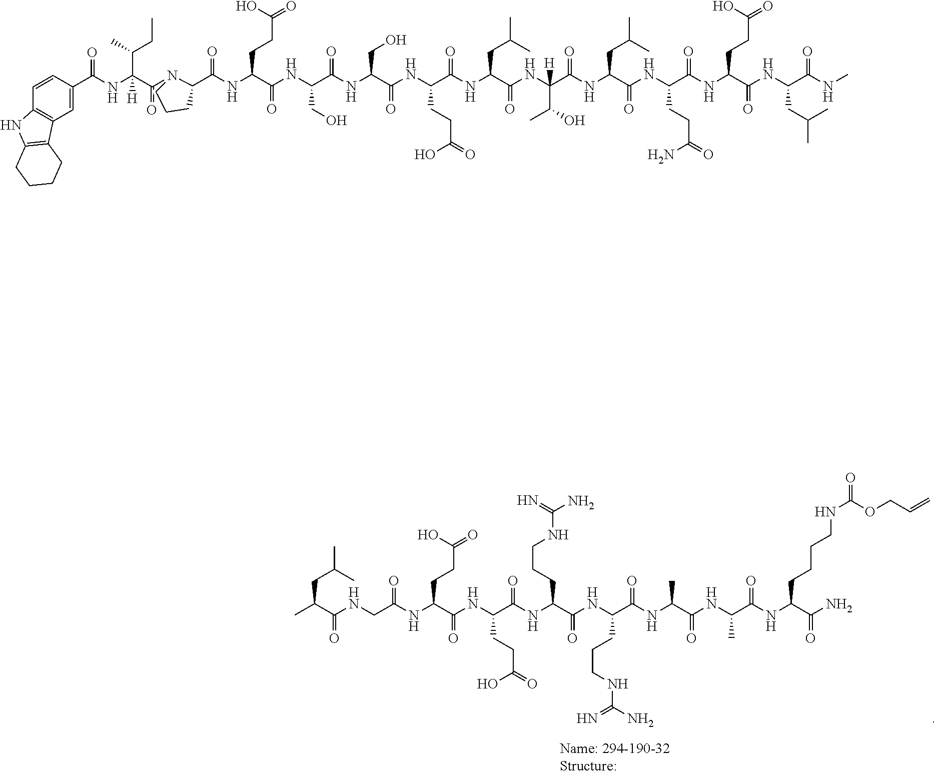

In another aspect, the disclosure provides for a synthetic peptide comprising the formula

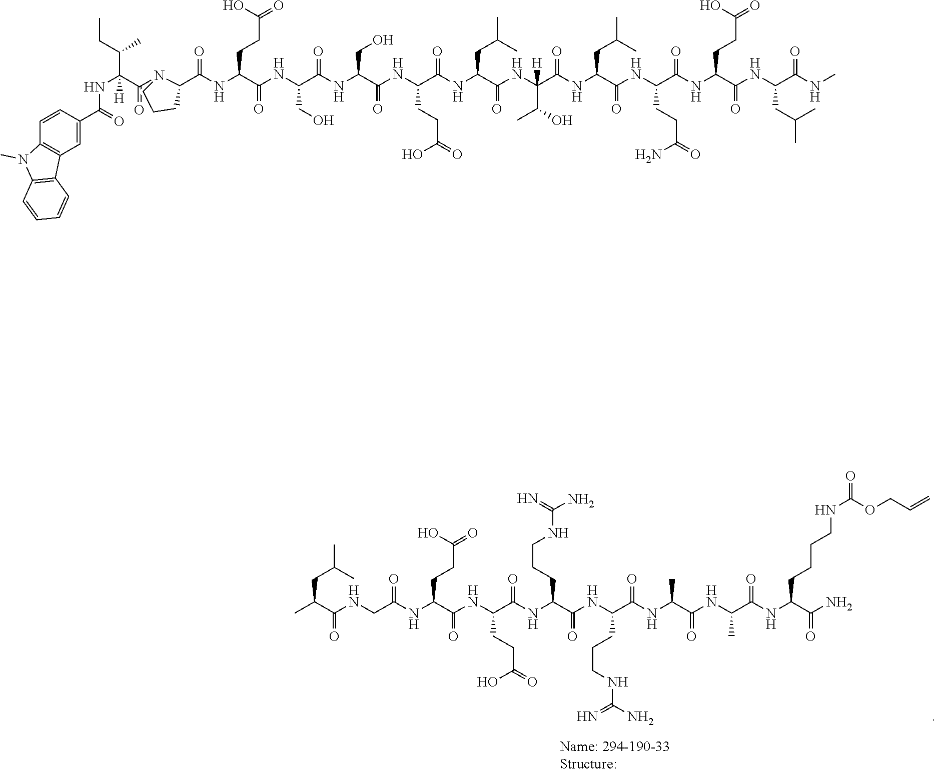

In another aspect, the disclosure provides for a synthetic peptide comprising the formula

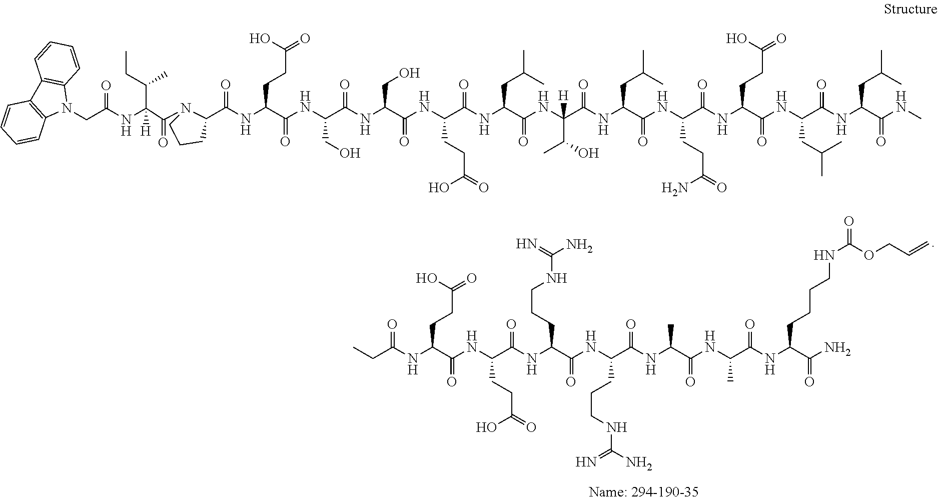

In another aspect, the disclosure provides for a synthetic peptide comprising the formula

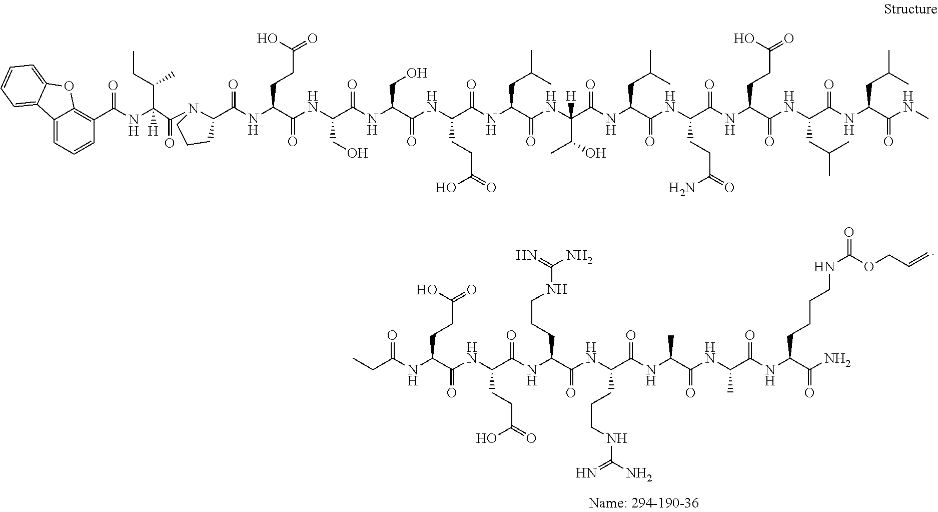

In some embodiments, the peptide comprises a substituted amino acid. In some embodiments, the substituted amino acid is a canonical amino acid. Canonical amino acids for use in substitutions are listed in TABLE A. In some embodiments, the canonical substituted amino acids are an Ala, a Ser, a Gln, or an Arg. TABLE A Canonical amino acids used in the peptides and peptidomimetics.

TABLE A

| Amino Acid | One Letter Code | Three Letter Code |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

In some aspects of the disclosure, the synthetic peptide or peptidomimetic comprises one or more non-canonical amino acids. Non-canonical amino acids that can be used for substitution are shown in TABLE B.

TABLE B Non-canonical amino acids for use in the peptides and peptidomimetics.

TABLE B

| Non canonical Amino Acid | Three Letter Code |
|---|---|
| 4-(Boc-amino)-L-Phe-OH | 4Af |
| 3-(4′-pyridyl)-L-Ala-OH | 4Py |
| L-α-aminoadipic acid(δtBu) | Aad |
| (4-aminomethyl) benzoic acid | Amb |
| L-2-aminooctanoic acid | Aoa |
| β-cyclobutyl-L-Ala-OH | Cba |
| β-cyclohexyl-L-Ala-OH | Cha |
| β-cyclopropyl-L-Ala-OH | Cpa |
| 2-(Boc)aminobutyric acid | Dab |
| 3,4-difluoro-L-Phe-OH | Dff |
| 3,4-dimethoxy-L-Phe-OH | Dmf |
| Nω-(Pbf)-L-homoArg-OH | hAr |
| homoPhe-OH (hPh), | hPh |
| L-hydroxyproline(tBu)-OH | Hyp^{t} |
| 4-fluoro-L-Phe-OH | Mff |
| L-Met sulfone-OH | Msn |
| 3-(2-naphtyl)-L-Ala-OH | Nal |
| L-ornithine(Boc)-OH | Orn |
| 4-phenylpiperidine-4-carboxylic acid | Php |
| 3,4,5-trifluoro-L-Phe-OH | Tff |
| 3-(4-thiazolyl)-L-Ala-OH | Tha |
| 4-phenyl phenylalanine | PhF |
| Napthylalanine | Naf |
| QuinolylAlanine | Qua |
| 6-Cl tryptophan | Clw |
| Cyclohexylglycine | Chg |
| Cyclopentylglycine | Ceg |
| d-Alanine | d-Ala |
| Amino isobutyric | Aib |
| Homocysteic acid | Hoc |
| L-2,3-diaminopropionic acid | Dap |
| Norvaline | Nva |
| | Aoc |
| Phenylglycine | Phg |
| | Hof |
| Cysteic acid | Cya |
| Hydroxyproline | Hyp |
| Cyclopropylglycine | Cpg |
| Cyclobutylglycine | Cbg |
| QuinolylAlanine | Qua |

In some embodiments the amino acids of the synthetic peptide are mixed canonical and non-canonical amino acids.

In some embodiments, hydroxyproline is substituted with a hydroxyproline derivative or protected analog (e.g., L-hydroxyproline(tBu)-OH).

In another aspect of the disclosure, the synthetic peptides and peptidomimetics are designed to bind HPV16E6. In some embodiments, the synthetic peptide binds HPV16E6 with low, medium, or high affinity. In some embodiments, the synthetic peptides and peptidomimetics bind HPV16E6 with higher affinity than SEQ ID NO: 1 or SEQ ID NO: 5. In some embodiments, the synthetic peptides are covalently bound to HPV16E6. In some embodiments, the covalent bond is between the synthetic peptide and HPV16E6 residues Cys-58, Arg-84, or Arg-13.

In some aspects, the disclosure is directed to libraries of synthetic disclosed herein. In some embodiments, the peptide library has the design X₁X₂X₃X₂QX₁X₂X₂CEER (SEQ ID NO: 6), wherein X₁ is Nva, Leu, Aoc, Cpa, Cba, Cha, Phg, Hof, or Naf; X₂ is Aad, Glu, or Cya; X₃ is Thr, Asn, Hyp, Cpg, Cbg, or Ceg; and X₄ is d-Ala, Aib, Gly, or Gln.

In some embodiments, the peptide library has the design Aad-X₁-X₂-Leu-Aib-Aad-Cba-Leu-Cys-X₃-X₃-X₃ (SEQ ID NO: 20) wherein X₁ is PhF, Naf, Qua, Clw, or Trp; X₂ is Chg or Ceg; and X₃ is d-Ala, Aib, Gly, Ser, Aad, Glu, Gln, Hoc, Val, Leu, Met, Pro, Dap, Lys, Arg, His, Trp, Phe, Tyr, or Null.

In some embodiments the library is 1 million, 2 million, 3 million, 4 million, 5 million, 6 million, 7 million, 8 million, 9 million, or 10 million peptides in size.

### III. Modifications

The central limitation in the development of peptide therapeutics is their short circulation time resulting from rapid enzymatic degradation and renal clearance. Methods to evade renal elimination by increasing the molecular weight have emerged, but extensive modifications can cause undesired steric hindrance during target binding. For small molecules, an alternative approach to modulate pharmacokinetic profiles and improve the potency and selectivity of a potential drug is the exploitation of covalent binding. Stability issues in peptides can be addressed via various strategies such as cyclization, incorporation of D- and non-canonical amino acids, and backbone modifications. A therapeutic small molecule ligand equipped with an electrophilic warhead binds covalently to nucleophilic groups of the target protein in a proximity-driven reaction. Irreversible covalent inhibition of an interaction can result in increased potency, selectivity, sustained pharmacodynamics, and could alleviate the effects of fast renal elimination. Therapeutic peptides may benefit from a covalent binding mode of action and alleviate pharmacokinetic limitations of this class of therapeutics.

In some aspects, the disclosure is directed to synthetic peptides that are chemically modified. In some embodiments, the peptide that is modified is selected from TABLE 1-TABLE 40. In some embodiments, the peptide that is modified is selected from SEQ ID NOS: 1-20. Modifications may comprise chemical modifications for example such as warheads, protective groups, and pegylation. In some embodiments, the modification is at the N- or C-terminus of the peptide. In some embodiments, the modification is on a side chain of an amino acid in the peptide. In some embodiments, the modification is acetylation, formylation, propanoylation, hexanoylation, or myristoylation. In some embodiments, the modification is an amidated C-terminus. In some embodiments, the modification is a substitution of one or more L-amino acid with a D-amino acid. In some embodiments, the modification is a substitution of one or more amino acid with a methyl-amino acid. In some embodiments, the modification is a substitution of an α-amino acid with a β-amino acids.

In some embodiments, the N- or C-terminus of the peptide is modified with a chemical moiety.

In some embodiments, the chemical moiety is attached at the N-terminus and is selected from the group of Fluorescein-5-Isothiocyanate, 9-fluorene acetamido, 1-fluorene acetamido, 1-Indane acetamido, 9-fluorenone-2-carboxamido, 9-fluorenone-1-carboxamido, 9-fluorenone-4-carboxamido, anthraquinone-2-carboxamido, xanthene-9-carboxamido, 1-anthracene carboxamido, 2-anthracene carboxamido, 1-adamantane carboxamido, Triphenyl acetamido, Diphenyl acetamido, 1-naphthyl carboxamido, 1,6-dihydrophenyl carboxamido, Pentafluorophenyl carboxamido, 6-hydroxy-2-naphthyl carboxamido, 1-pyrenebutyl carboxamido, 5-Acenaphthene carboxamide, 7-Methoxycoumarin-4-acetamido, 4-phenyl-phenylalanine, and Cyclohexyl-alanine.

In some embodiments, the chemical moiety is attached at the C-terminus and is selected from the group of Fluorescein-5-Isothiocyanate, fluorene-9-acetamido, fluorene-1-acetamido, 1-Indanecarboxamido, 9-fluorenone-2-carboxamido, 9-fluorenone-1-carboxamido, 9-fluorenone-4-carboxamido, anthraquinone-2-carboxamido, xanthene-9-carboxamido, 1-anthracene carboxamido, 2-anthracene carboxamido, 1-adamantane carboxamido, triphenylacetamido, diphenylacetamido, 1-naphthyl carboxamido, and Exo-norbornene-carboxamido. In some embodiments, the chemical moiety is selected from the group of 9-fluorenyl, 1-fluorenyl, 1-Indanyl, 9-oxofluoren-3-yl, 9-oxofluoren-4-yl, 9-oxofluoren-1-yl, 2-anthraquinonyl, 9-xanthyl, 1-anthracenyl, 2-anthracenyl, Adamantyl, Triphenylmethyl, Diphenylmethyl, 1-naphthyl, 2,6-dihydroxy-phenyl, Pentafluoro-phenyl, 6-Hydroxynaphthyl, 1-pyrenebutyl, 5-acenaphthyl, Coumarin, Biphenyl, Cyclohexyl, Norborenyl, and Fluorescein.

In some embodiments, a first chemical moiety is attached at the N-terminus of the peptide and a second chemical moiety is attached at the C-terminus of the peptide. In some embodiments, a first chemical moiety is selected from the group of Biotin-PEG4, Fluorene, Anthracene, and Fluorene-(Biotin-PEG4-)K, and the second chemical moiety is selected from the group of 9-fluorene, 5-Acenaphthene, 1-naphthanlene, 2-anthraquinone, and 1-Anthracene.

In some embodiments, the N-terminus of the peptide is further modified with a modifier selected from 2-Phenyl-4-quinolinecarboxylic acid, 1-(Phenylsulfonyl)-1H-indole-2-carboxylic acid, 6-Fluoro-2-naphthoic acid, 4-phthalimidobenzoic acid, Xanthene 9-carboxylic acid, 1-Pyrenebutyric acid, 5-Acenaphthenecarboxylic acid, 1-Phenyl-1H-indole-2-carboxylic acid, Indole-2-carboxlic acid, quinoline-4-carboxylic acid, and 10-(Carboxymethyl)-9(10H)acridone.

### Warheads

In some aspects, the disclosure is directed to a synthetic peptide comprising a warhead. In some embodiments, the peptide is modified with a warhead that is selected from TABLE 2C, TABLE 2D, or TABLE 2E. In some embodiments, the peptide that is modified is selected from SEQ ID NOS: 1-20. In some embodiments, the warhead facilitates a covalent bond to a cognate protein after a chemical reaction. In some embodiments, the warhead is at the N- or C-terminus of the peptide or peptidomimetic. In some embodiments, the warhead is on a side chain of an amino acid in the peptide or peptidomimetic In some embodiments, the warhead is on a Cys, Lys, Tyr, His, Ser, or Thr.

In some embodiments, the warhead is on a Cys, and the Cys is a position 9 of the peptide with respect to SEQ ID NO: 3. In some embodiments, the warhead is conjugated to the Cys via the Sulfur atom of the Cys.

In some embodiments, the warhead is selected from the group of phenylacrylamide (Ph-acr), Dap-acrylamide (dap-acr), Dab-acrylamide (dab-acr), Dap-propiolamide (dap-ppa), and dehydroalanine Dha.

In some embodiments, the warhead is selected from the group of phenyl fluorosulfate (FS), phenyl sulfonyl fluoride (SF), phenyl Carbamate (p-PhC), phenyl Carbamate (m-PhC), and disulfide (DS).

In some embodiments, the peptide with a warhead has the following formula

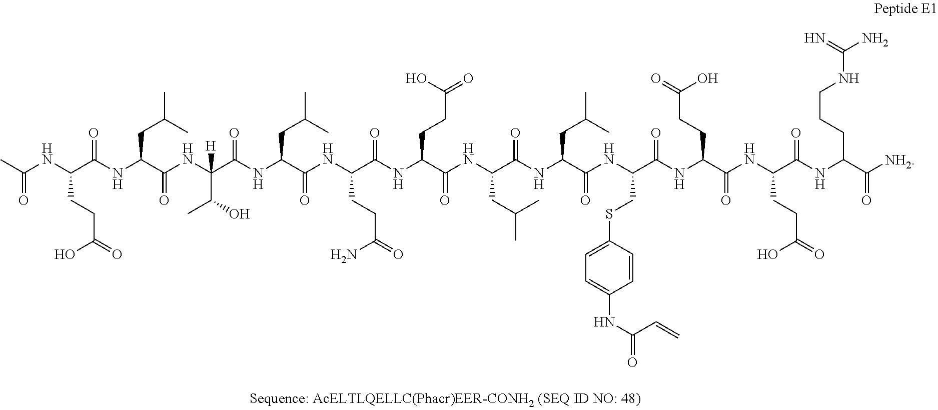 [SMILES: C=CC(=O)Nc1ccc(SC[C@H](NC(=O)[C@H](CC(C)C)NC(=O)[C@H](CC(C)C)NC(=O)[C@H](CCC(=O)O)NC(=O)[C@H](CCC(N)=O)NC(=O)[C@H](CC(C)C)NC(=O)[C@@H](NC(=O)[C@H](CC(C)C)NC(=O)[C@H](CCC(=O)O)NC(C)=O)[C@@H](C)O)C(=O)N[C@@H](CCC(=O)O)C(=O)N[C@@H](CCC(=O)O)C(=O)NC(CCCNC(=N)N)C(N)=O)cc1]

In some embodiments, the peptide with a warhead has the following formula

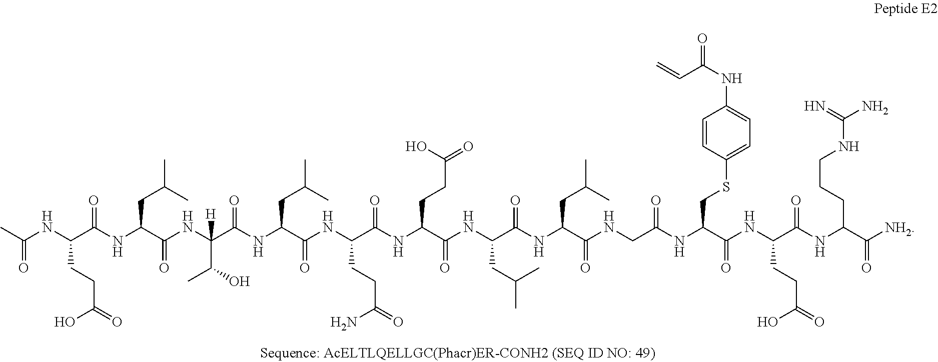 [SMILES: C=CC(=O)Nc1ccc(SC[C@H](NC(=O)CNC(=O)[C@H](CC(C)C)NC(=O)[C@H](CC(C)C)NC(=O)[C@H](CCC(=O)O)NC(=O)[C@H](CCC(N)=O)NC(=O)[C@H](CC(C)C)NC(=O)[C@@H](NC(=O)[C@H](CC(C)C)NC(=O)[C@H](CCC(=O)O)NC(C)=O)[C@@H](C)O)C(=O)N[C@@H](CCC(=O)O)C(=O)NC(CCCNC(=N)N)C(N)=O)cc1]

In some embodiments, the peptide with a warhead has the following formula

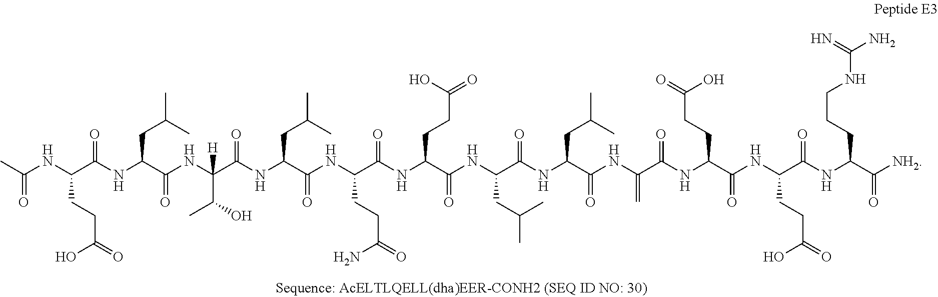 [SMILES: C=C(NC(=O)[C@H](CC(C)C)NC(=O)[C@H](CC(C)C)NC(=O)[C@H](CCC(=O)O)NC(=O)[C@H](CCC(N)=O)NC(=O)[C@H](CC(C)C)NC(=O)[C@@H](NC(=O)[C@H](CC(C)C)NC(=O)[C@H](CCC(=O)O)NC(C)=O)[C@@H](C)O)C(=O)N[C@@H](CCC(=O)O)C(=O)N[C@@H](CCC(=O)O)C(=O)N[C@@H](CCCNC(=N)N)C(N)=O]

In some embodiments, the peptide with a warhead has the following formula

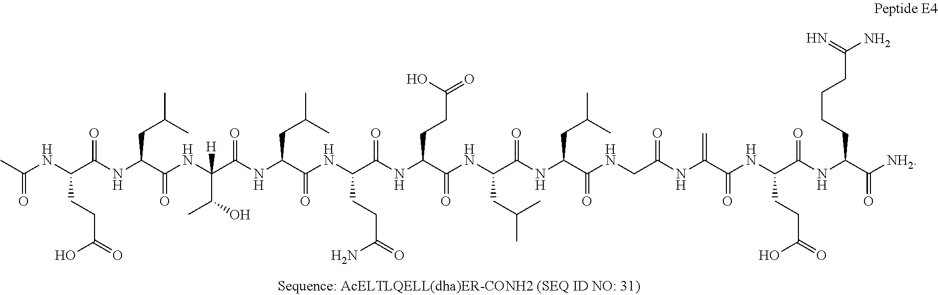 [SMILES: C=C(NC(=O)CNC(=O)[C@H](CC(C)C)NC(=O)[C@H](CC(C)C)NC(=O)[C@H](CCC(=O)O)NC(=O)[C@H](CCC(N)=O)NC(=O)[C@H](CC(C)C)NC(=O)[C@@H](NC(=O)[C@H](CC(C)C)NC(=O)[C@H](CCC(=O)O)NC(C)=O)[C@@H](C)O)C(=O)N[C@@H](CCC(=O)O)C(=O)N[C@@H](CCCCC(=N)N)C(N)=O]

In some embodiments, the peptide with a warhead has the following formula

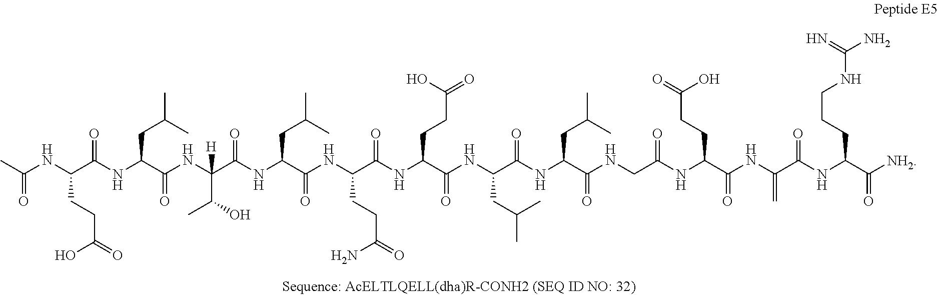 [SMILES: C=C(NC(=O)[C@H](CCC(=O)O)NC(=O)CNC(=O)[C@H](CC(C)C)NC(=O)[C@H](CC(C)C)NC(=O)[C@H](CCC(=O)O)NC(=O)[C@H](CCC(N)=O)NC(=O)[C@H](CC(C)C)NC(=O)[C@@H](NC(=O)[C@H](CC(C)C)NC(=O)[C@H](CCC(=O)O)NC(C)=O)[C@@H](C)O)C(=O)N[C@@H](CCCNC(=N)N)C(N)=O]

In some embodiments, the peptide with a warhead has the following formula

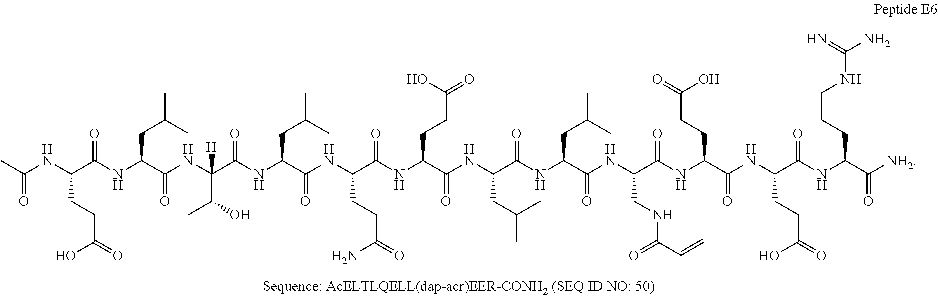 [SMILES: C=CC(=O)NC[C@H](NC(=O)[C@H](CC(C)C)NC(=O)[C@H](CC(C)C)NC(=O)[C@H](CCC(=O)O)NC(=O)[C@H](CCC(N)=O)NC(=O)[C@H](CC(C)C)NC(=O)[C@@H](NC(=O)[C@H](CC(C)C)NC(=O)[C@H](CCC(=O)O)NC(C)=O)[C@@H](C)O)C(=O)N[C@@H](CCC(=O)O)C(=O)N[C@@H](CCC(=O)O)C(=O)N[C@@H](CCCNC(=N)N)C(N)=O]

In some embodiments, the peptide with a warhead has the following formula

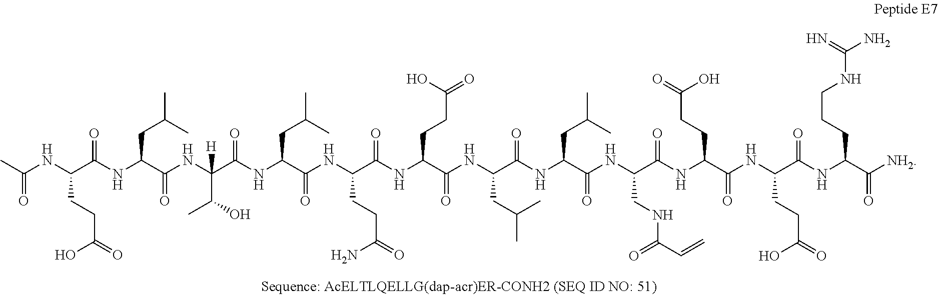 [SMILES: C=CC(=O)NC[C@H](NC(=O)[C@H](CC(C)C)NC(=O)[C@H](CC(C)C)NC(=O)[C@H](CCC(=O)O)NC(=O)[C@H](CCC(N)=O)NC(=O)[C@H](CC(C)C)NC(=O)[C@@H](NC(=O)[C@H](CC(C)C)NC(=O)[C@H](CCC(=O)O)NC(C)=O)[C@@H](C)O)C(=O)N[C@@H](CCC(=O)O)C(=O)N[C@@H](CCC(=O)O)C(=O)N[C@@H](CCCNC(=N)N)C(N)=O]

In some embodiments, the peptide with a warhead has the following formula Peptide E8

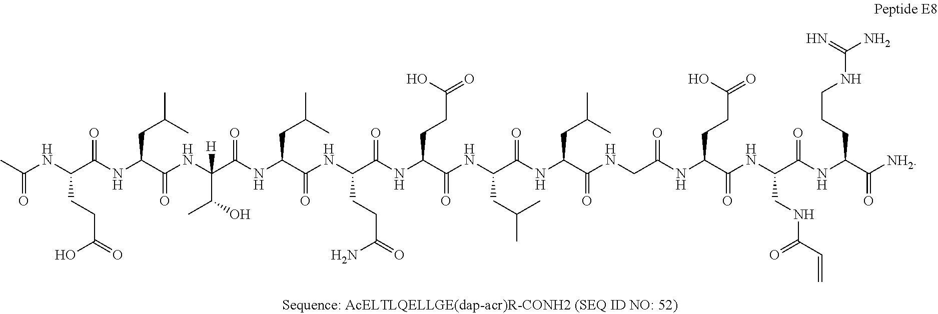 [SMILES: C=CC(=O)NC[C@H](NC(=O)[C@H](CCC(=O)O)NC(=O)CNC(=O)[C@H](CC(C)C)NC(=O)[C@H](CC(C)C)NC(=O)[C@H](CCC(=O)O)NC(=O)[C@H](CCC(N)=O)NC(=O)[C@H](CC(C)C)NC(=O)[C@@H](NC(=O)[C@H](CC(C)C)NC(=O)[C@H](CCC(=O)O)NC(C)=O)[C@@H](C)O)C(=O)N[C@@H](CCCNC(=N)N)C(N)=O]

In some embodiments, the peptide with a warhead has the following formula

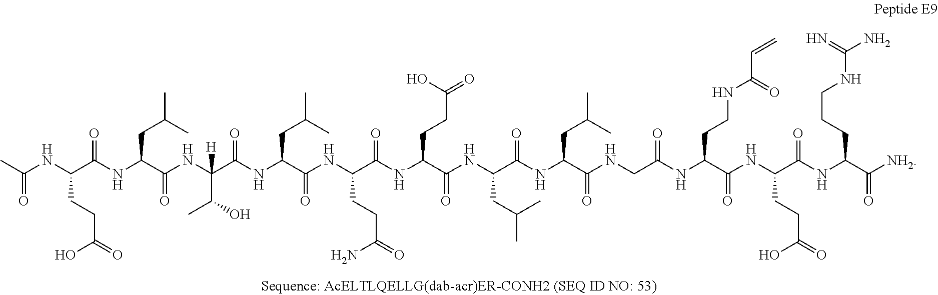 [SMILES: C=CC(=O)NCC[C@H](NC(=O)CNC(=O)[C@H](CC(C)C)NC(=O)[C@H](CC(C)C)NC(=O)[C@H](CCC(=O)O)NC(=O)[C@H](CCC(N)=O)NC(=O)[C@H](CC(C)C)NC(=O)[C@@H](NC(=O)[C@H](CC(C)C)NC(=O)[C@H](CCC(=O)O)NC(C)=O)[C@@H](C)O)C(=O)N[C@@H](CCC(=O)O)C(=O)N[C@@H](CCCNC(=N)N)C(N)=O]

In some embodiments, the peptide with a warhead has the following formula

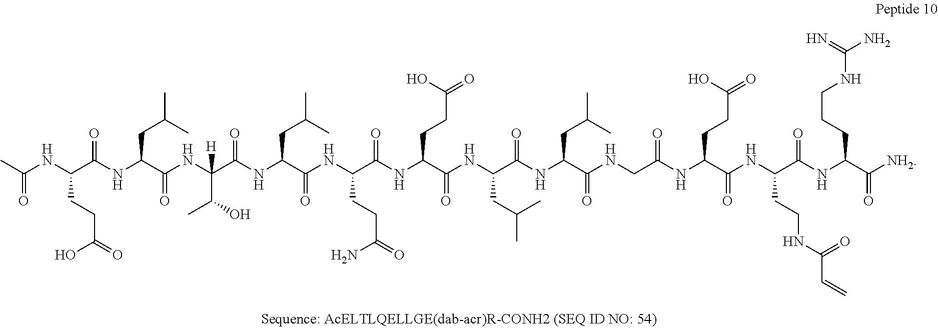 [SMILES: C=CC(=O)NCC[C@H](NC(=O)[C@H](CCC(=O)O)NC(=O)CNC(=O)[C@H](CC(C)C)NC(=O)[C@H](CC(C)C)NC(=O)[C@H](CCC(=O)O)NC(=O)[C@H](CCC(N)=O)NC(=O)[C@H](CC(C)C)NC(=O)[C@@H](NC(=O)[C@H](CC(C)C)NC(=O)[C@H](CCC(=O)O)NC(C)=O)[C@@H](C)O)C(=O)N[C@@H](CCCNC(=N)N)C(N)=O]

In some embodiments, the peptide with a warhead has the following formula

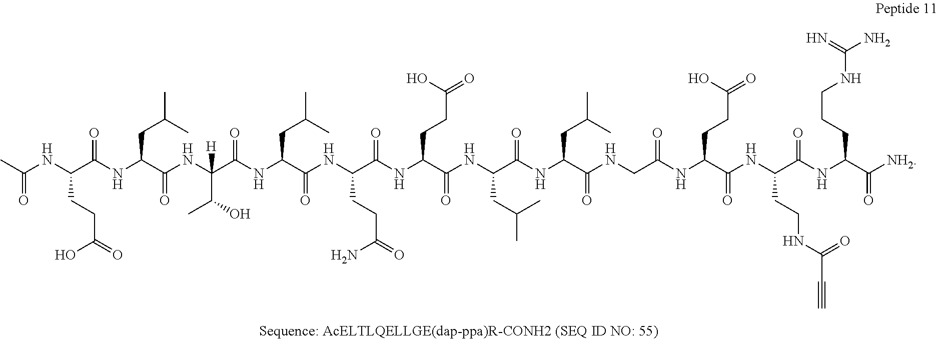 [SMILES: C#CC(=O)NCC[C@H](NC(=O)[C@H](CCC(=O)O)NC(=O)CNC(=O)[C@H](CC(C)C)NC(=O)[C@H](CC(C)C)NC(=O)[C@H](CCC(=O)O)NC(=O)[C@H](CCC(N)=O)NC(=O)[C@H](CC(C)C)NC(=O)[C@@H](NC(=O)[C@H](CC(C)C)NC(=O)[C@H](CCC(=O)O)NC(C)=O)[C@@H](C)O)C(=O)N[C@@H](CCCNC(=N)N)C(N)=O]

### IV. Complexes

In some aspects, the disclosure is directed to synthetic peptides that are bound in a complex with HPV16E6 to form peptide/HPV16E6. In some embodiments, the peptide that is complexed with HPV16E6 is selected from TABLE 1 to TABLE 40. In some embodiments, the peptide that is complexed with HPV16E6 is selected from SEQ ID NOS: 1-20.

In some embodiments, the synthetic peptides are designed to bind HPV16E6. In some embodiments, the synthetic peptides bind HPV16E6 with low, medium, or high affinity. In some embodiments, the synthetic peptides bind HPV16E6 with higher affinity than SEQ ID NO: 1 or SEQ ID NO: 5. In some embodiments, the peptides are covalently bound to HPV16E6. In some embodiments, the covalent bond is between the synthetic peptides and HPV16E6 residues Cys-58, Arg-84, or Arg-13

In some embodiments, the peptide/HPV16E6 is modulated in binding of E3 ubiquitin ligase E6AP. In some embodiments, the peptide/HPV16E6 is inhibited in binding or engaging of E3 ubiquitin ligase E6AP. In some embodiments, the peptide/HPV16E6 is soluble in a cell. In some embodiments, the cell is a cancer cell. In some embodiments, the HPV16E6 is located in a cancer cell.

### V. Preparation of Peptides

Methods for producing synthetic peptide or peptidomimetic of the disclosure are known in the art such as solid phase peptide synthesis (SPPS), Fmoc-based synthesis, and Boc-based synthesis by an automatic peptide synthesizer. For example, peptides can be chemically synthesized using the sequence information provided herein and using peptide synthesis methods known in the art. The produced synthetic peptide or peptidomimetic can be modified during or after peptide synthesis with several modifications, for example with a warhead, a protective group, or pegylation. Alternatively or additionally, the peptide or peptidomimetic may be modified at its amino terminus or carboxy terminus or protected by various organic groups for protecting the peptide from protein-cleaving enzymes in vivo while increasing its stability. The produced synthetic peptide or peptidomimetic can then be purified further. Purification strategies for peptides or peptidomimetics are known in the art, and include FPLC and HPLC based methods.

### VI. Pharmaceutical Compositions

For therapeutic use, a synthetic peptide or peptidomimetic disclosed herein preferably is combined with a pharmaceutically acceptable carrier and/or an excipient. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA

Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing a synthetic peptide disclosed herein can be presented in a dosage unit form and can be prepared by any suitable method. A pharmaceutical composition should be formulated to be compatible with its intended route of administration, e.g., oral administration. The pharmaceutical compositions may be in a variety of forms. These include, for example, liquid, semi-solid and solid dosage forms, such as liquid solutions, dispersions or suspensions, tablets, pills, powders, liposomes and suppositories. The preferred form will depend upon the intended mode of administration and therapeutic application.

The composition can be formulated as a solution, microemulsion, dispersion, liposome, or other ordered structure suitable for stable storage at high concentration. Sterile solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

### VII. Methods of Use

The synthetic peptides of the disclosure can be used in a variety of in vitro and in vivo methods, as research reagents, for diagnostic purposes, and for therapeutic uses, based on the binding specificity of the synthetic peptides to HPV 16E6 and on the effect on HPV 16E6 functions of the peptides.

The synthetic peptides and peptidomimetics disclosed herein are designed to be bound covalently or non-covalently by HPV 16E6. In some embodiments, the synthetic peptides can be used to modulate HPV 16E6 function. In some embodiments, the synthetic peptides and peptidomimetics in complex with HPV 16E6 modulate HPV 16E6 engagement with E6AP and its ubiquitination of p53. In some embodiments, the synthetic peptides in complex with HPV 16E6 block or inhibit HPV 16E6 engagement with E6AP in cells.

Methods for testing for HPV 16E6-E6AP engagement and subsequent cell signaling are known in the art, for example by p53 ubiquitination and degradation assays.

### VIII. Kits

In some embodiments, any of the synthetic peptides disclosed herein is assembled into a pharmaceutical or diagnostic or research kit to facilitate their use in therapeutic, diagnostic or research applications. A kit may include one or more containers housing any of the systems or vectors disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

### EXAMPLES

Below are examples of specific embodiments for carrying out what is disclosed herein. The examples are offered for illustrative purposes only and are not intended to limit scope.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, PROTEINS: STRUCTURES AND MOLECULAR PROPERTIES (W.H. Freeman and Company, 1993); A. L. Lehninger, BIOCHEMISTRY (Worth Publishers, Inc., current addition); Sambrook, et al. MOLECULAR CLONING: A LABORATORY MANUAL (2nd Edition, 1989); METHODS IN ENZYMOLOGY (S. Colowick and N. Kaplan eds., Academic Press, Inc.); REMINGTON'S PHARMACEUTICAL SCIENCES, 18th Edition (Easton, Pennsylvania: Mack Publishing Company, 1990); Carey and Sundberg ADVANCED ORGANIC CHEMISTRY 3^{rd} Ed. (Plenum Press) Vols A and B (1992).

Additional data and methods are provided in Ye, Xiyun, et al. “Discovery of reactive peptide inhibitors of human papillomavirus oncoprotein E6.” Chemical Science 14.44 (2023): 12484-12497, the entire contents of which are incorporated by reference herein for all purposes.

Unless otherwise stated, all reagents and chemicals were obtained from commercial sources and used without further purification.

### Example 1—Synthesis of Peptides

This example describes the synthesis of peptides used in the following examples.

### Fast Flow Synthesis of Peptides

H-Rink Amide-ChemMatrix resin (200 mg, 0.49 mmol/g, 0.10 mmol) was used to prepare peptide-α-carboxamides. Peptides containing noncanonical amino acids were prepared by manual SPPS. Peptides without noncanonical amino acids were prepared by fully automated SPPS^{[1]}. Upon completion, resins were washed with dichloromethane (DCM) three times and dried under reduced pressure.

### Automated Flow Peptide Synthesis (AFPS) Set-Up

All peptides were synthesized on automated-flow systems built in the Pentelute lab (“Amidator” and “Peptidator”), which are similar to the published AFPS system. The synthesis conditions were published previously^{[2]}:

The following settings were used for protein synthesis: flowrate=40 mL/min, temperature=90° C. (loop) and 85-90° C. (reactor). The 50 ml/min pump head pumps 400 μL of liquid per pump stroke; the 5 mL/min pump head pumps 40 μL of liquid per pump stroke. The standard synthetic cycle involves a first step of prewashing the resin at elevated temperatures for 60 s at 40 mL/min. During the coupling step, three HPLC pumps are used: a 50 mL/min pump head pumps the activating agent, a second 50 ml/min pump head pumps the amino acid and a 5 mL/min pump head pumps DIEA. The first two pumps are activated for 8 pumping strokes to prime the coupling agent and amino acid before the DIEA pump is activated. The three pumps are then actuated together for a period of 7 pumping strokes, after which the activating agent pump and amino acid pump are switched using a rotary valve to select DMF. The three pumps are actuated together for a final 8 pumping strokes, after which the DIEA pump is shut off, and the other two pumps continue to wash the resin for another 40 pump strokes. During the deprotection step, two HPLC pumps are used. Using a rotary valve, one HPLC pump selects deprotection stock solution and DMF. The pumps are activated for 13 pump strokes. Both solutions are mixed in a 1:1 ratio. Next, the rotary valves select DMF for both HPLC pumps, and the resin is washed for an additional 40 pump strokes. The coupling-deprotection cycle is repeated for all additional monomers.

### Method for Peptide Acetylation

A 100 mg portion of peptidyl resin from section 1.3 was placed into a 5 mL Torviq fritted syringe and subsequently swelled in DMF. After removing DMF, a solution of Ac₂O, DIEA, and DMF (2 mL, 85:315:1600, v/v) were added to the peptidyl resin. The resulting mixture was occasionally agitated for 45 min. After draining the solution, the remaining resin was washed with DMF three times, DCM three times, and dried under reduced pressure.

### Method for Alloc Deprotection

Peptidyl resin (~10 μmol theoretical loading) was washed with DCM (3×5 mL) and then treated with Pd(PPh₃)₄ (11.0 mg, 10 mol, 1 equiv) in DCM/piperidine (8:2, 1 mL) for 30 minutes at room temperature under exclusion of light. The resin was then drained and washed with DCM (3×5 mL).

### Method for Biotin Labeling

Peptidyl resin (~10 mol theoretical loading) was loaded into a fritted syringe (6 mL), swollen in DMF (4 mL) for 5 minutes, and then drained. Biotin-PEG4-propionic acid (Biotin-PEG4-OH, 22 mg, 50 μmol, 5 equivalents) and HATU (17 mg, 45 μmol, 4.5 equivalents) were dissolved in DMF (500 μL), activated with DIEA (19 mg, 26 μL, 150 μmol), added to the peptidyl resin and incubated for 30 minutes under exclusion of light. After this time, the resin was drained, washed with DMF (3×5 mL), and stored until cleavage.

### Method for TAMRA Labeling

Peptidyl resin (~10 μmol theoretical loading) was loaded into a fritted syringe (6 mL), swollen in DMF (4 mL) for 5 minutes, and then drained. 5-Carboxytetramethylrhodamine (5-TAMRA, 22 mg, 50 μmol, 5 equivalents) and HATU (17 mg, 45 μmol, 4.5 equivalents) were dissolved in DMF (500 μL), activated with DIEA (19 mg, 26 μL, 150 μmol), added to the peptidyl resin and incubated for 30 minutes under exclusion of light. After this time, the resin was drained, washed with DMF (3×5 mL), and stored until cleavage.

### Method for FITC Labeling

Peptidyl resin (~10 μmol theoretical loading) was loaded into a fritted syringe (6 mL), swollen in DMF (4 mL) for 5 minutes, and then drained. 5-Carboxytetramethylrhodamine (Fluorescein isomer I, 22 mg, 50 μmol, 5 equivalents) was dissolved in DMF (500 μL), activated with DIEA (19 mg, 26 μL, 150 μmol), added to the peptidyl resin and incubated for 30 minutes under exclusion of light. After this time, the resin was drained, washed with DMF (3×5 mL) and stored until cleavage.

### Cleavage of Peptides

The synthesized peptide was cleaved from the resin and globally deprotected by treating the peptidyl resin with a cleavage cocktail containing 94% TFA, 2.5% water, and 2.5% TIPS (v v), for 2 h at room temperature. TFA was removed under a gentle stream of nitrogen gas, and the crude peptide was precipitated by adding cold Et₂O (−80° C.). After centrifugation at 3220 rcf for 3 min, the supernatant was removed, and the precipitated peptide was triturated three times with cold Et₂O. The resulting material was dissolved in 50% MeCN in water with 0.1% TFA and lyophilized as crude.

### Pd Mediated Conjugation

In a 50 mL falcon tube: Dissolve peptide (5.0 mg, 2.75 mmol, 1.0 equiv) in water (8.5 mL) and 500 mM HEPES (1.5 mL, pH=8.0). In a 20 mL glass vial: Dissolve Pd OAC (5.0 mg, 6.19 mmol, 2.25 equiv) in MeCN and add to peptide solution over 30 seconds (final volume=15 mL, final peptide concentration=184 μM). Mix by vortexing and let stand for 20 minutes. Add 10 mL AcOH and 30 mL H₂O and mix and then purify by reversed phase flash chromatography using a Sfar Bio C18 D (300 Å 20 μm, 6 g) column (mobile phase 5% MeCN/H₂O to 55% MeCN in H₂O+0.1% TFA).

### Dehydroalanine Formation

In a 1.5 mL microcentrifuge tube, cysteine-containing peptide (7 mg) was dissolved in DMF (0.5 mL), and to this, a 10 mg/mL potassium carbonate solution (3.1 mg, 22.3 μmol, 5 equiv) was added. To this mixture, a 10 mg/mL solution of Diethylmeso-2,5-dibromoadipate (151 μL, 1.1 equiv) was added. The mixture was mixed by vortexing for 5 seconds and allowed to react for 4 h. The reaction mixture was diluted with 5% MeCN in H₂O+0.1% TFA and purified by reversed-phase HPLC (Zorbax 300SB-C3, 300A, 5 μm, 9.4 mm×250 mm) Mobile phase 5% to 55% MeCN in H₂O+0.1% TFA.

### Purification of the Crude Peptide.

Crude peptides were purified by a Biotage Selekt flash purification system. Water with 0.1% TFA (solvent A) and MeCN with 0.1% TFA (solvent B) was utilized as mobile phases for purifications. The crude peptide was dissolved in a minimal amount of 10% MeCN in water with 0.1% TFA and then loaded onto a 10 g Biotage SNAP Bio C4 20 μm column. The purification was performed using a gradient as follows: 10% B for 2 column volume (CV), the linear ramp from 30% B to 50% B for 20 CV, 25 mL/min flow rate.

### In Solution Disulfide Dimer Formation

In a 15 mL falcon tube: Dissolve peptide (5.0 mg, 2.75 mmol, 1.0 equiv) in 50% MeCN in H₂O+0.1% TFA (1 mL). Slowly add 50 equiv of 0.3M I₂ in MeOH into the peptide solution. Then quench the excessive oxidant with 1M of L-Ascorbic acid in water. Mix by vortexing and then purified by reversed-phase flash chromatography using a Sfar Bio C14 D (300 Å 20 μm, 10 g) column (mobile phase 20% MeCN/H₂O to 30% MeCN in H₂O+0.1% TFA, 10 column volume).

### On-Resin Mixed Disulfide Formation

To 50 mg resin (0.025 mmol, 1 eq.) add freshly prepared 1M Iodine in DMF solution (50 eq., stir for 2 min and drain. Add freshly prepared 1M Cysteamine-HCl in DMF (10 eq.) and DIPEA (5 eq.), stir for 10 min. Wash with DMF. Cleavage solution of disulfides: neat TFA, 1 h, RT.

### Example 2—LC-MS Characterization of Peptides

### LC-MS Methods

LC-MS characterizations were carried out using an Agilent 6550 quadrupole time-of-flight LC-MS. Total ion current (TIC) chromatograms were plotted. Mass spectra were integrated over the principal TIC peaks. High-performance liquid chromatography was done by the following methods: (solvent A: water with 0.1% formic acid; solvent B: MeCN with 0.1% formic acid).

Method A: Column: Phenomenex Aeris C4 column (1.0×150 mm, 5 μm particle size, 300 Å pore size) Gradient: 1% B (0-2 min), linearly ramp from 1% B to 91% B (2-10 min). The flow rate is 100 μL/min. MS acquisition is from 2 to 10 min.

Method B: Column: Phenomenex Aeris C4 column (1.0×150 mm, 5 μm particle size, 300 Å pore size) Gradient: 1% B (0-2 min), linearly ramp from 1% B to 91% B (2-8 min), 61% B to 95% B (8-10 min). The flow rate is 100 μL/min. MS acquisition is from 2 to 8 min.

Method C: Column: Agilent Zorbax 300SB C3 column (2.1×150 mm, 5 μm particle size, 300 Å pore size) Gradient: 1% B (0-2 min), linearly ramp from 1% B to 91% B (2-12 min), 91% B to 91% B (12-13 min). The flow rate is 500 μL/min. MS acquisition is from 4 to 12 min.

Method D: Column: Phenomenex Jupiter C4 column (1.0×150 mm, 5 μm particle size, 300 Å pore size) Gradient: 1% B (0-2 min), linearly ramp from 1% B to 91% B (2-18 min), 91% B to 91% B (18-21 min). The flow rate is 100 μL/min. MS acquisition is from 4 to 18 min.

Method E: Column: Agilent Zorbax 300SB C3 column (2.1×150 mm, 5 m particle size, 300 Å pore size) Gradient: 1% B (0-1 min), linearly ramp from 1% B to 91% B (1-11 min), 91% B to 91% B (11-15 min). The flow rate is 500 μL/min. MS acquisition is from 0 to 11 min.

### Example 3—Expression and Purification of Proteins

### E6AP

E6AP (residues 1-875) protein with a C-terminal TEV-6×His (SEQ ID NO: 36)-Avi sequence was subcloned into a pFastBac1 vector. Bacmid and viruses of E6AP prepared as described by vendor's instructions were amplified in Sf9 cells. P2 viruses at 2 uL/mL virus to media were used to infect Sf21 cells for protein expression. Cells were harvested 48 hours post-infection.

Sf21 cells were lysed by French Press in 50 mM HEPES pH 7.5, 500 mM NaCl, 5 mM Imidazole, 5% glycerol, 1 mM PMSF, and cOmplete Protease Inhibitor. The supernatant was collected after centrifugation at 39,800 RCF for 30 minutes and loaded onto Ni Resin and washed with 10 CVs of 50 mM HEPES pH 7.5, 500 mM NaCl, 5% glycerol, 1 mM PMSF, and 20 mM imidazole before eluting with the same buffer supplemented with 500 mM imidazole. The elution was diluted five-fold with 50 mM Tris-HCl pH 7.5 before loading onto a Mono Q 10/100 GL column and eluted with a 20 CV linear gradient (Buffer A: 50 mM Tris-HCl (pH 7.5), 100 mM NaCl, 5% glycerol, 1 mM PMSF; Buffer B: 50 mM Tris-HCl (pH 7.5), 1 M NaCl, 5% glycerol, 1 mM PMSF). Fractions containing E6AP as determined by SDS-PAGE and Coomassie staining were pooled and concentrated and loaded onto a HiLoad 16/600 Superdex 200 pg column equilibrated in 25 mM HEPES pH 7.5, 150 mM NaCl, 1 mM TCEP. (FIG. 36A).

E6AP Amino Acid Sequence:
(SEQ ID NO: 56)
MEKLHQCYWKSGEPQSDDIEASRMKRAAAKHLIERYYHQLTEGCGNEACTN
EFCASCPTFLRMDNNAAAIKALELYKINAKLCDPHPSKKGASSAYLENSKG
APNNSCSEIKMNKKGARIDFKDVTYLTEEKVYEILELCREREDYSPLIRVI
GRVFSSAEALVQSFRKVKQHTKEELKSLQAKDEDKDEDEKEKAACSAAAME
EDSEASSSRIGDSSQGDNNLQKLGPDDVSVDIDAIRRVYTRLLSNEKIETA
FLNALVYLSPNVECDLTYHNVYSRDPNYLNLFIIVMENRNLHSPEYLEMAL
PLFCKAMSKLPLAAQGKLIRLWSKYNADQIRRMMETFQQLITYKVISNEFN
SRNLVNDDDAIVAASKCLKMVYYANVVGGEVDTNHNEEDDEEPIPESSELT
LQELLGEERRNKKGPRVDPLETELGVKTLDCRKPLIPFEEFINEPLNEVLE
MDKDYTFFKVETENKFSFMTCPFILNAVTKNLGLYYDNRIRMYSERRITVL
YSLVQGQQLNPYLRLKVRRDHIIDDALVRLEMIAMENPADLKKQLYVEFEG
EQGVDEGGVSKEFFQLVVEEIFNPDIGMFTYDESTKLFWFNPSSFETEGQF
TLIGIVLGLAIYNNCILDVHFPMVVYRKLMGKKGTFRDLGDSHPVLYQSLK
DLLEYEGNVEDDMMITFQISQTDLFGNPMMYDLKENGDKIPITNENRKEFV
NLYSDYILNKSVEKQFKAFRRGFHMVTNESPLKYLFRPEEIELLICGSRNL
DFQALEETTEYDGGYTRDSVLIREFWEIVHSFTDEQKRLFLQFTTGTDRAP
VGGLGKLKMIIAKNGPDTERLPTSHTCFNVLLLPEYSSKEKLKERLLKAIT
YAKGFGMLENLYFQGHHHHHHGLNDIFEAQKIEWHE*

### MBP-16E6

HPV16E6 (residues 8-158) with a N-terminal HisMBP and four cysteine (SEQ ID NO: 885) to serine point mutations to increase solubility was subcloned into a pET45b vector. The plasmid was transformed into BL21(DE3) competent cells. The cells were grown in LB media supplemented with 50 μg/mL carbenicillin shaking at 190 RPM in a 37° C. incubator to an optical density of 0.6 at 600 nm before induction with IPTG at a final concentration of 0.5 mM. Protein expression was allowed to proceed for 16 hours shaking at 190 RPM in an 18° C. incubator before harvest.

BL21(DE3) cells were resuspended in 50 mM Tris-HCl pH 7.5, 500 mM NaCl, 2 mM DTT, and cOmplete Protease Inhibitor before lysis by sonification. The supernatant was collected after centrifugation at 30,000 RCF for 45 minutes and loaded onto a MBPTrap HP column equilibrated in the same resuspension buffer and washed for 30 CV. The protein was eluted with 50 mM Tris-HCl pH 7.5, 500 mM NaCl, 2 mM DTT, and 15 mM maltose. The elution was further purified by size exclusion chromatography on a HiLoad 26/600 Superdex 200 pg column equilibrated in PBS supplemented with 1 mM DTT. (FIG. 36B).

MBP-16E6 amino acid sequence:
(SEQ ID NO: 57)
MAHHHHHHPMKIEEGKLVIWINGDKGYNGLAEVGKKFEKDTGIKVTVEH
PDKLEEKFPQVAATGDGPDIIFWAHDRFGGYAQSGLLAEITPDKAFQDK
LYPFTWDAVRYNGKLIAYPIAVEALSLIYNKDLLPNPPKTWEEIPALDK
ELKAKGKSALMFNLQEPYFTWPLIAADGGYAFKYENGKYDIKDVGVDNA
GAKAGLTFLVDLIKNKHMNADTDYSIAEAAFNKGETAMTINGPWAWSNI
DTSKVNYGVTVLPTFKGQPSKPFVGVLSAGINAASPNKELAKEFLENYL
LTDEGLEAVNKDKPLGAVALKSYEEELAKDPRIAATMENAQKGEIMPNI
PQMSAFWYAVRTAVINAASGRQTVDEALKDAQTNSSSNNNNNNNNNNPM
SENLYFQGAMFQDPQERPRKLPQLCTELQTTIHDIILECVYCKQQLLRR
EVYDFAFRDLCIVYRDGNPYAVCDKCLKFYSKISEYRHYSYSLYGTTLE
QQYNKPLSDLLIRCINCQKPLSPEEKQRHLDKKQRFHNIRGRWTGRCMS
CSRSSRTRRETQL*

Recombinant SUMO-MDM2 was expressed as previously reported. In brief, the 25-109MDM2 gene was synthesized and cloned into a n expression vector. A SUMO tag was incorporated using the Champion™ pET SUMO Expression System. SUMO-25-109MDM2 was expressed in Rosetta (DE3) pLysS cells. The bacteria were inoculated to reach OD600=0.5 at 37° C., induced with 0.4 mM IPTG for 4 hours, and pelleted. Approximately 1 L broth produced 10 g cell pellet, which was resuspended in 30 mL of 50 mM Tris-HCl, 150 mM NaCl, pH 7.5 buffer containing 40 mg lysozyme, 1 mg Roche DNAase I, and one tablet of protease inhibitor cocktail, and then sonicated for three times for 20 s. The suspension was then centrifuged at 30,000 rcf for 30 min to clarify the lysate. The supernatant was loaded onto a 5 mL HisTrap FF crude Ni-NTA columns, and washed sequentially with 30 mL of 20 mM Tris-HCl, 150 mM NaCl, pH 8.5 and then 30 mL 20 mM Tris-HCl 150 mM NaCl, 80 mM imidazole pH 8.5. The protein was eluted with 10 mL 20 mM Tris-HCl, 500 mM imidazole, 500 mM NaCl, pH 8.5. The eluted protein was buffer exchanged into 20 mM Tris-HCl, 50 mM NaCl, pH 8.5 using a HiPrep 26/10 Desalting column. The resulting protein solution was purified the same day using a 5 mL HiTrap Q HP anion exchange columns with a linear NaCl gradient (B % graded from 50% to 25%, B=20 mM Tris-HCl, 500 mM NaCl, pH 8.5). Fractions containing pure SUMO-25-109MDM2, as determined by SDS-PAGE, were collected, and concentrated to 0.5 mg/mL using a 10,000 Da Centrifuge Filter, and immediately flash frozen and stored at −80° C.

### Example 4—Design of High-Affinity Peptide Binders to E6

This example describes the design of high affinity peptide binders to HPV16 E6 based on the E6AP protein and subsequent determination of binding affinity of the peptides to HPV16E6 as measured by BLI.

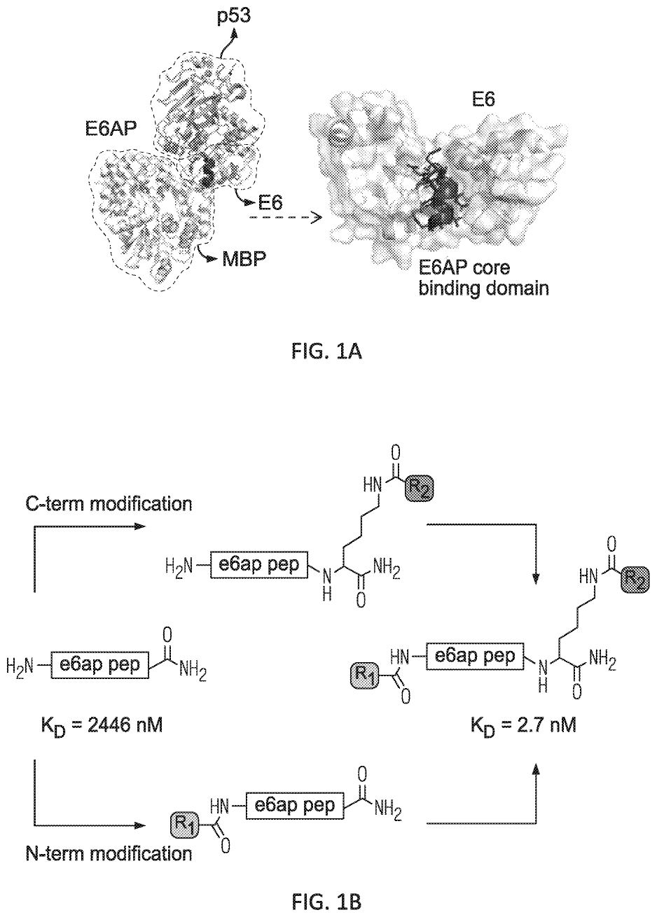

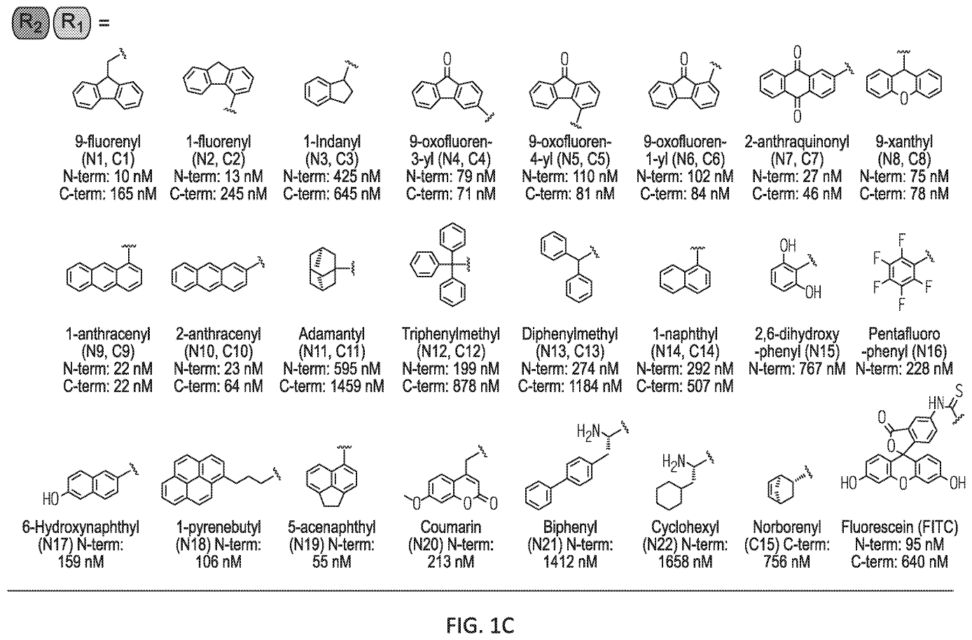

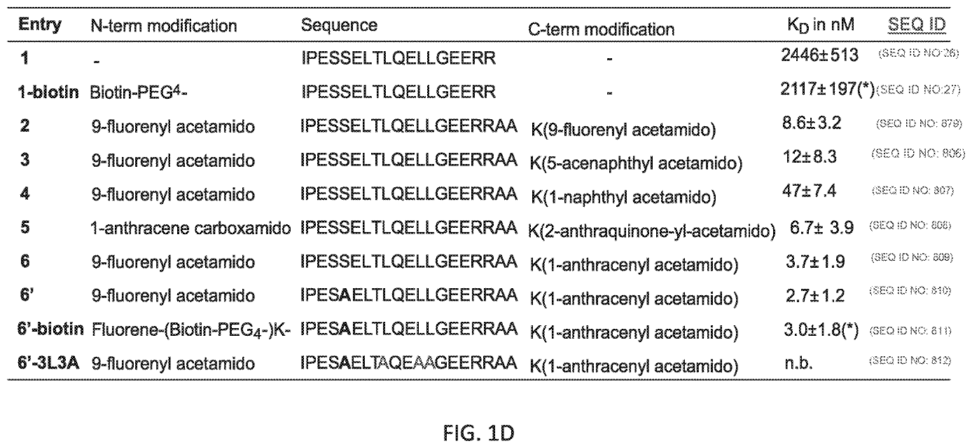

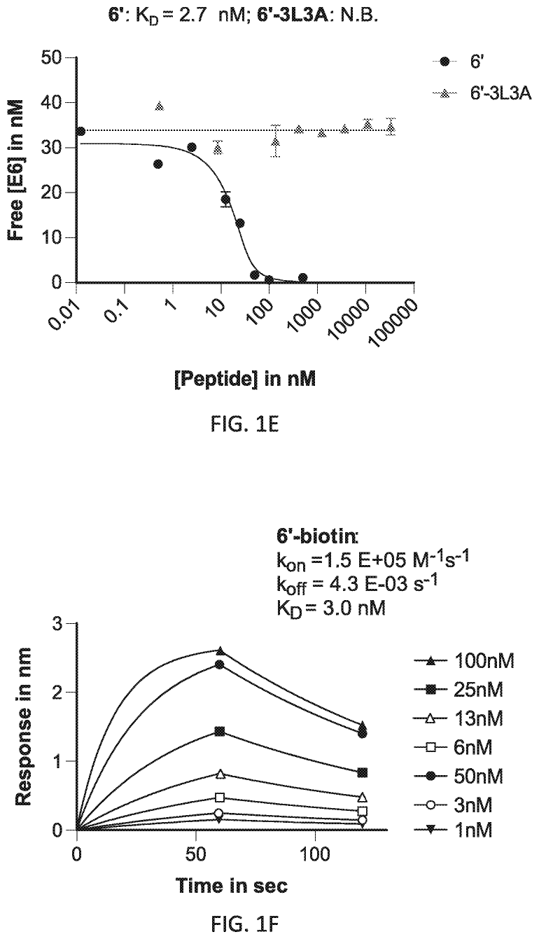

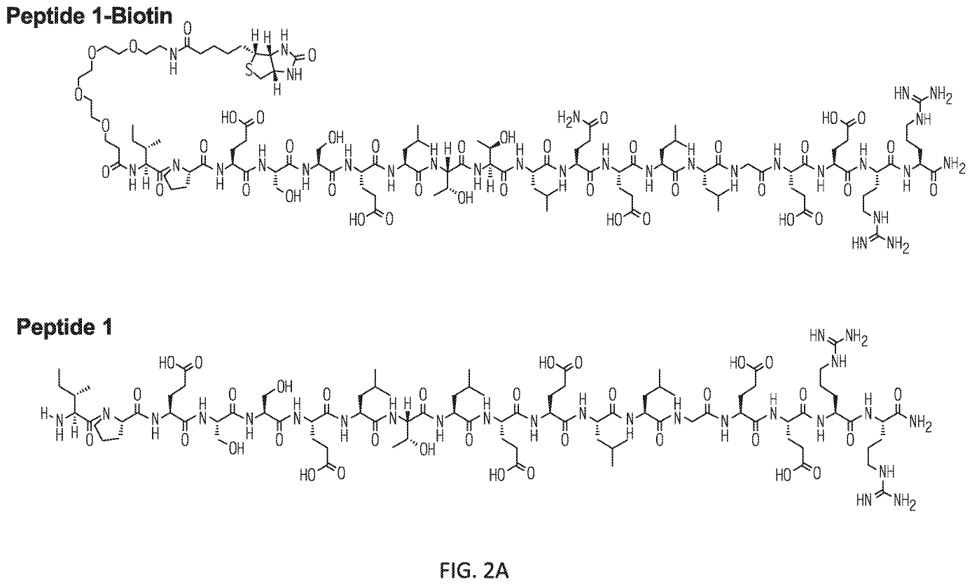

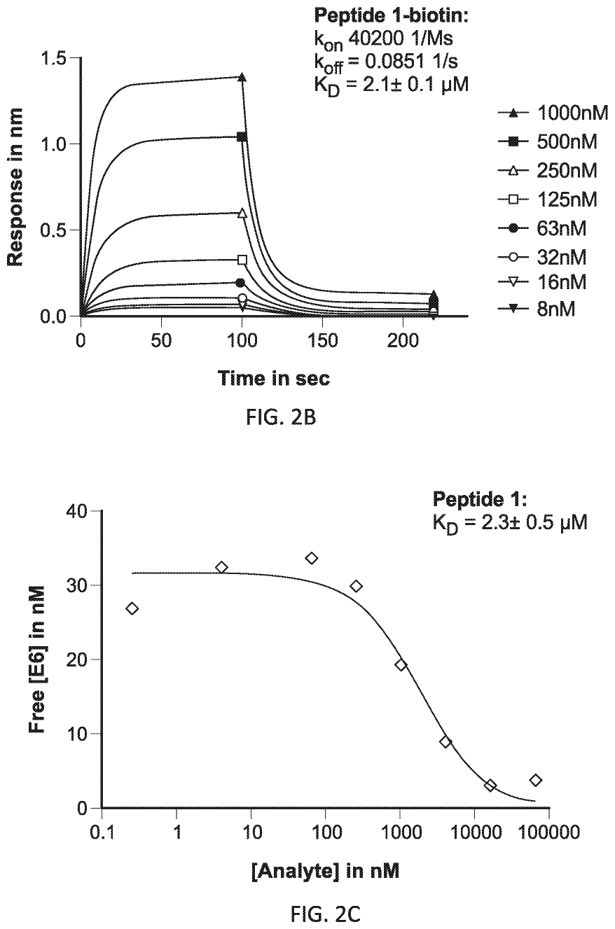

Briefly, the interaction between 16E6 and E6AP was analyzed in an 16E6-E6AP-p53 ternary complex crystal structure (the crystal structure is represented in PDB: 4XR8) FIG. 1A. A 17-mer peptide (Peptide 1, SEQ ID NO: 1, sequence: IPESSELTLQELLGEER, FIG. 1D and FIG. 2A) that covers residues 401-417 of E6AP and includes the LXXLL motif, was chosen as a starting point for a binder.

The binding affinity of peptide 1 to HPV16E6 was assessed by bio-layer interferometry (BLI) using recombinant MBP-16E6 4C4S protein (MBP-16E6, which contains a maltose-binding protein (MBP) tag and four cysteine (SEQ ID NO: 885) to serine mutations to stabilize and solubilize HPV16 E6.

### Direct Binding Measurement by BLI

Streptavidin sensors were soaked in blocking buffer (1×PBS supplemented with 0.05% Tween-20 and 1 mg/mL bovine serum albumin) for 5 min. After immobilizing the 1-biotin peptide (200 nM) onto streptavidin sensors, serial dilutions of MBP-16E6 in the blocking buffer were analyzed for binding. The response was recorded at equilibrium after 2 min. Association lasted for 100 seconds, and dissociation lasted for another 120 seconds. The curve was reported by GatorPlus software (version 2.7.3.1013) and replotted by Prism 8 software.

### Competition Binding Assay by BLI

A competition binding assay was performed as described below using GatorPlus bio-layer interferometry(GatorBio) to estimate the binding affinity of peptides.

Calibration curve: Streptavidin sensors were soaked in blocking buffer (PBS supplemented with 0.05% Tween-20 and 1 mg/mL bovine serum albumin) for 5 min. After immobilizing the PEG4-Biotinylated 1 peptide (200 nM of Biotin-PEG4-IPESSELTLQELLGEER (SEQ ID NO: 43)) (FIG. 2A) onto streptavidin sensors, 1:1 serial dilutions from 1000 nM of E6 in blocking buffer were analyzed for binding (All concentrations: 1000 nM, 500 nM, 250 nM, 125 nM, 62 nM, 31 nM, 15 nM, and 7.8 nM). The response was recorded at equilibrium after 2 min. A curve of sensor response (nm) vs. E6 concentration (nM) was generated to calibrate the free E6 concentration in the solution observed in the competition assay. The curve was generated using Prism 8 software.

Competition assay. Various concentrations of peptides were incubated in wells with E6 protein in the blocking buffer for 30 min. The PEG4-Biotinylated 1 peptide was immobilized onto streptavidin sensors and dipped into preincubated sample wells. The association events were measured at 30° C., 1,000 rpm. Response at equilibrium after 2 min was recorded. Based on the binding response (nm) values, the concentration of ‘free’ E6 was interpolated for each sample using the calibration curve. The apparent dissociation constant, KD, can be obtained from the non-linear regression analysis using the equation:

$$[ Y ] = 0.5 \times [ b - K\_d - [ X ] + √ ( ( [ X ] + K\_d - b ) \hat{} 2 + 4 b \times K\_d ) ]$$

where [Y] is the free [E6] in nM, [X] is the total [peptide] in nM, KD is the binding dissociation constant to be fitted by the equation, and b is the maximal possible E6 concentration to be fitted by the equation. By fitting the free [E6] and [peptide] to the equation, a binding constant with a fitting error was generated by Prism 8 software.

The BLI binding assay was run in competition mode by immobilizing biotinylated 1 (1-Biotin) onto streptavidin biosensor tips and immersing them across a serial dilution of unlabeled Peptide 1 until equilibrium was reached. The results show, that Peptide 1 competed MBP-16E6 binding to 1-Biotin, revealing a competition K_{D} of 2.4 μM (FIGS. 2B and 2C). The competition K_{D} values reported for the peptide variants discussed below were determined by BLI in a similar manner.

Additionally, peptide 1 was labelled N or C terminally with FITC and binding to 16E6 was measured with BLI. The results show increased binding affinity with N-terminal fluorescein isothiocyanate (FITC)-labeled peptide 1 (N-FITC) compared to the C-terminal FITC-labeled peptide 1 (C-FITC). The N-FITC peptide showed a competition K_{D} of 95 nM, while C-FITC peptide 1 had a competition K_{D} of 640 nM.

### Example 5—Alanine Scan of Peptide 1

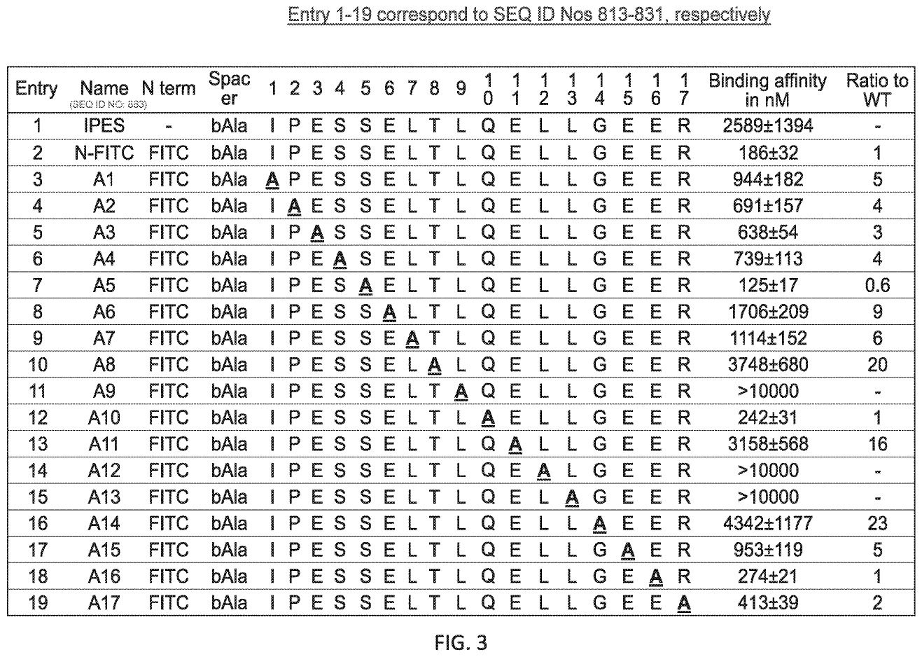

To determine critical residues in the E6AP peptide designs, an alanine (Ala) scan was performed and 17 single Ala mutants of N-FITC, peptides A1 to A17, respectively were synthesized (FIG. 3). Alanine scanning showed the residues that are critical for binding activity. Substituting residues Leu9, Leu11, and Leu12 to alanine or removing the N-terminal modification significantly decreased or disrupted binding. Four residues, Glu6, Thr8, Glu11, and Gly14, showed medium alanine tolerance.

The result show that hotspot residues Leu9, Leu12, and Leu13 to be critical for binding, as the A9, A12, and A13 peptides did not show measurable binding to MBP-16E6. This observation aligns with L12A and L13A single mutated peptides, which showed decrease E6 binding. Peptides simultaneously containing the L9A, L12A, and L13A mutations (or 3L3A triple mutants) were used as negative control peptides (FIG. 1D).

### Example 6—Design of N- or C-Terminal Modified High-Affinity Peptide Binders to E6

Additional peptides with N- and/or C-terminal modifications were designed for analysis (Peptides N1-N22, SEQ ID NO: 2-23, Peptides C1-C15, SEQ ID NO: 24-). A chemical library of small molecules used for modifying E6AP-based peptides is shown in FIG. 1C.

To determine the structure-affinity relationships (SAR) contributing to the binding enhancement of N or C terminal FITC modifications, FITC was replaced with a panel of small molecules at the C- or N-terminus of parent peptide 1 (FIGS. 1B, C and TABLE 1 and TABLE 2A). Peptide binding to E6 was measured with BLI as described in Example 4.

TABLE 1
N-terminal modified E6AP peptides. Binding affinity was measured by BLI
competition assay.

| | | | | Ratio to | SEQ ID |
|---|---|---|---|---|---|
| Name | N-Term | Sequence | K_{D} in nM | wild-type | NOs: |
| N- | Fluorescein-5- | IPESSELTLQELLGEERR | 95 ± 17 | 25.7 | 58 |
| FITC | Isothiocyanate | | | | |
| N1 | 9-fluorene acetamido | IPESSELTLQELLGEERR | 10 ± 6.8 | 244.6 | 59 |
| N2 | 1-fluorene acetamido | IPESSELTLQELLGEERR | 13 ± 6.3 | 188.1 | 60 |
| N3 | 1-Indane acetamido | IPESSELTLQELLGEERR | 425 ± 78 | 5.7 | 61 |
| N4 | 9-fluorenone-2- | IPESSELTLQELLGEERR | 79 ± 14 | 30.9 | 62 |
| | carboxamido | | | | |
| N5 | 9-fluorenone-1- | IPESSELTLQELLGEERR | 110 ± 21 | 22.2 | 63 |
| | carboxamido | | | | |
| N6 | 9-fluorenone-4- | IPESSELTLQELLGEERR | 102 ± 18 | 23.9 | 64 |
| | carboxamido | | | | |
| N7 | anthraquinone-2- | IPESSELTLQELLGEERR | 27 ± 6.3 | 90.5 | 65 |
| | carboxamido | | | | |
| N8 | xanthene-9-carboxamido | IPESSELTLQELLGEERR | 75 ± 19 | 32.6 | 66 |
| N9 | 1-anthracene | IPESSELTLQELLGEERR | 22 ± 7.2 | 111.1 | 67 |
| | carboxamido | | | | |
| N10 | 2-anthracene | IPESSELTLQELLGEERR | 23 ± 9.0 | 106.3 | 68 |
| | carboxamido | | | | |
| N11 | 1-adamantane | IPESSELTLQELLGEERR | 595 ± 152 | 4.1 | 69 |
| | carboxamido | | | | |
| N12 | Triphenyl acetamido | IPESSELTLQELLGEERR | 199 ± 37 | 12.2 | 70 |
| N13 | Diphenyl acetamido | IPESSELTLQELLGEERR | 274 ± 51 | 8.9 | 71 |
| N14 | 1-naphthyl carboxamido | IPESSELTLQELLGEERR | 292 ± 54 | 8.3 | 72 |
| N15 | 1,6-dihydrophenyl | IPESSELTLQELLGEERR | 767 ± 295 | 3.1 | 73 |
| | carboxamido | | | | |
| N16 | Pentafluorophenyl | IPESSELTLQELLGEERR | 228 ± 74 | 10.7 | 74 |
| | carboxamido | | | | |
| N17 | 6-hydroxy-2-naphthyl | IPESSELTLQELLGEERR | 159 ± 47 | 15.3 | 75 |
| | carboxamido | | | | |
| N18 | 1-pyrenebutyl | IPESSELTLQELLGEERR | 106 ± 27 | 23 | 76 |
| | carboxamido | | | | |
| N19 | 5-Acenaphthene | IPESSELTLQELLGEERR | 55 ± 12 | 44.4 | 77 |
| | carboxamide | | | | |
| N20 | 7-Methoxycoumarin-4- | IPESSELTLQELLGEERR | 213 ± 47 | 11.4 | 78 |
| | acetamido | | | | |
| N21 | 4-phenyl-phenylalanine | IPESSELTLQELLGEERR | 1412 ± 757 | 1.7 | 79 |
| N22 | Cyclohexyl-alanine | IPESSELTLQELLGEERR | 1658 ± 705 | 1.4 | 80 |

Error distribution is reported as the standard error of the mean (SEM) of curve fitting reported by Prism 8 software. Wild-type (WT) peptide was used as a reference. K_{D} values are measured by BLI binding assay in a competition mode (N = 2 or 3). The ratio is calculated by dividing competition K_{D} in this row to the competition Kp of WT showed in the first row.

TABLE 2A
C-terminal modified E6AP peptides. Binding affinity was measured by BLI
competition assay.

| | | | | Ratio to | SEQ ID |
|---|---|---|---|---|---|
| Name | Sequence | C-term | K_{D} in nM | wild-type | Nos: |
| C- | Ac- | Lys(Fluorescein-5- | 640 ± 82 | 3.8 | 81 |
| FITC | IPESSELTLQELLGEERRAA | Isothiocyanate) | | | |
| C1 | Ac- | Lys(fluorene-9-acetamido) | 165 ± 35 | 9.9 | 82 |
| | IPESSELTLQELLGEERRAA | | | | |
| C2 | Ac- | Lys(fluorene-1-acetamido) | 245 ± 59 | 9.9 | 83 |
| | IPESSELTLQELLGEERRAA | | | | |
| C3 | Ac- | Lys(1-Indanecarboxamido) | 645 ± 92 | 3.7 | 84 |
| | IPESSELTLQELLGEERRAA | | | | |
| C4 | Ac- | carboxamido) | 71 ± 14 | 34.4 | 85 |
| | IPESSELTLQELLGEERRAA | Lys(9-fluorenone-2- | | | |
| C5 | Ac- | Lys(9-fluorenone-1- | 81 ± 13 | 30.1 | 86 |
| | IPESSELTLQELLGEERRAA | carboxamido) | | | |
| C6 | Ac- | Lys(9-fluorenone-4- | 84 ± 13 | 29.1 | 87 |
| | IPESSELTLQELLGEERRAA | carboxamido) | | | |
| C7 | Ac- | Lys(anthraquinone-2- | 46 ± 5.5 | 53.1 | 88 |
| | IPESSELTLQELLGEERRAA | carboxamido) | | | |
| C8 | Ac- | carboxamido) | 78 ± 18 | 31.3 | 89 |
| | IPESSELTLQELLGEERRAA | Lys(xanthene-9- | | | |
| C9 | Ac- | Lys(1-anthracene | 22 ± 5.8 | 111.1 | 90 |
| | IPESSELTLQELLGEERRAA | carboxamido) | | | |
| C10 | Ac- | Lys(2-anthracene | 64 ± 10 | 38.2 | 91 |
| | IPESSELTLQELLGEERRAA | carboxamido) | | | |
| C11 | Ac- | Lys(1-adamantane | 1459 ± 474 | 1.6 | 92 |
| | IPESSELTLQELLGEERRAA | carboxamido) | | | |
| C12 | Ac- | Lys(triphenylacetamido) | 878 ± 125 | 2.7 | 93 |
| | IPESSELTLQELLGEERRAA | | | | |
| C13 | Ac- | Lys(diphenylacetamido) | 1184 ± 264 | 2 | 94 |
| | IPESSELTLQELLGEERRAA | | | | |
| C14 | Ac- | Lys(1-naphthyl carboxamido) | 507 ± 65 | 4.8 | 95 |
| | IPESSELTLQELLGEERRAA | | | | |
| C15 | Ac- | Lys(Exo-norbornene- | 756 ± 128 | 3.2 | 96 |
| | IPESSELTLQELLGEERRAA | carboxamido) | | | |

Error distribution is reported as the standard error of the mean (SEM) of curve fitting reported by Prism 8 software. Wild-type (WT) peptide was used as a reference. K_{D} values are measured by BLI binding assay in a competition mode (N = 2 or 3). The ratio is calculated by dividing competition K_{D} in this row to the competition Kp of WT showed in the first row.

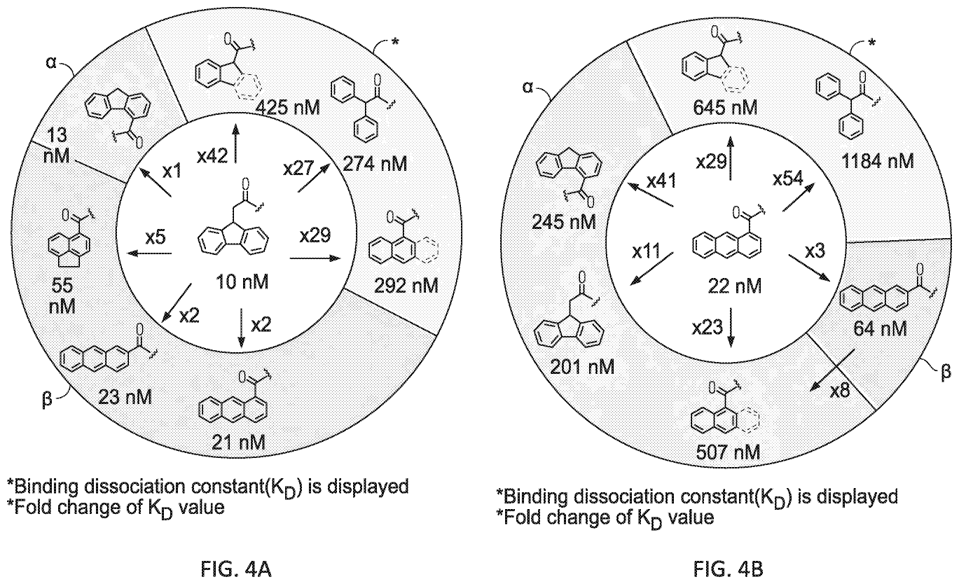

The results show that from the 36 synthesized peptides, 8 terminally modified peptides displayed K_{D}<70 nM against MBP-16E6 (Peptides N1, N2, N7, N9, N10, N19, C4, C9, and C10). At the N-terminus, planar and tricyclic arenes enhanced binding by 50 to 250-fold relative to unmodified peptide 1, while bulky, hydrophobic, or aliphatic small molecules modestly improved affinity by less than 5-fold (FIG. 1C). The position of fluorene ring substitution had no effect on N- or C-terminal modifications. At the N-terminus, the largest ~250-fold increase in binding affinity was observed with a fluorene modification (peptide N1). Removal of a phenyl ring from fluorene (N1, K_{D} of 10 nM) to indane (N3, K_{D} of 425 nM) decreased the binding by ~42-fold. Compared to fluorene-modified N1, biphenyl-containing N21 (K_{D} of 1412 nM) decreased the binding by ~27-fold. These results suggest that N-terminal planar tricyclic aromatic scaffolds are favorable motifs to enhance binding of parent peptide 1 to 16E6 (FIG. 4A). The greatest improvements at the C-terminus, from 30 to 111-fold, were found with planar, tricyclic, and polyarenes, while bulky, non-conjugated aromatic or aliphatic small molecules did not significantly improve the binding compared to parent peptide 1 (FIG. 1C and TABLE 2A). The best performing C-terminal modification was anthracene, which showed a competition K_{D} value of 22 nM, enhancing binding affinity to MBP-16E6 by ~111-fold compared to the parent peptide 1 (FIG. 4B, TABLE 2B).

TABLE 2B

| | N-term | | | | SEQ |
|---|---|---|---|---|---|
| | modifi- | | C-term | | ID |
| Entry | cation | Sequence | modification | KD, nM | Nos: |
| 1 | — | IPESSELTLQELLGEERR | — | 2446 ± 513 | 26 |
| 1- | Biotin- | IPESSELTLQELLGEERR | — | 2117 ± 197(*) | 27 |
| biotin | PEG4- | | | | |
| 2 | Fluorene | IPESSELTLQELLGEERRAA | K(9-fluorene) | 8.6 ± 3.2 | 97 |
| 3 | Fluorene | IPESSELTLQELLGEERRAA | K(5- | 12 ± 8.3 | 198 |
| | | | Acenaphthene) | | |
| 4 | Fluorene | IPESSELTLQELLGEERRAA | K(1- | 47 ± 7.4 | 99 |
| | | | naphthanlene) | | |
| 5 | Anthracene | IPESSELTLQELLGEERRAA | K(2- | 6.7 ± 3.9 | 100 |
| | | | anthraquinone) | | |
| 6 | Fluorene | IPESSELTLQELLGEERRAA | K(1- | 3.7 ± 1.9 | 2 |
| | | | Anthracene) | | |
| 6′ | Fluorene | IPESAELTLQELLGEERRAA | K(1- | 2.7 ± 1.2 | 3 |
| | | | Anthracene) | | |
| 6′- | Fluorene- | IPESAELTLQELLGEERRAA | K(1- | 3.0 ± 1.8(*) | 103 |
| biotin | (Biotin- | | Anthracene) | | |
| | PEG4-)K- | | | | |
| 6′- | Fluorene | IPESAELTAQEAAGEERRA | K(1- | n.b. | 104 |
| 3L3A | | A | Anthracene) | | |

Combining the N- and C-terminal modifications with the highest 16E6 binding affinity generated double-modified peptide binders that displayed improvements over single modifications-likely due to synergistic effect from binding features of the two-terminal modifications (FIG. 1D). Peptide 6 (SEQ ID NO: 2) contained a N-terminal fluorene, and a C-terminal anthracene and showed the lowest competition K_{D} of 3.7 nM to MBP-16E6 and was selected for further terminal modifications. Additional optimization was achieved by substituting Ser5 to Ala, as previous Ala scan studies revealed a 2-fold increase in binding affinity (FIG. 3), resulting in peptide 6′ (SEQ ID NO: 3) with a competition K_{D} of 2.7 nM.

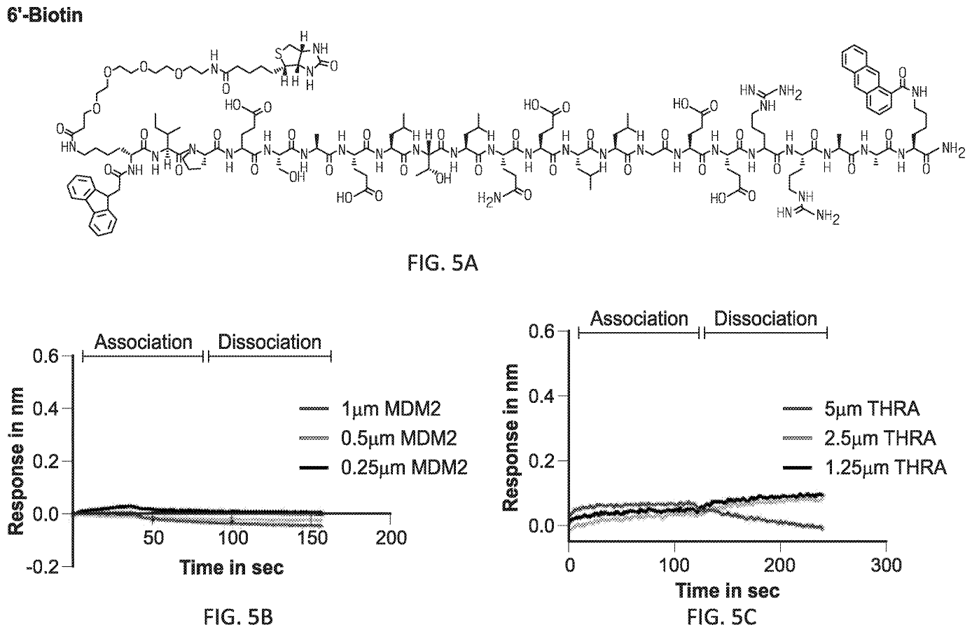

To determine the binding affinity and selectivity of 6′, biotinylated peptide 6′-biotin and its 3L3A derivative 6′-3L3A were synthesized. 6′-3L3A displayed no observable binding towards MBP-16E6 (FIG. 1E). 6′-biotin showed a direct binding K_{D} of 3.0±1.8 nM to MBP-16E6(FIG. 1F). These results demonstrate that peptide 6′ binding to MBP-16E6 is sequence-specific and requires the tri-leucine hotspot motif LXXLL. To assess target selectivity, peptide 6′-biotin (FIG. 5A) was immobilized and its affinity towards the unrelated proteins murine double minute 2 (MDM2) and an LXXLL motif-binding protein thyroid hormone receptor alpha (THRA) were measured by BLI. No appreciable signal during the association or dissociation steps was observed by BLI (FIGS. 5B and 5C), indicating that peptide 6′ selectively binds MBP-16E6.

### Example 7—Development of Modified E6 Binding Peptides for Crosslinking to MBP-16E6

This example describes the design of E6 binding peptides that are modified with a warhead for covalent binding. Irreversible covalent inhibition is an effective strategy to increase drug potency and selectivity toward inhibiting ‘undruggable’ targets.

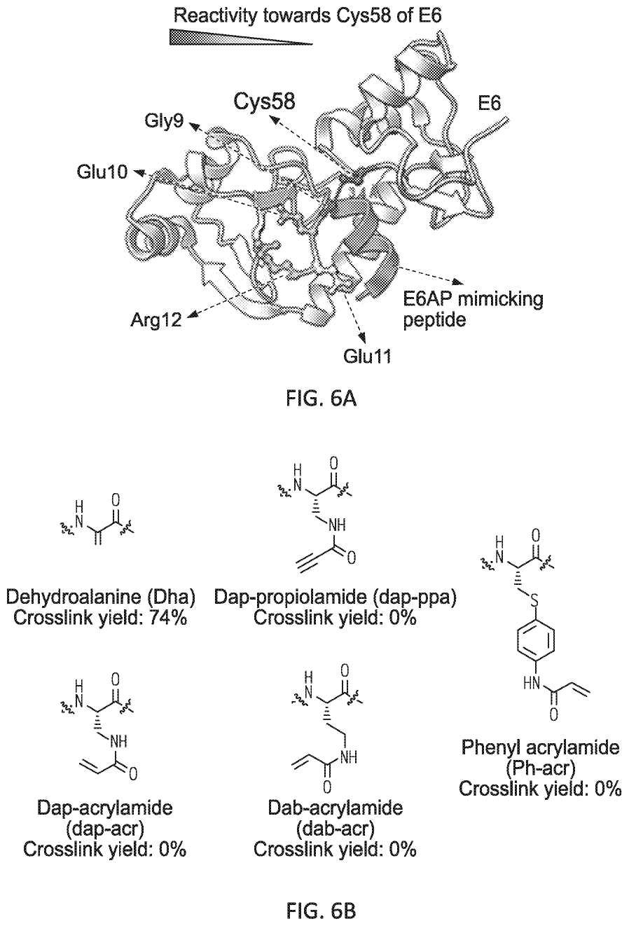

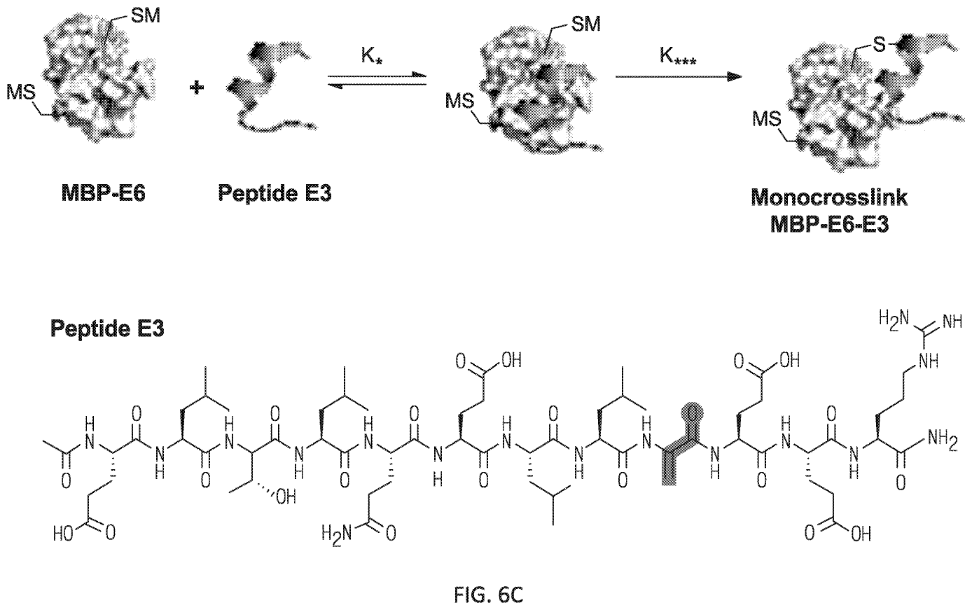

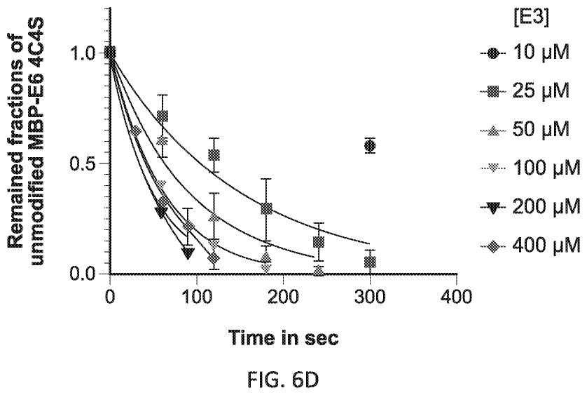

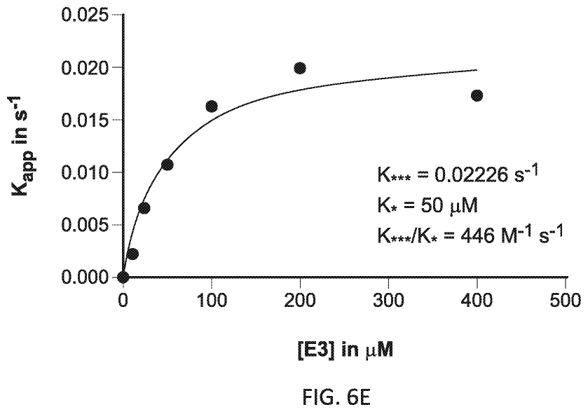

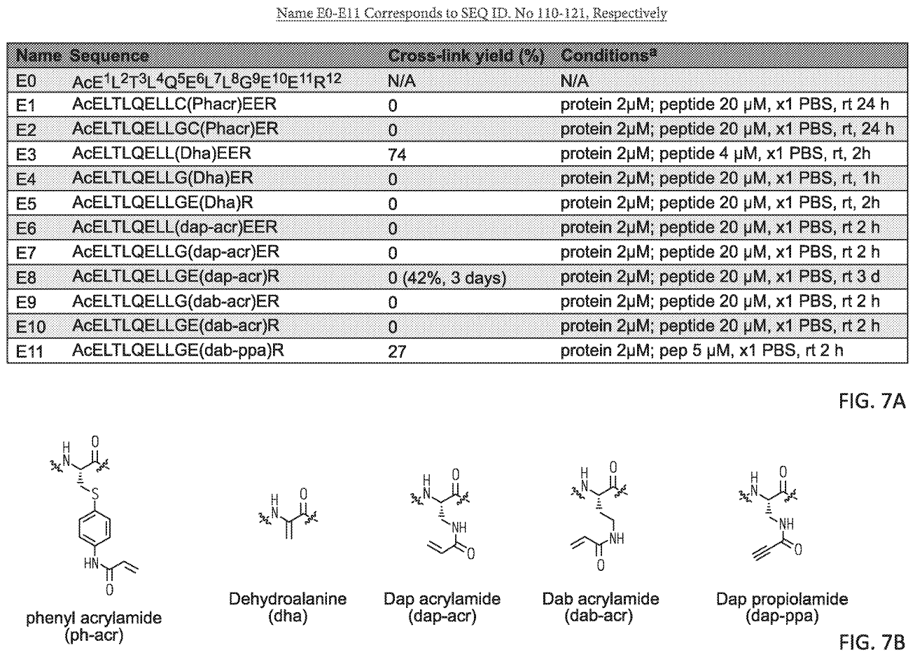

Briefly, the binding interface of 16E6 contains the nucleophilic Cys58 residue in proximity to the E6AP LXXLL peptide binding pocket (FIG. 6A). To exploit this finding, peptide reactides E1-E11 (FIG. 7A) comprising a warhead for covalent binding, were based on peptide E0, a truncated version of peptide 1. Reactides E1-11 were synthesized, with a Cys-reactive acrylamide replacing Gly9, Glu10, or Glu11 of E0 as shown (FIG. 6B) and FIG. 7A). Multiple electrophiles including phenylacrylamide (Ph-acr), Dap-acrylamide (dap-acr), Dab-acrylamide (dab-acr), Dap-propiolamide (dap-ppa), and dehydroalanine Dha (FIG. 7B) were tested as a warhead.

Dehydroalanine (Dha) Formula I is a cysteine targeting, monocrosslink with irreversible reaction and relatively mild chemistry. The resulting bond is stable to reducing in vivo environment.

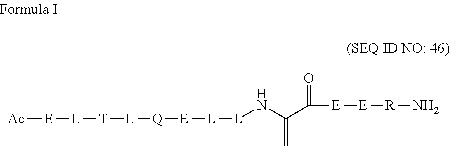

Liquid chromatography-mass spectrometry (LC-MS) monitored conjugation between MBP-16E6 protein and the reactides E1-E11 was measured, and the area under the total ion peak was used to estimate crosslink yield.

The results show, that Gly9 was found to be the closest residue to Cys58 and its substitution with Dha gave the highest crosslink yield of 74% (peptide E3, or ELT-G9Dha) (FIG. 6C).

Additional kinetic studies were performed with reactide peptide E3 to estimate the binding constant Kᵢ and first-order rate constant (kinact) involved in the two-stage bind-and-react strategy. Crosslink yield was monitored overtime by LC-MS (FIG. 6D and FIG. 6E). Kinetic parameters were estimated assuming a steady-state approximation, and a kinact value of 0.022 s-1 and Kᵢ value of 50 μM were obtained. The kinact/Kᵢ ratio was calculated to be 446 M-1 s-1, indicating that improvements in binding affinity will produce a more efficient covalent inhibitor. For comparison, the FDA-approved small molecular drug nirmatrelvir showed a kinact/Ki value of 55 mM-1 s-1.

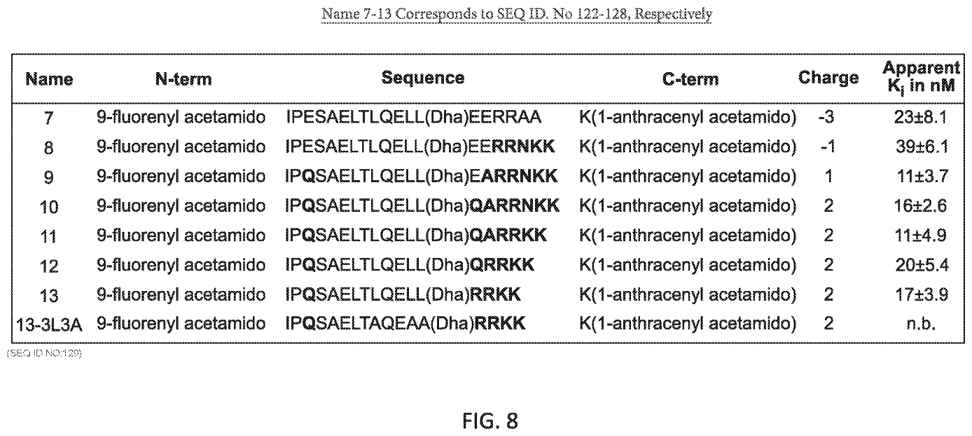

Additional longer reactide peptides based on peptide 6′ (Example 6) were designed. First Dha was installed onto peptide 6′ to obtain reactide 7, which has a net charge of −3 that may be detrimental to cell permeability (FIG. 8). To increase positive charge, improve solubility, retain binding affinity to 16E6, and further reduce molecular weight, additional residue substitutions and truncations on 7 were performed (peptides 8-13, FIG. 8). The LXXLL proximal RRNKK (SEQ ID NO: 105) [residues 417-421] segment from the E6AP protein was appended to the C-terminus of reactide 7 to increase the total charge from −3 to −1 (reactide 8). Next, Glu3Gln, Glu15Gln, and Glu16Ala mutations were applied to increase the total charge to +2, resulting in peptides 9 and 10 (FIG. 8). To reduce molecular weight, Ala13, Gln14, and Asn19 residues were omitted from peptide 10 to generate 11, 12, and 13 (Fluorene-IPQSAELTLQELL(DHA)RRKKK(Anthracene) (SEQ ID NO: 4)) (FIG. 8). Reactides were evaluated by the BLI competition assay against 1-biotin to estimate their inhibitory constant, reported here as an apparent Ki value.

TABLE 2C

| | | | | | | SEQ |
|---|---|---|---|---|---|---|
| | | | | | | ID |
| Name | N-term | Sequence | C-term | Charge | K_{D} in nM | Nos: |
| 6′ | Fluorene | IPESAELTLQELLGEERRAA | K(Anthracene) | -3 | 1.4 ± 0.6 | 106 |
| 7 | Fluorene | IPESAELTLQELL(DHA)EERRAA | K(Anthracene) | -3 | 23 ± 8.1 | 107 |
| 8 | Fluorene | IPESAELTLQELL(DHA)EERRNKK | K(Anthracene) | -1 | 39 ± 6.1 | 108 |
| 13 | Fluorene | IPQSAELTLQELL(DHA)RRKK | K(Anthracene) | 2 | 17 ± 3.9 | 4 |
| 13- | Fluorene | IPQSAELTAQEAA(DHA)RRKK | K(Anthracene) | 2 | n.b. | 109 |
| 3L3A | | | | | | |

TABLE 2D

| Name | Sequence | SEQ ID Nos: |
|---|---|---|
| E0 | ACE¹L²T³L⁴Q⁵E⁶L⁷L⁸G⁹E¹⁰E¹¹R¹² | 110 |
| E1 | AcELTLQELLC(Phacr)EER | 111 |
| E2 | AcELTLQELLGC(Phacr)ER | 112 |
| E3 | AcELTLQELL(Dha)EER | 113 |
| E4 | AcELTLQELLG(Dha)ER | 114 |
| E5 | AcELTLQELLGE(Dha)R | 115 |
| E6 | AcELTLQELL(dap-acr)EER | 116 |
| E7 | AcELTLQELLG(dap-acr)ER | 117 |
| E8 | AcELTLQELLGE(dap-acr)R | 118 |
| E9 | AcELTLQELLG(dab-acr)ER | 119 |
| E10 | AcELTLQELLGE(dab-acr)R | 120 |
| E11 | AcELTLQELLGE(dab-ppa)R | 121 |

TABLE 2E

| | | | | | | SEQ |
|---|---|---|---|---|---|---|
| | | | | | Apparent | ID |
| Name | N-term | Sequence | C-term | Charge | Kᵢ in nM | Nos: |
| 7 | 9- | IPESAELTLQELL(Dha)EERRAA | K(1- | −3 | 23 ± 8.1 | 122 |
| | fluorenyl | | anthracenyl | | | |
| | acetamido | | acetamido) | | | |
| 8 | 9- | IPESAELTLQELL(Dha)EERRNKK | K(1- | −1 | 39 ± 6.1 | 123 |
| | fluorenyl | | anthracenyl | | | |
| | acetamido | | acetamido) | | | |
| 9 | 9- | IPQSAELTLQELL(Dha)EARRNKK | K(1- | 1 | 11 ± 3.7 | 124 |
| | fluorenyl | | anthracenyl | | | |
| | acetamido | | acetamido) | | | |
| 10 | 9- | IPQSAELTLQELL(Dha)QARRNKK | K(1- | 2 | 16 ± 2.6 | 125 |
| | fluorenyl | | anthracenyl | | | |
| | acetamido | | acetamido) | | | |
| 11 | 9- | IPQSAELTLQELL(Dha)QARRKK | K(1- | 2 | 11 ± 4.9 | 126 |
| | fluorenyl | | anthracenyl | | | |
| | acetamido | | acetamido) | | | |
| 12 | 9- | IPQSAELTLQELL(Dha)QRRKK | K(1- | 2 | 20 ± 5.4 | 127 |
| | fluorenyl | | anthracenyl | | | |
| | acetamido | | acetamido) | | | |
| 13 | 9- | IPQSAELTLQELL(Dha)RRKK | K(1- | 2 | 17 ± 3.9 | 128 |
| | fluorenyl | | anthracenyl | | | |
| | acetamido | | acetamido) | | | |
| 13- | 9- | IPQSAELTAQEAA(Dha)RRKK | K(1- | 2 | n.b. | 129 |
| 3L3A | fluorenyl | | anthracenyl | | | |
| | acetamido | | acetamido) | | | |

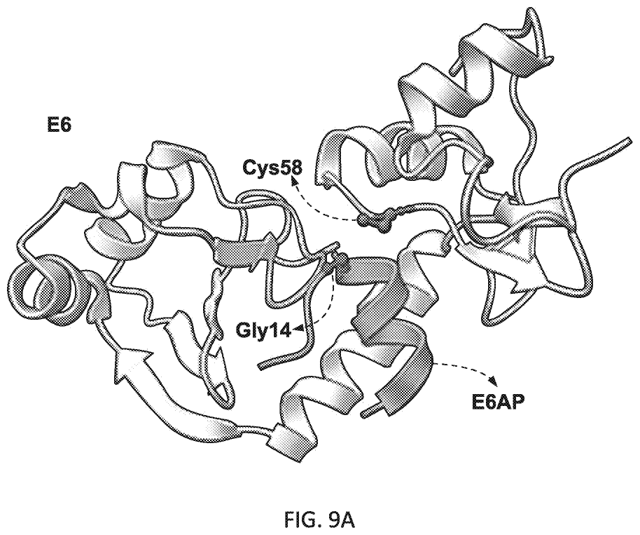

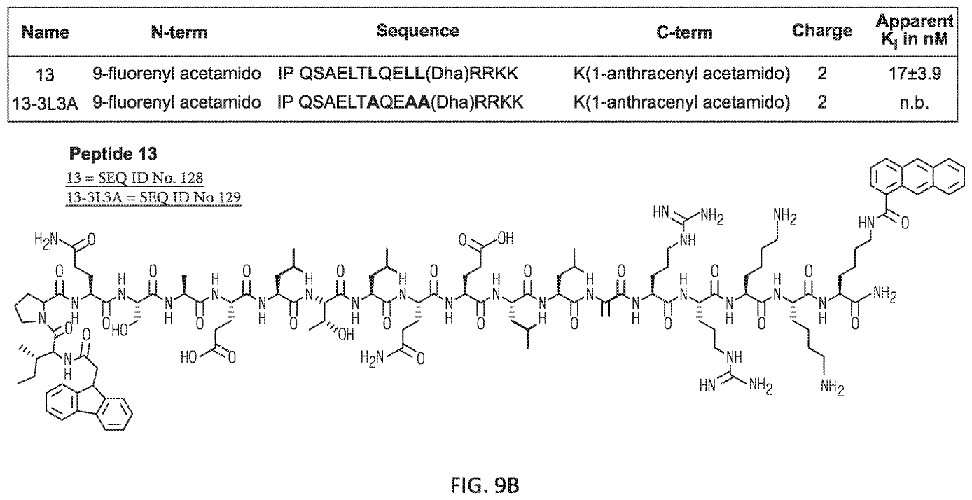

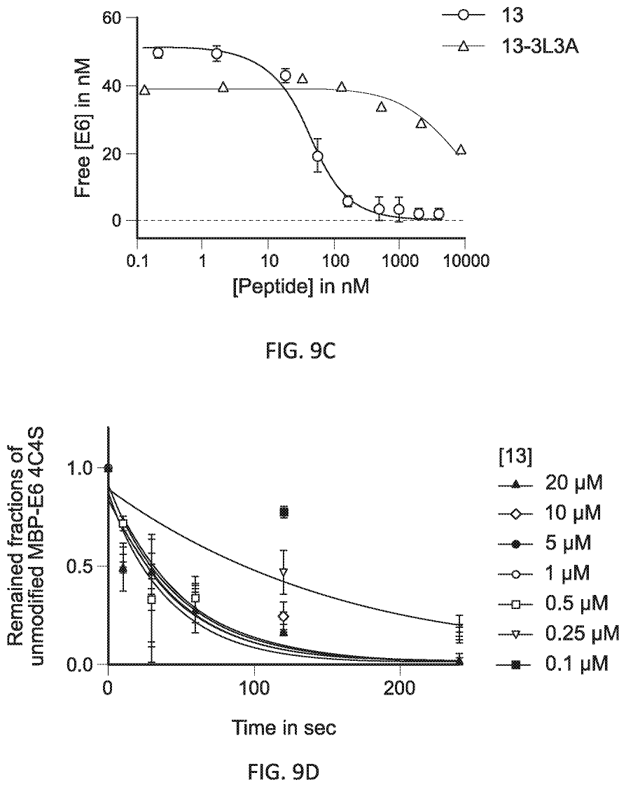

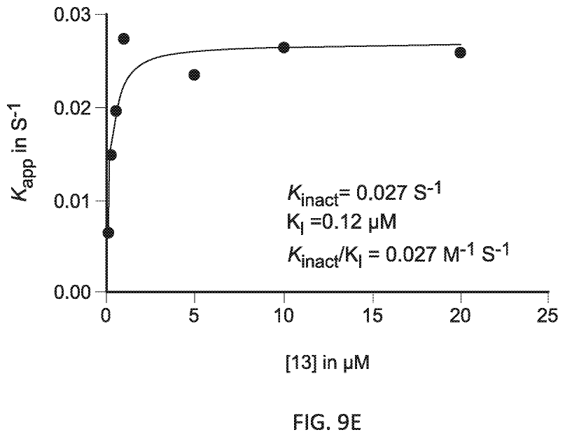

The results show that peptides 8-13 have comparable binding affinity to the parent reactide 7, with an apparent Ki ranging from 11 nM to 39 nM (FIG. 8). Peptide reactide 13 was selected for further characterization since it has the smallest molecular weight, highest net charge, and relatively low apparent Ki=17±3.9 nM (FIGS. 9A, 9B and 9C). The negative control derivative of peptide 13, (13-3L3A) showed no observable binding to MBP-16E6 protein. Kinetic study was performed with peptide 13 and revealed a kinact of 0.027 s-1, a Ki value of 120 nM and a kinact/Ki ratio of 270 mM-1 s-1 (FIGS. 9D and 9E). Reactide 13 represents a 504-fold improvement over E3.

Despite increasing numbers of reports describe peptide-based covalent inhibitors it is under-exploited to target challenging protein-protein interactions. One major drawback for this strategy is off-target crosslinking which can be mitigated through a bind-and-react strategy. With this approach, the risks of non-specific crosslinking and non-selective inhibition are reduced through specific binding of the base peptide sequence which brings the reactive warhead and its target residue in close proximity. This may allow for the use of less reactive groups to alleviate non-selective modification.

Dha is the simplest dehydroamino acid is found in some microbial peptides. Due to its electrophilic nature and lack of geometric isomers resulting from the methylidene group, it has been utilized as a reactive probe targeting enzymatic mechanisms. Dha is used as an electrophilic warhead to generate a reactide for targeting a cancer-relevant PPI. Reactivity of the reactide was fine-tuned by adjusting the electrophile and distance between the warhead and peptide backbone.

Crosslinking specificity of the reactides was further tested in cell media. High crosslinking efficiency was achieved with Dha placed directly onto the peptide backbone to fit in the tight binding pocket at the target protein surface containing Cys58, as suggested by the co-crystal structure. The reactide was stable and not reactive to 0.4 mM free Cys and GST in cell media.

### Example 8—Crosslinking of Reactide 13 to MBP-16E6

This example described the crosslinking of reactide 13(SEQ ID NO: 4) to MBP-16E6.

Briefly, a solution of cross-linker (250 μM solution in 10% DMSO/H₂O, 2 to 10 equiv, 0.66 μL) was diluted with H₂O (30 μL) and 10×PBS (4.1 μL, pH=7.4). A 30 μM solution of MBP-6 (2.4 μL, 1 equiv) and then incubated at room temperature for 2 h. An 8 μL aliquot of the reaction mixture was removed and quenched with 92 μL of 50:50 MeCN/H₂O+0.1% TFA and analyzed by LC/MS. Yields were obtained by extracting all protein-containing species' total ion current (TIC) spectra in the chromatogram utilizing Agilent MassHunter Bioconfirm Software 10.0. The extracted chromatograms were deconvoluted utilizing a maximum entropy algorithm, and the abundance of each species was determined using total ion count.

$$\% yield = \frac{{P}_{c}}{{P}_{c} + {P}_{0}} \times 100$$

where P_{c} is the peak area of the peptide-protein conjugate, and Po is the peak area of the unmodified protein

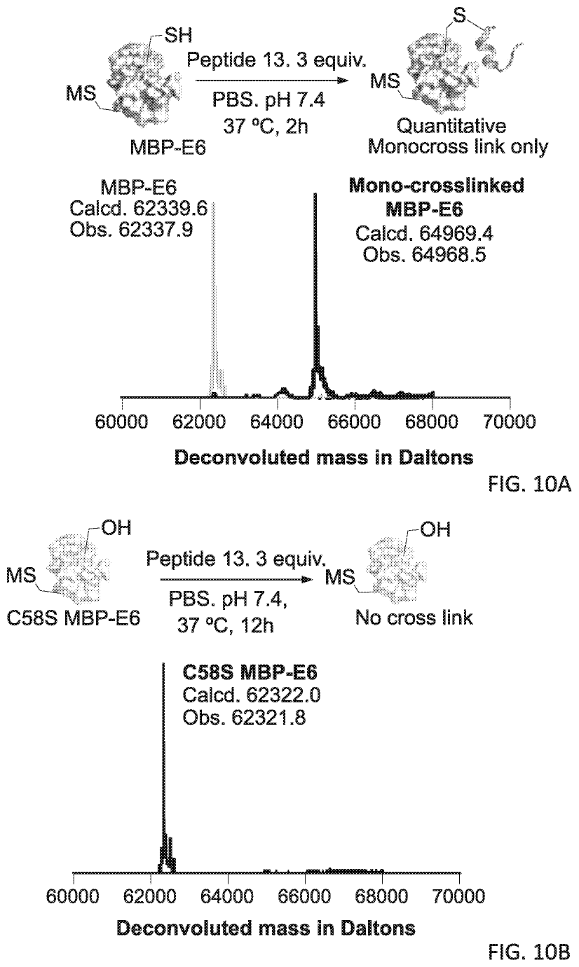

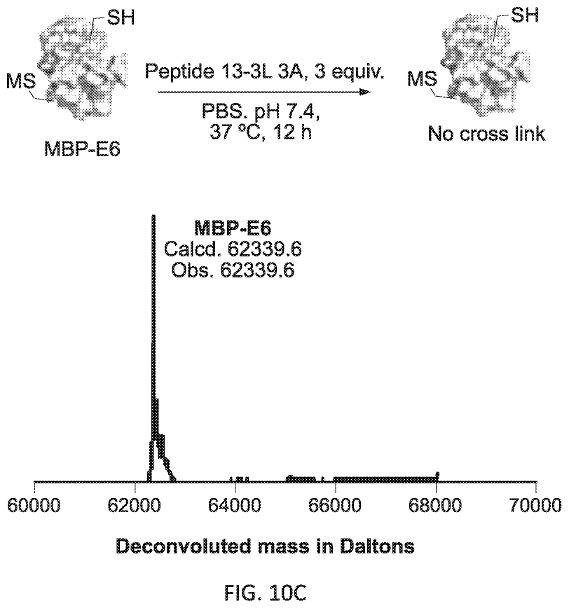

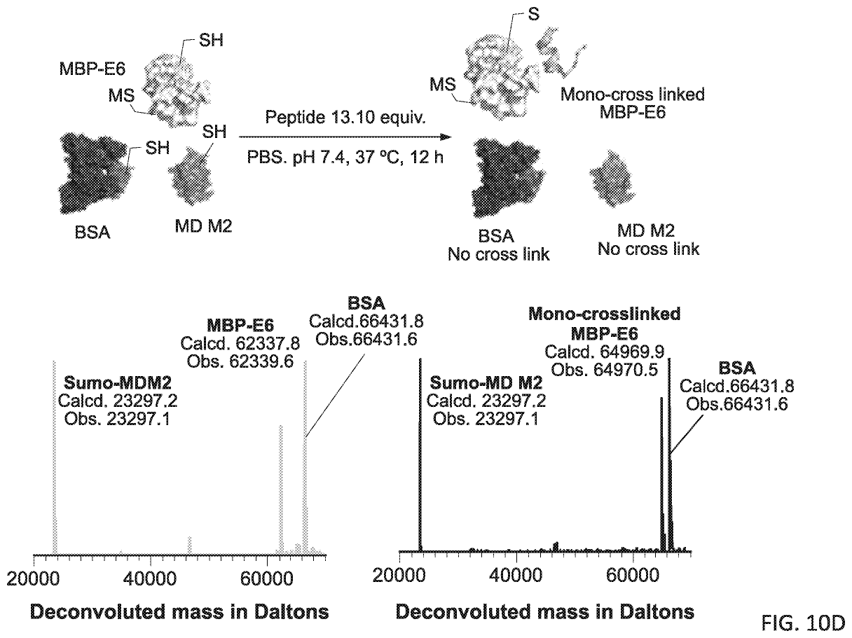

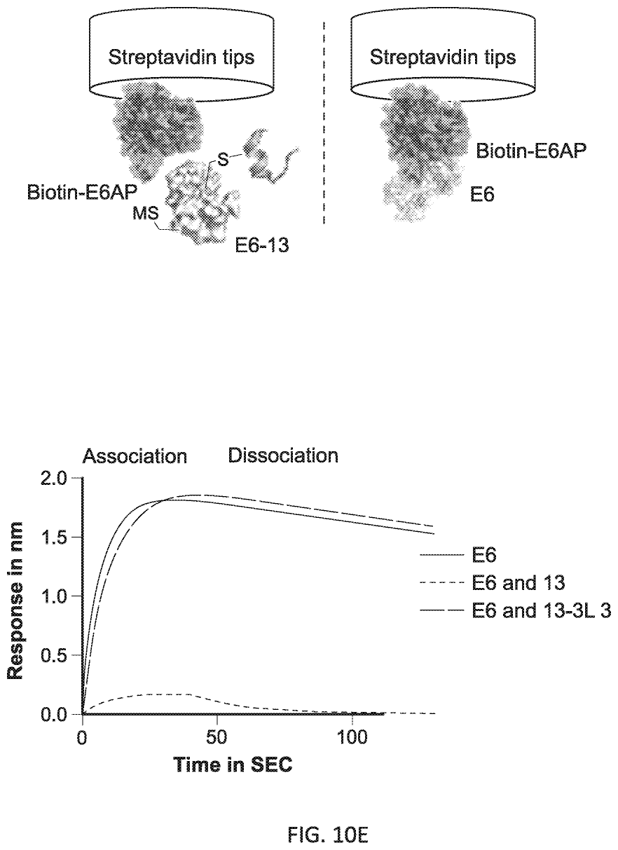

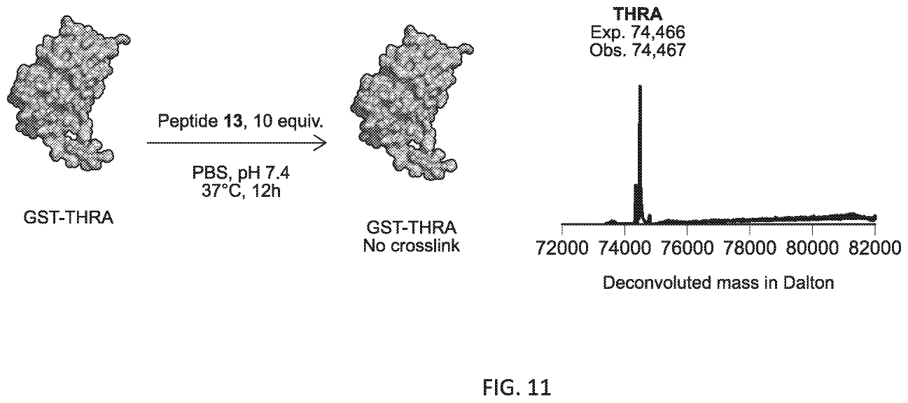

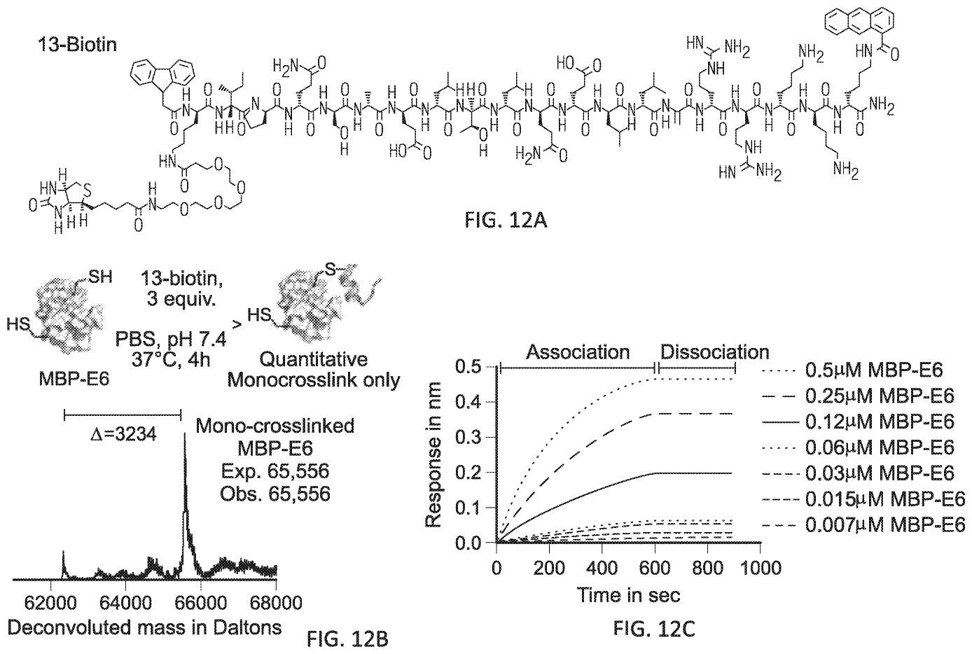

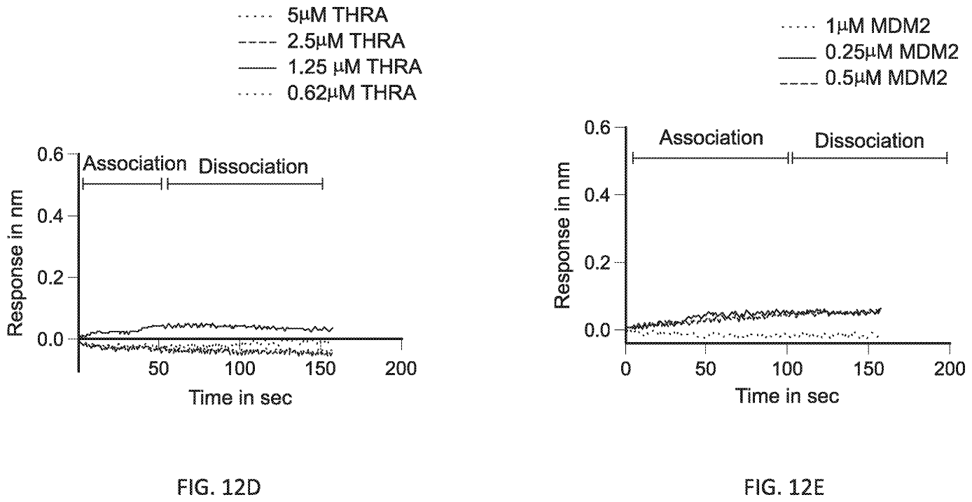

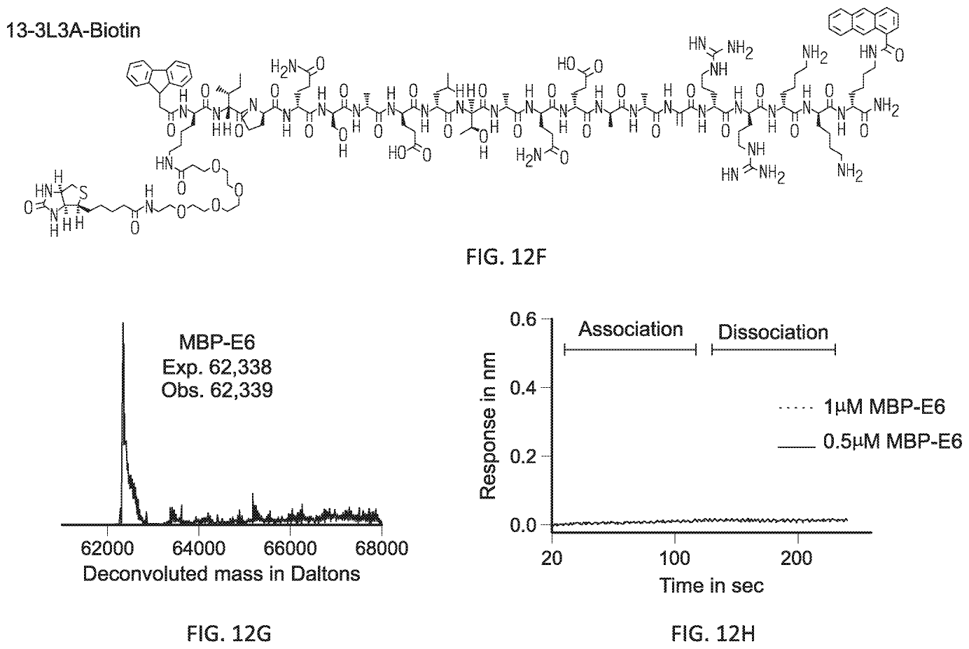

When peptide 13 (3 μM) and MBP-16E6 (1 μM) were incubated in PBS at 37° C. for two hours, protein deconvolution mass spectra revealed >99% mono-crosslinking of 13 to MBP-16E6 (FIG. 10A). This was achieved despite the presence of 10 cysteine residues on MBP-16E6. To confirm site-selectivity, 13 was incubated with the MBP-16E6 mutant C58S for 12 h in PBS at 37° C. No crosslinking was observed between 13 and MBP-16E6 C58S (FIG. 10B), which supports Cys58 as the sole site of MBP-16E6 modification. 13 showed no crosslink to THRA (FIG. 11) Additionally, the negative control 13-3L3A showed no observable-crosslinking to MBP-16E6 protein (FIG. 10C), supporting peptide 13 specificities. Therefore, 13-3L3A was used as a negative control for subsequent peptides.

### Crosslinking of Reactide 13 to MBP-16E6 in a Protein Mixture

To investigate whether peptide 13 selectively crosslinks to 16E6 in a protein mixture modification of Sumo-MDM2 which contains one cysteine and bovine serum albumin (BSA) which has 35 cysteines were monitored. A mixture of three proteins, including 16E6 (1 μM), BSA (1 μM), and Sumo-MDM2 (1 μM) was incubated with peptide 13 (10 μM) at 37° C. for 12 h and resolved by LC-MS (FIG. 10D). The results indicate the 16E6 is mono-crosslinked with >95% conversion with no observable modifications to BSA or MDM2. These results demonstrate crosslinking of peptide 13 is specific to MBP-16E6 and depends on the hotspot motif LXXLL.

### Assessment of 13-Biotin and 13-TAMRA Specificity in HT1080 Cells

HT1080 cells (CCL-121, ATCC) were cultured until they reached ~80% confluency, at which point they were harvested using trypsinization and subsequent centrifugation at 500×g for 5 minutes. Resulting cell pellets were washed with 1×PBS and lysed via the introduction of cold Pierce IP lysis buffer (catalog no. 87787), supplemented with 1× Halt Protease Phosphatase inhibitor cocktail (ThermoFisher, catalog no. 1861284). This lysis process was conducted on ice for 10 minutes, with periodic pipetting to ensure adequate mixing. Post-lysis, the supernatant was cleared by centrifuging at maximum speed using a bench-top Eppendorf centrifuge (model #5425R) for 10 minutes at 4° C. After removing the supernatant, the resulting lysates were quantified using the Pierce 660 nm Protein Assay (catalog no. 22660), with BSA standards serving as a reference.

HT1080 lysate (50 μg) was supplemented with either MBP-16E6 (2 μM) or MBP-16E6 C58S (2 μM), followed by an overnight incubation at 4° C. with the presence of 13-TAMRA (10 μM), 13-biotin (10 μM), or 13-3L3A-biotin (10 μM). Each reaction was diluted in 1×PBS supplemented with 1 mM DTT and 5% glycerol to a final volume of 20 μL while maintaining DMSO concentration constant at 0.5%. Resulting reactions were subjected to 4-20% Criterion TGX polyacrylamide gel electrophoresis (BioRad). Samples containing 13-TAMRA were visualized on an Amersham ImageQuant 800 using the Cy2 settings. Protein ladder (LI-COR; catalog no. 928-60000) was also imaged using IR short and IR long settings. Following this, all gels underwent transfer onto 0.2 m nitrocellulose (catalog no. 1704159, BioRad) using the ‘Mixed Mw Turbo’ mode of the Trans-Blot Turbo Transfer System (catalog no. 1704150, BioRad). Post-transfer, membranes were rinsed with ultrapure water, stained with Ponceau S staining solution (Thermo Scientific; catalog no. A40000278) for 5 minutes, washed again, and imaged on a BioRad ChemiDoc MP Imaging System with visible light.

Membranes were then blocked with Intercept (TBS) blocking buffer (catalog no. 9276001, LI-COR), followed by overnight incubation at 4° C. with shaking in Intercept (T20, TBS) Antibody Diluent buffer (catalog no. 92765001, LI-COR) incubated with α-HPV16E6 antibody (catalog no. GTX132686, Genetex, lot no. 44601) and α-GAPDH (catalog no. MA5-15738, lot no. WL332983), each diluted to 1:1,000. Blots were then washed four times for 10 minutes each with TBS-T, and incubated with goat-anti-mouse (680 nm conjugate; catalog no. 925-68070, lot no. D10901-11) and goat-anti-rabbit (800 nm conjugate; catalog no. 926-32211, lot no. D20322-15) secondary antibodies for 2 hours at room temperature with shaking. This was followed by an additional series of four 10-minute washes with TBS-T.

13-biotin and 13-3L3A-biotin blots were visualized using the ODYSSEY CLx LI-COR instrument with Image Studio software (version no. 5.2). The scanning parameters included automated intensity, 169 μm pixel size, and 0.0 mm offset. After imaging for GAPDH and MBP-16E6, the 13-biotin and 13-3L3A-biotin blots were incubated with IRDye 680 RD streptavidin (LI-COR, catalog no. 926-68079, lot no. D20803-07), at a 1:2,000 dilution in Intercept antibody diluent for 30 minutes. The blots were then washed four times for 10 minutes each with TBS-T and subsequently imaged. These initial conditions led to signal saturation, prompting the blots to be stripped using the NewBlot Nitrocellulose stripping buffer (LI-COR, catalog no. 928-40030, lot no. D30111-04) for 5 minutes, following the manufacturer's instructions. After stripping, the blots were re-imaged as previously described, resulting in an unsaturated signal.

Blots containing 13-TAMRA were visualized using the ODYSSEY M LI-COR instrument paired with LI-COR acquisition 1.1 software. Scanning was carried out in the 700, 800, and 488 nm channels using the ‘membrane’ assay settings and 100 μm resolution. To ensure reproducibility, all experiments were conducted at least three times. The figures herein represent typical gels and blots from these repeated experiments.

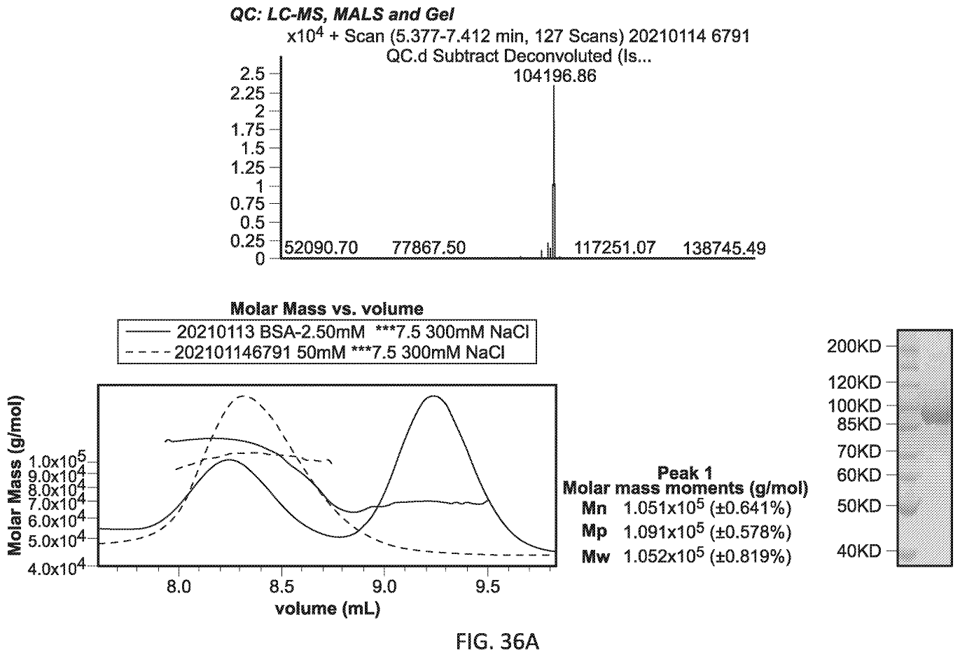

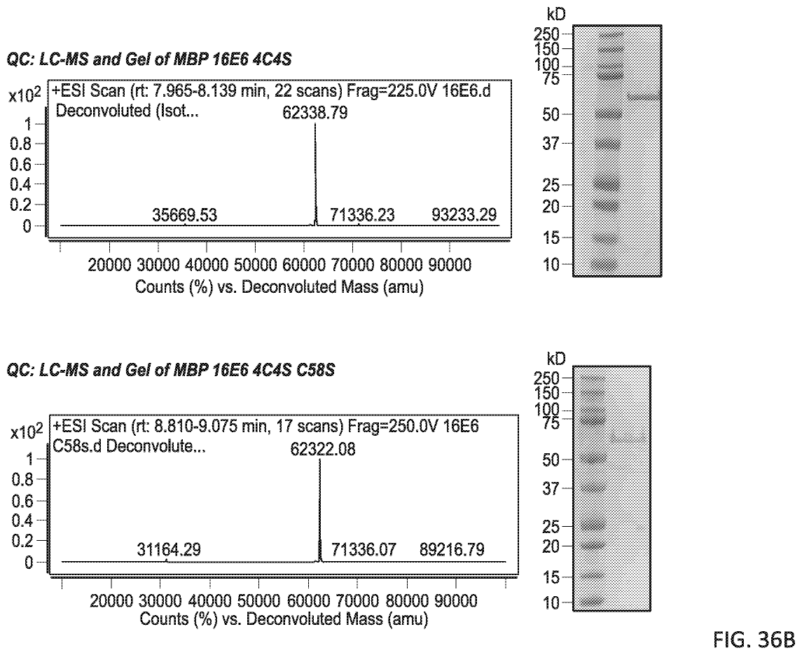

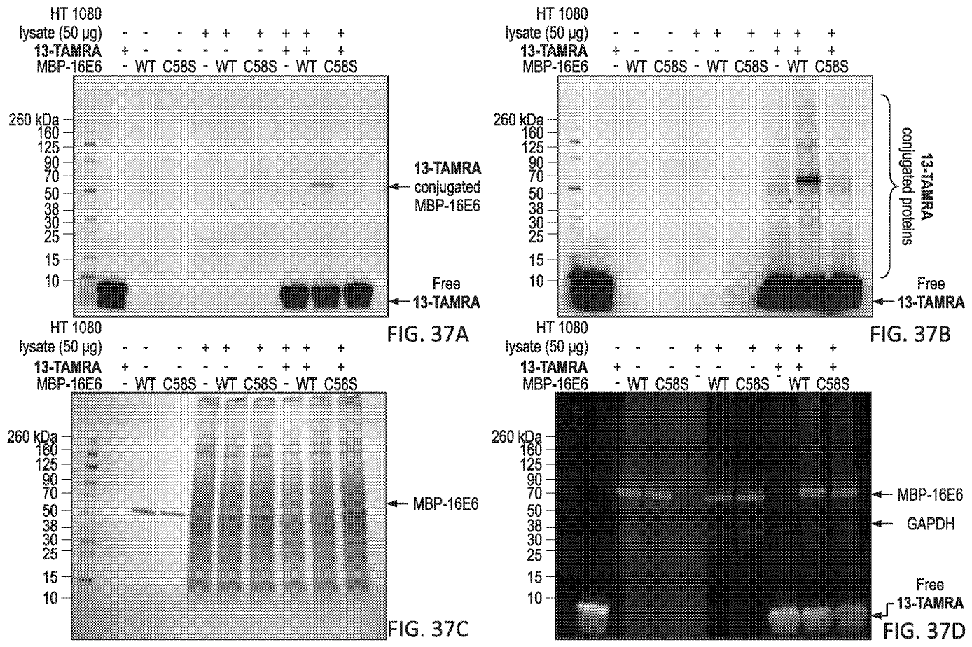

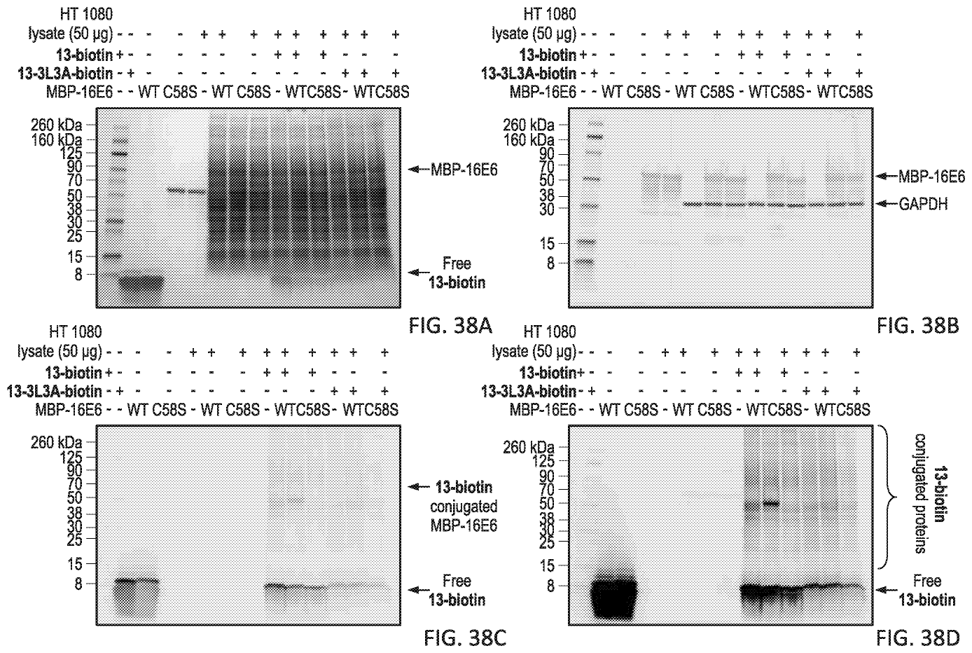

To investigate selectivity of peptide 13 in the context of the proteome, the HPV negative lysate was first incubated from HT1080 cells supplemented with recombinant MBP-16E6 or MBP-16E6 C58S then spiked in 10 μM of 13-TAMRA at 4° C. overnight. After resolution of the lysate by SDS-PAGE, the gel was scanned in the TAMRA channel which displayed selective covalent binding for MBP-16E6 over MBP-16E6 C58S (FIGS. 37A-37D). Despite this high concentration used in the assay (>500-fold higher than 13's binding affinity to 16E6), only a modest degree of fluorescent signal was distributed across the proteome with a long exposure time suggesting that 13-TAMRA has a minor degree of non-specific covalent modifications (FIG. 37B). As an orthogonal approach, 13-biotin (10 μM) or 13-3L3A-biotin (10) were incubated with 50 μg of lysate and resolved by SDS-PAGE followed by western blot analysis probing with fluorescently labeled streptavidin which recognizes biotin (FIGS. 38A-38D). A signal at ~60 kDa corresponding to MBP-16E6 was detected in the active 13-biotin reactide lane, but not in any of the other conditions, supporting 13-biotin's selectivity (FIGS. 38A-38C). However, general background levels of signal were also observed in both 13-biotin and 13-3L3A-biotin at 10 μM, both of which contain the reactive Dha warhead, suggesting that 13-biotin and 13-3L3A-biotin at 10 μM have a modest degree of non-specific covalent modifications. Background signal was not observed in peptide-free conditions suggesting that the signal originates from the peptide (FIG. 38D).

LC-MS/MS Analysis of Pull-Down by Biotinylated Peptides from Cells

To gain insight into the identity of the reactome, 13-biotin, 13-3L3A-biotin (5 PM) or DMSO was incubated in 2 mg of HPV16+ CaSki or HPV-HT1080 lysate overnight at 4° C. Subsequently, the biotinylated peptides were enriched using streptavidin-conjugated magnetic beads followed by trypsin digestion and analysis by LC-MS/MS to identify peptide-associated proteins.

HT1080 and CaSki (CRL-1550, ATCC) cells were grown to 70-80% confluency and harvested by trypsinization and centrifugation (500×g, 5 min). Pellets were subsequently washed with 1×PBS and lysed by the addition of cold Pierce IP lysis buffer (cat. no. 87787) supplemented with 1×Halt protease phosphatase inhibitor cocktail (ThermoFisher, cat. no. 1861284) for 10 minutes on ice, with intermittent pipetting to mix.

The supernatant was cleared by centrifugation at max speed on a bench-top centrifuge (10 min, 4° C.). Supernatant was removed and lysates quantified using a 660 nm protein assay (Pierce, cat. no. 22660). Each lysate replicate (2 mg) was incubated with either DMSO (final conc. 0.5%), 5 μM of 13-biotin or 5 μM of 13-3L3A-biotin in a final volume of 200 μL in IP lysis buffer (Pierce) overnight at 4° C. For each condition, 200 μL of streptavidin magnetic beads (Pierce, cat. no. 88816) were prepared according to the manufacturer's instructions and incubated with the biotin-peptide treated lysate for 2 h at RT with rotation. Beads were subsequently washed 3×1 mL with RIPA buffer (ThermoScientific; cat. no. 8990) with the addition of 1×Halt Protease Phosphatase inhibitor cocktail, and 3×1 mL with 50 mM HEPPS, pH 8.5 buffer. After the final wash, beads were resuspended in 50 mM HEPPS (pH 8.5) for on-bead reduction, alkylation and trypsin digestion. The samples were reduced with 5 mM BondBreaker TCEP (lh, RT, shaking), then alkylated with 10 mM iodoacetamide (30 min in the dark, RT, with shaking). Trypsin was added at a final concentration of 0.5 mg/ml for overnight digestion at 37° C. with shaking. Peptides were collected in new tubes and placed on ice until ready for Tandem mass tag (TMT) labeling.

TMTpro reagents were resuspended in anhydrous acetonitrile to a concentration of 20 mg/mL, then 15 μl of each TMTpro reagent (126, 127n, 127c, 128n, 128c, 129n, 129c, 130n, 130c, 131n, 131c, 132n) was used to label the individual samples. Following incubation at 25° C. for 1 hour, the reaction was quenched with 5% hydroxylamine to a final concentration of 0.3% hydroxylamine. The samples were acidified to a final concentration of 0.5% TFA and then all samples combined. The combined sample was desalted with StageTip as previously described and dried under vacuum^{[87]}.

Peptides were analyzed on an Orbitrap Fusion Lumos mass spectrometer coupled to an EASY-nLC 1200 (Thermo Fisher Scientific). Peptides were separated on an IonOpticks Aurora Ultimate C18 column (1.7 μm particle size, 120 Å pore size, 25 cm length×75 μm internal diameter) with the system operating at a flow rate of 300 nL/min and the column heated to 60° C. Peptides were eluted into the mass spectrometer using a 180-minute method with acetonitrile increasing over a 165-minute linear gradient from 8 to 30% in 0.125% formic acid.

Data-dependent acquisition (DDA) mode was used for mass spectrometry data collection. A high resolution MS1 scan was collected in the Orbitrap (500-1200 m/z range, 60,000 resolution, AGC 5×105, 30% RF lens, 100 ms max. injection time), and the top 10 precursors were selected for MS2 followed by MS3 analysis. For MS2, ions were isolated using a 0.5 m/z window. The MS2 scan was performed in the quadrupole ion trap (CID, AGC 1×104, 34% fixed collision energy, 35 ms max. injection time). The MS3 scan was analyzed in the Orbitrap (HCD, 60k resolution, max. AGC 5×104, 45% normalized collision energy, 250 ms max. injection time). For TMT reporter ion quantification, up to 6 fragment ions from each MS2 spectra were selected for MS3 analysis using synchronous precursor selection (SPS).

An in-house software pipeline was used to process all proteomics data^{[88]}. Raw files were converted to mzXML files and searched against a composite human UniProt database containing forward and reverse sequences with HPV16 and HPV18 E6 sequences added (P03126 and P06463) using the Sequest algorithm. Database searching matched MS/MS spectra with fully tryptic peptides from this composite dataset with a precursor ion tolerance of 20 ppm and product ion tolerance of 0.6 Da. TMTpro modification of peptide N-termini and lysine residues (+304.207146 Da) and carbamidomethylation of cysteine residues (+57.021464 Da) were set as static modifications. Oxidation of methionine residues (+15.994914 Da) was set as a differential modification. Peptide spectral matches were filtered to a 1% false discovery rate (FDR) using linear discriminant analysis (LDA) as previously described^{[88]}. Non-unique peptides that matched to multiple proteins were assigned to proteins that contained the largest number of matched redundant peptide sequences using the principle of Occam's razor^{[88]}. TMT reporter ion intensities were quantified by extracting the most intense ion within a 0.003 m/z window at the predicted m/z value for each reporter ion. Peptide intensities and signal-to-noise ratios were exported and analyzed using the msTrawler software package. Default settings were used with the exception of turning off the column/TMT channel normalization since differences in abundance between samples was to be expected^{[89]}.

The mass spectrometry proteomics data have been deposited to the ProteomeXchange Consortium via the PRIDE partner repository with the dataset identifier PXD044352.

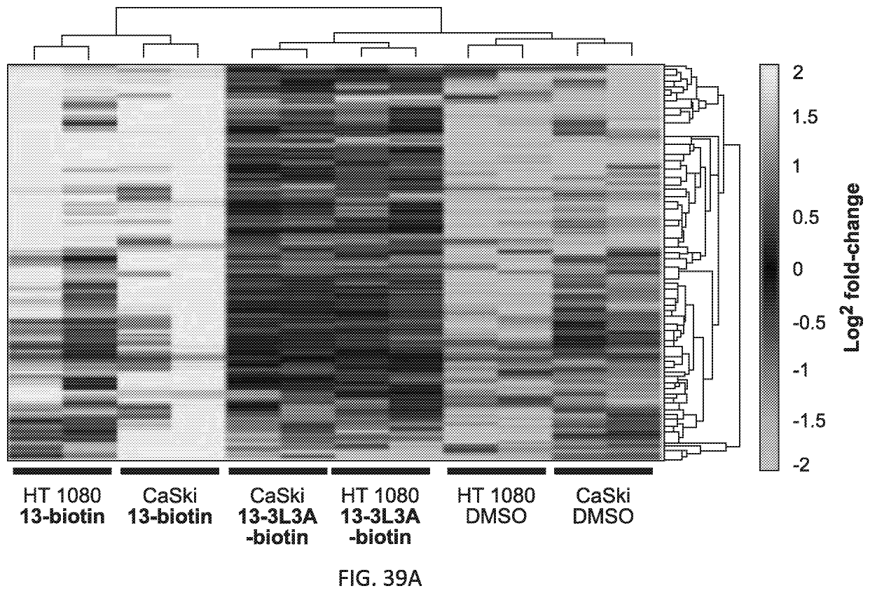

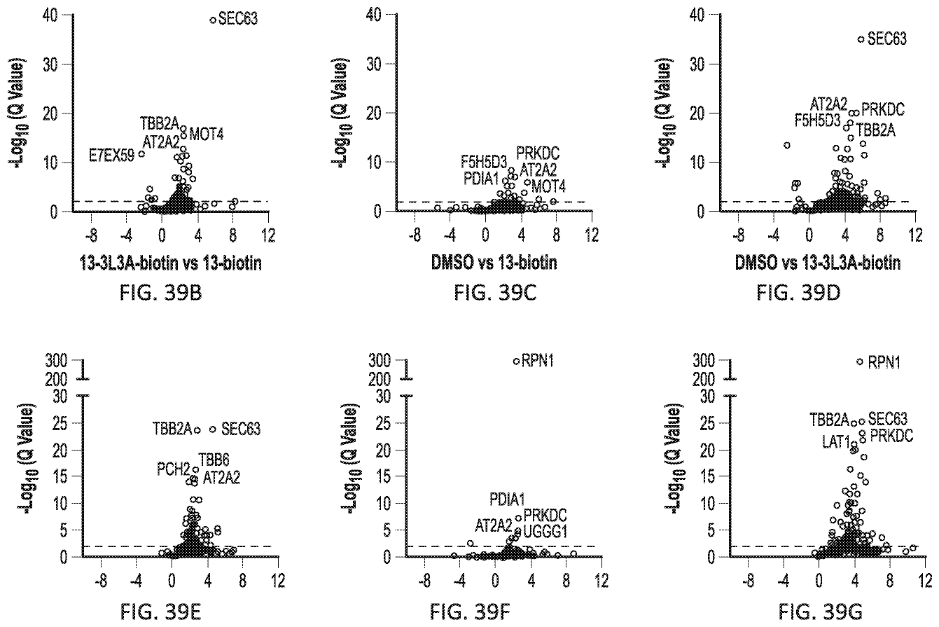

Approximately 372 proteins were identified across all tested conditions in both the HT1080 and CaSki cells, with 13-biotin showing the highest magnitude of enrichment (FIG. 39A). Subsequently, pairwise analysis was conducted across the conditions for both HT1080 (FIGS. 39B-39D) or HPV16+ CaSki cells (FIGS. 39E-39G) which did not reveal clear enrichment patterns; however, a major signal was observed for Rpn1 which has been reported to interact with E6AP.⁷⁹ Of note, HPV16 E6 was not identified in CaSki cells, which may be attributed to the low abundance of this protein or inaccessibility of the peptide binding site in a cellular environment. Nevertheless, 13-biotin or 13-3L3A-biotin had only modest nonspecific enrichment of other proteins in both cell lines. Taken together, these data suggest that 13-biotin recognizes supplemented MBP-16E6 but not MBP-16E6 C58S with a minor to modest degree of non-specific conjugation to other proteins from lysate.

### Example 9—Disruption of 16E6/E6AP Interaction by Reactide 13

This example describes the determination of the disruption of 16E6/E6AP interaction by reactide 13, by a 16E6/E6AP BLI binding assay using immobilizing biotinylated E6AP protein onto streptavidin tips.

Briefly, E6AP protein was biotinylated by a commercial BirA kit. In brief, 40 μL of E6AP was added directly into a tube containing 20 μl of 1 mg/ml BirA biotin-protein ligase. Then the solution was added with 5 μL of BiomixB (10×concentration: 100 mM Adenosine 5′-triphosphate, 100 mM MgOAc, 500 μM d-biotin), followed by the addition of 5 μL of BIO200 (d-biotin; 10× concentration: 500 μM). The whole mixture was incubated at 4° C. for 16 hrs, followed by a spinning filter through 0.2 μm. Excess biotin was purified away on Superdex200 or AdvanceBio 150 mm (Agilent, MA) equilibrated with PBS+1 mM DTT. Biotin-E6AP fractions were collected and frozen at −80° C. before use. 50 μL of 12 μM MBP-E6 in PBS was added with 0.5 μL of 10 mM reactive peptide of interest (final concentration 100 μM). The mixture was incubated at 4° C. for 16 hrs. One μM of Biotin-E6AP protein was immobilized onto streptavidin sensors soaked in a blocking buffer. The sensors were then dipped into the mixture of MBP-E6 and reactive peptide. The signal response was recorded.

The 16E6/E6AP interaction was evaluated as the response (in nm) during the association step when dipped into recombinant MBP-16E6 solution (1 μM). MBP-16E6 conjugated with reactide 13 had a significantly decreased BLI response signal to the immobilized E6AP protein, while the control reactide 13-3L3A had no impact (FIG. 10E). The decrease in BLI response signal indicates that the 16E6-13 conjugate loses its E6AP-binding ability, as its E6AP binding pocket was occupied by 13.

### Example 10—Structural and Molecular Modeling of E6-Peptide-13 Complex

This example describes molecular modeling of the mechanism by which reactide 13 disrupts the 16E6/E6AP interaction using molecular docking and molecular dynamics (MD) simulations.

The MBP tag may affect the conformation of the isolated E6AP-based peptide from the ternary complex structure with 16E6 and p53 (PDB: 4XR8). Therefore, the MBP tag and p53 protein was removed from the structure and a 1.1 μs simulation of the 16E6-bound E6AP-LXXLL peptide was performed.

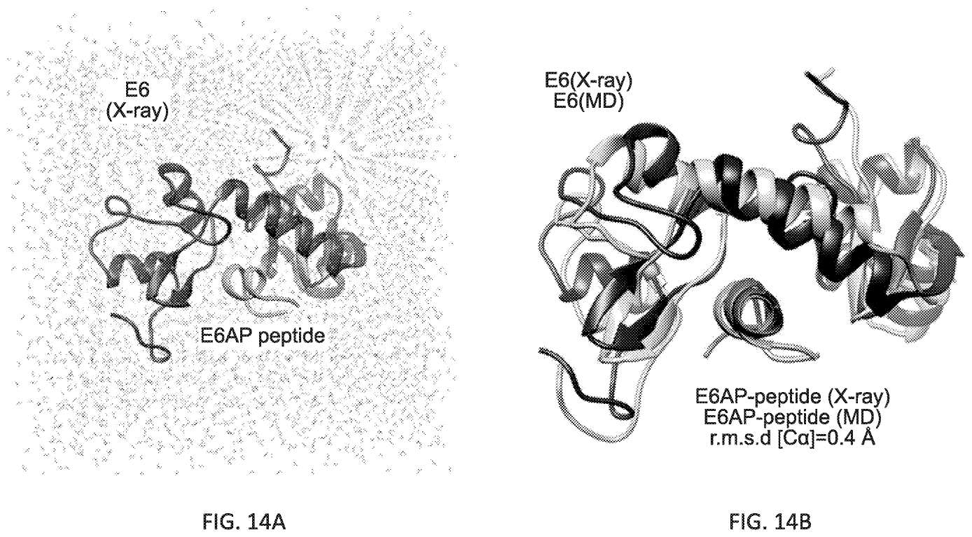

Briefly, peptide-13 consists of a flexible N-terminal region (9-fluorenyl acetamido-IPQSA (SEQ ID NO: 37), the native E6AP core (ELTLQEELL (SEQ ID NO: 38)), and flexible C-terminal (Dha-RRKK-K (1-anthracenyl acetamido) (SEQ ID NO: 39) segments, as shown by its sequence (FIG. 9B). The E6AP peptide, which interacts with the E6 protein and functions as a ubiquitin ligase, has an alpha-helical conformation according to the x-ray E6-MBP(E6AP)-P53 ternary complex (PDB ID: 4XR8). To investigate whether the native core sequence of E6AP in Peptide-13 also adopts an alpha helix fold, the x-ray complex was used as a starting point, with removed MBP and P53 proteins, and the E6-bound E6AP peptide was kept. A MD simulation in a box (60.0 Å*60.0 Å*60.0 Å) of water (~6K water molecules) and ions was performed and found that the E6AP peptide remained stable (FIG. 14), with an r.m.s.d. of 0.4 Å (measured using the Ca atoms) compared to the E6AP peptide in the ternary complex.

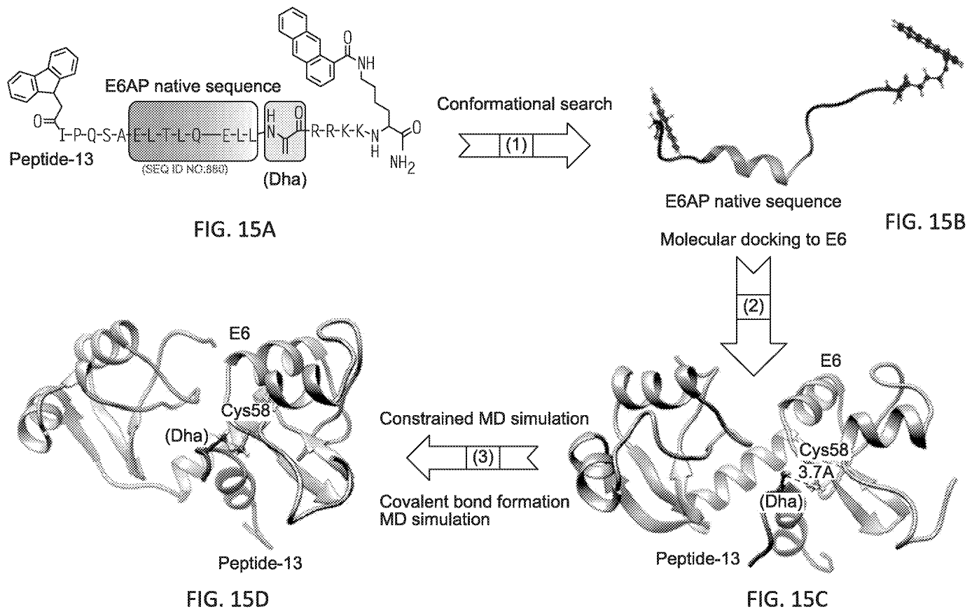

Additional modeling using Molecular Operating Environment (MOE) software (MOE, Canada), MOE docking module for molecular docking, and MD simulations to further refine the E6-Peptide-13 complexes in the presence of water and ions were used. Briefly, using the MOE software, the alpha helix E6AP core was expanded upon by incorporating the flexible N-terminal and C-terminal sequences to construct the Peptide 13 molecule (FIG. 15B). To optimize the Peptide 13 structure, the LowModeMD algorithm^{[90]}, which is integrated into MOE, was employed. The sampling was limited by imposing the following parameters defined in the MOE package: {Rejection Limit=100; Iteration Limit=10000; RMS Gradient=0.005, MM Iteration Limit=500; RMSD limit=0.75 Å; Energy Window=10.0 kcal/mol; Conformational Limit=10,000}. Additionally, the backbone atoms of the E6AP peptide were treated as a rigid segment to maintain the alpha-helix conformation during the calculations.

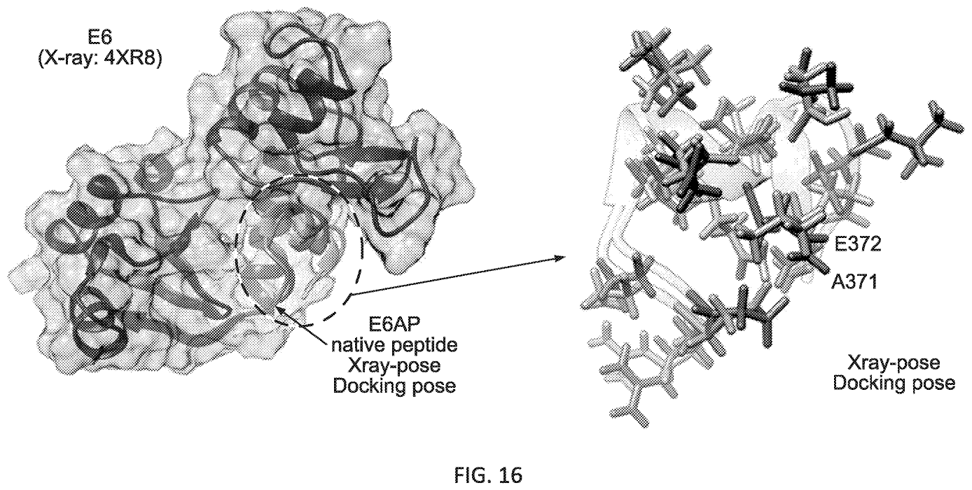

To determine how Peptide 13 binds to the 16E6 protein (FIGS. 15C and 15D), the MOE docking module was used for molecular docking, which is known to be reliable for capturing peptide/receptor interactions^{[91,92]}. To ensure the accuracy of the approach, molecular docking was first performed to determine the binding mode of the native E6AP peptide to the crystallographic 16E6 protein, using the X-ray E6-bound native E6AP peptide (PDB ID: 4XR8) as the starting point after eliminating the MBP and p53 from the ternary complex. A docking protocol was employed that started with the triangular matcher algorithm to quickly generate 1,000 poses of docked peptides based on the receptor shape. From the resulting docked poses, the London dG scoring function was used to screen among the resulting poses to keep the top 100 structures for further refinement. Then, the rigid receptor replacement method combined with the GBVI/WSA dG scoring function was used to refine the resulting poses, obtaining an E6-bound E6AP peptide that closely matched the crystallographic binding mode of E6AP (FIG. 16). Similarly, the same docking procedure was followed to obtain top 3 docking poses of 16E6-Peptide-13 complexes using the pre-generated conformations of peptide 13 (described above) to dock to the binding pocket of the native E6AP peptide in the 16E6.

Next, ~60 ns of constrained MD simulations was performed to further refine the E6-Peptide-13 complexes in the presence of water and ions. The goal was to push the peptide-13 warhead (Dha) toward the 16E6 Cys58 hotspot and create a covalent link between the two. To achieve this, a harmonic restraint was placed at 3.0 Å between Cys58 (S atom) and Dha (C atom), with a force constant of 0.36 kcal mol-1 Å-2. These simulations allowed to prepare the 16E6-Peptide-13 constructs for the formation of the covalent link between Dha and Cys58.

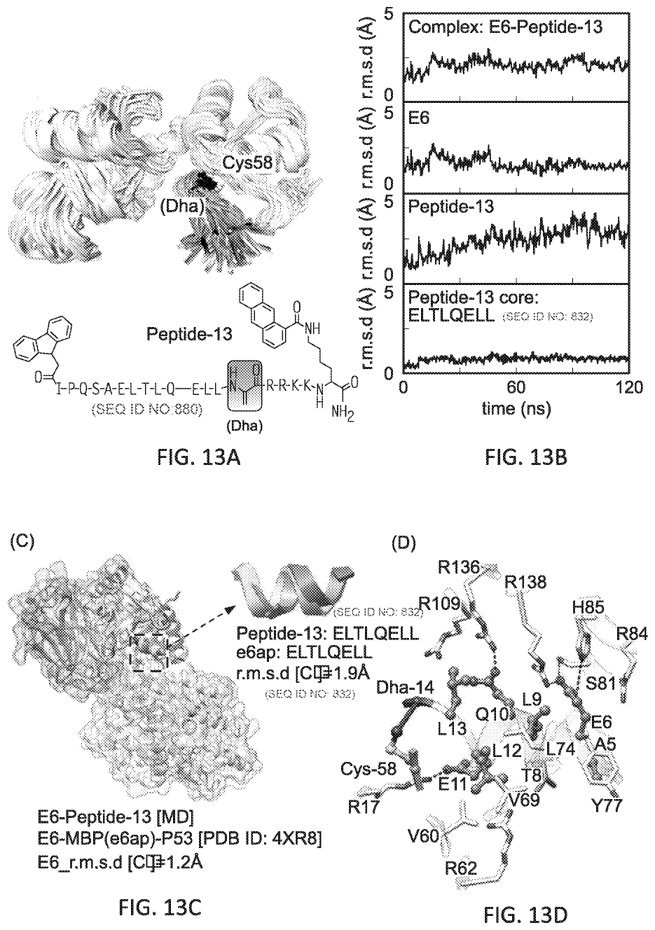

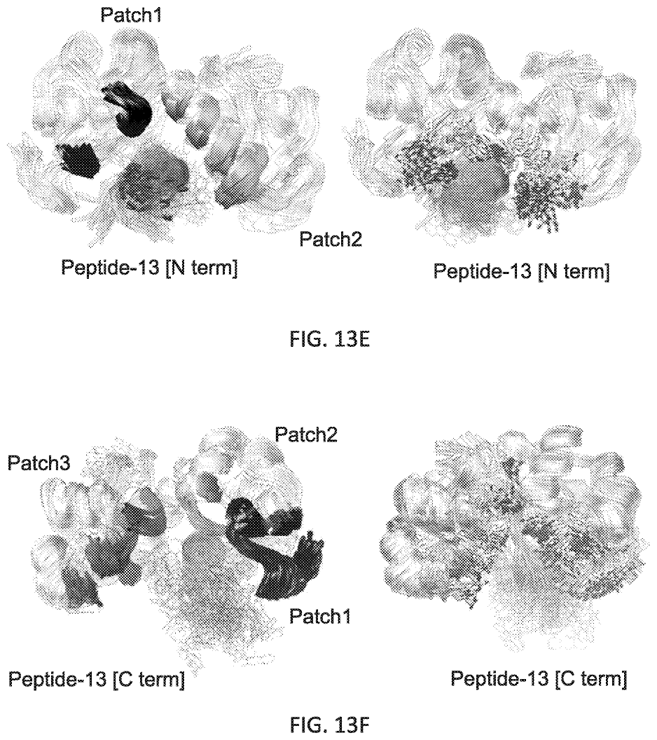

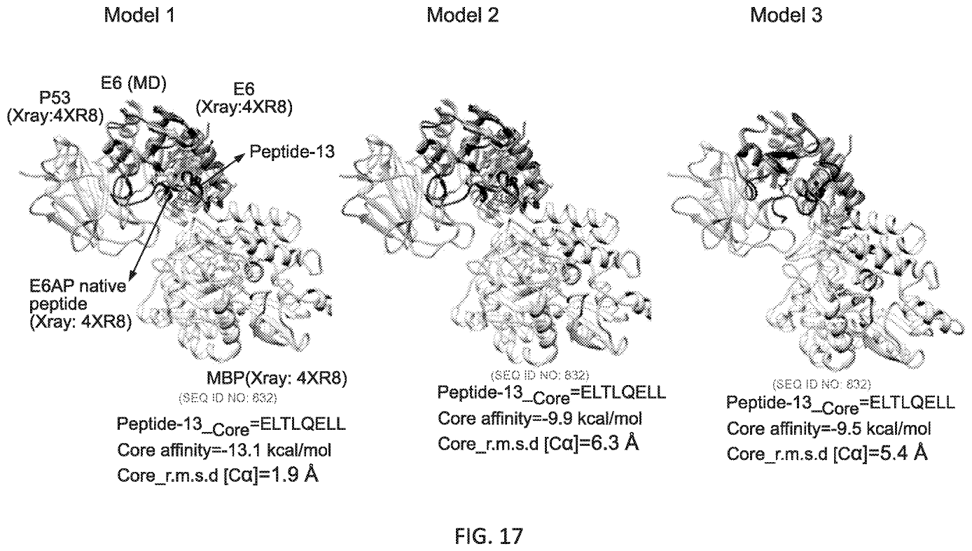

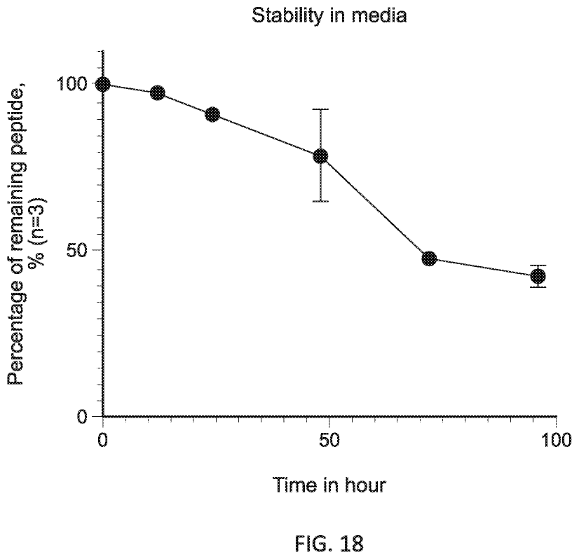

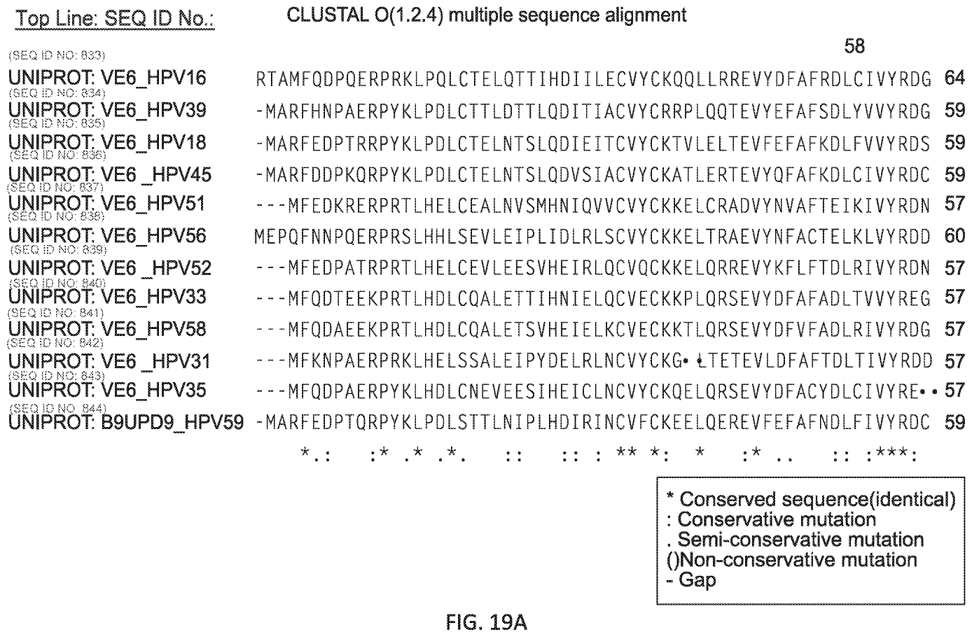

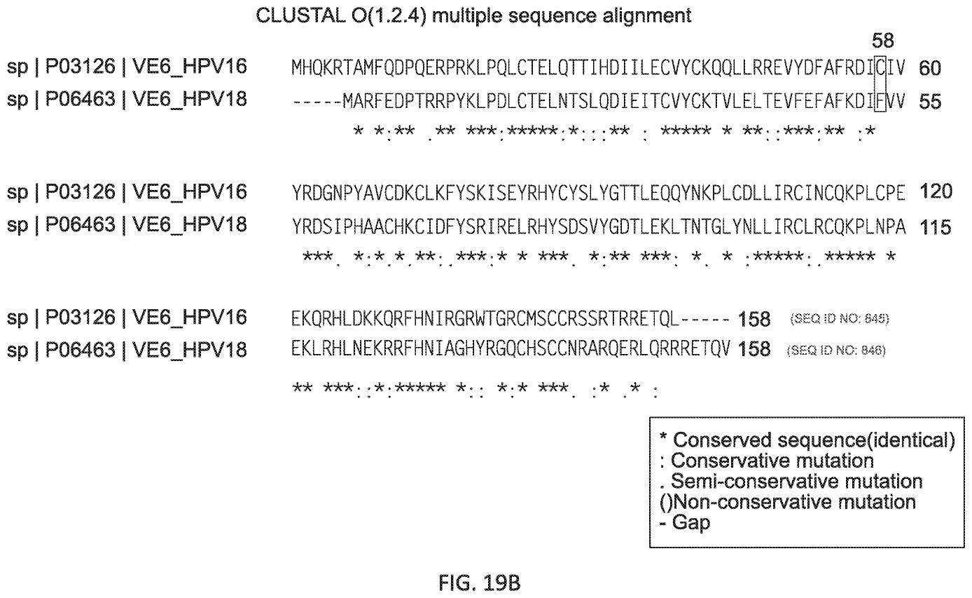

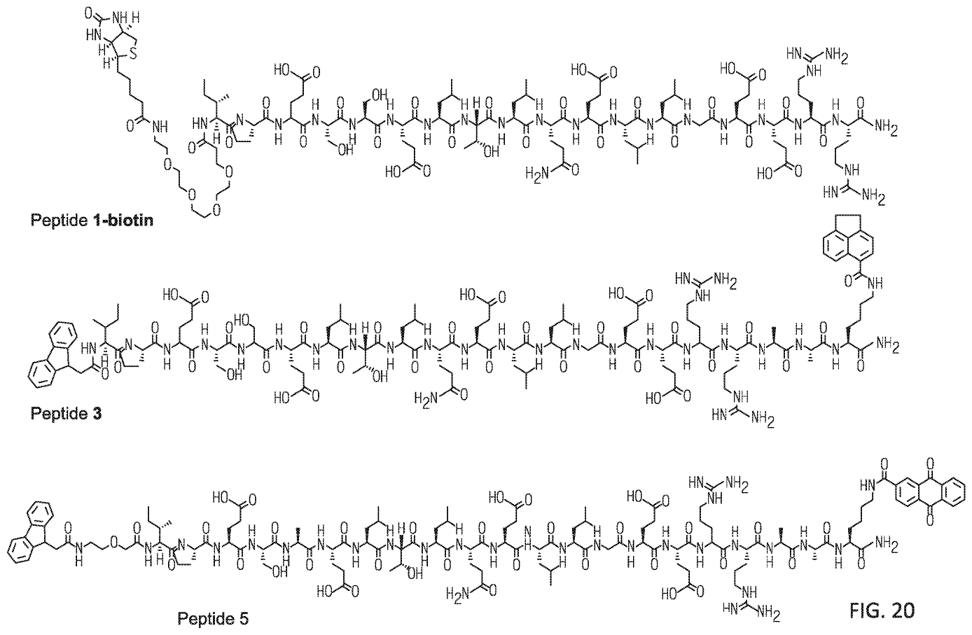

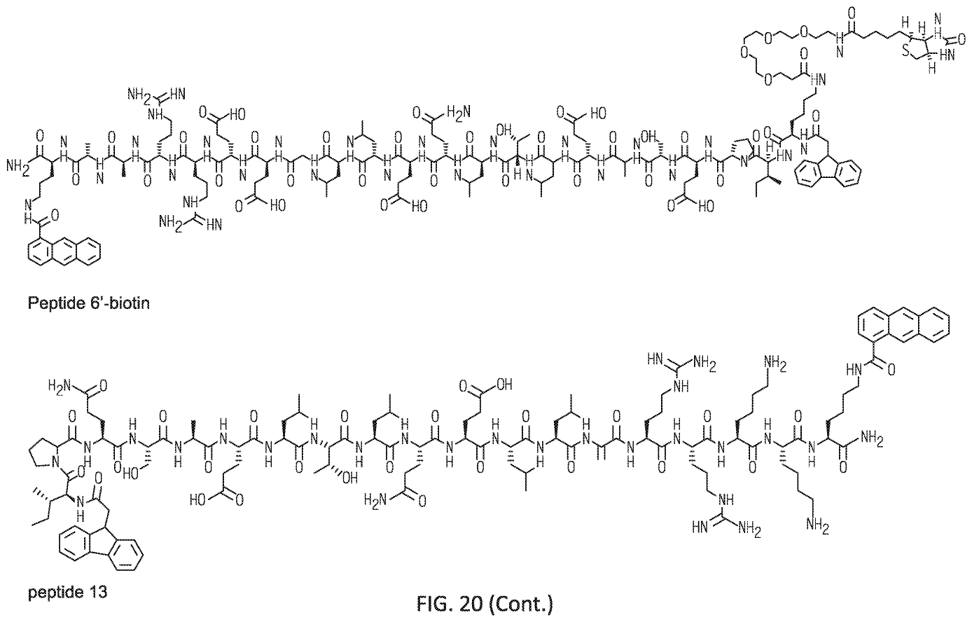

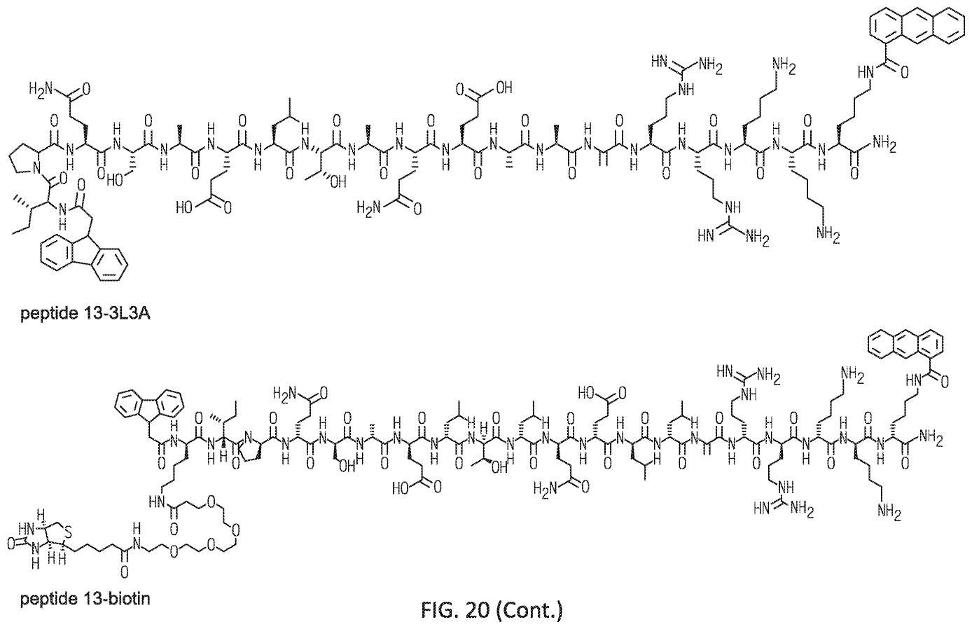

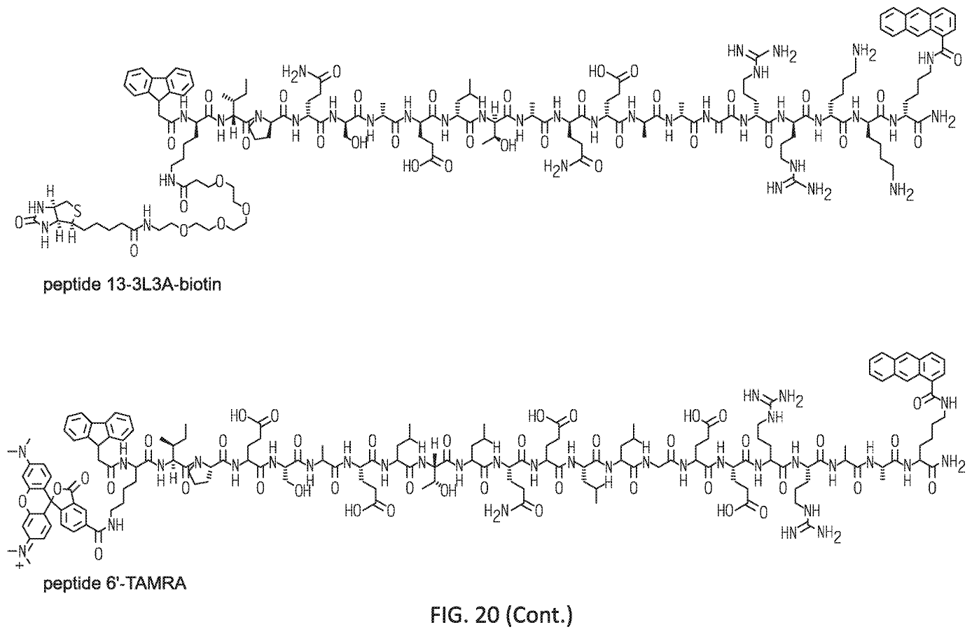

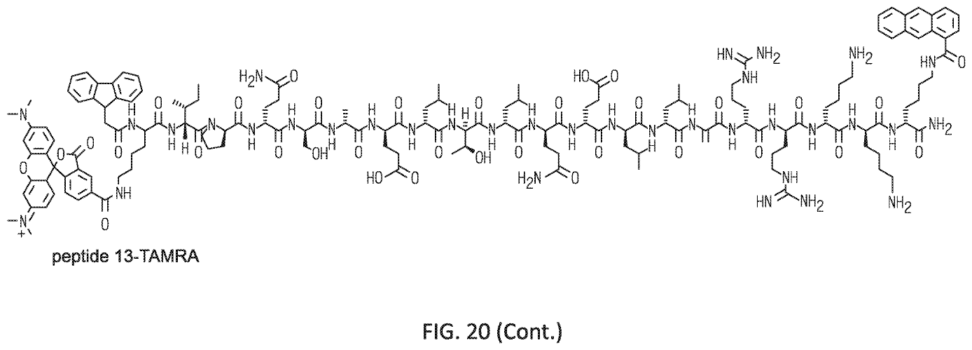

Subsequently, Cys58 and Dha were covalently linked using MOE followed by energetically minimizing the covalent link and the 16E6-Pepitde-13 complexes. Each of three complexes were then subjected to a ~120 ns classical MD simulation to characterize the binding mode of Peptide-13 covalently linked to the 16E6 protein. The resulting final complexes closely match the crystallographic E6AP binding peptide (FIG. 17). For further analysis, the structure featuring the largest binding affinity between the peptide-13 core sequence and E6 while exhibiting the lowest RMSD of the peptide-13 core sequence compared to the crystallographic native E6AP peptide was selected. For the results presented in FIGS. 13A-13F, GROMOS algorithm^{[93]} with RMSD cutoff=0.18 Å was used to cluster the entire MD trajectory of the best 16E6-Peptide-13 complex.

A model of peptide 13 was built by incorporating the flexible N- and C-terminal sequences into the alpha helix of the E6AP LXXLL peptide and extensive conformational sampling calculations to optimize the reactide 13 model were performed. The covalent bond formed between 13 and 16E6 Cys58 was not considered when initiating the molecular docking to avoid any biases in the calculations. The docking result suggested that the primary driver of 13 binding to 16E6 are electrostatic and van der Waals interactions (FIG. 13A). By refining the top candidates obtained from the docking calculations using MD simulations, the Dha (C atoms) resides within 3.7A from Cys58 (S atoms), confirming optimal placement for 13 to cross-link with 16E6. The thioester bond between 16E6 and Dha acts as a covalent lock, securing 13 at the 16E6/E6AP interface. Trajectory r.m.s.d analysis (FIG. 13B) for the 16E6 and 13 complex and 13 alone indicated that the covalent bond between Cys58 and Dha is instrumental in stabilizing 13 and effectively restricts the flexibility of the E6AP-LXXLL core sequence with an r.m.s.d of 1.5±0.3 Å. The molecular simulations reveal that reactide 13 occupies the same position as the parent E6AP LXXLL peptide (r.m.s.d=1.9 Å, measured for Ca atoms), leading to the blocking of E6AP/16E6 binding (FIG. 13C).

The modeling suggests important side chain interactions between 13 and 16E6 basic residues, including E6AP Glu6-16E6 Arg138 and E6AP Glu11-16E6 Arg11. 16E6 Arg138 is crucial as Arg138 mutation reduces E6AP recruitment and interactions substantially. In addition, 16E6 Arg17 forms a salt bridge with the C-terminus of the E6AP core peptide. The Glu6 carboxylate group is essential for 13 binding and establishes an additional network of hydrogen bonding with Ser81 and His85 from 16E6 (FIG. 13D). 16E6 Arg136 forms a hydrogen bond with Gln10 in the E6AP core. The keystone 16E6 Arg109 interacts with Leu13 through hydrophobic interactions, consistent with crystallographic observations. Notably, the alanine substitutions of both Arg136 and Arg109 led to impaired peptide binding, highlighting their crucial roles in stabilizing the 16E6/E6AP complex, though R136 exhibited conformational disorder and was oriented away from the peptide towards MBP. Collectively, 13 selectively targets all residues appearing in the binding pocket of E6 to disrupt the binding interface of 16E6 and E6AP.

The N- and C-terminal groups are located on flexible loops and dynamically bind to 16E6, making it challenging to identify static interactions similar to those found for the core motif. However, despite their dynamic binding, the N- and C-terminal groups still interact with important surfaces of E6. This flexibility and ability to cover essential surfaces of 16E6 may prevent 16E6 from recruiting E6AP (FIGS. 13E and 13F). Notably, the binding interface of the N and C-terminal groups includes several key residues that are essential in the 16E6-E6AP complex. Therefore, these results suggest that hydrophobic modifications contribute to the inhibition of 16E6.

### MD Simulation Protocol

Before running molecular docking and MD simulations, the MOE preparation module was used to refine the crystallographic E6 protein and the missing sidechains/residues were added. The 16E6-pepitde complexes were then immersed in a water/ion (150 mM excess ions) box and underwent 500 steps of energy minimization using the steepest descents algorithm in GROMACS^{[94]}. The refinement was followed by an MD simulation in a canonical ensemble, where the system was heated gradually from 0 K to 310 K in 20 ps, followed by MD simulations in an isobaric-isothermal ensemble for an aggregated 80 ps, during which the pressure was maintained at 1 bar to relax the simulation box. Throughout these pre-equilibration steps, the positional restraints were placed on all heavy atoms, gradually reduced to 0 kcal·mol-1Å2 for the final equilibration step. Finally, the 16E6-pepitde constructs were optimized by removing the positional restraints.

In all simulations, the AMBER-14 force field parameter set^{[95]} was used to describe 16E6, Peptide-13, E6AP peptide, and ions. Non-canonical amino acid residues were parameterized with the Antechamber program^{[96]} to ensure compatibility with the AMBER force field, while the force field parameters for two tagged molecules were borrowed from the Generalized AMBER force field (GAFF)^{[97]}. The TIP3P model was utilized to describe water.

The temperature was maintained at 310 K using a velocity-rescale^{[98]} thermostat with a damping constant of 1.0 ps for temperature coupling and the pressure was controlled at 1 bar using a Parrinello-Rahman barostat algorithm^{[99]} with a 5.0 ps damping constant for the pressure coupling. Isotropic pressure coupling was used during this calculation. The Lennard-Jones cutoff radius was 12 Å, where the interaction was smoothly shifted to 0 after 10 Å. Periodic boundary conditions were applied to all three directions. The Particle Mesh Ewald algorithm^{[100]} was used to calculate long-range coulombic interactions with a real cutoff radius of 10 Å and a grid spacing of 1.2 Å. A compressibility of 4.5×10-5 bar-1 was used to relax the box volume. In all the above simulations, water OH bonds were constrained by the SETTLE algorithm^{[101]}. The remaining H-bonds were constrained using the P-LINCS algorithm^{[102]}. All MD simulations were carried out using GROMACS, with constrained MD simulations aided by PULMED^{[103}].

### Example 11—Identification Of Reactive Peptides Mimicking E6AP

This example describes the design and identification of reactive peptides based on E6AP that target HPV16 E6 (E6).

Briefly, the structure of the complex formed between E6, E6AP, and p53 was analyzed (PDB: 4XR8) and a truncation study on E6-E6AP interaction sites revealed the main recognition domain-a peptide fragment of E6AP (residues 406-417), termed ELT peptide: ELTLQELLGEER (SEQ ID NO: 5).

Binding was measured with BLI assay as described above and fluorescent polarization (FP) assay.

### Fluorescent Polarization(FP) Direct Binding Assay

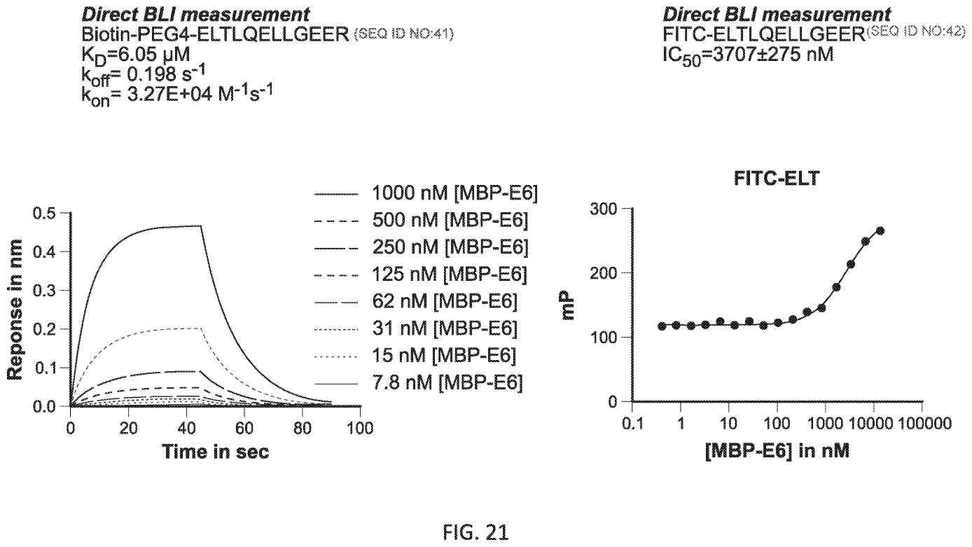

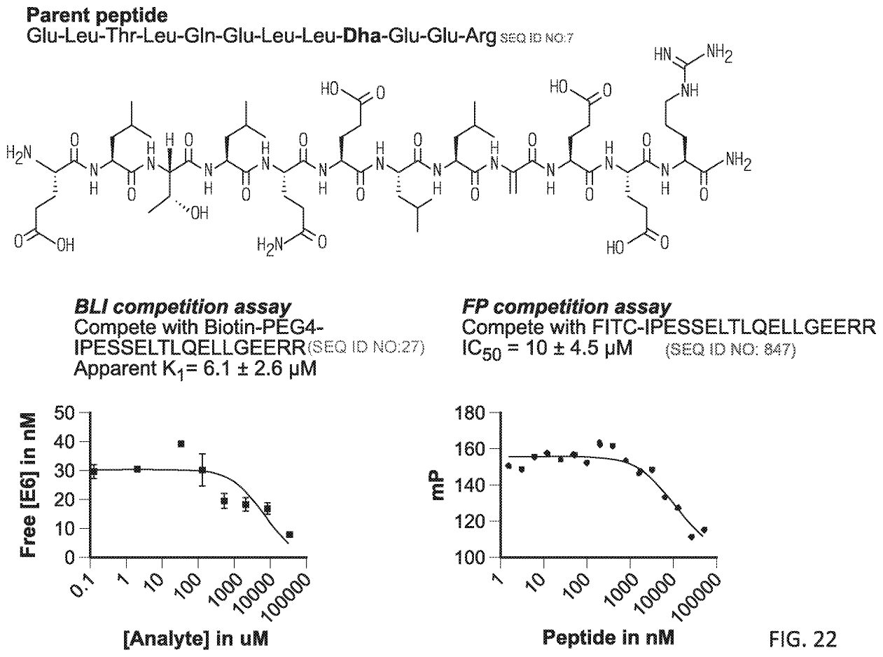

Direct FP binding assay of FITC-ELT (FITC-ELTLQELLGEER (SEQ ID NO: 42)) and FITC-IPESS (SEQ ID NO: 40) (FITC-IPESSELTLQELLGEER (SEQ ID NO: 44)) (FIG. 21 and FIG. 22). FP was recorded after incubating various concentration of MBP 16E6 with 100 nM FITC-ELTLQELLGEER (SEQ ID NO: 42). FP was performed with a BioTek H1 plate reader. Excitation: 485/20, Emission: 528/20. (Green) Filter set. Read Height: 10.5 mm. Gain sets to 50. Temperature 27° C.

The results show, that ELT peptide had a mid-micro molar binding affinity to 16E6, about 6 μM by direct biolayer interferometry (BLI) assay with biotin-ELT, and about 3.7 μM by direct fluorescent polarization (FP) assay with FITC-ELT to the recombinant MBP-16E6 4C4S protein (MBP-16E6) (FIG. 21). The MBP-16E6 was stabilized and solubilized by a maltose-binding protein (MBP) tag and four cysteine (SEQ ID NO: 885) to serine.

The peptide fragment contains a common binding motif LXXLL that are critical for binding (see also Example 7).

To overcome the fast koff rate and disrupt the 16E6-E6AP interaction, E3 (ELT-G9Dha) was developed, a cysteine reactive peptide containing dehydroalanine (Dha) that mimics E6AP. This peptide showed an apparent Kᵢ of 10 μM by FP and apparent Kᵢ of 2 μM by BLI (FIG. 21).

To dissociate the peptide-protein complex, the Dha warhead that forms irreversible bonds was replaced with a cysteine that is capable of forming reversible disulfide bonds. Disulfide dimers ELT-G9DS and ELT-E10DS, were prepared which showed crosslink yields of 75% and 45%, respectively. Despite not having the highest crosslink yield, ELT-G9DS was chosen to match with ELT-G9Dha (ELT with Dha substitution).

Three different small molecule-peptide mix disulfides were prepared: cysteine (Cys), cysteamine (βME) and N-acetyl cysteamine (NACβME). Using an on-resin synthesis, a mix cysteamine-peptide disulfide was produced on resin with quantitative yield. Among the disulfides, NME disulfide (βME-DS) showed the fastest crosslink yield of 86%, while NACβME disulfide (NACβME-DS) yielded 37%, which was twice that of the free cysteine yield of 16%.

After screening the ELT-G9DS and ELT-E10DS disulfides mix with small molecules, cysteine peptides were identified. In the resynthesis, Cys was replaced with Dha substituted Gly13.

### Example 12—Design and Screening of a 1.42-Million-Member Peptide Library

This example describes the design of a peptide library based on the ELT peptide of E6AP and screen of the library for high affinity binders to 16E6.

Briefly, a library with 1.42 million members was designed based on the ELT peptide ELTLQELLGEER (SEQ ID NO: 5).

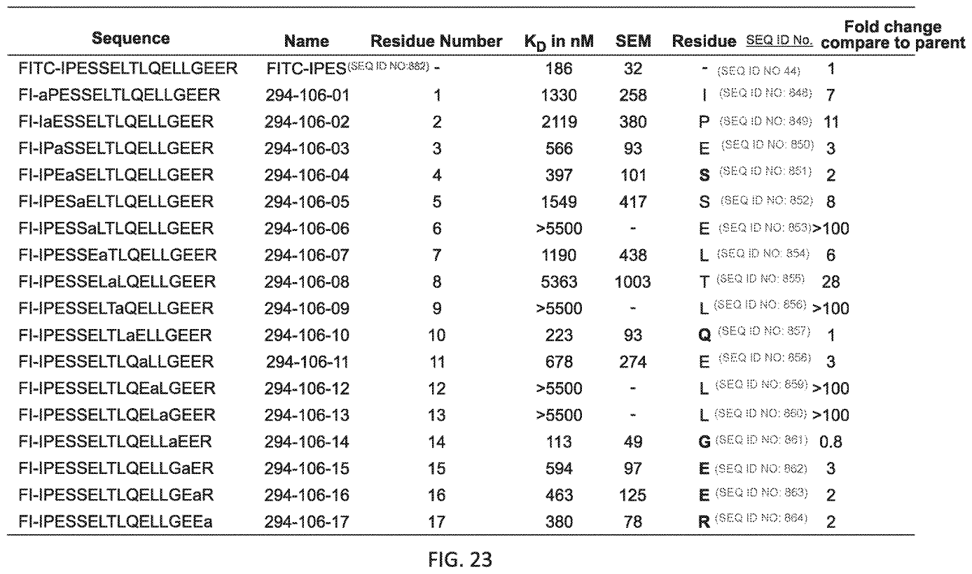

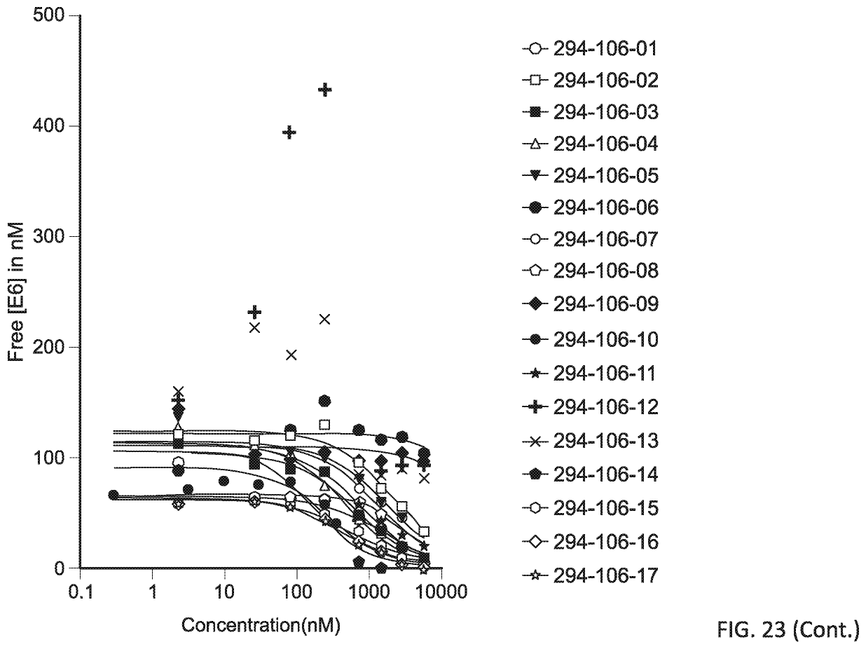

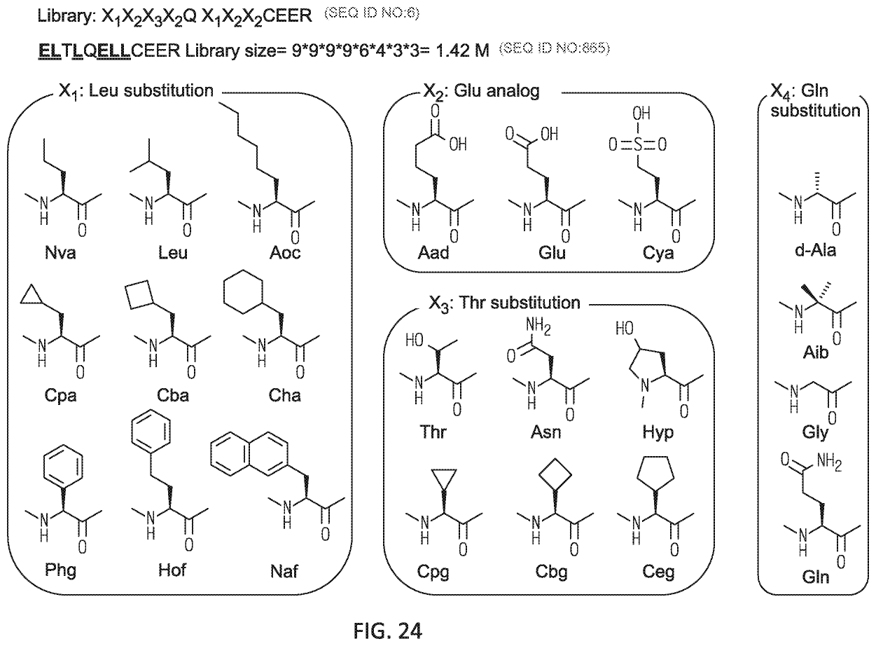

Results from the Alanine scan showed that alanine mutations in the residues E1 (E1A) and E6 (E6A) of the ELT peptide decreased binding affinity to MBP-16E6 by 9-fold and 16-fold respectively (FIG. 23). These two positions were replaced with Glutamic acid analogs. The _{D}-alanine scanning showed Q5a and Q5A mutations of the ELT peptide did not affect binding, therefore, the Q5 position was mutated to Alanine analogs, including Gly, d-ala and Aib. The four Leu residues were mutated to aliphatic and aromatic sidechains. Thr3 position was replaced by Thr analogs and β-substituted amino acids. The C-term CEER residues were kept constant, with a positively charged Arginine can increase sequencing efficiency. The L-alanine scanning identified hotspot residues Leu4, Leu7 and Leu8 that were critical for binding. L4A, L7A and L8A simultaneous mutated peptides (or 3L3A) were used as negative control peptides. The library had the consensus sequence X₁X₂X₃X₂QX₁X₂X₂CEER (SEQ ID NO: 6) with X₁, X₂, X₃, and X₄ chosen from FIG. 24 (X₁ (Nva, Leu, Aoc, Cpa, Cba, Cha, Phg, Hof, and Naf), X₂(Aad, Glu, and Cya), X₃ (Thr, Asn, Hyp, Cpg, Cbg, and Ceg), and X₄ (d-Ala, Aib, Gly, and Gln)). Residue G9 replaced with a Cys.

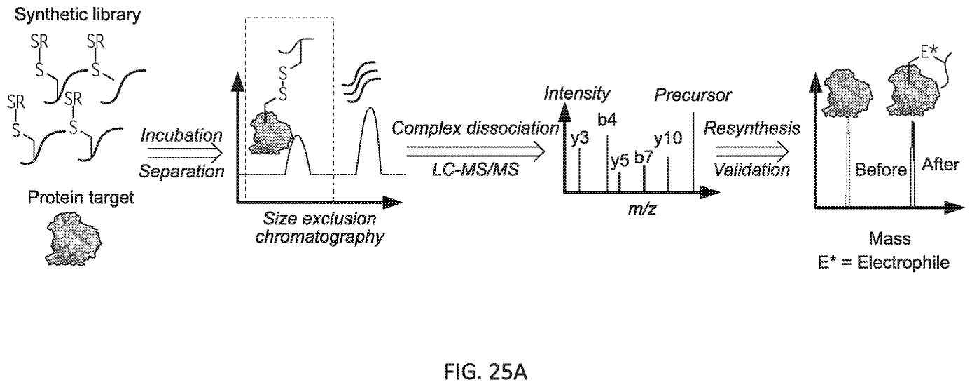

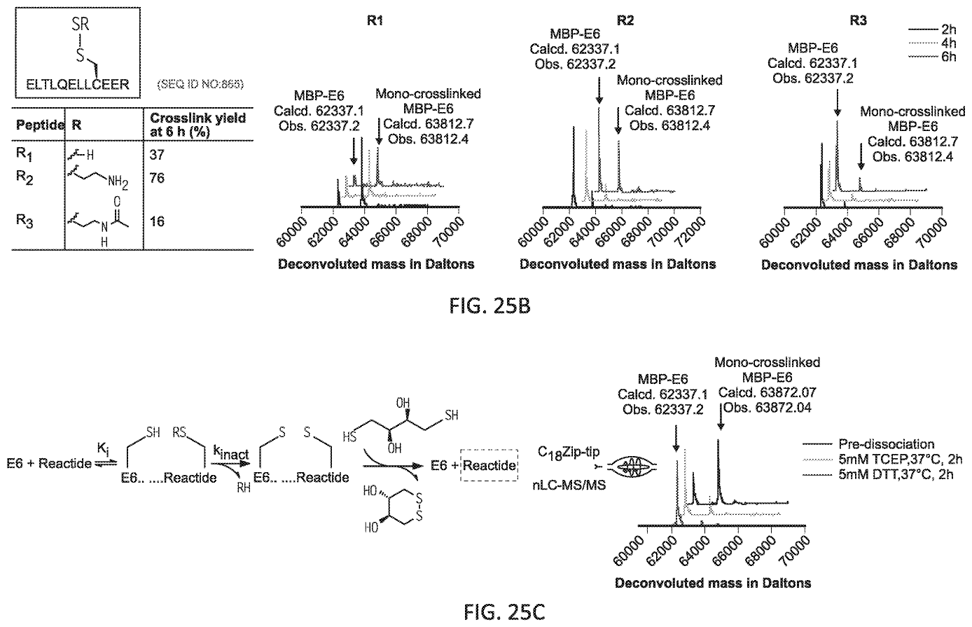

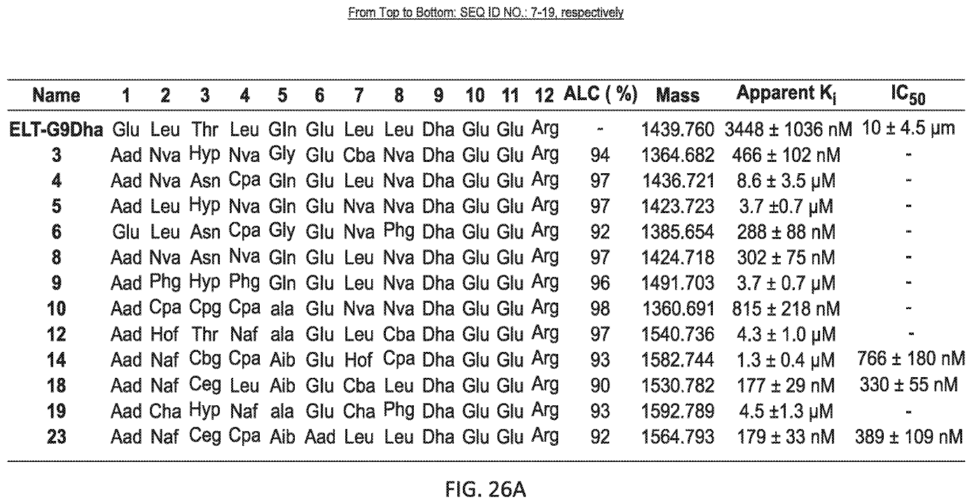

The ELT peptide library was screened with the ReAct-ASMS method (FIG. 25). Briefly, the affinity selection platform, named Reversibly Active Affinity Selection Mass Spectrometry (ReAct-ASMS), can be used to rapidly identify high-affinity covalent crosslinkers from synthetic libraries of reactive peptides. This approach has several advantages, including label-free screening using High Performance Size Exclusion Chromatography (HPSEC)-ASMS, which eliminates the need to functionalize or label recombinant proteins with affinity tags like biotin, IgA, or GST. The flow-based SEC separation allows for rapid and robust separation of bound and unbound molecules with affinity ranges of 200 μM-1 μM. This platform enables affinity maturation on covalent PPI peptide inhibitors using non-canonical amino acids.

To mature the E6AP-mimicking peptide, ReAct-ASMS libraries were prepared with fast crosslinker-βME-DS and slow crosslinker NACβME-DS. Library I-βME and library I-NACβME were individually screened against MBP-16E6. With the same amount of incubation time, library 1-βME generate an average of 7 hits with ALC>90, 7-fold more than library I-NACβME with an average of 1 hit with ALC>90, with frequent identification of parent peptide, which was supplemented as a competitor in the screening. The finding suggested that fast crosslinker generated more hits and was select for further investigation.

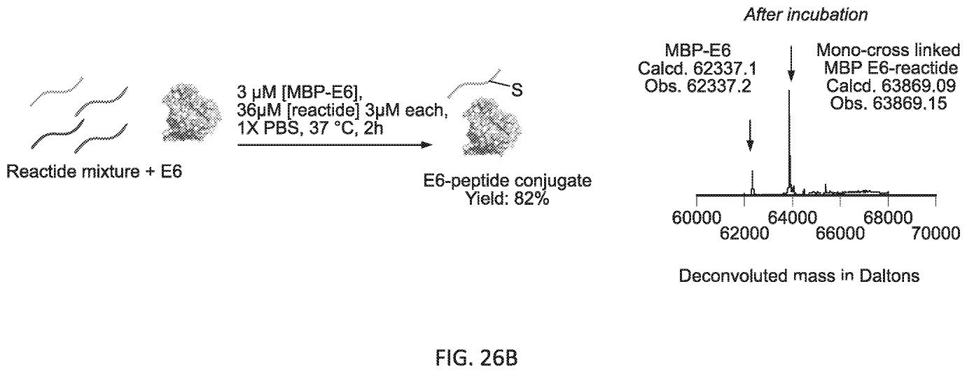

ReAct-ASMS with MBP-16E6 identified peptides from the X₁X₂X₃X₂QX₁X₂X₂CEER (SEQ ID NO: 6) library. Three ReAct-ASMS conditions with different ratio of libraries and MBP-16E6 protein were mixed, incubated, and separated by HPSEC. The breakthrough MBP-16E6 protein fraction was collected, dissociated by reductant treatment, and analyzed by an Orbi-trap nLC-MS/MS. De novo peptide sequencing was constructed by PEAKS studio software and filtered by python scripts. Among all library 1-βME selection conditions, 14.4 hits were identified in the loose condition (>1 ng per member, n=10) and 2.6 hits in the stringent condition (0.1-0.6 ng per member, n=14) on average. Twenty-three hit peptides were select from the most stringent condition with average local confidence (ALC) >90. Eleven peptides were shortlisted for resynthesis, with Cysteine being replaced by Dha for irreversible inhibition of target protein (FIG. 26A).

Additional peptide libraries screened are listed in TABLES 3 to 31.

### ASMS of MBP 16E6

TABLE 3

| | | | | | | | | | | Portion | SEQ |
|---|---|---|---|---|---|---|---|---|---|---|---|
| | | | ALC | | | | | | Cond- | of | ID |
| Entry | Scan | peptide | (%) | m/z | Z | RT | Mass | Ppm | ition | library | Nos: |
| 1 | 9333 | pVsVAEiVCE | 84 | 700.3484 | 2 | 38.18 | 1398.6887 | −4.6 | 1.3 | 1/9 | 130 |
| | | ERU | | | | | | | (2) | | |
| 2 | 9562 | phsVQEiVCEE | 95 | 741.8667 | 2 | 39.48 | 1481.7222 | −2.2 | 1.1 | 1/9 | 131 |
| | | RU | | | | | | | (3) | | |
| 3 | 9163 | pVsVGEhVCE | 94 | 700.3491 | 2 | 38.08 | 1398.6821 | 1 | 1.1 | 1/9 | 132 |
| | | ERU | | | | | | | (3) | | |
| 4 | 9147 | pVNiQELVCE | 97 | 736.3646 | 2 | 29.43 | 1470.7209 | −4.3 | 1.1 | 1/9 | 133 |
| | | ERU | | | | | | | (2) | | |
| 5 | 7912 | pLsVQEVVCE | 97 | 729.8662 | 2 | 27.8 | 1457.7227 | −3.3 | 1.2 | 1/9 | 134 |
| | | ERU | | | | | | | (2) | | |
| 6 | 4538 | ELNiGEVyCE | 92 | 710.8311 | 2 | 20.89 | 1419.6538 | −4.4 | 1.3 | 1/9 | 135 |
| | | ERU | | | | | | | (1) | | |
| 7 | 9034 | pibiAEkyCEER | 94 | 743.3746 | 2 | 27.03 | 1484.7568 | −15 | 1.1 | 1/9 | 136 |
| | | U | | | | | | | (2) | | |
| 8 | 5422 | pVNVQELVC | 97 | 730.3646 | 2 | 21.78 | 1458.718 | −2.3 | 1.1 | 1/9 | 137 |
| | | EERU | | | | | | | (3) | | |
| 9 | 5021 | pysyQELVCEE | 96 | 763.8513 | 2 | 21 | 1525.7026 | −9.6 | 1.1 | 1/9 | 138 |
| | | RU | | | | | | | (3) | | |
| 10 | 5682 | pidiAEVVCEE | 98 | 698.3507 | 2 | 20.45 | 1394.6912 | −3.1 | 2.1 | 2/9 | 139 |
| | | RU | | | | | | | | | |
| 11 | 8228 | pmTowEiLCEE | 95 | 788.3776 | 2 | 30.42 | 1574.7373 | 2.1 | 2.2 | 2/9 | 140 |
| | | RU | | | | | | | | | |
| 12 | 9476 | pmToAELhCE | 97 | 788.3777 | 2 | 30.6 | 1574.7363 | 2.9 | 2.4 | 2/9 | 141 |
| | | ERU | | | | | | | | | |
| 13 | 10656 | pmbhwEmhCE | 96 | 788.3971 | 2 | 32.57 | 1574.7593 | 12.9 | 2.4 | 2/9 | 142 |
| | | ERU | | | | | | | | | |
| 14 | 10257 | pobiwEmiCEE | 93 | 792.382 | 2 | 31.88 | 1582.744 | 3.3 | 2.4 | 2/9 | 143 |
| | | RU | | | | | | | | | |
| 15 | 9409 | pLdowEhLCEE | 92 | 769.389 | 2 | 31.58 | 1536.7515 | 7.8 | 3.2 | 4/9 | 144 |
| | | RU | | | | | | | | | |
| 16 | 10046 | pjsoAEykCEE | 95 | 808.4194 | 2 | 38.85 | 1614.794 | 18.7 | 3.1 | 4/9 | 145 |
| | | RU | | | | | | | | | |
| 17 | 10318 | pmbhAEoLCE | 91 | 793.3899 | 2 | 39.49 | 1584.756 | 5.9 | 3.1 | 4/9 | 146 |
| | | ERU | | | | | | | | | |
| 18 | 10894 | poxLwEhLCEE | 90 | 783.4056 | 2 | 40.53 | 1564.782 | 9.7 | 3.1 | 4/9 | 147 |
| | | RU | | | | | | | | | |
| 19 | 9547 | pjsoAEjyCEER | 93 | 814.3953 | 2 | 39.33 | 1626.789 | −7.7 | 3.2 | 4/9 | 148 |
| | | U | | | | | | | | | |
| 20 | 16198 | pjNjwEiLCEE | 90 | 768.9105 | 2 | 38.58 | 1535.8096 | −2.1 | 5.5 | 1 | 149 |
| | | RU | | | | | | | | | |
| 21 | 14050 | pkTmwEhLCE | 93 | 767.4019 | 2 | 37.67 | 1532.7891 | 0.1 | 4.6 | 1 | 150 |
| | | ERU | | | | | | | | | |
| 22 | 14080 | pidowEhLCEE | 91 | 768.3787 | 2 | 37.63 | 1534.7388 | 2.7 | 4.6 | 1 | 151 |
| | | RU | | | | | | | | | |
| 23 | 11093 | poxiwpLLCEE | 92 | 783.4053 | 2 | 33.49 | 1564.7925 | 2.2 | 2.4 | 2/9 | 152 |
| | | RU | | | | | | | | | |
| 24 | 9393 | pmdhAEoLCE | 81 | 786.3811 | 2 | 31.89 | 1570.7358 | 7.5 | 3.1 | 4/9 | 153 |
| | | ERU | | | | | | | | | |
| 25 | 8437 | pLTowELLCE | 94 | 765.385 | 2 | 29.86 | 1528.7556 | −0.1 | 3.2 | 4/9 | 154 |
| | | ERU | | | | | | | | | |
| 26 | 13931 | pLsjAzkyCEER | 83 | 784.3939 | 2 | 38.95 | 1566.7615 | 7.5 | 4.1 | 1 | 155 |
| | | U | | | | | | | | | |
| 27 | 9200 | poNVwEkyCE | 92 | 788.8787 | 2 | 38.7 | 1575.75 | 4.3 | 3.2 | 4/9 | 156 |
| | | ERU | | | | | | | | | |
| 28 | 6954 | zisVQELyCEE | 87 | 763.8475 | 2 | 25.11 | 1525.6604 | 13.1 | 5.5 | 1 | 157 |
| | | RU | | | | | | | | | |
| 29 | 13352 | poTmGEmLCE | 88 | 799.3697 | 2 | 38.08 | 1596.7188 | 3.8 | 4.5 | 1 | 158 |
| | | ERU | | | | | | | | | |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine (Cha): j, Octanonic (Aoc): k, Homo phenylalanine (Hof): m, Phenylglycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 4
Quality control of peptide E6 library I

| | | ALC | | | | | | Unique- | SEQ ID |
|---|---|---|---|---|---|---|---|---|---|
| Scan | Peptide | (%) | m/z | Z | RT | Mass | ppm | ness | Nos: |
| 2982 | phgjQEhVCEERU | 98 | 781.9299 | 2 | 14.99 | 1561.811 | 22 | UNIQUE | 159 |
| 2720 | pVbmAEmVCEERU | 97 | 755.3904 | 2 | 13.52 | 1508.725 | 27.2 | UNIQUE | 160 |
| 2808 | pmTmQEmLCEERU | 97 | 816.9062 | 2 | 14.06 | 1631.753 | 27.7 | UNIQUE | 161 |
| 3181 | pLNkAEmkCEERU | 97 | 774.9245 | 2 | 16.21 | 1547.818 | 10.7 | UNIQUE | 162 |
| 3411 | poTkAEokCEERU | 97 | 828.4283 | 2 | 17.49 | 1654.823 | 11.5 | UNIQUE | 163 |
| 3113 | pkNjQEVoCEERU | 96 | 820.4309 | 2 | 14.27 | 1638.821 | 16 | UNIQUE | 164 |
| 3374 | pkbjAELjCEERU | 96 | 775.4514 | 2 | 17.3 | 1548.877 | 7.5 | UNIQUE | 165 |
| 3472 | podjGEojCEERU | 96 | 831.4292 | 2 | 17.9 | 1660.795 | 29.2 | UNIQUE | 166 |
| 2685 | pLgVAEhhCEERU | 95 | 733.4041 | 2 | 13.45 | 1464.755 | 26.5 | UNIQUE | 167 |
| 2484 | pkTnQEVhCEERU | 88 | 767.8989 | 2 | 12.13 | 1533.765 | 12 | UNIQUE | 168 |
| 3170 | EjghQELoCEERU | 88 | 817.928 | 2 | 16.14 | 1633.807 | 20.8 | UNIQUE | 169 |
| 2858 | pkdoGEVnCEERU | 87 | 766.3824 | 2 | 14.65 | 1530.728 | 14.6 | UNIQUE | 170 |

Identified Binders from Peptide E6 Library I-bME (ALC>80)

TABLE 5
Condition I-1.1(1)

| | | ALC | | | | | | Unique- | SEQ ID |
|---|---|---|---|---|---|---|---|---|---|
| Scan | Peptide | (%) | m/z | Z | RT | Mass | ppm | ness | Nos: |
| 4538 | ELNiGEVyCEERU | 92 | 710.8311 | 2 | 20.89 | 1419.6538 | −4.4 | UNIQUE | 135 |
| 5608 | ELTLQELLCEERU | 91 | 737.8823 | 2 | 23.03 | 1473.7498 | 0.2 | UNIQUE | 171 |
| 3927 | ELsVGEyVCEERU | 89 | 704.3334 | 2 | 19.65 | 1406.6555 | −2.4 | UNIQUE | 172 |
| 4572 | pysyQELVCEERU | 89 | 763.852 | 2 | 20.98 | 1525.7026 | −8.7 | UNIQUE | 138 |
| 4871 | ELsyQEiLCEERU | 89 | 752.8513 | 2 | 21.56 | 1503.7114 | −15.6 | UNIQUE | 173 |
| 4264 | ELsVAEhVCEERU | 88 | 707.3535 | 2 | 20.37 | 1412.6934 | −0.6 | UNIQUE | 174 |
| 4329 | ELNyQELVCEERU | 88 | 747.3564 | 2 | 20.45 | 1492.7036 | −3.5 | UNIQUE | 175 |
| 4478 | pLsVQEVVCEERU | 83 | 729.867 | 2 | 20.86 | 1457.7227 | −2.2 | UNIQUE | 134 |
| 4128 | EVdiQEVVCEERU | 82 | 713.8547 | 2 | 20.01 | 1425.6897 | 3.7 | UNIQUE | 176 |
| 3974 | EVNiGELVCEERU | 78 | 693.8393 | 2 | 19.67 | 1385.6638 | 0.2 | UNIQUE | 177 |
| 4217 | EishAEVVCEERU | 76 | 706.3481 | 2 | 20.27 | 1410.6807 | 0.8 | UNIQUE | 178 |
| 4545 | ELsVAEiVCEERU | 76 | 700.3492 | 2 | 21.02 | 1398.6843 | −0.4 | UNIQUE | 179 |
| 9179 | pLdhwpiLCEREU | 72 | 733.3881 | 2 | 29.4 | 1464.7615 | 0.1 | UNIQUE | 180 |
| 5524 | pVbyQEVyCEERU | 67 | 755.8575 | 2 | 22.82 | 1509.7065 | −4 | UNIQUE | 181 |
| 5431 | pVshQELyCEERU | 63 | 759.8658 | 2 | 23.19 | 1517.7249 | −5.1 | UNIQUE | 182 |
| 4236 | pVsVAEiVCEERU | 59 | 700.348 | 2 | 20.14 | 1398.6887 | −5.3 | UNIQUE | 130 |
| 5198 | pVTyAEiVCEERU | 56 | 711.3437 | 2 | 22.19 | 1420.6787 | −4.1 | UNIQUE | 183 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine (Cha): j, Octanonic (Aoc): k, Homo phenylalanine (Hof): m, Phenylglycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 6
Condition I-1.2(1)

| | | | | | | | | | SEQ |
|---|---|---|---|---|---|---|---|---|---|
| | | ALC | | | | | | Unique- | ID |
| Scan | Peptide | (%) | m/z | Z | RT | Mass | ppm | ness | Nos: |
| 4501 | ELTLQELLCEERU | 92 | 737.8818 | 2 | 23.06 | 1473.7498 | −0.5 | UNIQUE | 171 |
| 7569 | ELxLQEjVCEERU | 84 | 762.9077 | 2 | 28.93 | 1523.7979 | 1.9 | UNIQUE | 184 |
| 6631 | pibVAEjyCEERU | 82 | 743.3734 | 2 | 27.06 | 1484.748 | −10.6 | UNIQUE | 185 |
| 5352 | EhbiAELyCEERU | 79 | 729.3621 | 2 | 25.02 | 1456.7058 | 2.6 | UNIQUE | 186 |
| 4297 | ELNhAEVyCEERU | 78 | 724.8461 | 2 | 22.61 | 1447.6787 | −0.8 | UNIQUE | 187 |
| 4991 | pibVQELVCEERU | 78 | 734.8774 | 2 | 24.31 | 1467.7451 | −3.3 | UNIQUE | 188 |
| 4448 | EhbiQEVVCEERU | 75 | 733.8693 | 2 | 23.06 | 1465.7217 | 1.6 | UNIQUE | 189 |
| 6785 | EhdoAELVCEERU | 71 | 748.3651 | 2 | 27.67 | 1494.7058 | 6.5 | UNIQUE | 190 |
| 4542 | ELbLAELVCEERU | 64 | 707.3766 | 2 | 23.28 | 1412.7319 | 4.7 | UNIQUE | 191 |
| 6046 | pVdjGELyCEREU | 62 | 730.366 | 2 | 25.81 | 1458.7251 | −5.3 | UNIQUE | 192 |
| 5217 | pLsLAEhyCEERU | 55 | 738.3633 | 2 | 24.75 | 1474.719 | −4.7 | UNIQUE | 193 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine (Cha): j, Octanonic (Aoc): k, Homo phenylalanine (Hof): m, Phenylglycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 7
Condition I-1.3(1)

| | | ALC | | | | | | Unique- | SEQ ID |
|---|---|---|---|---|---|---|---|---|---|
| Scan | Peptide | (%) | m/z | Z | RT | Mass | ppm | ness | Nos: |
| F1:5490 | ELTLQELLCEERU | 94 | 737.8824 | 2 | 23.04 | 1473.7498 | 0.4 | UNIQUE | 171 |
| F3:9034 | pibiAEkyCEERU | 94 | 743.3746 | 2 | 27.03 | 1484.7568 | −15 | UNIQUE | 136 |
| F3:5019 | pysiQEyVCEERU | 93 | 762.8431 | 2 | 19.88 | 1523.6902 | −12.1 | UNIQUE | 194 |
| F3:5640 | pVsVQELyCEERU | 93 | 746.8583 | 2 | 21.08 | 1491.7126 | −7.1 | UNIQUE | 195 |
| F1:6460 | ELNhAEVLCEER | 90 | 714.8624 | 2 | 25.06 | 1427.7043 | 4.2 | UNIQUE | 196 |
| | U | | | | | | | | |
| F3:6967 | ELNVQEhLCEER | 90 | 743.373 | 2 | 23.34 | 1484.7258 | 3.8 | UNIQUE | 197 |
| | U | | | | | | | | |
| F1:8095 | phdLwEhyCEERU | 89 | 743.3732 | 2 | 27.7 | 1484.7249 | 4.8 | UNIQUE | 198 |
| F1:7769 | pLxiAEhLCEREU | 88 | 733.3926 | 2 | 27.02 | 1464.7615 | 6.2 | UNIQUE | 199 |
| F1:9875 | pjsowEVyCEERU | 88 | 794.379 | 2 | 30.82 | 1586.7485 | −3.2 | UNIQUE | 200 |
| F3:5581 | pLsVQEyiCEERU | 88 | 752.8579 | 2 | 20.94 | 1503.7158 | −9.7 | UNIQUE | 201 |
| F1:7102 | pibVAEmVCEERU | 87 | 730.3654 | 2 | 26.39 | 1458.718 | −1.3 | UNIQUE | 202 |
| F3:5629 | pysyQELVCEERU | 87 | 763.85 | 2 | 21.02 | 1525.7026 | −11.3 | UNIQUE | 138 |
| F3:6301 | pVNLQEiVCEERU | 87 | 736.3638 | 2 | 21.93 | 1470.7209 | −5.4 | UNIQUE | 203 |
| F3:8342 | pLbVAEiLCEREU | 87 | 713.3744 | 2 | 25.83 | 1424.7395 | −3.7 | UNIQUE | 204 |
| F3:8578 | pmNiAEhVCEERU | 87 | 744.863 | 2 | 26.17 | 1487.7061 | 3.6 | UNIQUE | 205 |
| F1:5286 | ELshAEVyCEERU | 86 | 724.3483 | 2 | 22.12 | 1446.6833 | −0.9 | UNIQUE | 206 |
| F1:8024 | pibVAEkyCEERU | 86 | 737.373 | 2 | 28.02 | 1472.7537 | −15 | UNIQUE | 207 |
| F3:5098 | EVdiQEVVCEERU | 86 | 713.8534 | 2 | 20.03 | 1425.6897 | 1.8 | UNIQUE | 176 |
| F3:6302 | EhNVGELyCEERU | 85 | 717.8378 | 2 | 22.21 | 1433.6631 | −1.5 | UNIQUE | 208 |
| F3:8562 | pLdiAEmiCEREU | 85 | 736.3683 | 2 | 26.15 | 1470.7168 | 3.6 | UNIQUE | 209 |
| F1:6834 | ELTLQELmCEER | 84 | 761.8759 | 2 | 25.38 | 1521.7441 | −4.6 | UNIQUE | 210 |
| | U | | | | | | | | |
| F3:5492 | pVTVQEVVCERE | 84 | 716.8593 | 2 | 20.79 | 1431.707 | −2.1 | UNIQUE | 211 |
| | U | | | | | | | | |
| F3:5935 | EidLQEVVCEERU | 84 | 720.8618 | 2 | 21.47 | 1439.7053 | 2.6 | UNIQUE | 212 |
| F3:6097 | pVNiGELyCEERU | 84 | 717.8397 | 2 | 21.86 | 1433.6738 | −6.3 | UNIQUE | 213 |
| F3:7597 | pVsLAELiCEERU | 84 | 714.3624 | 2 | 23.91 | 1426.72 | −6.8 | UNIQUE | 214 |
| F2:7569 | ELxLQEjVCEERU | 84 | 762.9077 | 2 | 28.93 | 1523.7979 | 1.9 | UNIQUE | 184 |
| F3:10665 | pjdLAEhLCEERU | 83 | 740.396 | 2 | 29.76 | 1478.7786 | −0.8 | UNIQUE | 215 |
| F1:7743 | ELbVwEVLCEER | 82 | 707.3734 | 2 | 26.97 | 1412.7263 | 4.2 | UNIQUE | 216 |
| | U | | | | | | | | |
| F2:6631 | pibVAEjyCEERU | 82 | 743.3734 | 2 | 27.06 | 1484.748 | −10.6 | UNIQUE | 185 |
| F1:6664 | pVxVQEhVCEERU | 81 | 741.8853 | 2 | 25.09 | 1481.7485 | 5 | UNIQUE | 217 |
| F3:9228 | pLbVwEhyCEERU | 81 | 737.3729 | 2 | 27.27 | 1472.7329 | −1.1 | UNIQUE | 218 |
| F3:6646 | piNyQEhVCEERU | 80 | 759.3557 | 2 | 22.38 | 1516.7075 | −7 | UNIQUE | 219 |
| F3:6361 | pVsVAEhyCEERU | 80 | 724.3474 | 2 | 22.73 | 1446.6877 | −5.2 | UNIQUE | 220 |
| F3:6737 | pVdiGEhVCEREU | 80 | 698.3519 | 2 | 23.07 | 1394.6846 | 3.4 | UNIQUE | 221 |
| F3:7024 | pLdLQEVyCEREU | 80 | 745.8787 | 2 | 23.48 | 1489.7278 | 10.1 | UNIQUE | 222 |
| F3:11227 | pjNhAEjVCEERU | 80 | 761.9014 | 2 | 30.72 | 1521.793 | −3.1 | UNIQUE | 223 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine (Cha): j, Octanonic (Aoc): k, Homo phenylalanine (Hof): m, Phenylglycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 8
Condition I-1.1(2)

| | | ALC | | | | | | Unique- | SEQ ID |
|---|---|---|---|---|---|---|---|---|---|
| Scan | | (%) | m/z | Z | RT | Mass | ppm | ness | No: |
| 4179 | ELTLQELLCEERU | 93 | 737.8824 | 2 | 23.05 | 1473.75 | 0.4 | UNIQUE | 171 |

TABLE 9
Condition I-1.3(2)

| | | | | | | | | | SEQ |
|---|---|---|---|---|---|---|---|---|---|
| | | ALC | | | | | | Unique- | ID |
| Scan | Peptide | (%) | m/z | Z | RT | Mass | ppm | ness | Nos: |
| 4437 | ELsyQELICEERU | 92 | 752.8592 | 2 | 21.76 | 1503.7114 | −5 | UNIQUE | 224 |
| 4462 | pLsiGELVCEERU | 91 | 707.3561 | 2 | 21.65 | 1412.7043 | −4.8 | UNIQUE | 225 |
| 5067 | ELTLQELLCEERU | 90 | 737.8831 | 2 | 23.04 | 1473.7498 | 1.2 | UNIQUE | 171 |
| 4941 | ELsVAELLCEERU | 89 | 708.3627 | 2 | 22.6 | 1414.7126 | −1.3 | UNIQUE | 226 |
| 5169 | ELNLQELyCEERU | 88 | 754.3666 | 2 | 23.2 | 1506.7192 | −0.4 | UNIQUE | 227 |
| 3678 | ELsVGEyVCEERU | 87 | 704.3317 | 2 | 19.61 | 1406.6555 | −4.8 | UNIQUE | 172 |
| 4143 | pLsVQEVVCEERU | 87 | 729.8661 | 2 | 20.83 | 1457.7227 | −3.5 | UNIQUE | 134 |
| 4889 | ELNhAEVyCEERU | 87 | 724.8474 | 2 | 22.6 | 1447.6787 | 1 | UNIQUE | 187 |
| 4909 | ELNVAELLCEERU | 87 | 708.8645 | 2 | 22.7 | 1415.7078 | 4.7 | UNIQUE | 228 |
| 4811 | pLsyQELVCEERU | 83 | 753.8658 | 2 | 22.42 | 1505.7283 | −7.5 | UNIQUE | 229 |
| 5926 | pLNhQELVCEERU | 83 | 750.3811 | 2 | 24.76 | 1498.7458 | 1.2 | UNIQUE | 230 |
| 5110 | ELNiAEyLCEERU | 76 | 724.8467 | 2 | 23.06 | 1447.6853 | −4.5 | UNIQUE | 231 |
| 6968 | ELbVwEVLCEERU | 76 | 707.3721 | 2 | 26.91 | 1412.7263 | 2.3 | UNIQUE | 216 |
| 4998 | pVTVAEVLCEREU | 75 | 695.3544 | 2 | 22.82 | 1388.7012 | −5 | UNIQUE | 232 |
| 4570 | pVsiAEVyCEERU | 71 | 717.34 | 2 | 21.88 | 1432.6787 | −9.2 | UNIQUE | 233 |
| 5023 | ELNLAEVVCEERU | 65 | 701.8561 | 2 | 22.88 | 1401.6921 | 4 | UNIQUE | 234 |
| 6856 | pVsLAEmLCEERU | 65 | 739.3693 | 2 | 26.67 | 1476.7268 | −1.8 | UNIQUE | 235 |
| 4440 | pysiAELVCEERU | 61 | 724.3484 | 2 | 21.57 | 1446.6943 | −8.3 | UNIQUE | 236 |
| 7199 | ELshAEjLCEERU | 55 | 741.395 | 2 | 27.38 | 1480.759 | 11 | UNIQUE | 237 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine (Cha): j, Octanonic (Aoc): k, Homo phenylalanine (Hof): m, Phenylglycine (Phg): y, Napthyl alanine (Naf): o, Aminoadipic acid (Aad): p, Homo cysteic acid (Hoc): z, Hydroxy1 proline (Hyp): s, Amino isobutyric (Aib): w, C-term amide: U.

TABLE 10
Condition I-1.3(2)

| | | | | | | | | | SEQ |
|---|---|---|---|---|---|---|---|---|---|
| | | ALC | | | | | | Unique- | ID |
| Scan | Peptide | (%) | m/z | Z | RT | Mass | ppm | ness | Nos: |
| 9034 | pibiAEkyCEERU | 94 | 743.3746 | 2 | 27.03 | 1484.7568 | −15 | UNIQUE | 136 |
| 5019 | pysiQEyVCEERU | 93 | 762.8431 | 2 | 19.88 | 1523.6902 | −12.1 | UNIQUE | 194 |
| 5640 | pVsVQELyCEERU | 93 | 746.8583 | 2 | 21.08 | 1491.7126 | −7.1 | UNIQUE | 195 |
| 6967 | ELNVQEhLCEERU | 90 | 743.373 | 2 | 23.34 | 1484.7258 | 3.8 | UNIQUE | 197 |
| 5581 | pLsVQEyiCEERU | 88 | 752.8579 | 2 | 20.94 | 1503.7158 | −9.7 | UNIQUE | 201 |
| 5629 | pysyQELVCEERU | 87 | 763.85 | 2 | 21.02 | 1525.7026 | −11.3 | UNIQUE | 138 |
| 6301 | pVNLQEiVCEERU | 87 | 736.3638 | 2 | 21.93 | 1470.7209 | −5.4 | UNIQUE | 203 |
| 8342 | pLbVAEILCEREU | 87 | 713.3744 | 2 | 25.83 | 1424.7395 | −3.7 | UNIQUE | 204 |
| 8578 | pmNiAEhVCEERU | 87 | 744.863 | 2 | 26.17 | 1487.7061 | 3.6 | UNIQUE | 205 |
| 5098 | EVdiQEVVCEERU | 86 | 713.8534 | 2 | 20.03 | 1425.6897 | 1.8 | UNIQUE | 176 |
| 6302 | EhNVGELyCEERU | 85 | 717.8378 | 2 | 22.21 | 1433.6631 | −1.5 | UNIQUE | 208 |
| 8562 | pLdiAEmiCEREU | 85 | 736.3683 | 2 | 26.15 | 1470.7168 | 3.6 | UNIQUE | 209 |
| 5492 | pVTVQEVVCEREU | 84 | 716.8593 | 2 | 20.79 | 1431.707 | −2.1 | UNIQUE | 211 |
| 5935 | EidLQEVVCEERU | 84 | 720.8618 | 2 | 21.47 | 1439.7053 | 2.6 | UNIQUE | 212 |
| 6097 | pVNiGELyCEERU | 84 | 717.8397 | 2 | 21.86 | 1433.6738 | −6.3 | UNIQUE | 213 |
| 7597 | pVsLAELiCEERU | 84 | 714.3624 | 2 | 23.91 | 1426.72 | −6.8 | UNIQUE | 214 |
| 10665 | pjdLAEhLCEERU | 83 | 740.396 | 2 | 29.76 | 1478.7786 | −0.8 | UNIQUE | 215 |
| 9228 | pLbVwEhyCEERU | 81 | 737.3729 | 2 | 27.27 | 1472.7329 | −1.1 | UNIQUE | 218 |
| 6646 | piNyQEhVCEERU | 80 | 759.3557 | 2 | 22.38 | 1516.7075 | −7 | UNIQUE | 219 |
| 6539 | phTiQEyLCEERU | 79 | 759.8644 | 2 | 22.69 | 1517.7278 | −8.9 | UNIQUE | 238 |
| 5251 | ELsVAEiVCEERU | 78 | 700.3485 | 2 | 20.13 | 1398.6843 | −1.3 | UNIQUE | 179 |
| 7274 | ELbVwEyVCEERU | 77 | 717.3662 | 2 | 23.91 | 1432.7007 | 12 | UNIQUE | 239 |
| 11040 | pLskAEikCEERU | 77 | 749.4062 | 2 | 30.4 | 1496.8157 | −11.9 | UNIQUE | 240 |
| 4440 | EisiAEiyCEERU | 75 | 722.3315 | 2 | 18.65 | 1442.6648 | −11.4 | UNIQUE | 241 |
| 5689 | piThGEVyCEERU | 75 | 717.3411 | 2 | 21.36 | 1432.675 | −5.2 | UNIQUE | 242 |
| 5738 | pisVAEVyCEERU | 75 | 717.3411 | 2 | 21.36 | 1432.6787 | −7.8 | UNIQUE | 243 |
| 6172 | pVsiAEVyCEERU | 75 | 717.3411 | 2 | 21.36 | 1432.6787 | −7.8 | UNIQUE | 233 |
| 5913 | pLsiQEVyCEERU | 75 | 752.859 | 2 | 21.7 | 1503.7158 | −8.2 | UNIQUE | 244 |
| 9070 | pVbVwEVLCEERU | 75 | 707.3724 | 2 | 27.01 | 1412.7307 | −0.3 | UNIQUE | 245 |
| 9506 | pidmAEVLCEERU | 75 | 730.3665 | 2 | 27.64 | 1458.7139 | 3.1 | UNIQUE | 246 |
| 10147 | pLbywELLCEERU | 75 | 738.3815 | 2 | 28.81 | 1474.752 | −2.4 | UNIQUE | 247 |
| 7189 | pLsiAELLCEERU | 74 | 721.37 | 2 | 23.74 | 1440.7356 | −7 | UNIQUE | 248 |
| 7704 | pLNVGEhLCEREU | 74 | 714.8629 | 2 | 24.66 | 1427.7087 | 1.7 | UNIQUE | 249 |
| 10120 | pVskAphyCEERU | 74 | 752.3773 | 2 | 28.83 | 1502.7634 | −15.6 | UNIQUE | 250 |
| 6344 | pVTyAEiyCEERU | 71 | 728.332 | 2 | 22.21 | 1454.6687 | −13.2 | UNIQUE | 251 |
| 9394 | ELxmApiVCEERU | 71 | 744.3802 | 2 | 27.58 | 1486.7437 | 1.5 | UNIQUE | 252 |
| 8560 | EjNVGELVCEERU | 70 | 714.8655 | 2 | 25.86 | 1427.7109 | 3.8 | UNIQUE | 253 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine (Cha): j, Octanonic (Aoc): k, Homo phenylalanine (Hof): m, Phenylglycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 11
Condition I-1.1(3)

| | | | | | | | | | SEQ |
|---|---|---|---|---|---|---|---|---|---|
| | | | | | | | | | ID |
| Scan | Peptide | ALC (%) | m/z | z | RT | Mass | ppm | Uniqueness | Nos: |
| 5700 | ELTLQELLCEERU | 94 | 737.8826 | 2 | 22.93 | 1473.7498 | 0.6 | UNIQUE | 171 |
| 7998 | pibiAEkyCEERU | 93 | 743.3803 | 2 | 25.85 | 1484.7568 | −7.3 | UNIQUE | 136 |
| 9958 | pVdowELLCEERU | 91 | 756.3802 | 2 | 30.26 | 1510.7395 | 4.3 | UNIQUE | 254 |
| 11145 | pobiwEjyCEERU | 90 | 799.3925 | 2 | 32.3 | 1596.7712 | −0.6 | UNIQUE | 255 |
| 10377 | pjdoAELVCEERU | 88 | 769.3892 | 2 | 30.91 | 1536.7637 | 0.1 | UNIQUE | 256 |
| 4886 | phsVQEiVCEERU | 86 | 741.8677 | 2 | 21.46 | 1481.7222 | −0.8 | UNIQUE | 131 |
| 4618 | pysVQELyCEERU | 85 | 763.8508 | 2 | 20.96 | 1525.7026 | −10.2 | UNIQUE | 257 |
| 8328 | phbVwEhyCEERU | 85 | 743.3736 | 2 | 27.62 | 1484.7292 | 2.3 | UNIQUE | 258 |
| 5246 | pLThQEVyCEERU | 83 | 753.863 | 2 | 22.14 | 1505.7249 | −8.9 | UNIQUE | 259 |
| 8635 | pixLAEhyCEERU | 83 | 743.3736 | 2 | 27.62 | 1484.7358 | −2.2 | UNIQUE | 260 |
| 10570 | poTVwEVICEERU | 82 | 771.3862 | 2 | 31.31 | 1540.7588 | −0.7 | UNIQUE | 261 |
| 5658 | pLshAEVyCEERU | 79 | 731.3577 | 2 | 23.36 | 1460.7034 | −1.8 | UNIQUE | 262 |
| 5183 | ELNyQELVCEERU | 78 | 747.3574 | 2 | 22.03 | 1492.7036 | −2.3 | UNIQUE | 175 |
| 6366 | ELNLQELLCEERU | 78 | 744.3705 | 2 | 24.18 | 1486.7449 | −12.4 | UNIQUE | 263 |
| 9936 | poNiAEjyCEERU | 78 | 793.8694 | 2 | 30.21 | 1585.7371 | −8.1 | UNIQUE | 264 |
| 5953 | pibVGELVCEREU | 75 | 699.3582 | 2 | 23.4 | 1396.708 | −4.5 | UNIQUE | 265 |
| 5178 | ELsyAEiVCEERU | 74 | 717.3408 | 2 | 22.02 | 1432.6743 | −5.1 | UNIQUE | 266 |
| 9338 | ELbjAEVLCEERU | 74 | 727.3896 | 2 | 29.18 | 1452.7664 | −1.1 | UNIQUE | 267 |
| 4839 | ELsVQEyLCEERU | 73 | 746.8593 | 2 | 21.36 | 1491.7083 | −2.8 | UNIQUE | 268 |
| 8971 | ELbjwEVyCEERU | 73 | 744.3825 | 2 | 28.58 | 1486.7507 | −0.2 | UNIQUE | 269 |
| 4244 | pisVQEiVCEERU | 72 | 734.8588 | 2 | 20.21 | 1467.7131 | −6.9 | UNIQUE | 270 |
| 10796 | ELTjwEkLCEREU | 69 | 750.4103 | 2 | 31.48 | 1498.8125 | −4.3 | UNIQUE | 271 |
| 6938 | ELTLQELmCEERU | 65 | 761.8746 | 2 | 25.26 | 1521.7441 | −6.2 | UNIQUE | 210 |
| 7065 | pLdiApVLCEERU | 65 | 713.374 | 2 | 25.76 | 1424.7395 | −4.2 | UNIQUE | 272 |
| 4783 | pisVAEyVCEERU | 58 | 717.3414 | 2 | 21.24 | 1432.6787 | −7.3 | UNIQUE | 273 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 12
Condition I-1.2(3)

| | | | | | | | | | SEQ ID |
|---|---|---|---|---|---|---|---|---|---|
| Scan | Peptide | ALC (%) | m/z | z | RT | Mass | ppm | Uniqueness | Nos: |
| 5594 | ELTLQELLCEERU | 97 | 737.8824 | 2 | 22.98 | 1473.7498 | 0.3 | UNIQUE | 171 |
| 5745 | ELTLQELLCEERU | 93 | 737.8824 | 2 | 22.98 | 1473.7498 | 0.3 | NOT UNIQUE | 171 |
| 6575 | pVTLQELmCEERU | 87 | 761.8745 | 2 | 24.65 | 1521.7483 | −9.2 | UNIQUE | 274 |
| 3929 | ELsVGEyVCEERU | 84 | 704.3328 | 2 | 19.61 | 1406.6555 | −3.1 | UNIQUE | 172 |
| 5460 | pVNiwEVyCEERU | 79 | 724.8474 | 2 | 22.67 | 1447.6838 | −2.5 | UNIQUE | 275 |
| 4147 | ELsVGpViCEERU | 73 | 700.3653 | 2 | 20.11 | 1398.6887 | 19.5 | UNIQUE | 276 |
| 8545 | pkTiAEmiCEERU | 72 | 752.3758 | 2 | 28.1 | 1502.7573 | −13.5 | UNIQUE | 277 |
| 4941 | piNiAEVyCEERU | 70 | 723.8376 | 2 | 21.7 | 1445.677 | −11.2 | UNIQUE | 278 |
| 9523 | pkdiwEiLCEERU | 67 | 733.3887 | 2 | 29.35 | 1464.7725 | −6.6 | UNIQUE | 279 |
| 7512 | pVdmApiVCEERU | 65 | 730.3654 | 2 | 26.3 | 1458.718 | −1.3 | UNIQUE | 280 |
| 6966 | ELTLQELmCEERU | 64 | 761.8745 | 2 | 24.65 | 1521.7441 | −6.4 | UNIQUE | 210 |
| 8585 | pLdiwphyCEERU | 64 | 743.374 | 2 | 28.02 | 1484.7358 | −1.6 | UNIQUE | 281 |
| 7290 | pLdiAEmiCEREU | 60 | 736.3693 | 2 | 25.8 | 1470.7168 | 4.9 | UNIQUE | 209 |
| 10577 | poxhGELICEERU | 54 | 768.382 | 2 | 31.55 | 1534.7432 | 4 | UNIQUE | 282 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 13
Condition I-1.3(3)

| | | | | | | | | | SEQ ID |
|---|---|---|---|---|---|---|---|---|---|
| Scan | Peptide | ALC (%) | m/z | z | RT | Mass | ppm | Uniqueness | Nos: |
| 5422 | pVNVQELVCEERU | 97 | 730.3646 | 2 | 21.78 | 1458.718 | −2.3 | UNIQUE | 137 |
| 5021 | pysyQELVCEERU | 96 | 763.8513 | 2 | 21 | 1525.7026 | −9.6 | UNIQUE | 138 |
| 5330 | pVsyQELiCEERU | 93 | 752.8584 | 2 | 21.14 | 1503.7158 | −9 | UNIQUE | 283 |
| 5463 | pVsiQEhiCEERU | 93 | 747.8659 | 2 | 21.41 | 1493.7253 | −5.4 | UNIQUE | 284 |
| 6069 | pVTiAEVLCEERU | 93 | 701.3555 | 2 | 22.92 | 1400.7043 | −5.6 | UNIQUE | 285 |
| 4696 | pVsVAEiVCEERU | 92 | 700.3647 | 2 | 20.39 | 1398.6887 | 18.7 | UNIQUE | 130 |
| 6611 | pLNVAEVLCEERU | 92 | 708.8611 | 2 | 23.89 | 1415.7122 | −3.2 | UNIQUE | 286 |
| 4182 | EVNVAEyVCEERU | 91 | 704.8303 | 2 | 18.93 | 1407.6509 | −3.5 | UNIQUE | 287 |
| 5410 | ELsLQELICEERU | 91 | 742.8738 | 2 | 21.73 | 1483.7371 | −2.7 | UNIQUE | 288 |
| 6287 | ELNVQEhLCEERU | 91 | 743.3726 | 2 | 23 | 1484.7258 | 3.2 | UNIQUE | 197 |
| 4530 | pisVQEiVCEERU | 90 | 734.8586 | 2 | 20.22 | 1467.7131 | −7.2 | UNIQUE | 270 |
| 4972 | pLsVQEyiCEERU | 90 | 752.8584 | 2 | 21.14 | 1503.7158 | −9 | UNIQUE | 201 |
| 5266 | pLsiQEyyCEERU | 90 | 769.8515 | 2 | 21.47 | 1537.7058 | −11.3 | UNIQUE | 289 |
| 4272 | ELsiGEVyCEERU | 89 | 710.3316 | 2 | 19.62 | 1418.6587 | −7.1 | UNIQUE | 290 |
| 5152 | pLsiQEVyCEERU | 89 | 752.8584 | 2 | 21.14 | 1503.7158 | −9 | UNIQUE | 244 |
| 5531 | pVTVGEILCEERU | 89 | 694.3491 | 2 | 21.98 | 1386.6887 | −3.7 | UNIQUE | 291 |
| 6218 | pVdiQEILCEREU | 89 | 733.8698 | 2 | 23.25 | 1465.7283 | −2.3 | UNIQUE | 292 |
| 7192 | pmdiAEVVCEERU | 89 | 723.3583 | 2 | 25.12 | 1444.698 | 2.8 | UNIQUE | 293 |
| 4399 | pysiQEyVCEERU | 88 | 762.8431 | 2 | 19.9 | 1523.6902 | −12.1 | UNIQUE | 194 |
| 4862 | pLsVQEVVCEERU | 88 | 729.8668 | 2 | 20.88 | 1457.7227 | −2.4 | UNIQUE | 134 |
| 6150 | pVNVwEiyCEERU | 88 | 724.8461 | 2 | 23.07 | 1447.6838 | −4.3 | UNIQUE | 294 |
| 6198 | ELbLAEVLCEERU | 87 | 707.3801 | 2 | 22.88 | 1412.7319 | 9.7 | UNIQUE | 295 |
| 3726 | EyNyGEVVCEERU | 86 | 714.8135 | 2 | 18.37 | 1427.6252 | −8.9 | UNIQUE | 296 |
| 4739 | ELNyQELVCEERU | 86 | 747.3569 | 2 | 20.5 | 1492.7036 | −2.9 | UNIQUE | 175 |
| 5686 | pisyQEihCEERU | 86 | 764.8566 | 2 | 22.21 | 1527.7153 | −11 | UNIQUE | 297 |
| 4402 | pVsVGEyVCEERU | 85 | 704.3328 | 2 | 19.63 | 1406.6599 | −6.4 | UNIQUE | 298 |
| 4536 | EhNyQEViCEERU | 85 | 752.3478 | 2 | 20.07 | 1502.6875 | −4.2 | UNIQUE | 299 |
| 3377 | EisVGEiiCEERU | 84 | 698.3324 | 2 | 17.51 | 1394.6592 | −6.4 | UNIQUE | 300 |
| 3628 | ELsVGEyVCEERU | 84 | 704.3303 | 2 | 18.2 | 1406.6555 | −6.8 | UNIQUE | 172 |
| 3878 | ELSVGEILCEERU | 84 | 700.3459 | 2 | 18.7 | 1398.6843 | −5 | UNIQUE | 301 |
| 4249 | EyNiQEVVCEERU | 84 | 739.3422 | 2 | 19.51 | 1476.6753 | −3.7 | UNIQUE | 302 |
| 3389 | EisVQEyVCEERU | 83 | 738.8434 | 2 | 17.64 | 1475.6802 | −5.4 | UNIQUE | 303 |
| 5296 | pysVQEhiCEERU | 83 | 758.8585 | 2 | 21.53 | 1515.7122 | −6.5 | UNIQUE | 304 |
| 6110 | pLsiwEVyCEERU | 83 | 731.356 | 2 | 23.25 | 1460.7043 | −4.8 | UNIQUE | 305 |
| 7693 | EhbVAEimCEREU | 83 | 736.3685 | 2 | 25.09 | 1470.7102 | 8.3 | UNIQUE | 306 |
| 4334 | EisVwEyVCEERU | 82 | 717.3403 | 2 | 19.77 | 1432.6687 | −1.9 | UNIQUE | 307 |
| 6104 | ELTLQELLCEREU | 81 | 737.8829 | 2 | 23.03 | 1473.7498 | 1.1 | UNIQUE | 308 |
| 7023 | pVbVQEihCEERU | 80 | 740.8767 | 2 | 24.34 | 1479.7417 | −1.9 | UNIQUE | 309 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 14
Condition I-2.1

| | | | | | | | | | SEQ |
|---|---|---|---|---|---|---|---|---|---|
| | | | | | | | | | ID |
| Scan | Peptide | ALC (%) | m/z | z | RT | Mass | ppm | Uniqueness | Nos: |
| 5682 | pidiAEVVCEERU | 98 | 698.3507 | 2 | 20.45 | 1394.691 | −3.1 | UNIQUE | 139 |
| 6021 | pLdVQEVVCEERU | 97 | 721.869 | 2 | 22.77 | 1441.722 | 0.8 | UNIQUE | 310 |
| 7362 | pLbVQEiLCEERU | 97 | 741.8849 | 2 | 25.17 | 1481.761 | −3.8 | UNIQUE | 311 |
| 5549 | pVdVAEiVCEERU | 96 | 692.3517 | 2 | 20.53 | 1382.688 | 0.4 | UNIQUE | 312 |
| 5427 | pVNiAEVVCEERU | 96 | 700.8486 | 2 | 21.88 | 1399.684 | −0.9 | UNIQUE | 313 |
| 6044 | pLTVGEVLCEERU | 96 | 695.3558 | 2 | 22.89 | 1388.701 | −2.9 | UNIQUE | 314 |
| 4637 | ELsiQEyVCEERU | 95 | 745.8514 | 2 | 19.9 | 1489.696 | −5 | UNIQUE | 315 |
| 5101 | pVsVQELVCEERU | 95 | 729.8668 | 2 | 21.14 | 1457.723 | −2.4 | UNIQUE | 316 |
| 5144 | pVsVQELICEERU | 95 | 735.8665 | 2 | 21.17 | 1469.726 | −5.1 | UNIQUE | 317 |
| 5842 | piNVQEILCEERU | 95 | 742.3641 | 2 | 22.45 | 1482.724 | −7.1 | UNIQUE | 318 |
| 6112 | pVdVAELICEERU | 95 | 699.3578 | 2 | 23 | 1396.704 | −2 | UNIQUE | 319 |
| 6455 | pibVGELVCEERU | 95 | 699.3578 | 2 | 23 | 1396.708 | −5 | UNIQUE | 320 |
| 6630 | pVNLQEhVCEERU | 95 | 743.3732 | 2 | 23.85 | 1484.73 | 1.2 | UNIQUE | 321 |
| 8171 | pVxLQELICEERU | 95 | 748.8925 | 2 | 26.77 | 1495.771 | −0.4 | UNIQUE | 322 |
| 4309 | pysVGEVICEERU | 94 | 710.3334 | 2 | 18.09 | 1418.663 | −7.7 | UNIQUE | 323 |
| 4900 | pisyQEiVCEERU | 94 | 751.8511 | 2 | 20.99 | 1501.703 | −10.3 | UNIQUE | 324 |
| 5119 | pysiQEyyCEERU | 94 | 779.8366 | 2 | 21.14 | 1557.68 | −13.8 | UNIQUE | 325 |
| 5690 | pysLQEyVCEERU | 94 | 763.8505 | 2 | 21.32 | 1525.703 | −10.6 | UNIQUE | 326 |
| 5603 | pVdiQEVyCEERU | 94 | 737.854 | 2 | 21.34 | 1473.7 | −4.1 | UNIQUE | 327 |
| 6235 | pVTLQELLCEERU | 94 | 737.8816 | 2 | 23.11 | 1473.754 | −3.7 | UNIQUE | 328 |
| 6287 | pLbVAEVICEERU | 94 | 706.3651 | 2 | 23.24 | 1410.724 | −5.9 | UNIQUE | 329 |
| 6508 | pixiAEiVCEERU | 94 | 718.3668 | 2 | 23.67 | 1434.724 | −3.6 | UNIQUE | 330 |
| 7168 | pLdhQEVyCEERU | 94 | 751.8698 | 2 | 24.14 | 1501.724 | 0.4 | UNIQUE | 331 |
| 7047 | pVbVQEiLCEERU | 94 | 734.8771 | 2 | 24.38 | 1467.745 | −3.8 | UNIQUE | 332 |
| 7699 | pjsVQEihCEERU | 94 | 768.8893 | 2 | 25.68 | 1535.772 | −5.3 | UNIQUE | 333 |
| 8052 | pydVAELLCEERU | 94 | 717.3583 | 2 | 26.33 | 1432.706 | −3 | UNIQUE | 334 |
| 3745 | EVsyQEVVCEERU | 93 | 732.8425 | 2 | 18.38 | 1463.677 | −4.4 | UNIQUE | 335 |
| 5642 | pysVQpiVCEERU | 93 | 752.8584 | 2 | 22.8 | 1503.72 | −11.9 | UNIQUE | 336 |
| 6109 | pLdiQEViCEREU | 93 | 733.8691 | 2 | 22.97 | 1465.728 | −3.2 | UNIQUE | 337 |
| 6773 | PVNVAELLCEERU | 93 | 708.8625 | 2 | 24.09 | 1415.712 | −1.2 | UNIQUE | 338 |
| 6843 | pLdiQELiCEERU | 93 | 740.8774 | 2 | 24.29 | 1479.744 | −2.4 | UNIQUE | 339 |
| 7386 | pVdhQEiLCEERU | 93 | 740.8774 | 2 | 24.29 | 1479.737 | 2 | UNIQUE | 340 |
| 7030 | pVThwEVVCEERU | 93 | 708.3618 | 2 | 24.91 | 1414.708 | 0.9 | UNIQUE | 341 |
| 7385 | pLbVwEViCEERU | 93 | 713.3732 | 2 | 25.17 | 1424.734 | −1.2 | UNIQUE | 342 |
| 7503 | pVbiAEVLCEERU | 93 | 706.3656 | 2 | 25.35 | 1410.724 | −5.1 | UNIQUE | 343 |
| 7481 | pVdLGELLCEERU | 93 | 700.3665 | 2 | 25.43 | 1398.716 | 1.5 | UNIQUE | 344 |
| 7551 | pVTLQELmCEERU | 93 | 761.8743 | 2 | 25.47 | 1521.748 | −9.4 | UNIQUE | 274 |
| 7581 | pVTLAELLCEERU | 93 | 709.371 | 2 | 25.55 | 1416.733 | −3.7 | UNIQUE | 345 |
| 7673 | pVbLAEiLCEERU | 93 | 713.3732 | 2 | 25.68 | 1424.74 | −5.4 | UNIQUE | 346 |
| 6091 | pLTVQEiLCEERU | 92 | 736.8727 | 2 | 22.37 | 1471.742 | −7.2 | UNIQUE | 347 |
| 5408 | pVsVAEhLCEERU | 92 | 714.3629 | 2 | 23.49 | 1426.713 | −1.5 | UNIQUE | 348 |
| 6800 | pmsLQEVyCEERU | 92 | 777.8666 | 2 | 24.09 | 1553.723 | −2.6 | UNIQUE | 349 |
| 6775 | pVbLAEVVCEERU | 92 | 700.3659 | 2 | 24.1 | 1398.721 | −2.5 | UNIQUE | 350 |
| 6640 | pLbiQEVyCEERU | 92 | 751.8698 | 2 | 24.14 | 1501.735 | −6.8 | UNIQUE | 351 |
| 6821 | pmsVQEiLCEERU | 92 | 766.8735 | 2 | 24.2 | 1531.736 | −2.2 | UNIQUE | 352 |
| 8001 | pixLAEVLCEERU | 92 | 720.3814 | 2 | 26.25 | 1438.75 | −0.9 | UNIQUE | 353 |
| 4856 | pysiAEViCEERU | 91 | 723.3399 | 2 | 19.81 | 1444.682 | −11.3 | UNIQUE | 354 |
| 5309 | piTyQEVyCEERU | 91 | 756.8427 | 2 | 20.25 | 1511.69 | −12.8 | UNIQUE | 355 |
| 5486 | pyNyQEVVCEERU | 91 | 757.3418 | 2 | 21.83 | 1512.682 | −8.8 | UNIQUE | 356 |
| 6031 | pLsyQELyCEERU | 91 | 770.8562 | 2 | 22.8 | 1539.718 | −13.3 | UNIQUE | 357 |
| 7576 | pVdhAEiLCEERU | 91 | 712.3662 | 2 | 23.5 | 1422.716 | 1.4 | UNIQUE | 358 |
| 6562 | pLsVAELLCEERU | 91 | 715.3708 | 2 | 23.73 | 1428.733 | −3.9 | UNIQUE | 359 |
| 6938 | pVsLAELLCEERU | 91 | 715.3708 | 2 | 24.34 | 1428.733 | −3.9 | UNIQUE | 360 |
| 7140 | pVbVAEVLCEERU | 91 | 700.3654 | 2 | 24.74 | 1398.721 | −3.2 | UNIQUE | 361 |
| 5940 | pisyAEiVCEERU | 90 | 723.3399 | 2 | 19.81 | 1444.682 | −11.3 | UNIQUE | 362 |
| 4608 | pVsVAEVICEERU | 90 | 700.3485 | 2 | 20.53 | 1398.689 | −4.5 | UNIQUE | 363 |
| 5552 | pysiAEVyCEERU | 90 | 734.3335 | 2 | 21.36 | 1466.669 | −11.1 | UNIQUE | 364 |
| 6632 | pidVAELyCEERU | 90 | 716.3501 | 2 | 23.5 | 1430.694 | −5.7 | UNIQUE | 365 |
| 6964 | pVxLAEiVCEREU | 90 | 713.3731 | 2 | 24.43 | 1424.734 | −1.4 | UNIQUE | 366 |
| 7914 | pidLwEVyCEERU | 90 | 723.3588 | 2 | 26.04 | 1444.704 | −0.6 | UNIQUE | 367 |
| 8066 | pVdVAEkyCEERU | 90 | 724.3657 | 2 | 26.36 | 1446.731 | −9.6 | UNIQUE | 368 |
| 3961 | ELsVAEIVCEERU | 89 | 700.3468 | 2 | 18.91 | 1398.684 | −3.8 | UNIQUE | 179 |
| 5664 | pVTiAEyVCEERU | 89 | 711.3398 | 2 | 20.25 | 1420.679 | −9.6 | UNIQUE | 369 |
| 5466 | pVsVQEhVCEREU | 89 | 735.8665 | 2 | 21.17 | 1469.719 | −0.6 | UNIQUE | 370 |
| 5548 | pVsVAEiLCEERU | 89 | 707.3553 | 2 | 22.06 | 1412.704 | −5.8 | UNIQUE | 371 |
| 5749 | pVsiAEVhCEERU | 89 | 713.3552 | 2 | 22.3 | 1424.701 | −3.4 | UNIQUE | 372 |
| 5867 | pVNiAEyVCEERU | 89 | 717.8387 | 2 | 22.5 | 1433.674 | −7.6 | UNIQUE | 373 |
| 6006 | pVsywEVyCEERU | 89 | 735.3391 | 2 | 22.73 | 1468.676 | −8.1 | UNIQUE | 374 |
| 6702 | pLshGEVLCEERU | 89 | 714.3629 | 2 | 23.49 | 1426.713 | −1.5 | UNIQUE | 375 |
| 7553 | ELbVwEVVCEERU | 89 | 700.3665 | 2 | 25.43 | 1398.711 | 5.5 | UNIQUE | 376 |
| 4147 | ELsVQEhVCEERU | 88 | 735.8665 | 2 | 19.23 | 1469.715 | 2.5 | UNIQUE | 377 |
| 6261 | pVdiGEhVCEERU | 88 | 698.3507 | 2 | 20.45 | 1394.685 | 1.6 | UNIQUE | 378 |
| 5182 | PVNVAEVyCEERU | 88 | 711.8387 | 2 | 21.25 | 1421.671 | −5.7 | UNIQUE | 379 |
| 5956 | pVsLQELyCEERU | 88 | 753.8615 | 2 | 21.26 | 1505.728 | −13.2 | UNIQUE | 380 |
| 5390 | pLTVGEVVCEERU | 88 | 688.3474 | 2 | 21.58 | 1374.686 | −3.8 | UNIQUE | 381 |
| 6052 | pyNVQEILCEERU | 88 | 753.3562 | 2 | 22.78 | 1504.711 | −8.7 | UNIQUE | 382 |
| 7338 | pLsiAELLCEERU | 88 | 721.3711 | 2 | 25.05 | 1440.736 | −5.5 | UNIQUE | 248 |
| 7577 | pLdVAELLCEERU | 88 | 707.373 | 2 | 25.51 | 1412.732 | −0.4 | UNIQUE | 383 |
| 4050 | ELsiAEiyCEERU | 87 | 723.3399 | 2 | 19.81 | 1444.677 | −8.3 | UNIQUE | 384 |
| 5303 | pVdiGEViCEERU | 87 | 691.3427 | 2 | 20.61 | 1380.676 | −3.5 | UNIQUE | 385 |
| 5003 | pVNyAEVVCEERU | 87 | 711.8385 | 2 | 20.91 | 1421.671 | −5.9 | UNIQUE | 386 |
| 5257 | pVsiQELiCEERU | 87 | 741.8661 | 2 | 21.8 | 1481.729 | −7.5 | UNIQUE | 387 |
| 6152 | pVdiGEyLCEERU | 87 | 709.3425 | 2 | 22.97 | 1416.678 | −5.4 | UNIQUE | 388 |
| 6390 | pLsiAEVLCEERU | 87 | 714.3629 | 2 | 23.49 | 1426.72 | −6.1 | UNIQUE | 389 |
| 6608 | pLbVQEiyCEERU | 87 | 751.8698 | 2 | 24.14 | 1501.735 | −6.8 | UNIQUE | 390 |
| 7597 | pLsVQEjVCEERU | 87 | 756.8895 | 2 | 25.84 | 1511.773 | −5.4 | UNIQUE | 391 |
| 4792 | ELsyQEiVCEERU | 86 | 745.8514 | 2 | 19.9 | 1489.696 | −5 | UNIQUE | 392 |
| 3608 | EVNyAEVVCEERU | 85 | 704.8315 | 2 | 18.15 | 1407.651 | −1.7 | UNIQUE | 393 |
| 3875 | ELSVAEVVCEERU | 85 | 694.3477 | 2 | 18.75 | 1386.681 | −0.3 | UNIQUE | 394 |
| 3983 | EisyQEyiCEERU | 85 | 761.8361 | 2 | 18.92 | 1521.673 | −10.2 | UNIQUE | 395 |
| 4168 | ELbVQEiyCEERU | 85 | 744.8719 | 2 | 19.07 | 1487.715 | 9.4 | UNIQUE | 396 |
| 4132 | ELsVQpViCEERU | 85 | 735.8665 | 2 | 19.23 | 1469.726 | −5 | UNIQUE | 397 |
| 5843 | EhTVAEVyCEERU | 85 | 711.3398 | 2 | 20.25 | 1420.668 | −1.9 | UNIQUE | 398 |
| 4789 | ELsVQELVCEERU | 85 | 729.8661 | 2 | 20.45 | 1457.718 | −0.4 | UNIQUE | 399 |
| 5688 | ELbVGEVyCEERU | 85 | 703.343 | 2 | 22.2 | 1404.675 | −2.6 | UNIQUE | 400 |
| 6594 | pLTVGELyCEERU | 85 | 712.3478 | 2 | 22.32 | 1422.691 | −7.2 | UNIQUE | 401 |
| 6055 | phTiAELyCEERU | 85 | 731.3553 | 2 | 23.36 | 1460.706 | −7 | UNIQUE | 402 |
| 6377 | pLsVAEhyCEERU | 85 | 731.3553 | 2 | 23.36 | 1460.703 | −4.9 | UNIQUE | 403 |
| 6900 | pVshAEhyCEERU | 85 | 737.3554 | 2 | 24.46 | 1472.7 | −2.3 | UNIQUE | 404 |
| 7388 | pydVAEVLCEERU | 85 | 710.3494 | 2 | 25.13 | 1418.691 | −4.6 | UNIQUE | 405 |
| 7769 | pLsVQEkiCEERU | 85 | 756.8895 | 2 | 25.84 | 1511.782 | −11.2 | UNIQUE | 406 |
| 3876 | EVsiAEVyCEERU | 84 | 710.3334 | 2 | 18.09 | 1418.659 | −4.6 | UNIQUE | 407 |
| 5031 | pysLQEVyCEERU | 84 | 763.8505 | 2 | 21.32 | 1525.703 | −10.6 | UNIQUE | 408 |
| 6680 | pVsLAEiyCEERU | 84 | 724.3479 | 2 | 21.68 | 1446.694 | −9 | UNIQUE | 409 |
| 5687 | EhdiQEVVCEERU | 84 | 726.8626 | 2 | 22.17 | 1451.702 | 6.2 | UNIQUE | 410 |
| 6952 | pibhQEVyCEERU | 84 | 757.8684 | 2 | 24.34 | 1513.732 | −6.2 | UNIQUE | 411 |
| 7722 | pVbiwEVVCEERU | 84 | 706.3656 | 2 | 25.35 | 1410.718 | −1 | UNIQUE | 412 |
| 8014 | pVxLGELyCEERU | 84 | 724.3632 | 2 | 26.27 | 1446.721 | −6.1 | UNIQUE | 413 |
| 4405 | pyNyQEVVCEERU | 83 | 757.3418 | 2 | 19.79 | 1512.682 | −8.8 | UNIQUE | 356 |
| 4446 | pVsyQEiyCEERU | 83 | 762.8431 | 2 | 19.9 | 1523.69 | −12.2 | UNIQUE | 414 |
| 4612 | EVdiQEVyCEERU | 83 | 730.8447 | 2 | 20.22 | 1459.68 | −3.3 | UNIQUE | 415 |
| 4569 | pysVAEVVCEERU | 83 | 711.3398 | 2 | 20.25 | 1420.676 | −7.4 | UNIQUE | 416 |
| 6550 | pVTLGELyCEREU | 83 | 712.3478 | 2 | 22.32 | 1422.691 | −7.2 | UNIQUE | 417 |
| 5798 | ELdVQpVVCEERU | 82 | 721.8693 | 2 | 22.38 | 1441.722 | 1.3 | UNIQUE | 418 |
| 6751 | pLbiAEiVCEREU | 82 | 712.3662 | 2 | 23.5 | 1422.727 | −6.3 | UNIQUE | 419 |
| 4952 | EhsVQELICEERU | 81 | 741.8661 | 2 | 21.8 | 1481.718 | −0.1 | UNIQUE | 420 |
| 6629 | pLsLQEILCEERU | 81 | 749.8813 | 2 | 23.84 | 1497.757 | −6 | UNIQUE | 421 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 15
Condition I-2.2

| | | | | | | | | | SEQ ID |
|---|---|---|---|---|---|---|---|---|---|
| Scan | Peptide | ALC (%) | m/z | z | RT | Mass | ppm | Uniqueness | Nos: |
| 7048 | pVTLQELLCEERU | 96 | 737.8822 | 2 | 23.13 | 1473.754 | −2.9 | UNIQUE | 328 |
| 6920 | pVTVAEVLCEERU | 95 | 695.356 | 2 | 22.95 | 1388.701 | −2.7 | UNIQUE | 422 |
| 8102 | pVTLAEhVCEERU | 95 | 708.363 | 2 | 24.42 | 1414.713 | −1.3 | UNIQUE | 423 |
| 8390 | pydVAEVLCEERU | 95 | 710.35 | 2 | 25.68 | 1418.691 | −3.7 | UNIQUE | 405 |
| 9549 | pLTVwEhLCEERU | 95 | 722.3782 | 2 | 26.86 | 1442.739 | 2 | UNIQUE | 424 |
| 6971 | pVdVAELICEERU | 94 | 699.3584 | 2 | 23.04 | 1396.704 | −1.2 | UNIQUE | 319 |
| 8030 | pVTiwEiLCEERU | 94 | 714.3623 | 2 | 24.13 | 1426.717 | −5.1 | UNIQUE | 425 |
| 8104 | pidLQELVCEERU | 94 | 734.8772 | 2 | 24.37 | 1467.741 | −0.8 | UNIQUE | 426 |
| 9013 | pLTiwEVLCEERU | 94 | 715.3677 | 2 | 25.25 | 1428.73 | −6.4 | UNIQUE | 427 |
| 12372 | pLbhAEkLCEERU | 94 | 741.4037 | 2 | 31.04 | 1480.804 | −7.6 | UNIQUE | 428 |
| 7128 | pLbVAEVICEERU | 93 | 706.3656 | 2 | 23.22 | 1410.724 | −5.1 | UNIQUE | 329 |
| 7200 | pVdhGEVVCEERU | 93 | 692.3505 | 2 | 23.68 | 1382.681 | 3.7 | UNIQUE | 429 |
| 7740 | pyNVAEhVCEERU | 93 | 724.8464 | 2 | 24.09 | 1447.683 | −3.1 | UNIQUE | 430 |
| 8052 | pVThAELyCEERU | 93 | 725.3535 | 2 | 24.4 | 1448.703 | −7.5 | UNIQUE | 431 |
| 8360 | pLbVAEILCEERU | 93 | 713.3731 | 2 | 25.07 | 1424.74 | −5.5 | UNIQUE | 432 |
| 8773 | pLbVQEhVCEERU | 93 | 741.8849 | 2 | 25.64 | 1481.754 | 0.8 | UNIQUE | 433 |
| 8955 | pLNVwELyCEERU | 93 | 732.8618 | 2 | 26.04 | 1463.712 | −2.2 | UNIQUE | 434 |
| 10114 | pLbVAELLCEERU | 93 | 714.3814 | 2 | 27.7 | 1426.752 | −2.6 | UNIQUE | 435 |
| 11186 | pmbVAEhLCEERU | 93 | 744.3804 | 2 | 28.24 | 1486.743 | 2.4 | UNIQUE | 436 |
| 10903 | pLbiwELhCEERU | 93 | 733.3891 | 2 | 28.43 | 1464.762 | 1.5 | UNIQUE | 437 |
| 10901 | pmdLwEhVCEERU | 93 | 744.3613 | 2 | 28.85 | 1486.733 | −16.7 | UNIQUE | 438 |
| 7273 | pVNiAEVLCEERU | 92 | 707.8549 | 2 | 23.43 | 1413.7 | −3 | UNIQUE | 439 |
| 7354 | pVdiAELyCEERU | 92 | 716.35 | 2 | 23.5 | 1430.694 | −5.8 | UNIQUE | 440 |
| 7517 | pidVAELyCEERU | 92 | 716.35 | 2 | 23.5 | 1430.694 | −5.8 | UNIQUE | 365 |
| 7516 | pVbiwEVyCEERU | 92 | 723.3582 | 2 | 23.8 | 1444.708 | −4.3 | UNIQUE | 441 |
| 7965 | pVxiAELVCEERU | 92 | 713.3735 | 2 | 24.47 | 1424.734 | −0.8 | UNIQUE | 442 |
| 8705 | pVNhAEVhCEERU | 92 | 720.8632 | 2 | 24.47 | 1439.705 | 4.6 | UNIQUE | 443 |
| 8412 | pLbVQEiLCEERU | 92 | 741.8849 | 2 | 25.64 | 1481.761 | −3.8 | UNIQUE | 311 |
| 8348 | pibhQELyCEERU | 92 | 764.877 | 2 | 25.64 | 1527.747 | −5.1 | UNIQUE | 444 |
| 9015 | pibVQEmiCEERU | 92 | 764.877 | 2 | 25.64 | 1527.743 | −2.1 | UNIQUE | 445 |
| 8776 | pVdyAELLCEERU | 92 | 717.3576 | 2 | 26.25 | 1432.706 | −3.9 | UNIQUE | 446 |
| 9985 | pLbLGEhLCEERU | 92 | 720.3804 | 2 | 27.14 | 1438.749 | −1.6 | UNIQUE | 447 |
| 10239 | pVbiwEhLCEERU | 92 | 726.3815 | 2 | 27.82 | 1450.746 | 1.7 | UNIQUE | 448 |
| 11482 | pVdiwEkLCEERU | 92 | 727.3887 | 2 | 28.2 | 1452.769 | −4.4 | UNIQUE | 449 |
| 10659 | pLdjAELyCEERU | 92 | 744.3804 | 2 | 28.24 | 1486.756 | −6.7 | UNIQUE | 450 |
| 6423 | phTVGEVVCEERU | 91 | 694.3477 | 2 | 22.21 | 1386.682 | −1 | UNIQUE | 451 |
| 7091 | pVdVAEVLCEERU | 91 | 693.3575 | 2 | 23.16 | 1384.701 | −0.1 | UNIQUE | 452 |
| 7115 | pVThAEVyCEERU | 91 | 718.3474 | 2 | 24.07 | 1434.688 | −5.2 | UNIQUE | 453 |
| 11282 | pLxLwEiLCEERU | 91 | 734.3959 | 2 | 29.42 | 1466.775 | 1.4 | UNIQUE | 454 |
| 5445 | pVTVGEiVCEERU | 90 | 687.3405 | 2 | 20.62 | 1372.673 | −4.9 | UNIQUE | 455 |
| 5791 | piTyQEVyCEERU | 90 | 756.8425 | 2 | 21.16 | 1511.69 | −13 | UNIQUE | 355 |
| 6150 | pVdVAEVVCEERU | 90 | 686.3501 | 2 | 21.72 | 1370.685 | 0.4 | UNIQUE | 456 |
| 6178 | ELdVAEVVCEERU | 90 | 686.3587 | 2 | 21.76 | 1370.681 | 16.2 | UNIQUE | 457 |
| 7886 | pLNLQEVLCEERU | 90 | 744.3784 | 2 | 24.35 | 1486.749 | −4.8 | UNIQUE | 458 |
| 8149 | ELbVAEVLCEERU | 90 | 700.3651 | 2 | 24.71 | 1398.716 | −0.5 | UNIQUE | 459 |
| 8624 | pVTLAELLCEERU | 90 | 709.3684 | 2 | 25.49 | 1416.733 | −7.3 | UNIQUE | 345 |
| 10134 | pLbiQEmLCEERU | 90 | 772.8922 | 2 | 28.04 | 1543.771 | −0.6 | UNIQUE | 460 |
| 5452 | pVNVGEiVCEERU | 89 | 693.8385 | 2 | 20.61 | 1385.668 | 4.2 | UNIQUE | 461 |
| 5820 | pVsVQEhVCEERU | 89 | 735.8658 | 2 | 21.17 | 1469.719 | −1.4 | UNIQUE | 462 |
| 6310 | pVTVQELVCEERU | 89 | 723.8661 | 2 | 21.68 | 1445.723 | −3.4 | UNIQUE | 463 |
| 6952 | pLsiAEhVCEERU | 89 | 720.364 | 2 | 22.95 | 1438.716 | −2 | UNIQUE | 464 |
| 7090 | PVNVAEhVCEERU | 89 | 707.8542 | 2 | 23.16 | 1413.693 | 0.8 | UNIQUE | 465 |
| 7258 | ELNLQELyCEERU | 89 | 754.3657 | 2 | 23.27 | 1506.719 | −1.6 | UNIQUE | 227 |
| 7145 | pVTyAEVLCEERU | 89 | 712.3483 | 2 | 23.75 | 1422.691 | −6.6 | UNIQUE | 466 |
| 7627 | pVTiQEmVCEERU | 89 | 753.8669 | 2 | 23.82 | 1505.72 | −0.6 | UNIQUE | 467 |
| 9883 | pkNiGELLCEERU | 89 | 728.8782 | 2 | 27.32 | 1455.755 | −9.1 | UNIQUE | 468 |
| 9479 | pLbhQELyCEERU | 89 | 765.885 | 2 | 27.36 | 1529.76 | −3 | UNIQUE | 469 |
| 11409 | pVbhAEVjCEERU | 89 | 733.3891 | 2 | 28.43 | 1464.767 | −2.4 | UNIQUE | 470 |
| 10907 | pLsVwEjhCEERU | 89 | 748.3928 | 2 | 28.46 | 1494.773 | −1.6 | UNIQUE | 471 |
| 7596 | pVThQELVCEERU | 88 | 736.874 | 2 | 23.88 | 1471.735 | −0.9 | UNIQUE | 472 |
| 7540 | pLbiQEVyCEERU | 88 | 751.8892 | 2 | 23.88 | 1501.735 | 19.1 | UNIQUE | 351 |
| 7598 | pVThAEyVCEERU | 88 | 718.3474 | 2 | 24.07 | 1434.688 | −5.2 | UNIQUE | 473 |
| 7787 | pVsVwEVyCEERU | 88 | 718.3474 | 2 | 24.07 | 1434.686 | −3.7 | UNIQUE | 474 |
| 7972 | pVbLQEVVCEERU | 88 | 728.8772 | 2 | 24.48 | 1455.742 | −1.6 | UNIQUE | 475 |
| 8204 | pisVQEmhCEERU | 88 | 772.8741 | 2 | 24.59 | 1543.732 | 1 | UNIQUE | 476 |
| 8074 | pVNmQELVCEERU | 88 | 761.3732 | 2 | 24.63 | 1520.728 | 2.6 | UNIQUE | 477 |
| 11065 | pLbLwEhLCEERU | 88 | 734.3958 | 2 | 29.1 | 1466.774 | 1.9 | UNIQUE | 478 |
| 12023 | pkdiwELLCEERU | 88 | 734.3967 | 2 | 30.52 | 1466.785 | −4.3 | UNIQUE | 479 |
| 5614 | pLTiQEVyCEERU | 87 | 746.8588 | 2 | 20.87 | 1491.716 | −8.5 | UNIQUE | 480 |
| 5721 | pVNiQEyiCEERU | 87 | 752.3486 | 2 | 21.05 | 1502.699 | −10.5 | UNIQUE | 481 |
| 5884 | pVsiQELyCEERU | 87 | 752.8585 | 2 | 21.3 | 1503.716 | −8.9 | UNIQUE | 482 |
| 6231 | pysiAEVyCEERU | 87 | 734.332 | 2 | 21.39 | 1466.669 | −13.1 | UNIQUE | 364 |
| 5966 | pVdiGEViCEERU | 87 | 691.3433 | 2 | 21.42 | 1380.676 | −2.6 | UNIQUE | 385 |
| 6583 | pidiAEViCEERU | 87 | 704.3678 | 2 | 22.33 | 1406.694 | 19.2 | UNIQUE | 483 |
| 7854 | pLTiAEhiCEERU | 87 | 720.364 | 2 | 22.95 | 1438.72 | −4.2 | UNIQUE | 484 |
| 7537 | ELTiQEmVCEERU | 87 | 753.8669 | 2 | 23.82 | 1505.716 | 2.3 | UNIQUE | 485 |
| 7819 | pisyAEILCEERU | 87 | 730.3663 | 2 | 24.25 | 1458.697 | 14.3 | UNIQUE | 486 |
| 7850 | pLdyQELyCEERU | 87 | 762.8604 | 2 | 24.29 | 1523.718 | −7.6 | UNIQUE | 487 |
| 7718 | pLsywEVyCEERU | 87 | 742.3463 | 2 | 24.4 | 1482.691 | −8.9 | UNIQUE | 488 |
| 9627 | pjdVQEhyCEERU | 87 | 771.884 | 2 | 26.87 | 1541.759 | −3.3 | UNIQUE | 489 |
| 11377 | ELbVwEVkCEERU | 87 | 721.3871 | 2 | 29.56 | 1440.766 | −4.6 | UNIQUE | 490 |
| 5591 | pVTiGEVyCEERU | 86 | 704.332 | 2 | 20.82 | 1406.663 | −9.7 | UNIQUE | 491 |
| 6846 | pVsiApyVCEERU | 86 | 724.348 | 2 | 21.64 | 1446.699 | −11.9 | UNIQUE | 492 |
| 6966 | pyNVGEILCEERU | 86 | 717.858 | 2 | 22.97 | 1433.674 | 19.3 | UNIQUE | 493 |
| 7827 | PLNVAEILCEERU | 86 | 714.8628 | 2 | 24.26 | 1427.715 | −3 | UNIQUE | 494 |
| 8335 | pLsVAEhLCEERU | 86 | 721.37 | 2 | 25.03 | 1440.729 | −2.5 | UNIQUE | 495 |
| 7787 | pVsLAEVyCEERU | 86 | 718.3654 | 2 | 25.18 | 1434.691 | 17.5 | UNIQUE | 496 |
| 8646 | pVshAEmiCEERU | 86 | 744.3634 | 2 | 25.59 | 1486.711 | 1.1 | UNIQUE | 497 |
| 8915 | ELNhAELVCEERU | 86 | 714.8633 | 2 | 25.89 | 1427.704 | 5.4 | UNIQUE | 498 |
| 8985 | ELTVAEKVCEERU | 86 | 709.3704 | 2 | 25.99 | 1416.737 | −7.5 | UNIQUE | 499 |
| 11083 | pLdmAELLCEERU | 86 | 738.3638 | 2 | 29.12 | 1474.742 | −19.6 | UNIQUE | 500 |
| 5247 | EhTyQEyVCEERU | 85 | 756.8427 | 2 | 19.8 | 1511.679 | −5.5 | UNIQUE | 501 |
| 4958 | pVsyQEiVCEERU | 85 | 745.8513 | 2 | 19.81 | 1489.7 | −8.1 | UNIQUE | 502 |
| 6209 | pyNyQEVVCEERU | 85 | 757.3423 | 2 | 21.75 | 1512.682 | −8.1 | UNIQUE | 356 |
| 7193 | pVTLAEVICEERU | 85 | 701.351 | 2 | 22.85 | 1400.704 | −12 | UNIQUE | 503 |
| 6573 | piTVAEVyCEERU | 85 | 711.3411 | 2 | 22.95 | 1420.679 | −7.8 | UNIQUE | 504 |
| 6737 | pmsiGEiVCEERU | 85 | 730.3486 | 2 | 23.09 | 1458.686 | −2.4 | UNIQUE | 505 |
| 7494 | pVbVAEyVCEERU | 85 | 710.3499 | 2 | 23.5 | 1418.695 | −6.9 | UNIQUE | 506 |
| 7768 | pmNLQEiyCEERU | 85 | 784.3651 | 2 | 24.15 | 1566.721 | −3.4 | UNIQUE | 507 |
| 11975 | pkdhGEmLCEERU | 85 | 751.3881 | 2 | 30.45 | 1500.763 | −0.7 | UNIQUE | 508 |
| 5151 | pysVAEVVCEERU | 84 | 711.3387 | 2 | 20.1 | 1420.676 | −8.9 | UNIQUE | 416 |
| 6026 | pVsVAEyyCEERU | 84 | 728.332 | 2 | 21.41 | 1454.666 | −11 | UNIQUE | 509 |
| 6440 | pVTLGEiyCEERU | 84 | 711.3403 | 2 | 22 | 1420.679 | −8.9 | UNIQUE | 510 |
| 7380 | pVshAEVLCEERU | 84 | 714.3623 | 2 | 24.13 | 1426.713 | −2.3 | UNIQUE | 511 |
| 8257 | pLTiAELyCEERU | 84 | 725.3535 | 2 | 24.4 | 1448.71 | −12.1 | UNIQUE | 512 |
| 7673 | pVsiwELyCEERU | 84 | 731.3552 | 2 | 24.6 | 1460.704 | −5.8 | UNIQUE | 513 |
| 9043 | ELdLwEVyCEERU | 84 | 717.3576 | 2 | 26.25 | 1432.696 | 3 | UNIQUE | 514 |
| 12128 | pLxVwEjyCEERU | 84 | 758.3953 | 2 | 30.68 | 1514.781 | −3.2 | UNIQUE | 515 |
| 12414 | pVxywphLCEERU | 84 | 751.3741 | 2 | 31.1 | 1500.763 | −19.4 | UNIQUE | 516 |
| 6503 | EhdiAEViCEERU | 83 | 704.3502 | 2 | 22.31 | 1406.683 | 1.8 | UNIQUE | 517 |
| 6533 | pVsiAEVhCEERU | 83 | 713.3552 | 2 | 22.35 | 1424.701 | −3.4 | UNIQUE | 372 |
| 6589 | piTLGEILCEERU | 83 | 707.3718 | 2 | 22.39 | 1412.707 | 15.4 | UNIQUE | 518 |
| 7256 | pVsywEVyCEERU | 83 | 735.3391 | 2 | 22.75 | 1468.676 | −8.1 | UNIQUE | 374 |
| 7618 | pLshGEVLCEERU | 83 | 714.3623 | 2 | 24.13 | 1426.713 | −2.3 | UNIQUE | 375 |
| 7253 | pLsVAEhyCEERU | 83 | 731.3552 | 2 | 24.6 | 1460.703 | −5.1 | UNIQUE | 403 |
| 8200 | pVNhApVVCEERU | 83 | 714.8624 | 2 | 24.82 | 1427.713 | −1.9 | UNIQUE | 805 |
| 8357 | pisywELICEERU | 83 | 737.3737 | 2 | 25.06 | 1472.707 | 17.3 | UNIQUE | 519 |
| 9476 | pVNhAEmVCEERU | 83 | 738.8621 | 2 | 26.71 | 1475.703 | 4.6 | UNIQUE | 520 |
| 10013 | pVdLwEhVCEERU | 83 | 713.3745 | 2 | 27.51 | 1424.723 | 8.1 | UNIQUE | 521 |
| 6518 | pisyAEiVCEERU | 82 | 723.3406 | 2 | 21.28 | 1444.682 | −10.4 | UNIQUE | 362 |
| 7025 | pVNyAELVCEERU | 82 | 718.8469 | 2 | 23.06 | 1435.687 | −5 | UNIQUE | 522 |
| 7275 | pysVAEmVCEERU | 82 | 742.3463 | 2 | 24.4 | 1482.686 | −5.1 | UNIQUE | 523 |
| 9525 | pVsjAEhVCEERU | 82 | 734.3782 | 2 | 26.56 | 1466.748 | −4.1 | UNIQUE | 524 |
| 5192 | pysiQEyyCEERU | 81 | 779.8344 | 2 | 20.18 | 1557.68 | −16.7 | UNIQUE | 325 |
| 7334 | pisVAELhCEERU | 81 | 720.364 | 2 | 22.95 | 1438.716 | −2 | UNIQUE | 525 |
| 8585 | ELsVwEVLCEERU | 81 | 708.363 | 2 | 24.42 | 1414.707 | 3.3 | UNIQUE | 526 |
| 6156 | pVdiAEVyCEERU | 80 | 709.3602 | 2 | 21.55 | 1416.678 | 19.5 | UNIQUE | 527 |
| 6288 | pVTVQEVLCEERU | 80 | 723.8661 | 2 | 21.68 | 1445.723 | −3.4 | UNIQUE | 528 |
| 8621 | pLsiApLyCEERU | 80 | 738.3619 | 2 | 25.89 | 1474.73 | −14 | UNIQUE | 529 |
| 11410 | phTmGEjLCEERU | 80 | 759.3866 | 2 | 28.75 | 1516.758 | 0.5 | UNIQUE | 530 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxy1 proline (Hyp): s, Amino isobutyric (Aib): w, C-term amide: U.

TABLE 16
Condition I-2.3

| | | | | | | | | | SEQ ID |
|---|---|---|---|---|---|---|---|---|---|
| Scan | Peptide | ALC (%) | m/z | z | RT | Mass | ppm | Uniqueness | Nos: |
| 8228 | pmTowEiLCEERU | 95 | 788.3776 | 2 | 30.42 | 1574.737 | 2.1 | UNIQUE | 140 |
| 7993 | piNoAEoVCEERU | 93 | 798.8622 | 2 | 30.05 | 1595.71 | −0.1 | UNIQUE | 531 |
| 4356 | ELTLQELLCEERU | 90 | 737.8828 | 2 | 23.26 | 1473.75 | 0.9 | UNIQUE | 171 |
| 8560 | pLNowEmyCEERU | 88 | 805.8696 | 2 | 30.84 | 1609.72 | 3.2 | UNIQUE | 532 |
| 9181 | pVxowEimCEERU | 88 | 793.3891 | 2 | 32.26 | 1584.751 | 7.8 | UNIQUE | 533 |
| 3734 | pVsiAELVCEERU | 87 | 707.3562 | 2 | 21.57 | 1412.704 | −4.6 | UNIQUE | 534 |
| 3449 | ELsiAEVVCEERU | 86 | 700.3478 | 2 | 20.34 | 1398.684 | −2.3 | UNIQUE | 535 |
| 4257 | ELbVQEVICEERU | 86 | 727.87 | 2 | 23 | 1453.725 | 0.1 | UNIQUE | 536 |
| 4490 | pVdhQEVVCEERU | 86 | 727.87 | 2 | 23 | 1453.719 | 4.8 | UNIQUE | 537 |
| 9008 | piNowpVmCEERU | 86 | 794.8772 | 2 | 31.19 | 1587.737 | 1.8 | UNIQUE | 538 |
| 9011 | poNLwEjyCEERU | 86 | 801.8846 | 2 | 31.83 | 1601.76 | −3 | UNIQUE | 539 |
| 9010 | ELxowphyCEERU | 86 | 793.3891 | 2 | 32.26 | 1584.756 | 4.9 | UNIQUE | 540 |
| 3699 | ELsVAELVCEERU | 85 | 701.3561 | 2 | 21.35 | 1400.697 | 0.4 | UNIQUE | 541 |
| 8896 | pVsoAEjmCEERU | 85 | 801.386 | 2 | 31.7 | 1600.759 | −0.7 | UNIQUE | 542 |
| 3604 | ELsVGEhVCEERU | 84 | 700.348 | 2 | 21.13 | 1398.678 | 2.6 | UNIQUE | 543 |
| 3866 | ELdVAEiVCEERU | 84 | 692.3517 | 2 | 21.98 | 1382.684 | 3.6 | UNIQUE | 544 |
| 3970 | pLTiQELyCEERU | 82 | 753.865 | 2 | 22.23 | 1505.731 | −10.6 | UNIQUE | 545 |
| 7814 | ELTowEmVCEERU | 82 | 775.3701 | 2 | 29.73 | 1548.714 | 7.2 | UNIQUE | 546 |
| 4015 | pVshGEVLCEERU | 81 | 707.3562 | 2 | 21.88 | 1412.698 | 0.1 | UNIQUE | 547 |
| 3750 | ELSVAEiVCEERU | 79 | 700.3461 | 2 | 21.63 | 1398.684 | −4.8 | UNIQUE | 179 |
| 8172 | poNiGEOLCEERU | 79 | 798.8615 | 2 | 30.36 | 1595.71 | −0.9 | UNIQUE | 548 |
| 7779 | ELTowEmiCEERU | 78 | 781.3682 | 2 | 29.68 | 1560.717 | 2.9 | UNIQUE | 549 |
| 7871 | pVsoQEoyCEERU | 78 | 837.8662 | 2 | 29.82 | 1673.723 | −3.2 | UNIQUE | 550 |
| 8879 | pLTowEjVCEERU | 77 | 778.4055 | 2 | 31.6 | 1554.774 | 14.2 | UNIQUE | 551 |
| 8281 | posVwEmyCEERU | 76 | 798.3642 | 2 | 30.56 | 1594.709 | 3.2 | UNIQUE | 552 |
| 4521 | pVThGELVCEERU | 74 | 701.3566 | 2 | 23.6 | 1400.698 | 0.7 | UNIQUE | 553 |
| 9580 | pkdmwEoLCEERU | 74 | 801.4038 | 2 | 32.89 | 1600.789 | 2.3 | UNIQUE | 554 |
| 3702 | ELsyAEVVCEERU | 72 | 711.3391 | 2 | 21.46 | 1420.671 | −5.3 | UNIQUE | 555 |
| 4919 | ELbVAELyCEERU | 71 | 717.3674 | 2 | 24.36 | 1432.706 | 9.8 | UNIQUE | 556 |
| 8646 | posmAEmyCEERU | 70 | 822.3638 | 2 | 31.17 | 1642.709 | 2.6 | UNIQUE | 557 |
| 4051 | pisVQELLCEERU | 69 | 742.8732 | 2 | 22.46 | 1483.742 | −6.4 | UNIQUE | 558 |
| 4045 | ELshGELVCEERU | 67 | 707.3562 | 2 | 21.88 | 1412.693 | 3.2 | UNIQUE | 559 |
| 3925 | pVsiAEVyCEERU | 65 | 717.3406 | 2 | 22.11 | 1432.679 | −8.5 | UNIQUE | 233 |
| 3807 | pysVGELVCEERU | 63 | 711.3393 | 2 | 21.75 | 1420.676 | −8.1 | UNIQUE | 560 |
| 4672 | EhNVAELICEERU | 63 | 713.856 | 2 | 23.93 | 1425.692 | 4 | UNIQUE | 561 |
| 8496 | pkNiGEhoCEERU | 57 | 776.8778 | 2 | 30.7 | 1551.749 | −5.1 | UNIQUE | 562 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 17
Condition I-2.4

| | | | | | | | | | SEQ |
|---|---|---|---|---|---|---|---|---|---|
| | | | | | | | | | ID |
| Scan | Peptide | ALC (%) | m/z | z | RT | Mass | ppm | Uniqueness | Nos: |
| 9476 | pmToAELhCEERU | 97 | 788.3777 | 2 | 30.6 | 1574.736 | 2.9 | UNIQUE | 141 |
| 10656 | pmbhwEmhCEERU | 96 | 788.3971 | 2 | 32.57 | 1574.759 | 12.9 | UNIQUE | 142 |
| 10257 | pobiwEmiCEERU | 93 | 792.382 | 2 | 31.88 | 1582.744 | 3.3 | UNIQUE | 143 |
| 11093 | poxiwpLLCEERU | 92 | 783.4053 | 2 | 33.49 | 1564.793 | 2.2 | UNIQUE | 152 |
| 9823 | pVxoGEmLCEERU | 91 | 780.3809 | 2 | 31.53 | 1558.738 | 5.9 | UNIQUE | 563 |
| 4168 | ELNiGEyVCEERU | 89 | 710.8318 | 2 | 21.08 | 1419.654 | −3.3 | UNIQUE | 564 |
| 11285 | pLxjwEmhCEERU | 89 | 785.4223 | 2 | 33.69 | 1568.813 | 11 | UNIQUE | 565 |
| 3930 | ELsVAEhVCEERU | 88 | 707.3558 | 2 | 20.54 | 1412.693 | 2.6 | UNIQUE | 174 |
| 4290 | ELsVQEiLCEERU | 88 | 735.8672 | 2 | 21.48 | 1469.721 | −1 | UNIQUE | 566 |
| 9403 | poTmwEVyCEERU | 86 | 792.3627 | 2 | 30.39 | 1582.709 | 1.3 | UNIQUE | 567 |
| 9854 | poNmApiLCEERU | 86 | 794.8766 | 2 | 31.08 | 1587.743 | −2.6 | UNIQUE | 568 |
| 9920 | pydowELLCEERU | 86 | 773.3727 | 2 | 31.29 | 1544.73 | 0.9 | UNIQUE | 569 |
| 3839 | pisVQEiVCEERU | 85 | 734.8586 | 2 | 20.35 | 1467.713 | −7.1 | UNIQUE | 270 |
| 4100 | ELTVQEVVCEERU | 83 | 716.8585 | 2 | 20.93 | 1431.703 | −0.2 | UNIQUE | 570 |
| 4837 | pLsiQEVLCEERU | 83 | 742.8745 | 2 | 22.47 | 1483.742 | −4.7 | UNIQUE | 571 |
| 10841 | pjsjGEohCEERU | 83 | 803.4042 | 2 | 33.3 | 1604.795 | −0.8 | UNIQUE | 572 |
| 9570 | pmNmAEmhCEERU | 82 | 800.878 | 2 | 30.76 | 1599.723 | 11.6 | UNIQUE | 573 |
| 9693 | pidLwELoCEERU | 82 | 762.3816 | 2 | 30.97 | 1522.742 | 4.1 | UNIQUE | 574 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 18
Condition I-3.1

| Scan | Peptide | ALC (%) | m/z | z | RT | Mass | ppm | Uniqueness | SEQ ID No: |
|---|---|---|---|---|---|---|---|---|---|
| 9393 | pmdhAEoLCEERU | 81 | 786.3811 | 2 | 31.89 | 1570.736 | 7.5 | UNIQUE | 153 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 19
Condition I-3.2

| | | | | | | | | | SEQ |
|---|---|---|---|---|---|---|---|---|---|
| | | | | | | | | | ID |
| Scan | Peptide | ALC (%) | m/z | z | RT | Mass | ppm | Uniqueness | Nos: |
| 8437 | pLTowELLCEERU | 94 | 765.385 | 2 | 29.86 | 1528.756 | −0.1 | UNIQUE | 154 |
| 4853 | ELTLQELLCEERU | 92 | 737.8824 | 2 | 23.23 | 1473.75 | 0.4 | UNIQUE | 171 |
| 9409 | pLdowEhLCEERU | 92 | 769.389 | 2 | 31.58 | 1536.752 | 7.8 | UNIQUE | 144 |
| 9636 | pobiwEmVCEERU | 92 | 786.3812 | 2 | 31.63 | 1570.741 | 4.3 | UNIQUE | 575 |
| 9489 | pmsoAphLCEERU | 92 | 801.3861 | 2 | 31.72 | 1600.756 | 0.8 | UNIQUE | 576 |
| 9677 | piboAEjLCEERU | 89 | 782.3972 | 2 | 32.08 | 1562.787 | −4.5 | UNIQUE | 577 |
| 9865 | pmbhAEoLCEERU | 89 | 793.3895 | 2 | 32.25 | 1584.756 | 5.4 | UNIQUE | 146 |
| 10023 | podhAEkyCEERU | 86 | 786.3805 | 2 | 32.64 | 1570.756 | −6 | UNIQUE | 578 |
| 8972 | phToGEjLCEERU | 84 | 777.3852 | 2 | 31.18 | 1552.761 | −3.2 | UNIQUE | 579 |
| 3558 | ELsVAEiVCEERU | 81 | 700.3474 | 2 | 20.28 | 1398.684 | −3 | UNIQUE | 179 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 20
Condition I-4.1

| | | | | | | | | | SEQ |
|---|---|---|---|---|---|---|---|---|---|
| | | | | | | | | | ID |
| Scan | Peptide | ALC (%) | m/z | z | RT | Mass | ppm | Uniqueness | Nos: |
| 8071 | ELTLQELLCEERU | 97 | 737.8833 | 2 | 27.92 | 1473.75 | 1.6 | UNIQUE | 171 |
| 8992 | pVTLQELLCEERU | 96 | 737.8823 | 2 | 30.01 | 1473.754 | −2.8 | UNIQUE | 328 |
| 9220 | EhTLQELLCEERU | 96 | 743.8822 | 2 | 30.37 | 1485.746 | 2.5 | UNIQUE | 580 |
| 13931 | pLsjAzkyCEERU | 83 | 784.3939 | 2 | 38.95 | 1566.762 | 7.5 | UNIQUE | 155 |
| 7913 | ELTVAEVLCEERU | 80 | 695.3576 | 2 | 27.53 | 1388.697 | 2.6 | UNIQUE | 581 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 21
Condition I-4.2

| | | | | | | | | | SEQ |
|---|---|---|---|---|---|---|---|---|---|
| | | | | | | | | | ID |
| Scan | Peptide | ALC (%) | m/z | z | RT | Mass | ppm | Uniqueness | Nos: |
| 4314 | pVTLQELLCEERU | 95 | 737.8819 | 2 | 22.07 | 1473.754 | −3.3 | UNIQUE | 328 |
| 4068 | pVNiQELVCEERU | 91 | 736.3662 | 2 | 21.64 | 1470.721 | −2.1 | UNIQUE | 133 |
| 3699 | ELSVAEVICEERU | 89 | 700.348 | 2 | 20.95 | 1398.684 | −2.1 | UNIQUE | 582 |
| 3966 | ELNVAELVCEERU | 86 | 701.8577 | 2 | 21.42 | 1401.692 | 6.3 | UNIQUE | 583 |
| 4103 | ELNyQELVCEERU | 86 | 747.3574 | 2 | 21.7 | 1492.704 | −2.2 | UNIQUE | 175 |
| 4284 | pVbVQEVICEERU | 86 | 727.869 | 2 | 22.22 | 1453.73 | −4.1 | UNIQUE | 584 |
| 8627 | pVNhAEmoCEERU | 83 | 787.8708 | 2 | 30.51 | 1573.716 | 7 | UNIQUE | 585 |
| 3690 | EVdiQEVVCEERU | 82 | 713.8542 | 2 | 20.88 | 1425.69 | 2.9 | UNIQUE | 176 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 22
Condition I-4.3

| | | | | | | | | | SEQ ID |
|---|---|---|---|---|---|---|---|---|---|
| Scan | Peptide | ALC (%) | m/z | z | RT | Mass | ppm | Uniqueness | Nos: |
| 6828 | pVTLQELLCEERU | 94 | 737.8832 | 2 | 23.36 | 1473.754 | −1.6 | UNIQUE | 328 |
| 9797 | ELNjGEmLCEERU | 89 | 752.8773 | 2 | 29.25 | 1503.737 | 2.3 | UNIQUE | 586 |
| 6358 | pVNiQELVCEERU | 88 | 736.3647 | 2 | 22.2 | 1470.721 | −4.2 | UNIQUE | 133 |
| 6742 | pVbVQEiVCEERU | 83 | 727.8724 | 2 | 23.12 | 1453.73 | 0.6 | UNIQUE | 587 |
| 6214 | pVsLGEILCEERU | 81 | 707.3553 | 2 | 21.93 | 1412.704 | −5.9 | UNIQUE | 588 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

### Condition I-4.4

No hit

TABLE 23
Condition I-3.3

| | | | | | | | | | SEQ ID |
|---|---|---|---|---|---|---|---|---|---|
| Scan | Peptide | ALC (%) | m/z | z | RT | Mass | ppm | Uniqueness | Nos: |
| 10046 | pjsoAEykCEERU | 95 | 808.4194 | 2 | 38.85 | 1614.794 | 18.7 | UNIQUE | 145 |
| 10318 | pmbhAEoLCEERU | 91 | 793.3899 | 2 | 39.49 | 1584.756 | 5.9 | UNIQUE | 146 |
| 10894 | poxLwEhLCEERU | 90 | 783.4056 | 2 | 40.53 | 1564.782 | 9.7 | UNIQUE | 147 |
| 11266 | pkNjAEikCEERU | 87 | 769.9201 | 2 | 41.26 | 1537.845 | −12.7 | UNIQUE | 589 |
| 10831 | posjAEijCEERU | 86 | 803.4053 | 2 | 40.54 | 1604.802 | −3.5 | UNIQUE | 590 |
| 10323 | pjdowEiyCEERU | 85 | 792.384 | 2 | 39.41 | 1582.751 | 1.4 | UNIQUE | 591 |
| 10431 | poxhwEhyCEERU | 85 | 799.3917 | 2 | 39.66 | 1596.752 | 10.5 | UNIQUE | 592 |
| 10288 | ELsjwEhkCEERU | 84 | 762.4107 | 2 | 39.34 | 1522.809 | −1.5 | UNIQUE | 593 |
| 11874 | pVbjAEjkCEERU | 84 | 768.4276 | 2 | 42.6 | 1534.861 | −13.1 | UNIQUE | 594 |
| 9287 | pmTmwEhLCEERU | 83 | 777.3856 | 2 | 37.7 | 1552.743 | 8.6 | UNIQUE | 595 |
| 9261 | pmToAEhLCEERU | 82 | 788.3744 | 2 | 37.7 | 1574.736 | −1.3 | UNIQUE | 614 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 24
Condition I-3.4

| | | | | | | | | | SEQ ID |
|---|---|---|---|---|---|---|---|---|---|
| Scan | Peptide | ALC (%) | m/z | z | RT | Mass | ppm | Uniqueness | Nos: |
| 9547 | pjsoAEjyCEERU | 93 | 814.3953 | 2 | 39.33 | 1626.789 | −7.7 | UNIQUE | 148 |
| 9200 | poNVwEkyCEERU | 92 | 788.8787 | 2 | 38.7 | 1575.75 | −4.3 | UNIQUE | 156 |
| 8719 | pmNjQELmCEERU | 90 | 819.4059 | 2 | 37.67 | 1636.788 | 5.7 | UNIQUE | 596 |
| 8709 | pLsoQEmmCEERU | 90 | 840.8908 | 2 | 37.76 | 1679.756 | 6.7 | UNIQUE | 597 |
| 8381 | pLTmwEhmCEERU | 89 | 777.3883 | 2 | 37.06 | 1552.743 | 12 | UNIQUE | 598 |
| 9216 | pjNoAEhLCEERU | 89 | 790.8961 | 2 | 38.73 | 1579.772 | 3.8 | UNIQUE | 599 |
| 9542 | pLbowEhLCEERU | 88 | 776.3961 | 2 | 39.4 | 1550.772 | 4 | UNIQUE | 600 |
| 9344 | pLNoAEkLCEERU | 87 | 778.8943 | 2 | 38.99 | 1555.781 | −4.3 | UNIQUE | 601 |
| 8871 | poTmAELyCEERU | 86 | 792.3645 | 2 | 38.02 | 1582.714 | 0.1 | UNIQUE | 602 |
| 9205 | pLdowEhLCEERU | 86 | 769.3888 | 2 | 38.68 | 1536.752 | 7.5 | UNIQUE | 144 |
| 8678 | pLsowEimCEERU | 85 | 794.3795 | 2 | 37.65 | 1586.737 | 4.5 | UNIQUE | 603 |
| 9029 | pmNhAEohCEERU | 85 | 800.8777 | 2 | 38.35 | 1599.728 | 8.1 | UNIQUE | 604 |
| 9030 | poTiwEjyCEERU | 85 | 794.379 | 2 | 38.35 | 1586.752 | −5.2 | UNIQUE | 605 |
| 9108 | piTkwELmCEERU | 84 | 760.3961 | 2 | 38.51 | 1518.78 | −1.6 | UNIQUE | 606 |
| 9218 | pVbowEhhCEERU | 84 | 775.3911 | 2 | 38.74 | 1548.752 | 10 | UNIQUE | 607 |
| 9872 | pobiwEjyCEERU | 84 | 799.3923 | 2 | 40.12 | 1596.771 | −0.8 | UNIQUE | 255 |
| 9265 | pmdowEiLCEERU | 83 | 786.3813 | 2 | 38.83 | 1570.737 | 7.2 | UNIQUE | 608 |
| 9616 | pidVwEojCEERU | 83 | 775.3905 | 2 | 39.53 | 1548.761 | 3.4 | UNIQUE | 609 |
| 9646 | pkToAELyCEERU | 83 | 782.3796 | 2 | 39.59 | 1562.76 | −9.8 | UNIQUE | 610 |
| 8536 | pmTywELjCEERU | 82 | 777.39 | 2 | 37.37 | 1552.761 | 2.7 | UNIQUE | 611 |
| 10254 | pLsjwEjmCEERU | 81 | 793.4211 | 2 | 41.51 | 1584.821 | 4.1 | UNIQUE | 612 |
| 9147 | pLToAEkLCEERU | 80 | 772.397 | 2 | 38.6 | 1542.786 | −4.1 | UNIQUE | 613 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 25
Condition I-4.5

| | | | | | | | | | SEQ ID |
|---|---|---|---|---|---|---|---|---|---|
| Scan | Peptide | ALC (%) | m/z | z | RT | Mass | ppm | Uniqueness | Nos: |
| 13352 | poTmGEmLCEERU | 88 | 799.3697 | 2 | 38.08 | 1596.719 | 3.8 | UNIQUE | 158 |
| 5211 | ELSVAEVICEERU | 87 | 700.3625 | 2 | 24.14 | 1398.684 | 18.7 | UNIQUE | 582 |
| 5528 | ELNyGELVCEERU | 85 | 711.8386 | 2 | 24.86 | 1421.667 | −2.7 | UNIQUE | 615 |
| 5425 | ELsVQEVLCEERU | 82 | 729.8652 | 2 | 24.63 | 1457.718 | −1.6 | UNIQUE | 616 |
| 6238 | ELNiAEyVCEERU | 82 | 717.8383 | 2 | 26.33 | 1433.67 | −5.4 | UNIQUE | 617 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 26
Condition I-4.6

| | | ALC | | | | | | Unique- | SEQ ID |
|---|---|---|---|---|---|---|---|---|---|
| 1 Scan | Peptide | (%) | m/z | Z | RT | Mass | ppm | ness | Nos: |
| 7516 | pVTLQELLCEERU | 94 | 737.8805 | 2 | 27.99 | 1473.754 | −5.2 | UNIQUE | 328 |
| 7531 | ELTLQELLCEERU | 94 | 737.8805 | 2 | 27.99 | 1473.75 | −2.3 | UNIQUE | 171 |
| 14050 | pkTmwEhLCEERU | 93 | 767.4019 | 2 | 37.67 | 1532.789 | 0.1 | UNIQUE | 150 |
| 6052 | ELsVQEVLCEERU | 92 | 729.8651 | 2 | 25.19 | 1457.718 | −1.9 | UNIQUE | 616 |
| 6677 | pVNiQELVCEERU | 92 | 736.3627 | 2 | 26.5 | 1470.721 | −6.9 | UNIQUE | 133 |
| 14080 | pidowEhLCEERU | 91 | 768.3787 | 2 | 37.63 | 1534.739 | 2.7 | UNIQUE | 151 |
| 6716 | ELNiAEyVCEERU | 89 | 717.8383 | 2 | 26.57 | 1433.67 | −5.3 | UNIQUE | 617 |
| 14044 | pVdowELhCEERU | 89 | 762.3799 | 2 | 37.63 | 1522.736 | 6.2 | UNIQUE | 618 |
| 5194 | pVsVGEVyCEERU | 88 | 704.3304 | 2 | 23.53 | 1406.66 | −9.7 | UNIQUE | 619 |
| 5307 | ELNVQEVVCEERU | 88 | 723.3579 | 2 | 23.78 | 1444.698 | 2.3 | UNIQUE | 620 |
| 6600 | pisVQELICEERU | 88 | 741.865 | 2 | 26.18 | 1481.729 | −9 | UNIQUE | 621 |
| 6644 | ELdVAEiVCEERU | 88 | 692.3489 | 2 | 26.42 | 1382.684 | −0.5 | UNIQUE | 544 |
| 14055 | EhdowEhLCEERU | 88 | 768.3787 | 2 | 37.63 | 1534.728 | 9.7 | UNIQUE | 622 |
| 6246 | ELsVAELVCEERU | 87 | 701.3533 | 2 | 25.68 | 1400.697 | −3.5 | UNIQUE | 541 |
| 7435 | pVdVAELICEERU | 86 | 699.358 | 2 | 27.85 | 1396.704 | −1.7 | UNIQUE | 319 |
| 5702 | ELsiQEVLCEERU | 85 | 735.8628 | 2 | 24.63 | 1469.721 | −7.1 | UNIQUE | 623 |
| 14577 | poNVwEmLCEERU | 85 | 788.8757 | 2 | 38.31 | 1575.73 | 4.6 | UNIQUE | 624 |
| 5109 | EVNVAEyVCEERU | 84 | 704.8279 | 2 | 23.32 | 1407.651 | −6.9 | UNIQUE | 287 |
| 6571 | pisyQELiCEERU | 83 | 758.8564 | 2 | 26.32 | 1515.719 | −13.5 | UNIQUE | 625 |
| 15573 | pobLwpVhCEERU | 83 | 776.3967 | 2 | 39.9 | 1550.776 | 2 | UNIQUE | 626 |
| 5935 | pVNVQEiyCEERU | 82 | 746.3484 | 2 | 25.09 | 1490.695 | −8.8 | UNIQUE | 627 |
| 6138 | ELNVAEyVCEERU | 82 | 711.8355 | 2 | 25.49 | 1421.667 | −7.1 | UNIQUE | 628 |
| 6807 | ELdiQEVyCEERU | 80 | 737.8516 | 2 | 26.69 | 1473.695 | −4.5 | UNIQUE | 629 |
| 7594 | pVsiApVyCEERU | 80 | 724.3705 | 2 | 28.67 | 1446.699 | 19.1 | UNIQUE | 630 |
| 13847 | pibiwEoLCEERU | 80 | 768.3806 | 2 | 37.36 | 1534.75 | −2 | UNIQUE | 631 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

Identified Binders from Peptide E6 Library I-NACbME (ALC>80)

TABLE 27
Condition I-5.1

| | | | | | | | | | SEQ |
|---|---|---|---|---|---|---|---|---|---|
| | | ALC | | | | | | Unique- | ID |
| Scan | Peptide | (%) | m/z | Z | RT | Mass | ppm | ness | NO: |
| 13649 | pkNhGEjLCEERU | 72 | 755.9042 | 2 | 37.83 | 1509.799 | −3.2 | UNIQUE | 632 |

TABLE 28
Condition I-5.2

| | | ALC | | | | | | Unique- | |
|---|---|---|---|---|---|---|---|---|---|
| Scan | Peptide | (%) | m/z | Z | RT | Mass | ppm | ness | |
| 6159 | ELTLQELLCEER | 84 | 737.8806 | 2 | 27.81 | 1473.75 | −2.1 | UNIQUE | SEQ ID |
| | U | | | | | | | | NO: 171 |

TABLE 29
Condition I-5.3

| | | | | | | | | | SEQ |
|---|---|---|---|---|---|---|---|---|---|
| | | ALC | | | | | | Unique- | ID |
| Scan | Peptide | (%) | m/z | Z | RT | Mass | ppm | ness | Nos: |
| 7670 | ELTLQELLCEERU | 94 | 737.8827 | 2 | 27.72 | 1473.75 | 0.7 | UNIQUE | 171 |
| 9310 | ELTLQELmCEERU | 92 | 761.875 | 2 | 30.44 | 1521.744 | −5.7 | UNIQUE | 210 |
| 6240 | ELsVQELyCEERU | 91 | 746.8595 | 2 | 25.2 | 1491.708 | −2.6 | UNIQUE | 633 |
| 6747 | ELNVQEVLCEERU | 89 | 730.3642 | 2 | 26.13 | 1458.714 | 0.2 | UNIQUE | 634 |
| 5843 | ELsVAEhVCEERU | 87 | 707.3544 | 2 | 24.37 | 1412.693 | 0.6 | UNIQUE | 174 |
| 14533 | pobiAEmLCEERU | 84 | 786.3826 | 2 | 38.29 | 1570.747 | 2.5 | UNIQUE | 635 |
| 15373 | posVwEjLCEERU | 84 | 784.3954 | 2 | 39.58 | 1566.774 | 1.2 | UNIQUE | 636 |
| 6725 | piNiAEVyCEERU | 80 | 723.837 | 2 | 26.03 | 1445.677 | −12.1 | UNIQUE | 278 |
| 13399 | pjbywEhLCEERU | 80 | 764.3967 | 2 | 37.64 | 1526.783 | −2.5 | UNIQUE | 637 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 30
Condition I-5.4

| | | | | | | | | | SEQ |
|---|---|---|---|---|---|---|---|---|---|
| | | ALC | | | | | | Unique- | ID |
| Scan | Peptide | (%) | m/z | Z | RT | Mass | ppm | ness | Nos: |
| 7495 | ELTLQELLCEERU | 92 | 737.8796 | 2 | 27.87 | 1473.75 | −3.5 | UNIQUE | 171 |
| 5200 | ELsVGEyVCEERU | 90 | 704.3286 | 2 | 23.47 | 1406.656 | −9.2 | UNIQUE | 172 |
| 5510 | EhsiQEVVCEERU | 90 | 734.8564 | 2 | 24.15 | 1467.702 | −2.6 | UNIQUE | 638 |
| 6598 | pVNVQELVCEERU | 89 | 730.3635 | 2 | 26.14 | 1458.718 | −3.8 | UNIQUE | 137 |
| 5969 | ELNVAEVyCEERU | 87 | 711.8357 | 2 | 24.98 | 1421.667 | −6.8 | UNIQUE | 639 |
| 5352 | pVsiGEyVCEERU | 86 | 710.3312 | 2 | 23.74 | 1418.663 | −10.8 | UNIQUE | 640 |
| 5337 | ELsVQEVVCEERU | 86 | 722.8566 | 2 | 23.75 | 1443.703 | −2.7 | UNIQUE | 641 |
| 4745 | ELsVAEVVCEERU | 84 | 694.3451 | 2 | 22.45 | 1386.681 | −4 | UNIQUE | 394 |
| 5404 | pysiQEVyCEERU | 83 | 762.8403 | 2 | 23.85 | 1523.69 | −15.9 | UNIQUE | 642 |
| 6967 | pLsVQEILCEERU | 82 | 742.8716 | 2 | 26.82 | 1483.742 | −8.7 | UNIQUE | 643 |
| 5472 | pVNiGEVyCEERU | 80 | 710.8295 | 2 | 24.06 | 1419.658 | −9.7 | UNIQUE | 644 |
| 6216 | pVsiQELyCEERU | 80 | 752.8563 | 2 | 25.61 | 1503.716 | −11.8 | UNIQUE | 482 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

TABLE 31
Condition I-5.5

| | | ALC | | | | | | Unique- | SEQ |
|---|---|---|---|---|---|---|---|---|---|
| Scan | Peptide | (%) | m/z | Z | RT | Mass | ppm | ness | ID Nos: |
| 8612 | ELTLQELLCEERU | 91 | 737.8793 | 2 | 27.77 | 1473.75 | −3.8 | UNIQUE | 171 |
| 16198 | pjNjwEiLCEERU | 90 | 768.9105 | 2 | 38.58 | 1535.81 | −2.1 | UNIQUE | 149 |
| 4901 | ELsVGEViCEERU | 87 | 693.3375 | 2 | 21.54 | 1384.669 | −5.9 | UNIQUE | 645 |
| 6496 | pVsVAEhVCEERU | 87 | 707.3535 | 2 | 24.38 | 1412.698 | −3.7 | UNIQUE | 646 |
| 6954 | zisVQELyCEERU | 87 | 763.8475 | 2 | 25.11 | 1525.66 | 13.1 | UNIQUE | 157 |
| 5088 | EVsVQEiVCEERU | 86 | 721.8486 | 2 | 21.78 | 1441.69 | −5.3 | UNIQUE | 647 |
| 6829 | pLTiGEVyCEERU | 84 | 711.3547 | 2 | 24.92 | 1420.679 | 11.3 | UNIQUE | 648 |
| 6973 | pVsVQELVCEERU | 83 | 729.8638 | 2 | 25.02 | 1457.723 | −6.6 | UNIQUE | 316 |
| 7809 | pLTiAEVyCEERU | 83 | 718.3619 | 2 | 26.5 | 1434.694 | 10.4 | UNIQUE | 649 |
| 5995 | ELsyQEiVCEERU | 81 | 745.8484 | 2 | 23.71 | 1489.696 | −9.1 | UNIQUE | 392 |
| 7881 | pLsiGELyCEERU | 81 | 724.3448 | 2 | 26.57 | 1446.694 | −13.3 | UNIQUE | 650 |
| 5795 | pysVGEiVCEERU | 80 | 710.3284 | 2 | 23.22 | 1418.663 | −14.7 | UNIQUE | 651 |
| 7449 | pLTVQEVVCEERU | 80 | 723.864 | 2 | 25.86 | 1445.723 | −6.4 | UNIQUE | 652 |
| 14900 | pLdjGEkyCEERU | 80 | 751.4075 | 2 | 36.7 | 1500.781 | 13.1 | UNIQUE | 653 |

Cyclobutylglycine (Cbg): b, Cyclopropylglycine (Cpg): d, Cyclopentylglycine (Ceg): x, Cyclopropylalanine (Cpa): i, Cyclobutylalanine (Cba): h, Cyclohexylalanine(Cha) :j, Octanonic (Aoc): k, Homo phenylalanine (Hof): m, Phenyl glycine (Phg): y, Napthyl alanine (Naf): o, Aminoadipic acid (Aad): p, Homo cysteic acid (Hoc): z, Hydroxyl proline (Hyp): s, Amino isobutyric (Aib): w, C-term amide: U.

### Example 13—Analysis of Peptide 18 HPV16E6 Binding in Crosslink Assays

To compare crosslink efficiency of peptides identified in the library screen, eleven identified peptides were synthesized as Dha peptides replacing the Cys with a DHA group. The peptides (FIG. 26A) and the parent ELT-DHA9G were mixed one to one with MBP-16E6 protein for 2 h, at 37° C. The mono-crosslinked MBP-16E6-peptide 18 conjugate was the only observed conjugate with a yield of 83%, indicating that peptide 18 outcompeted other peptides in the mixture to react with MBP-16E6 (FIG. 26B).

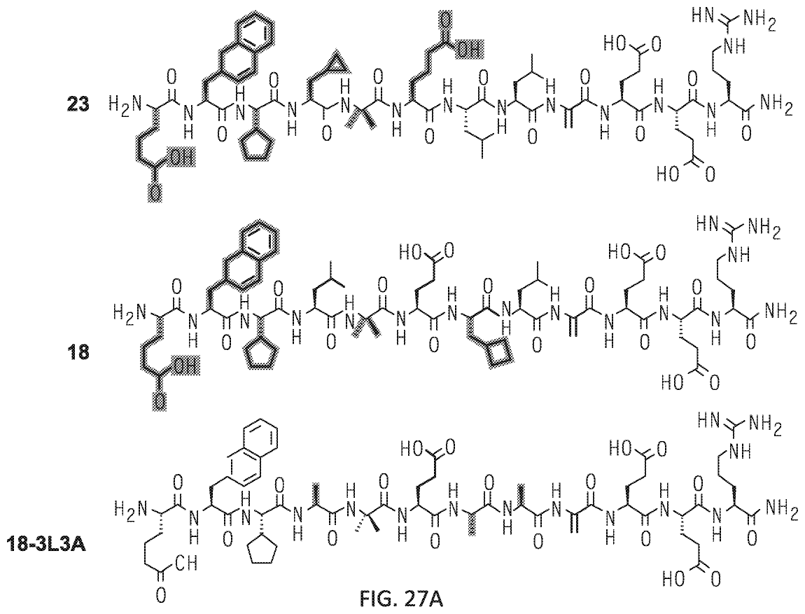

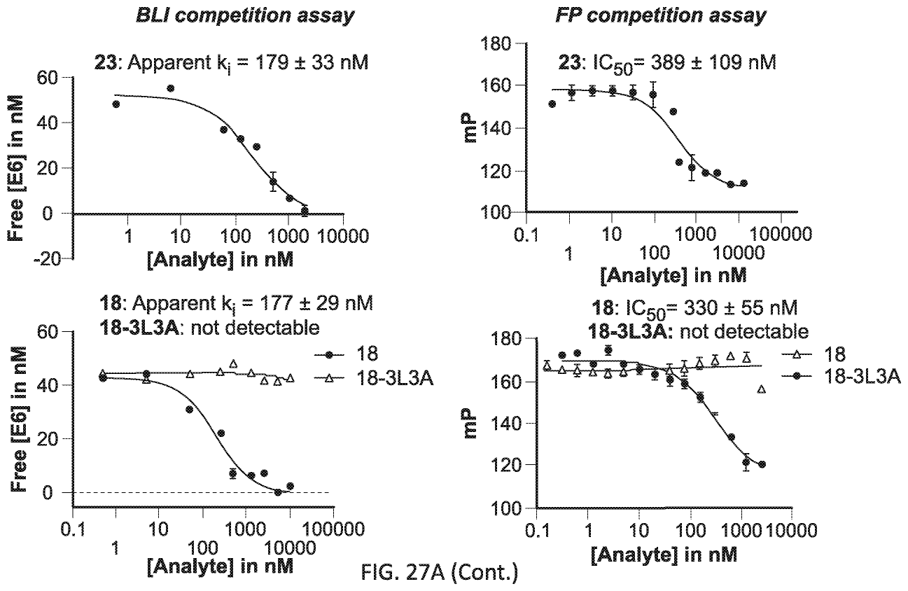

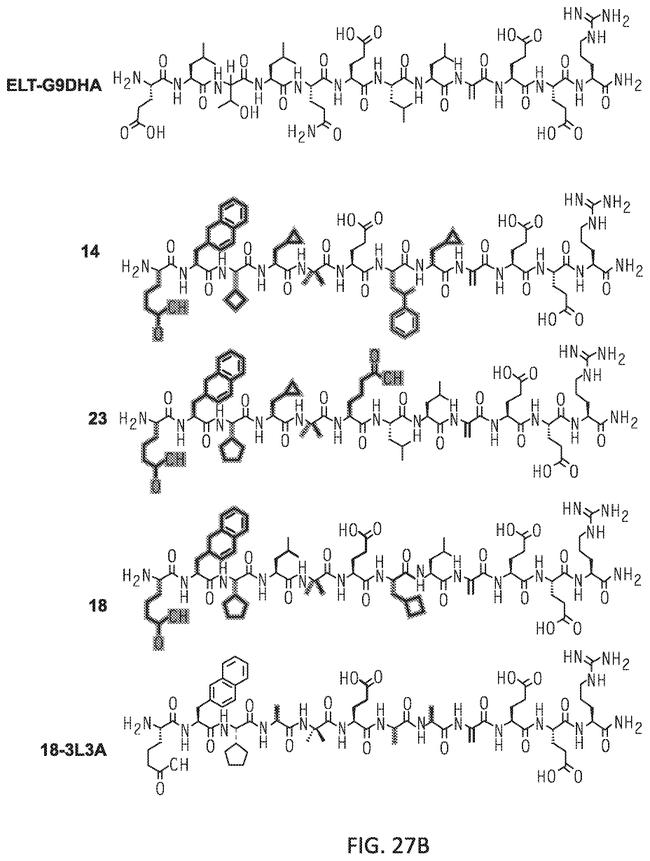

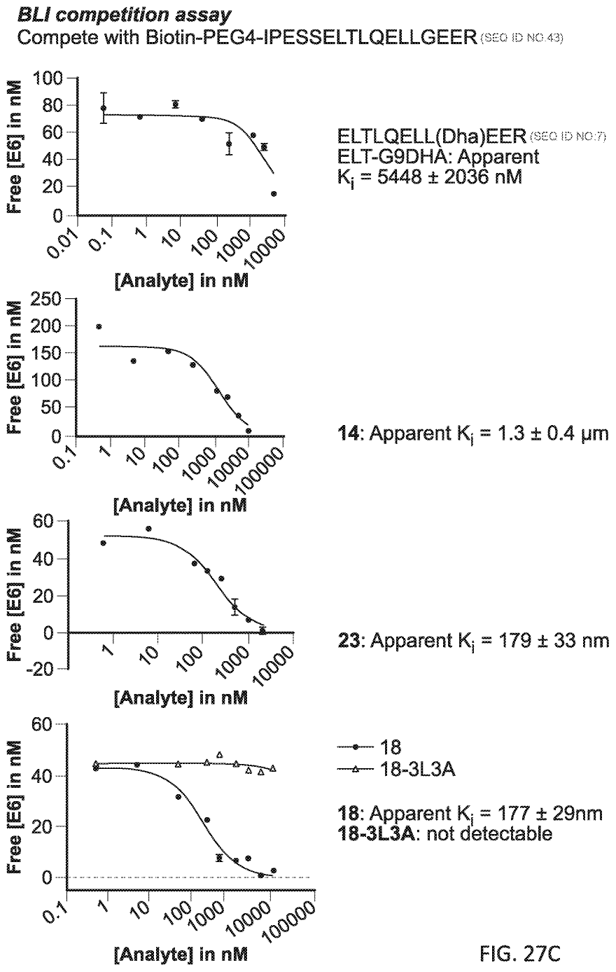

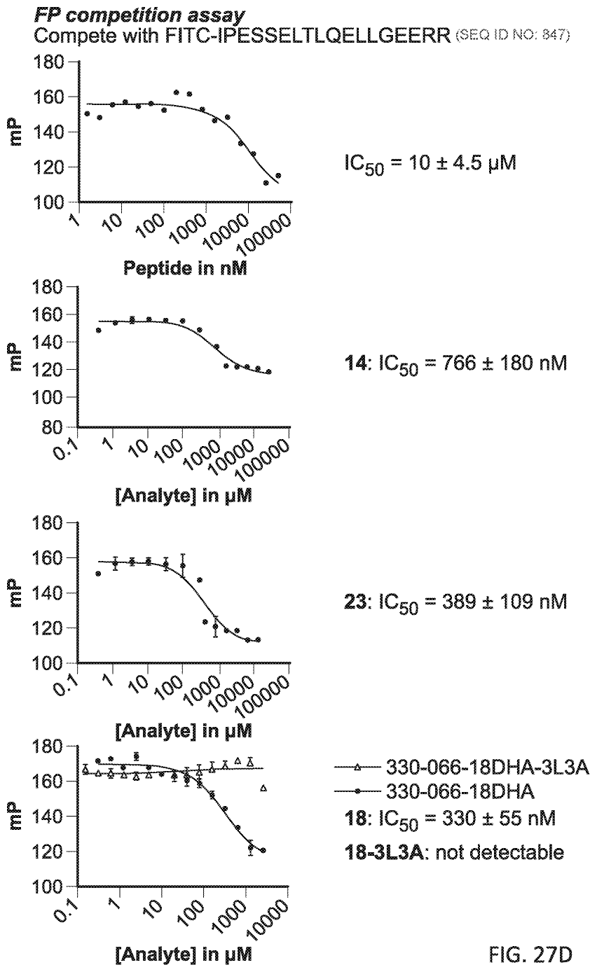

The DHA reactides were further evaluated by the BLI competition assay against peptide 1-biotin to estimate their inhibitory constant, which is report here as an apparent Kᵢ value. Six peptides showed an improved apparent Kᵢ, peptide 3 (Kᵢ of 466 nM), peptide 6 (Kᵢ of 288 nM), peptide 8 (Kᵢ of 302 nM), peptide 10 (Kᵢ of 815 nM), peptide 18 (Kᵢ of 177 nM) and peptide 23 (Kᵢ of 179 nM). The other peptides showed comparable binding affinity to the parent reactide ELT-DHA9G (FIGS. 27A-D).

Orthogonally, a fluorescent polarization assay was performed to ELT-DHA9G (IC₅₀ of 10 μM), 14 (IC₅₀ of 766 nM), 18 (IC₅₀ of 330 nM), and 23 (IC₅₀ of 389 nM). In both competitions based binding assays, peptide 18 demonstrated the highest inhibitory effect to MBP-16E6, therefore, was selected for further validation.

Peptide 18 and its structural analog peptide 23 showed improved 50-fold improved affinity compared to parent reactide ELT-DHA9G. 18 and 23 only differ in their aliphatic side chains in position 4 and 7, where Leu was replaced with cyclobutyl-alanine or cyclopropyl-alanine (FIG. 27A). The negative control derivative of 18, (18-3L3A) showed no observable binding to MBP-16E6 protein. Three major structural changes, Leu2Naf, Thr3Ceg and Gln5Aib were observed with 18, which make 18 distinct from the parent peptide ELT-DHA9G. Noticeably, substitution Leu8Hof in peptide 14 showed decrease 2-fold lower binding affinity compared to peptide 18, may showed that the position 8 has lower tolerance for aromatic modification. Compared to the Thr3Ceg in peptide 18, Thr3Cbg in 14, position 3 may require a larger ring in the cyclic sidechain to improve affinity (FIGS. 27-B-D).

TABLE 40

| | Position | | | | | | | | | | | | SEQ |
|---|---|---|---|---|---|---|---|---|---|---|---|---|---|
| Name | 1 | 2 | 3 | 4 | 5 | 6 | 7 | 8 | 9 | 10 | 11 | 12 | ID NO: |
| ELT-G9Dha | Glu | Leu | Thr | Leu | Gln | Glu | Leu | Leu | Dha | Glu | Glu | Arg | 7 |
| 3 | Aad | Nva | Hyp | Nva | Gly | Glu | Cba | Nva | Dha | Glu | Glu | Arg | 8 |
| 4 | Aad | Nva | Asn | Cpa | Gln | Glu | Leu | Nva | Dha | Glu | Glu | Arg | 9 |
| 5 | Aad | Leu | Hyp | Nva | Gln | Glu | Nva | Nva | Dha | Glu | Glu | Arg | 10 |
| 6 | Glu | Leu | Asn | Cpa | Gly | Glu | Nva | Phg | Dha | Glu | Glu | Arg | 11 |
| 8 | Aad | Nva | Asn | Nva | Gln | Glu | Leu | Nva | Dha | Glu | Glu | Arg | 12 |
| 9 | Aad | Phg | Hyp | Phg | Gln | Glu | Leu | Nva | Dha | Glu | Glu | Arg | 13 |
| 10 | Aad | Cpa | Cpg | Cpa | ala | Glu | Nva | Nva | Dha | Glu | Glu | Arg | 14 |
| 12 | Aad | Hof | Thr | Naf | ala | Glu | Leu | Cba | Dha | Glu | Glu | Arg | 15 |
| 14 | Aad | Naf | Cbg | Cpa | Aib | Glu | Hof | Cpa | Dha | Glu | Glu | Arg | 16 |
| 18 | Aad | Naf | Ceg | Leu | Aib | Glu | Cba | Leu | Dha | Glu | Glu | Arg | 17 |
| 19 | Aad | Cha | Hyp | Naf | ala | Glu | Cha | Phg | Dha | Glu | Glu | Arg | 18 |
| 23 | Aad | Naf | Ceg | Cpa | Aib | Aad | Leu | Leu | Dha | Glu | Glu | Arg | 19 |

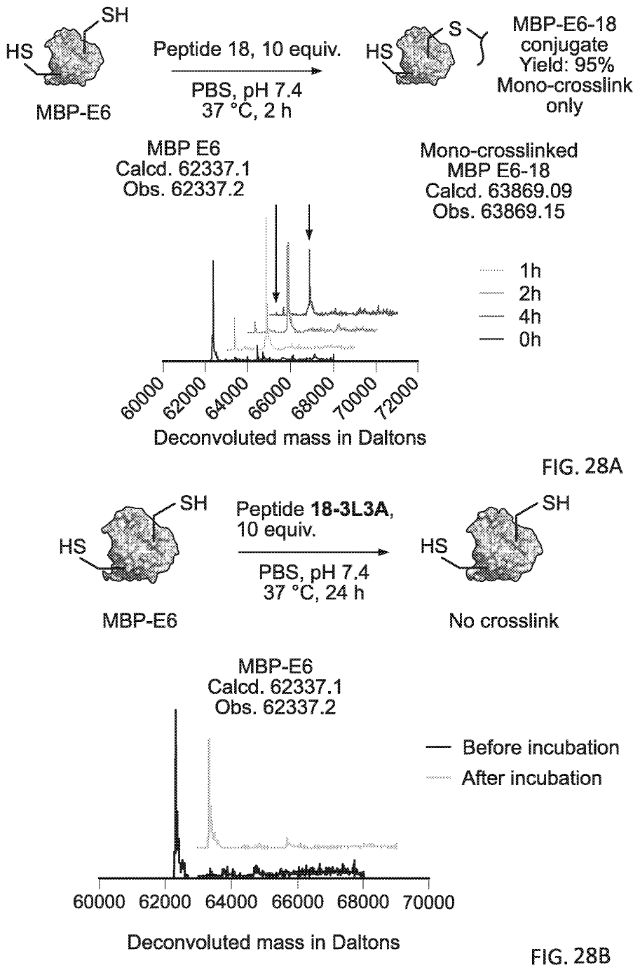

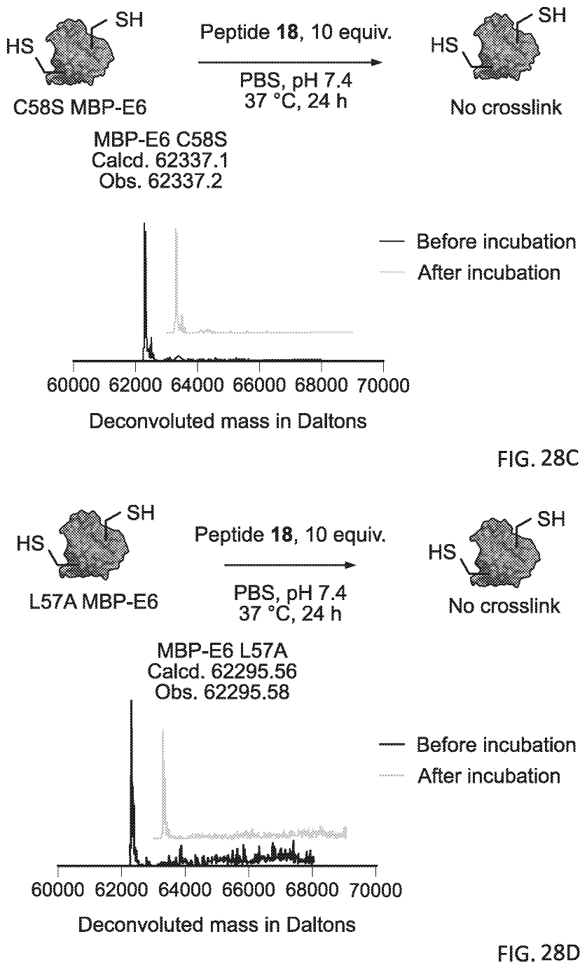

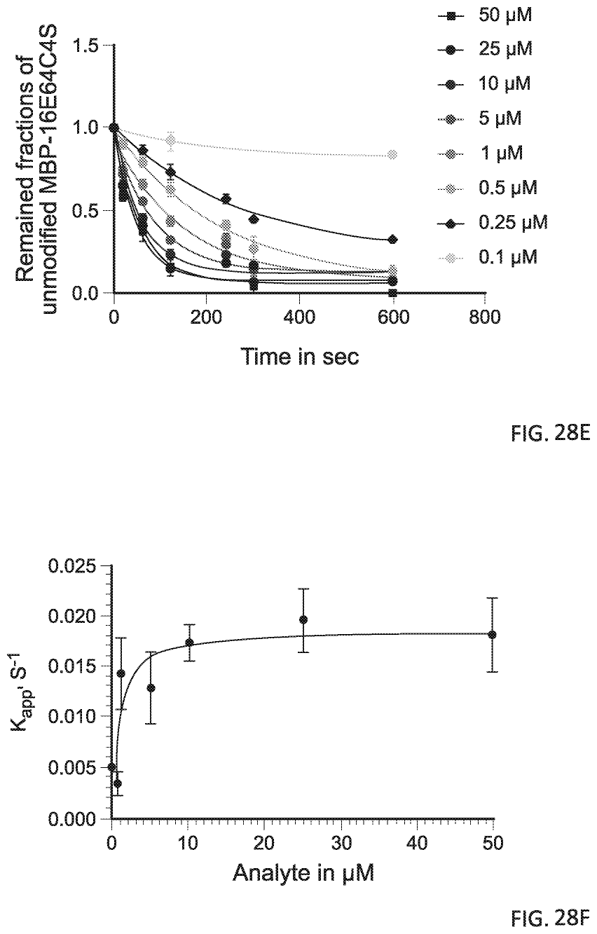

Reactide 18 was further evaluated for crosslinking MBP-16E6. Reactide 18 uses a two-stage bind-and-react strategy to covalently crosslink to MBP-16E6 and block the E6AP binding site. Reactide 18 (10 μM) and MBP-16E6 (1 μM) were incubated in PBS at 37° C. for two hours, and subsequent protein deconvolution mass spectra revealed >95% mono-crosslinking of 18 to MBP-16E6 (FIG. 28A). This was achieved despite the presence of 10 cysteine residues on MBP-16E6. with selectively crosslinks to MBP-16E6.

The negative control 18-3L3A showed no observable crosslinking to MBP-16E6 protein (FIG. 28B), supporting reactide 18 specificities. Therefore, 18-3L3A was used as a negative control for subsequent peptides. To confirm site-selectivity, reactide 18 was incubated with the MBP-16E6 mutant C58S for 12 h in PBS at 37° C. No crosslinking was observed between reactide 18 and MBP-16E6 C58S (FIG. 28C), which supports Cys58 as the sole site of MBP-16E6 modification. L57A mutant is a critical residue that are important for full-lengthen E6AP/16E6 PPI (cite), no crosslinking was observed between reactide 18 and MBP-16E6 L57A (FIG. 28D), indicating that E6AP binding activity is required for reactide 18 to crosslink.

A kinetic study was performed with reactide 18 to estimate the binding constant Kᵢ and first-order rate constant (kinact) involved in the two-stage bind-and-react strategy. Crosslink yield was monitored over time by LC-MS (FIG. 28E and FIG. 28F). Kinetic parameters were estimated assuming a steady-state approximation, and a kinact of 0.018 s-1, a Kᵢ value of 792 nM and a kinact/KI ratio of 22.7 mM-1 s-1. Reactide 18 represents a 50-fold improvement over ELT-DHA9G (kinact of 0.022 s-1, Kᵢ of 50 μM and kinact/Kᵢ ratio of 446 M-1 s-1).

### Example 14—N-Terminally Modified Libraries of Peptides

A library of E6AP-mimicking peptides (IPESS (SEQ ID NO: 884) peptide) with N-terminal small molecule modifications was further synthesized and screened for binding of 16E6.

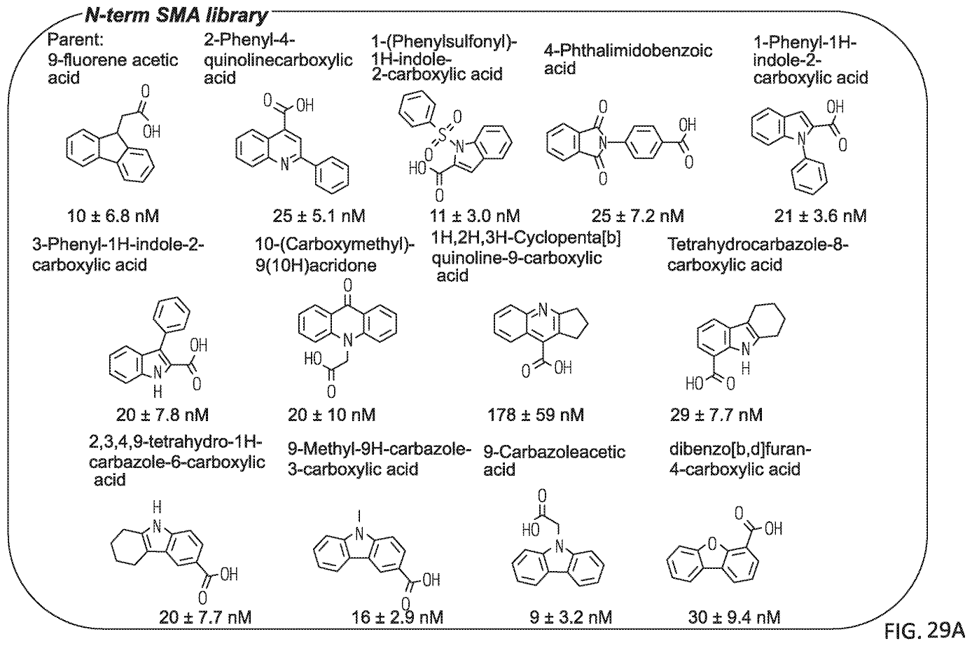

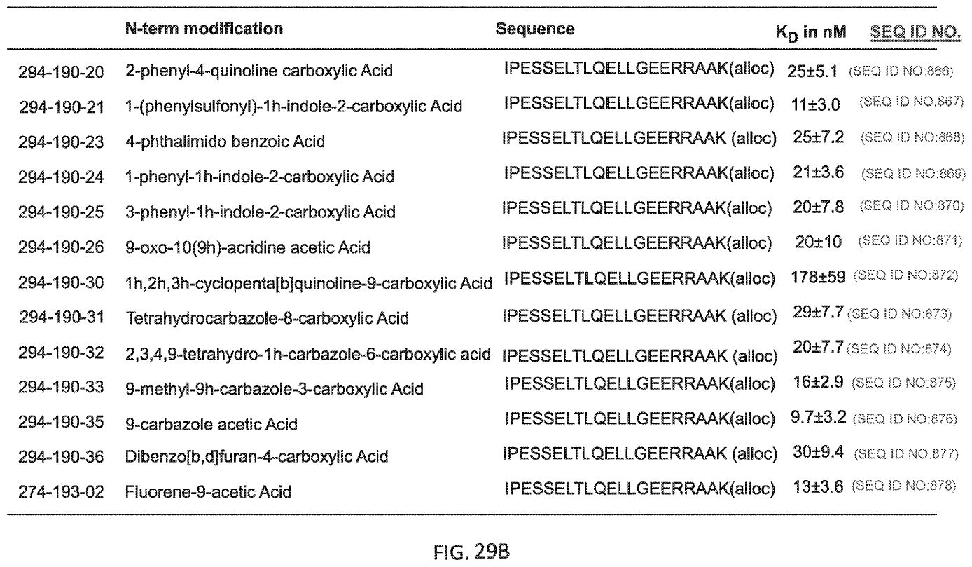

Exemplary small molecule moieties for N-terminal modifications are shown in FIG. 29A-29B. Exemplary small molecule moieties comprised: 2-Phenyl-4-quinolinecarboxylic acid, 1-(Phenylsulfonyl)-1H-indole-2-carboxylic acid, 6-Fluoro-2-naphthoic acid, 4-phthalimidobenzoic acid, Xanthene 9-carboxylic acid, 1-Pyrenebutyric acid, 5-Acenaphthenecarboxylic acid, 1-Phenyl-1H-indole-2-carboxylic acid, Indole-2-carboxlic acid, quinoline-4-carboxylic acid, and 10-(Carboxymethyl)-9(10H)acridone.

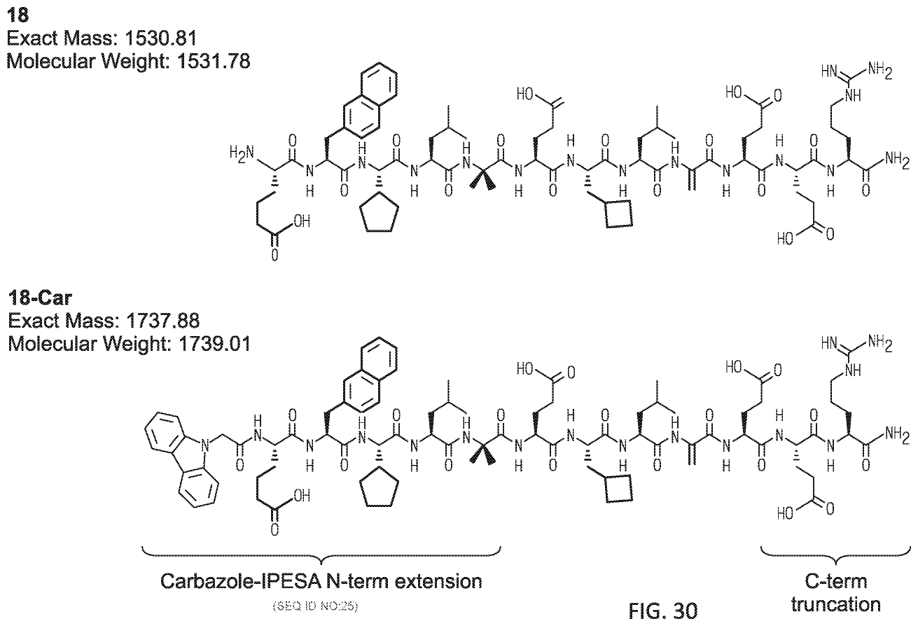

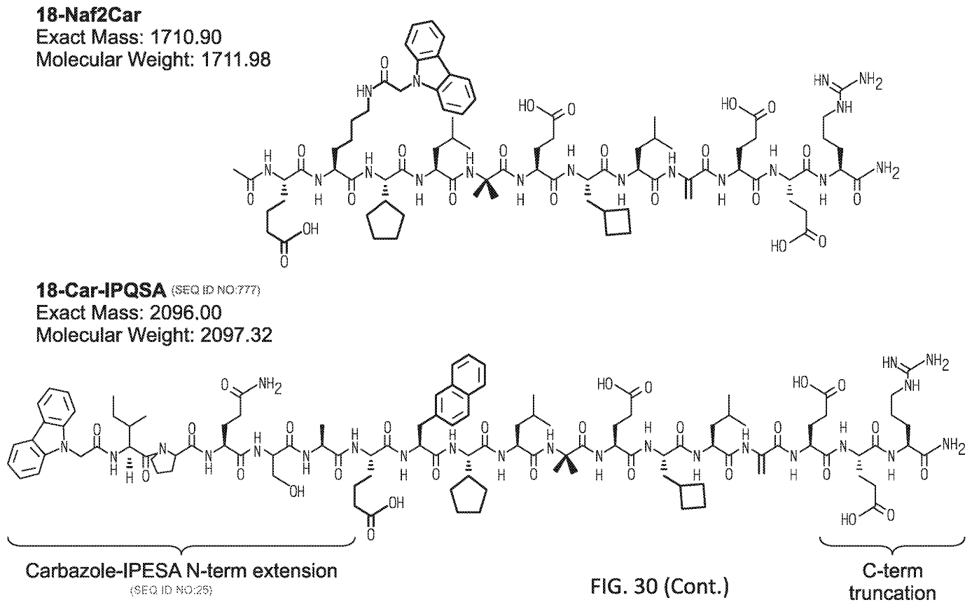

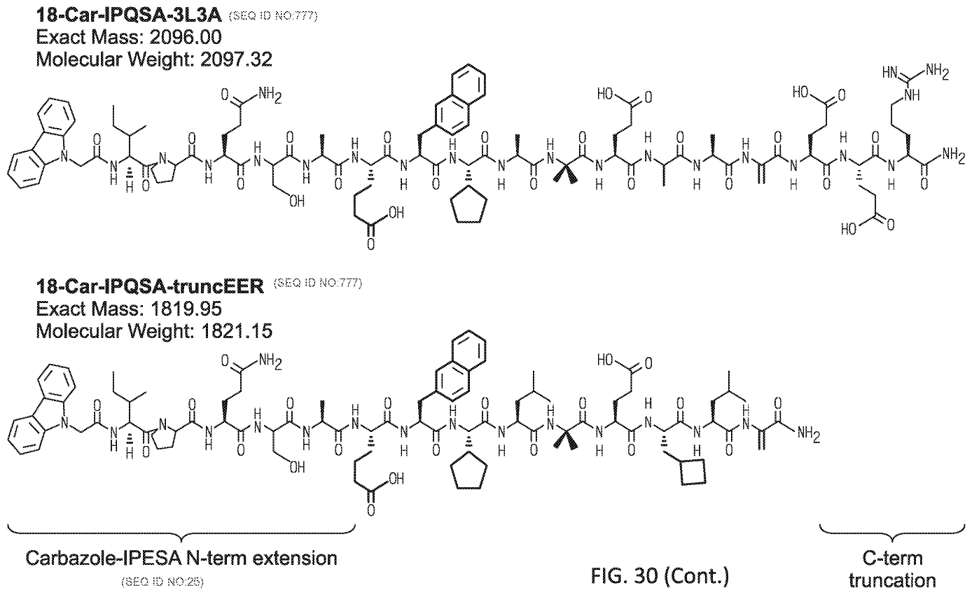

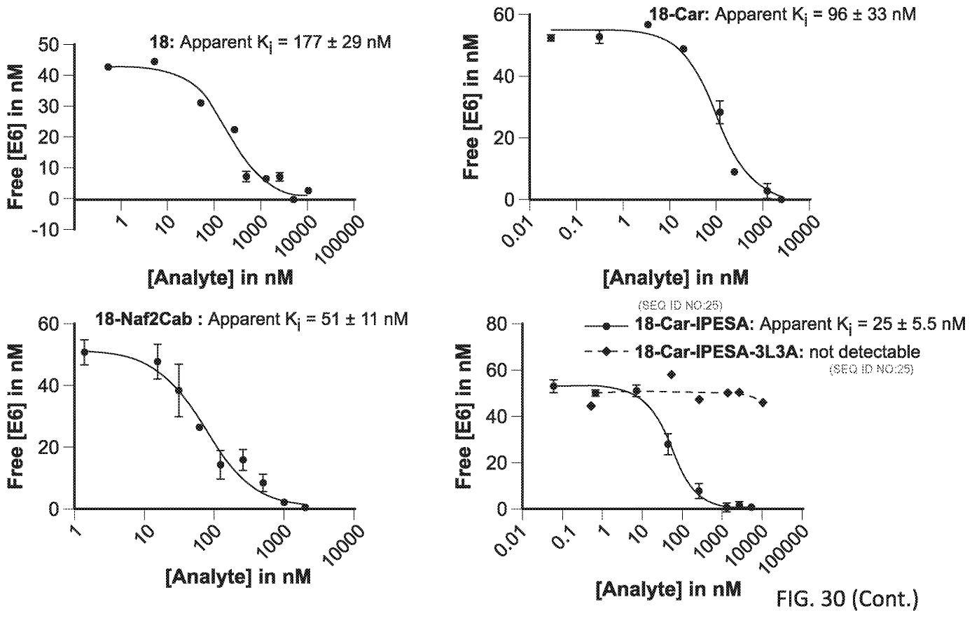

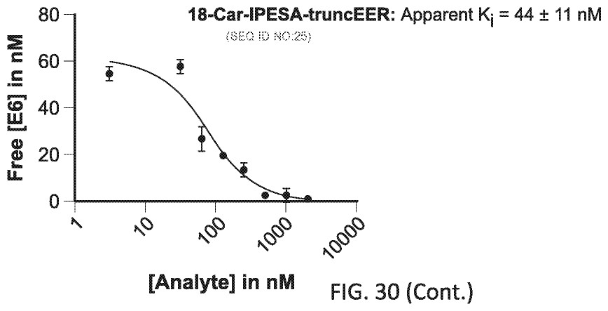

Among these moieties, fluorene showed the highest improvement. An analog campaign of fluorene reveals that N-terminal carbazole maintained the binding affinity improvement (FIG. 30). As carbazole derivatives are a common pharmacophore seen in FDA-approved drugs, i.e., Carbazomycin A, Staurosporinone, and Alectini46. Carbazole was thereafter, chosen as the small molecule to modify peptide 18.

A library of N-terminally modified peptides based on peptide 18 was further synthesized and screened for binding of 16E6. The results show, that N-term small molecule modification improve 18's binding to MBP-16E6 (FIG. 30). N-term carbazole modification improved peptide 18's binding to 16E6 by 3-fold (18-Car showed an apparent Kᵢ of 96±33 nM). Substitution of the Naf2 position with Lys (Carbazole acetamido) improved binding, for example 18-Naf2Cab showed an apparent Kᵢ of 51±11 nM.

N-term elongated peptides of the E6 binder were previously shown to have a binding K_{D} of 1-10 nM. Reactide 18 with the N-terminal Carbazole-IPQSA (SEQ ID NO: 777) modification (18-Car-IPQSA (SEQ ID NO: 777)) showed an apparent Kᵢ of 25±5.5 nM. This N-term extension improved reactide 18's binding to 16E6 by 7-fold. The negative control derivative of 18-Car-IPQSA (SEQ ID NO:777), (18-Car-IPESA (SEQ ID NO: 25)-3L3A) showed no detectable binding to MBP-16E6 protein. Further truncation of C-term EER residues from the peptide do not significantly affect binding where 18-Car-IPQSA (SEQ ID NO: 777)-truncEER showed an apparent Kᵢ of 44±11 nM.

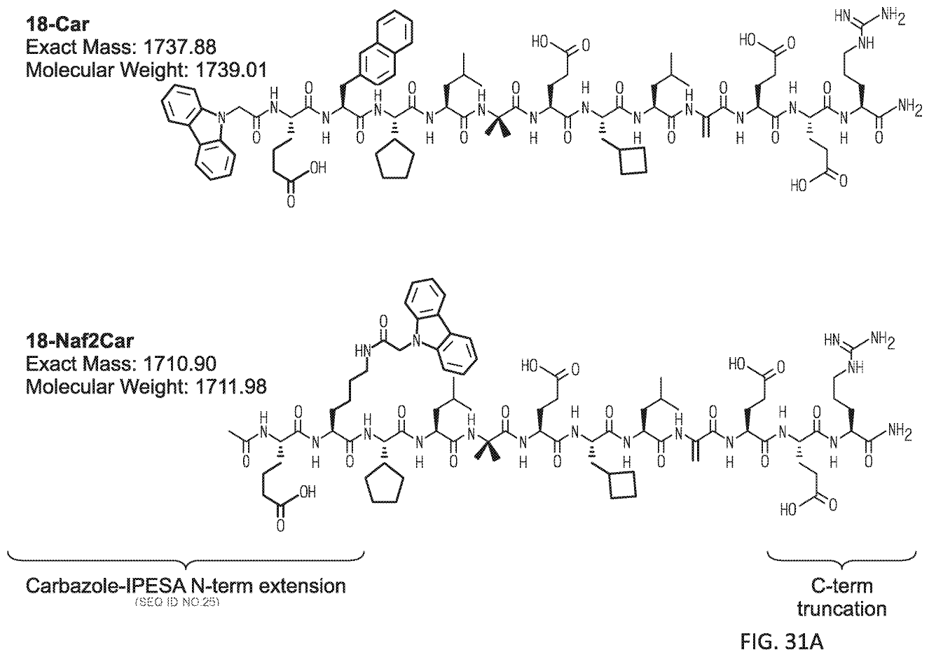

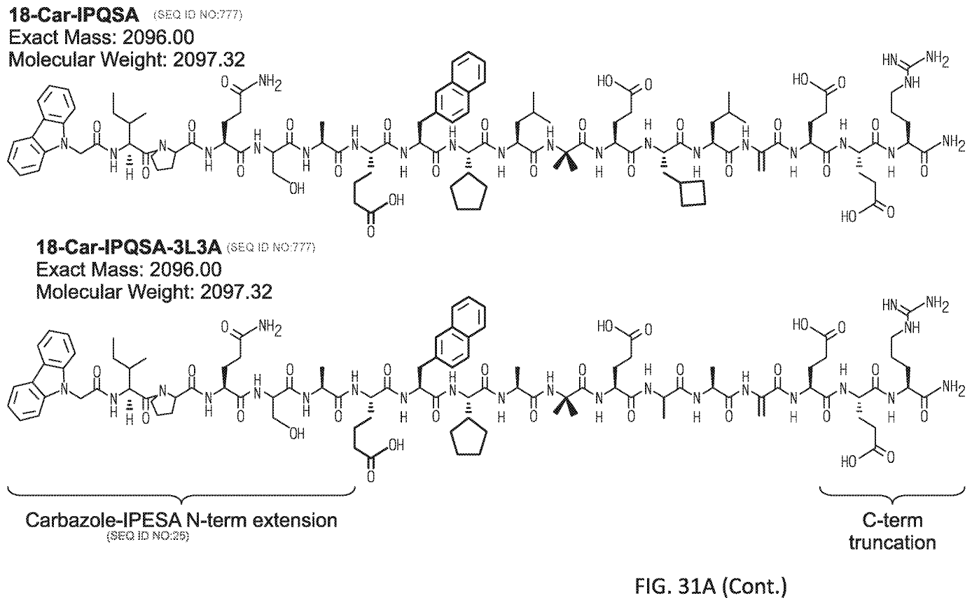

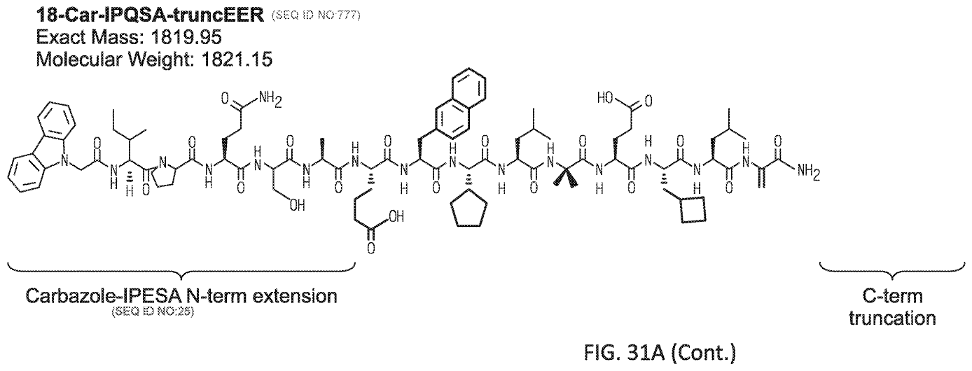

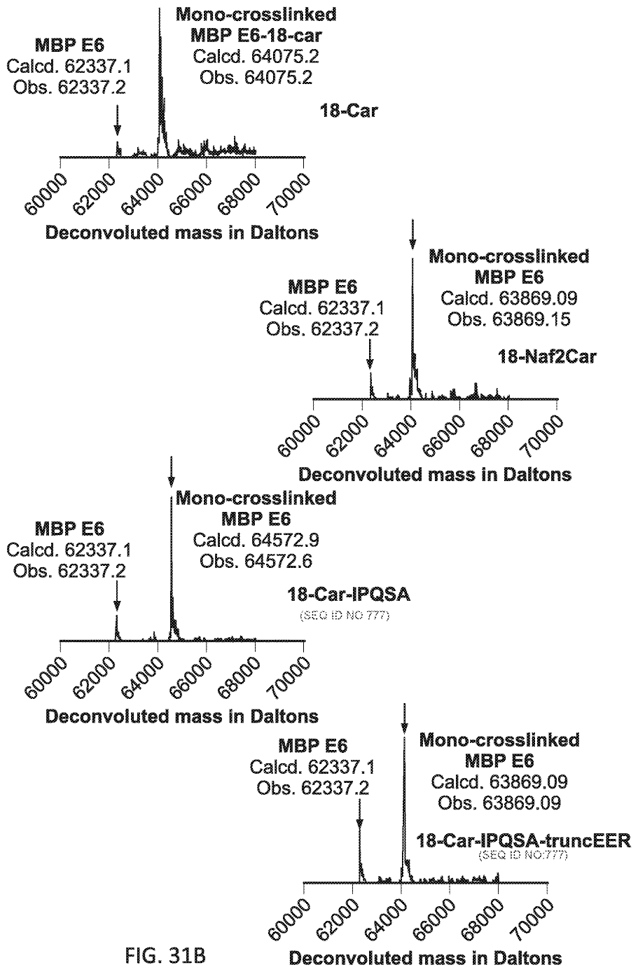

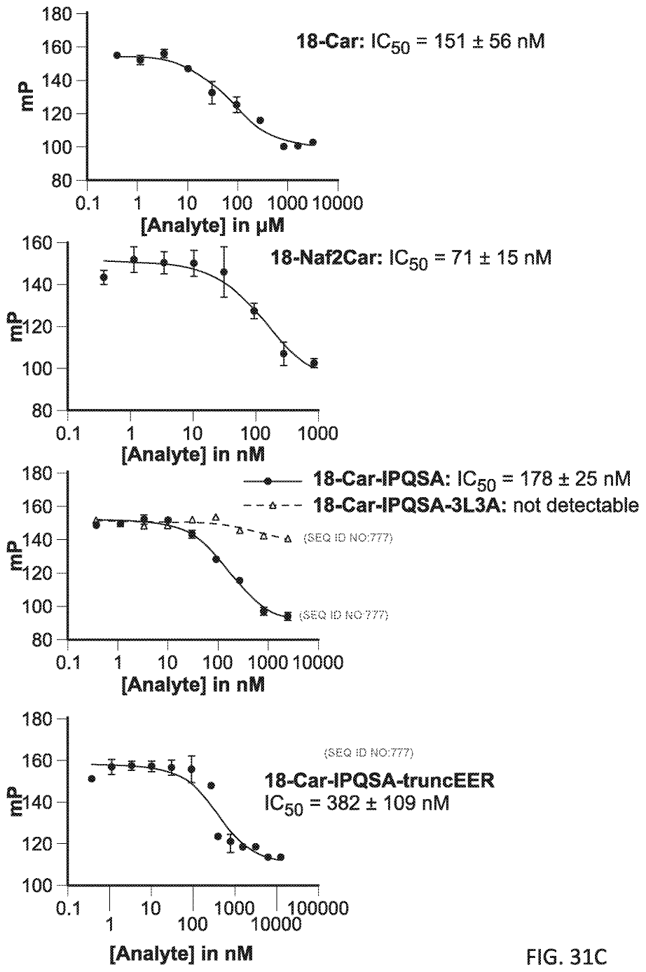

Crosslink assay was performed with peptide 18 analogs. (FIG. 31A-31C). 18-Car, 18-Naf2Cab, 18-Car-IPQSA (SEQ ID NO: 777) and 18-Car-IPQSA (SEQ ID NO: 777)-truncEER achieved quantitatively mono-crosslink in 2 h at 37° C., 10 eq. to protein 16E6. The negative control derivative of 18-Car-IPESA(SEQ ID NO: 25)-3L3A showed no crosslink to MBP-16E6 protein supporting 18-Car-IPESA (SEQ ID NO: 25)specificities. Therefore, N-term extension and small molecule modifications can be viable approach to improve binding affinity and binding specificity. The structure-activity relationship (SAR) study expands the molecular diversity of peptide 18.

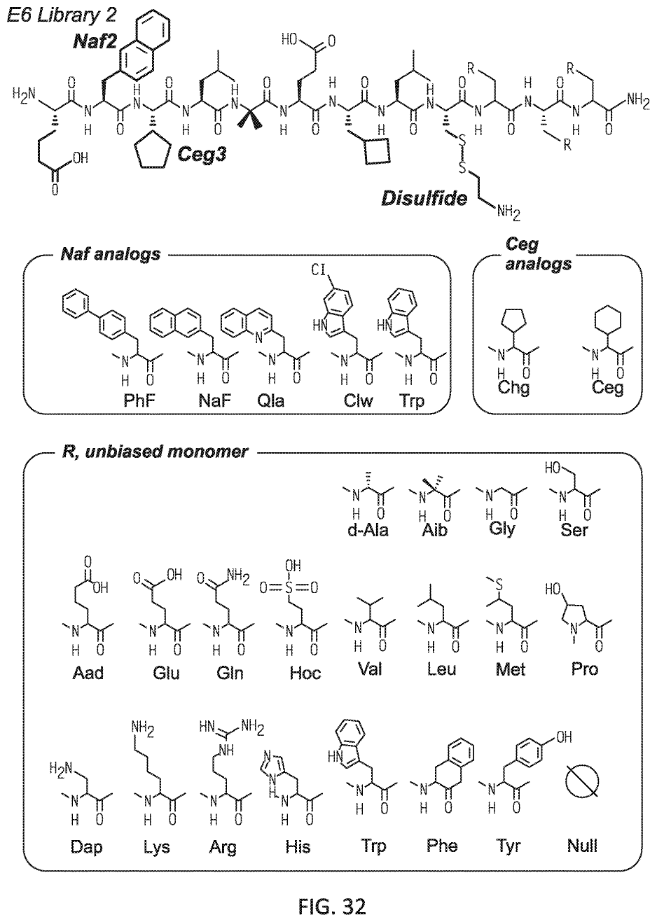

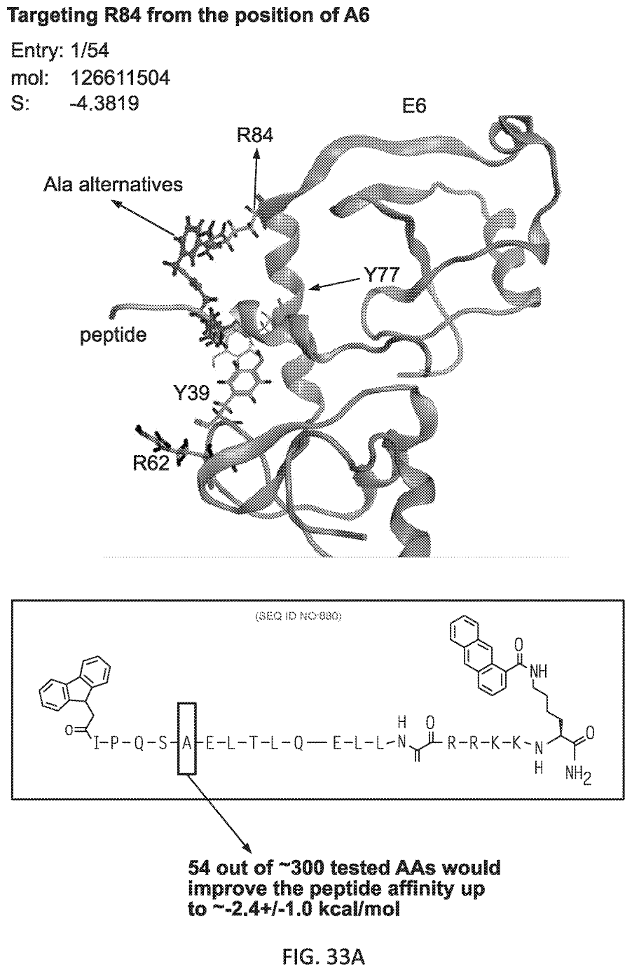

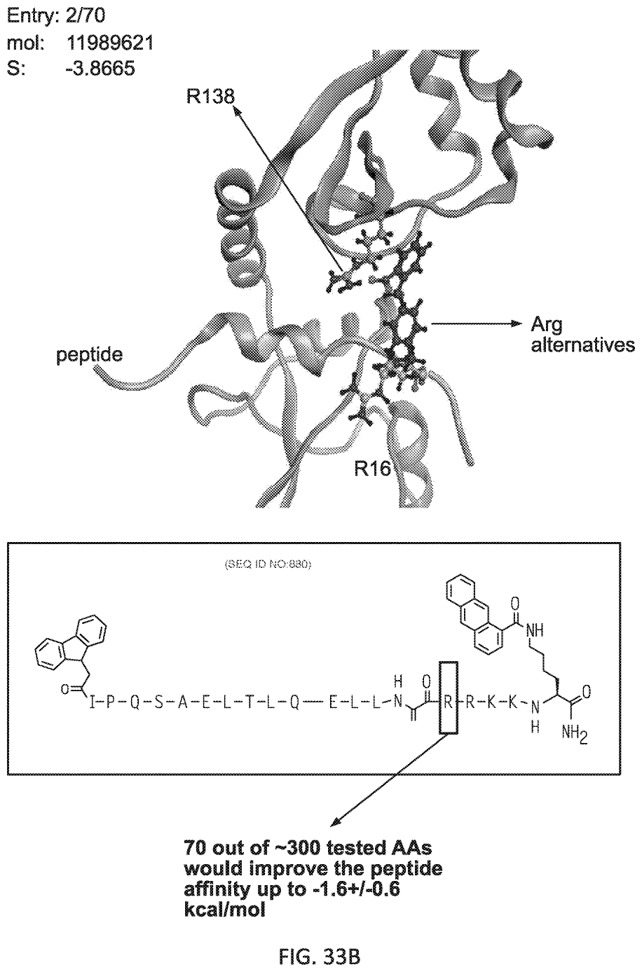

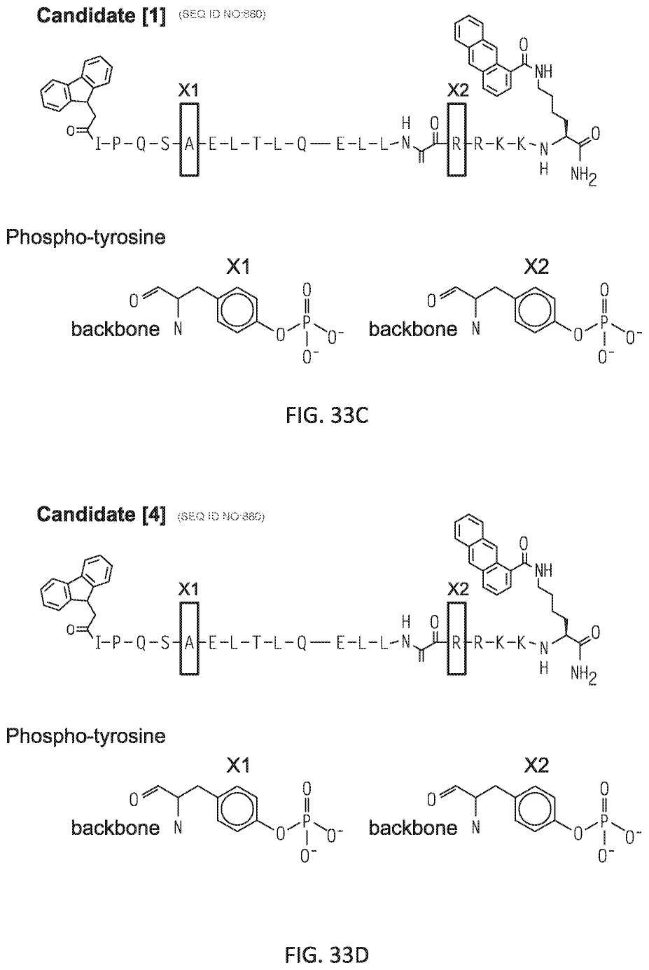

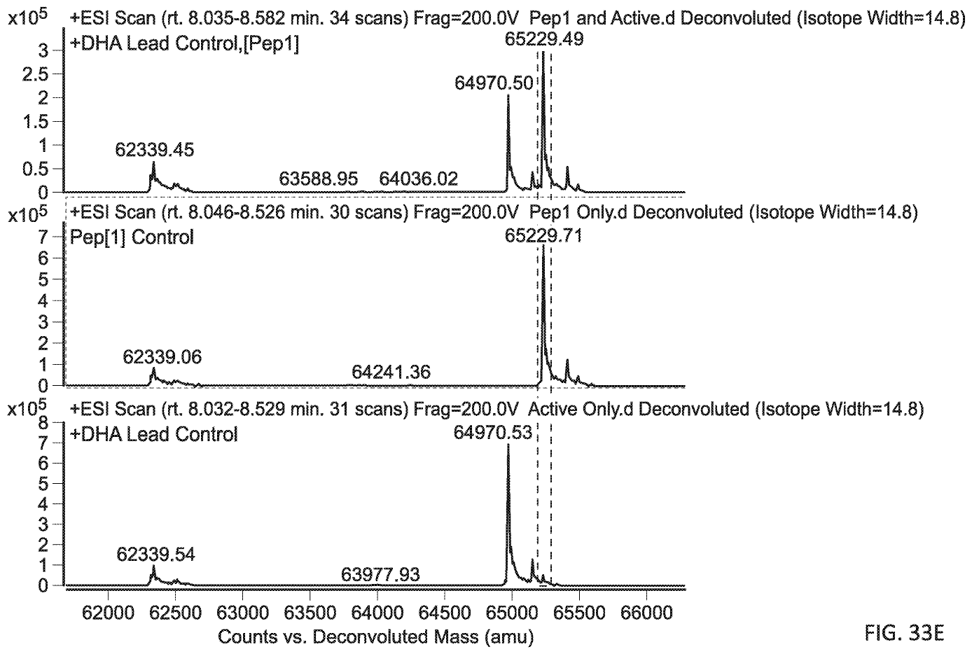

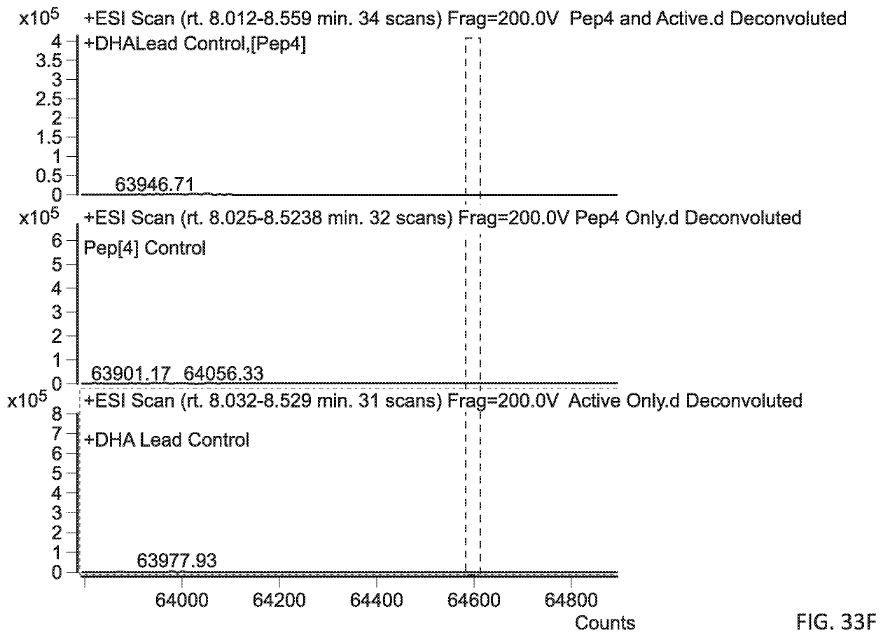

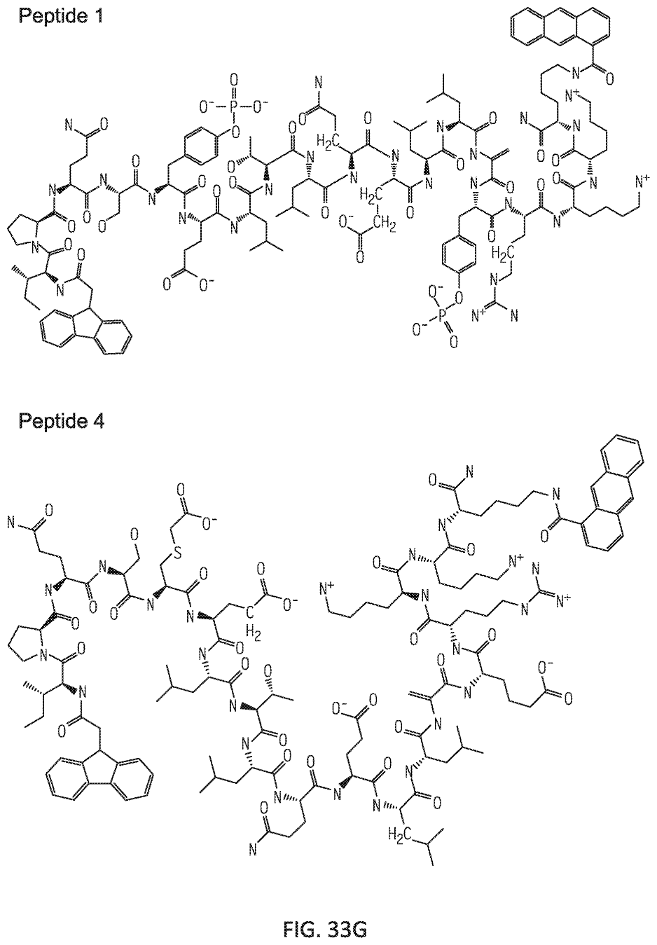

To further modify peptide 18, a peptide 18 based E6 library II-βME was designed (Aad-X₁-X₂-Leu-Aib-Aad-Cba-Leu-Cys-X₃-X₃-X₃) (SEQ ID NO: 20) composed of 80,000 members (FIG. 32, Tables 32-39). The X₁ Naf2 position was mutated to Naphthalene analogs (PhF, Naf, Qua, Clw, and Trp) based on the SAR findings. The X₂ Ceg3 position was mutated to Chg and Ceg. The last three residues positions X₃ were mutated to d-Ala, Aib, Gly, Ser, Aad, Glu, Gln, Hoc, Val, Leu, Met, Pro, Dap, Lys, Arg, His, Trp, Phe, Tyr, and Null. The library was designed with a focus on the C-term EER residues, which were randomized into 20 unbiased monomer sets a special ‘null’ monomer. The ‘null’ was skipped for coupling to create a set of C-term truncated peptides. Seven rounds of ReAct-ASMS were performed on 16E6 with five conditions. Only conditions with more than 1 ng per member enriched binders, 2-20 unique hits with ALC >90 were identified.

TABLE 32
Quality control of E6 library II

| | | ALC | | | | | | Unique- | SEQ ID |
|---|---|---|---|---|---|---|---|---|---|
| Scan | Peptide | (%) | m/z | Z | RT | Mass | ppm | ness | Nos: |
| 11378 | boiLdExLCKbp | 97 | 775.4165 | 2 | 38.76 | 1548.812 | 4.2 | UNIQUE | 654 |
| 11595 | boiLAExLCVRd | 92 | 746.4147 | 2 | 38.73 | 1490.818 | −2.4 | UNIQUE | 655 |
| 7451 | boxLdExLCKKQ | 91 | 768.4343 | 2 | 32.96 | 1534.84 | 8.9 | UNIQUE | 656 |
| 12032 | bjxLdExLCKAp | 91 | 745.4066 | 2 | 39.43 | 1488.784 | 9.5 | UNIQUE | 657 |
| 12044 | boiLAExLCVRG | 90 | 732.4033 | 2 | 40.04 | 1462.793 | −0.5 | UNIQUE | 658 |
| 11196 | boiLAExLCREp | 88 | 775.4046 | 2 | 38.17 | 1548.788 | 4.1 | UNIQUE | 659 |
| 7874 | boiLAEiLCKKp | 84 | 767.9349 | 2 | 33.67 | 1533.855 | 0.5 | UNIQUE | 660 |
| 8173 | boiLdExLCRRd | 83 | 781.938 | 2 | 34.12 | 1561.861 | 0.2 | UNIQUE | 661 |
| 12303 | boiLdExLCYQL | 83 | 785.4209 | 2 | 39.62 | 1568.818 | 6 | UNIQUE | 662 |
| 9876 | bWiLAExLCHbp | 82 | 767.3911 | 2 | 36.45 | 1532.764 | 2.4 | UNIQUE | 663 |
| 8367 | boiLdExLCRKb | 80 | 796.9442 | 2 | 34.39 | 1591.87 | 2.1 | UNIQUE | 664 |
| 11611 | boiLtExLCQLE | 80 | 768.9289 | 2 | 38.72 | 1535.815 | 18.4 | UNIQUE | 665 |
| 7439 | boxLdExLCKQK | 79 | 768.4328 | 2 | 32.94 | 1534.84 | 7 | UNIQUE | 666 |
| 10952 | boiLAEiLCKQp | 79 | 767.9165 | 2 | 37.85 | 1533.818 | 0.2 | UNIQUE | 667 |
| 11141 | bWiLAExLCALW | 79 | 755.9039 | 2 | 38.09 | 1509.8 | −4.5 | UNIQUE | 668 |
| 12710 | boiLdExLCVER | 79 | 775.4237 | 2 | 40.02 | 1548.824 | 5.7 | UNIQUE | 669 |
| 13073 | boiLAEiLCRAd | 79 | 739.4093 | 2 | 40.7 | 1476.807 | −2.1 | UNIQUE | 670 |
| 10933 | boiLdExLCKEM | 76 | 777.4058 | 2 | 37.79 | 1552.79 | 4.7 | UNIQUE | 671 |
| 11581 | boiLtExLCdER | 74 | 768.9104 | 2 | 38.68 | 1535.821 | −9.3 | UNIQUE | 672 |
| 12218 | boiLdExLCVKG | 74 | 725.4096 | 2 | 39.51 | 1448.797 | 5.5 | UNIQUE | 673 |
| 12042 | boiLdExLCHbp | 73 | 779.8938 | 2 | 39.28 | 1557.776 | −2 | UNIQUE | 674 |
| 9906 | bWxLtExLCHAQ | 72 | 739.386 | 2 | 36.49 | 1476.762 | −3.2 | UNIQUE | 675 |
| 11829 | boiLAEiLCpEp | 71 | 760.8815 | 2 | 39 | 1519.75 | −1.1 | UNIQUE | 676 |
| 12036 | boiLAEiLCVKp | 71 | 753.4219 | 2 | 39.43 | 1504.828 | 0.9 | UNIQUE | 677 |
| 12736 | bjiLdExLCpKE | 71 | 781.4 | 2 | 40.18 | 1560.81 | −15.8 | UNIQUE | 678 |
| 8374 | boiLdExLCdRR | 70 | 781.9387 | 2 | 34.36 | 1561.861 | 1.2 | UNIQUE | 679 |
| 11162 | boxLtExLCHGQ | 70 | 737.8794 | 2 | 38.12 | 1473.749 | −2.9 | UNIQUE | 680 |
| 11776 | bWiLdExLCHVQ | 70 | 759.9087 | 2 | 38.93 | 1517.796 | 4.7 | UNIQUE | 681 |
| 8539 | boiLtExLCQAK | 69 | 747.4119 | 2 | 34.64 | 1492.82 | −7.5 | UNIQUE | 682 |
| 11766 | bWiLdExLCpYt | 69 | 758.9056 | 2 | 38.92 | 1515.781 | 10.5 | UNIQUE | 683 |
| 10756 | boxLtExLCYpp | 67 | 771.381 | 2 | 37.5 | 1540.759 | −7.1 | UNIQUE | 684 |
| 12949 | bjiLAExLCVRp | 67 | 773.4269 | 2 | 40.5 | 1544.832 | 4.6 | UNIQUE | 685 |
| 7736 | boxLdEiLCKtp | 65 | 746.9164 | 2 | 33.46 | 1491.815 | 2.5 | UNIQUE | 686 |
| 8770 | boiLAExLCHKV | 65 | 758.4207 | 2 | 34.95 | 1514.824 | 1.8 | UNIQUE | 687 |
| 11228 | boiLdExLCHQQ | 64 | 779.8985 | 2 | 38.26 | 1557.788 | −3.5 | UNIQUE | 688 |
| 12400 | boiLAEiLCVpt | 64 | 732.4171 | 2 | 39.76 | 1462.798 | 14.9 | UNIQUE | 689 |
| 10909 | boiLAExLCFWd | 60 | 785.4053 | 2 | 37.78 | 1568.797 | −0.3 | UNIQUE | 690 |

Position 3 Cyclopentylglycine (Ceg): x, Cyclohexylglycine (Chg): i, position 7 Cyclobutylalanine (Cba): x, 4-phenyl phenylalanine(Phf): j, 6-C1 tryptophan (CIW): m, QuinolylAlanine (Qua): n, Napthylalanine(Naf): o, Aminoadipic acid (Aad): b, Homocysteic acid (Hoc): z, Hydroxyproline (Hyp): p, Amino isobutyric (Aib): d

TABLE 33
Identified binders from E6 library II

| | | | Number of | Number of |
|---|---|---|---|---|
| | | MBP | binder ALC > 90 | binder ALC > 80 |
| Name | Library (1.2M) | 16E6 | (12-mer, 11-mer) | (12-mer, 11-mer) |
| II-1 | 40 μg library | 40 μg | 0, 0 | 0, 0 |
| | (0.5 ng/member) | | | |
| II-2 | 80 μg library | 40 μg | 20, 2 | 36, 3 |
| | (1 ng/member) | | | |
| II-3.1 | 100 μg library | 20 μg | 13, 0 | 31, 3 |
| | (1.25 ng/member) | | | |
| II-3.2 | 100 μg library | 20 μg | 2, 1 | 6, 1 |
| | (1.25 ng/member) | | | |
| II-3.3 | 100 μg library | 20 μg | 15, 0 | 22, 0 |
| | (1.25 ng/member) | | | |
| II-4 | 200 μg library | 20 μg | 7, 0 | 14, 2 |
| | (2.5 ng/member) | | | |
| II-5 | 300 μg library | 20 μg | 2, 0 | 3, 3 |
| | (3.75 ng/member) | | | |

330-091-01ASMS: Condition II-1: 40 μg library (0.5 ng/member), MBP-E6 40 μg (No hit)
330-091-02ASMS: Condition II-2: 80 μg library (1 ng/member), MBP-E6 40 μg
330-092-01ASMS: Condition II-3.1: 100 μg library (1.25 ng/member), MBP-E6 20 μg
330-092-02ASMS: Condition II-3.2: 100 μg library (1.25 ng/member), MBP-E6 20 μg
330-092-03ASMS: Condition II-3.3: 100 μg library (1.25 ng/member), MBP-E6 20 μg
330-092-05ASMS: Condition II-4: 200 μg library (2.5 ng/member), MBP-E6 20 μg
330-092-04ASMS: Condition II-5: 300 μg library (3.75 ng/member), MBP-E6 20 μg

TABLE 34
Condition II-2

| | Peptide | ALC | | | | | | Unique- | SEQ ID |
|---|---|---|---|---|---|---|---|---|---|
| Scan | 12-mer | (%) | m/z | Z | RT | Mass | ppm | ness | Nos: |
| 9211 | bWiLdExLCbHp | 97 | 774.4008 | 2 | 37.49 | 1546.774 | 8.4 | UNIQUE | 691 |
| 9069 | bWiLdExLCdRE | 91 | 762.9122 | 2 | 37.18 | 1523.801 | 6.2 | UNIQUE | 692 |
| 12509 | WjxLdExLCAKK | 88 | 774.4422 | 2 | 43.54 | 1546.854 | 10.6 | UNIQUE | 693 |
| | Peptide | ALC | | | | | | Unique- | SEQ ID |
| Scan | 11-mer | (%) | m/z | Z | RT | Mass | ppm | ness | Nos: |
| 10770 | boxLdExLCRH | 87 | 722.8878 | 2 | 40.28 | 1443.752 | 6.2 | UNIQUE | 694 |
| 11931 | bjxLdExLCKR | 84 | 731.4149 | 2 | 42.35 | 1460.806 | 6.5 | UNIQUE | 695 |
| 11284 | RnxLdExLCRK | 80 | 725.413 | 2 | 41.19 | 1448.816 | −3.5 | UNIQUE | 696 |

Position 3 Cyclopentylglycine (Ceg): x, CycloHexylglycine (Chg): i, position 7 Cyclobutylalanine (Cba): x, 4-phenyl phenylalanine(Phf): j, 6-C1 tryptophan (CIW): m, QuinolylAlanine (Qua): n, Napthylalanine(Naf): o, Aminoadipic acid (Aad): b, Homocysteic acid (Hoc): z, Hydroxyproline (Hyp): p, Amino isobutyric (Aib): d

TABLE 35
Condition II-3.1

| | Peptide | ALC | | | | | | Unique- | SEQ ID |
|---|---|---|---|---|---|---|---|---|---|
| Scan | 12-mer | (%) | m/z | Z | RT | Mass | ppm | ness | Nos: |
| 7898 | bWiLdExLCKbp | 99 | 769.9179 | 2 | 36.73 | 1537.81 | 7.3 | UNIQUE | 698 |
| 10213 | boiLdExLCVAK | 97 | 732.4222 | 2 | 40.88 | 1462.812 | 12.1 | UNIQUE | 699 |
| 9661 | boxLdExLCVGK | 94 | 718.3994 | 2 | 39.91 | 1434.777 | 5.3 | UNIQUE | 700 |
| 8506 | boxLdExLCQRd | 93 | 760.9122 | 2 | 37.84 | 1519.799 | 7.5 | UNIQUE | 701 |
| 7767 | bWiLdExLCQRd | 92 | 762.4196 | 2 | 36.52 | 1522.816 | 5.4 | UNIQUE | 702 |
| 8651 | boxLdExLCKbp | 90 | 768.4146 | 2 | 38.16 | 1534.792 | 14.5 | UNIQUE | 703 |
| 9083 | boxLdExLCGRd | 90 | 725.3954 | 2 | 38.86 | 1448.762 | 10.2 | UNIQUE | 704 |
| 7502 | bWxLdExLCKFH | 89 | 776.9178 | 2 | 36 | 1551.812 | 5.9 | UNIQUE | 705 |
| 8603 | boxLdExLCdRG | 88 | 725.3952 | 2 | 38.08 | 1448.762 | 9.9 | UNIQUE | 706 |
| 10546 | boiLdExLCAVK | 87 | 732.4175 | 2 | 41.5 | 1462.812 | 5.6 | UNIQUE | 707 |
| 7990 | bWiLdExLCRGp | 86 | 740.9042 | 2 | 36.95 | 1479.775 | 12.9 | UNIQUE | 708 |
| 7914 | bWxLdExLCdMY | 84 | 760.385 | 2 | 36.82 | 1518.741 | 9.7 | UNIQUE | 709 |
| 9029 | boiLdExLCKEp | 81 | 768.4141 | 2 | 38.81 | 1534.792 | 14.1 | UNIQUE | 710 |
| 9110 | boiLdExLCKbp | 81 | 775.4219 | 2 | 38.96 | 1548.812 | 11.1 | UNIQUE | 654 |
| | Peptide | ALC | | | | | | Unique- | SEQ ID |
| Scan | 11-mer | (%) | m/z | Z | RT | Mass | ppm | ness | Nos: |
| 9688 | boiLAEiLCQR | 83 | 725.3954 | 2 | 38.86 | 1448.782 | −3.6 | UNIQUE | 697 |
| 9838 | boiLAExLCRR | 81 | 732.4131 | 2 | 40.21 | 1462.804 | 5.2 | UNIQUE | 752 |

Position 3 Cyclopentylglycine (Ceg): x, CycloHexylglycine (Chg): i, position 7 Cyclobutylalanine (Cba): x, 4-phenyl phenylalanine(Phf): j, 6-C1 tryptophan (CIW): m, QuinolylAlanine (Qua): n, Napthylalanine(Naf): o, Aminoadipic acid (Aad): b, Homocysteic acid (Hoc): z, Hydroxyproline (Hyp): p, Amino isobutyric (Aib): d

TABLE 36
Condition II-3.2

| | Peptide | ALC | | | | | | Unique- | SEQ ID |
|---|---|---|---|---|---|---|---|---|---|
| Scan | 12-mer | (%) | m/z | Z | RT | Mass | ppm | ness | Nos: |
| 8113 | bWiLdExLCGKb | 99 | 741.8891 | 2 | 38.09 | 1481.789 | −16.8 | UNIQUE | 711 |
| 8633 | bWiLdExLCAbR | 98 | 762.918 | 2 | 38.97 | 1523.811 | 7.2 | UNIQUE | 712 |
| 6980 | bWxLdExLCKQA | 97 | 734.4079 | 2 | 35.94 | 1466.78 | 14.3 | UNIQUE | 713 |
| 8011 | boxLdExLCbKQ | 96 | 775.9196 | 2 | 37.89 | 1549.808 | 10.9 | UNIQUE | 714 |
| 8987 | bWiLdExLCKVb | 95 | 762.9294 | 2 | 39.64 | 1523.836 | 5.6 | UNIQUE | 715 |
| 9600 | bWiLdExLCbVR | 95 | 776.933 | 2 | 40.71 | 1551.842 | 6.2 | UNIQUE | 716 |
| 7306 | bWiLdExLCQRd | 94 | 762.4192 | 2 | 36.19 | 1522.816 | 4.9 | UNIQUE | 702 |
| 8404 | boxLdExLCbQH | 94 | 780.4025 | 2 | 38.6 | 1558.772 | 11.8 | UNIQUE | 717 |
| 10027 | boiLdExLCVAK | 94 | 732.4146 | 2 | 41.5 | 1462.812 | 1.7 | UNIQUE | 699 |
| 8459 | boxLdExLCbHQ | 93 | 780.4025 | 2 | 38.6 | 1558.772 | 11.8 | UNIQUE | 718 |
| 8699 | boxLdExLCbGH | 91 | 744.8862 | 2 | 39.12 | 1487.735 | 15.5 | UNIQUE | 719 |
| 8877 | boiLdExLCREE | 91 | 790.4153 | 2 | 39.52 | 1578.798 | 11.4 | UNIQUE | 720 |
| 9188 | boiLdExLCVWb | 91 | 797.4235 | 2 | 39.68 | 1592.822 | 6.5 | UNIQUE | 721 |
| 8480 | boiLdExLCLFH | 90 | 781.9266 | 2 | 38.74 | 1561.823 | 9.8 | UNIQUE | 722 |
| 8880 | boxLdExLCKEF | 90 | 778.4177 | 2 | 39.45 | 1554.798 | 14.8 | UNIQUE | 723 |
| 9013 | boiLdExLCRbE | 87 | 797.4235 | 2 | 39.68 | 1592.818 | 9 | UNIQUE | 724 |
| 9187 | boiLdExLCLWb | 87 | 804.4321 | 2 | 39.99 | 1606.838 | 7.4 | UNIQUE | 725 |
| 10876 | boiLdExLCWHH | 85 | 813.4177 | 2 | 43.06 | 1624.809 | 7.3 | UNIQUE | 726 |
| 8133 | boxLdExLCMVp | 83 | 747.8912 | 2 | 38.48 | 1493.743 | 16.3 | UNIQUE | 727 |
| 8233 | boxLdExLCbKG | 82 | 740.4008 | 2 | 38.31 | 1478.771 | 11 | UNIQUE | 728 |
| 8060 | boxLdExLCdRG | 80 | 725.3955 | 2 | 37.99 | 1448.762 | 10.3 | UNIQUE | 706 |
| 12918 | bWiLdExLCGVE | 80 | 720.3849 | 2 | 46.32 | 1438.742 | 9.1 | UNIQUE | 729 |

Position 3 Cyclopentylglycine (Ceg): x, CycloHexylglycine (Chg): i, position 7 Cyclobutylalanine (Cba): x, 4-phenyl phenylalanine(Phf): j, 6-C1 tryptophan (C1W): m, QuinolylAlanine (Qua): n, Napthylalanine(Naf): o, Aminoadipic acid (Aad): b, Homocysteic acid (Hoc): z, Hydroxyproline (Hyp): p, Amino isobutyric (Aib): d

TABLE 37
Condition II-3.3

| | Peptide | ALC | | | | | | Unique- | SEQ ID |
|---|---|---|---|---|---|---|---|---|---|
| Scan | 12-mer | (%) | m/z | Z | RT | Mass | ppm | ness | Nos: |
| 6562 | boiLAExLCKVp | 92 | 746.4188 | 2 | 39.59 | 1490.808 | 10.1 | UNIQUE | 730 |
| 6680 | bjxLdExLCKbp | 90 | 781.4214 | 2 | 39.64 | 1560.81 | 11.6 | UNIQUE | 731 |
| 6363 | boxLdEiLCdKQ | 88 | 753.9178 | 2 | 38.98 | 1505.813 | 5.7 | UNIQUE | 732 |
| 6385 | boxLdEiLCdKQ | 87 | 753.9267 | 2 | 39.04 | 1505.813 | 17.5 | NOT UNIQUE | 732 |
| 6245 | boiLdExLCdRp | 85 | 760.4169 | 2 | 38.33 | 1518.803 | 10.8 | UNIQUE | 733 |
| 6433 | boxLdExLCKAG | 83 | 704.3904 | 2 | 39.12 | 1406.745 | 14.9 | UNIQUE | 734 |
| 6117 | boxLdExLCdRG | 78 | 725.3954 | 2 | 38.17 | 1448.762 | 10.3 | UNIQUE | 706 |
| 8532 | boiLdExLCVpQ | 68 | 753.4082 | 2 | 46.14 | 1504.782 | 13.5 | UNIQUE | 735 |
| 8104 | boiLdExLCHHH | 63 | 788.9081 | 2 | 44.76 | 1575.789 | 8.4 | UNIQUE | 736 |
| 6326 | boxLVExLCtYE | 58 | 772.403 | 2 | 38.97 | 1542.784 | 4.8 | UNIQUE | 737 |
| | Peptide | ALC | | | | | | Unique- | SEQ ID |
| Scan | 11-mer | (%) | m/z | Z | RT | Mass | ppm | ness | NO: |
| 6403 | boxLdExLCKQ | 95 | 704.3729 | 2 | 39.09 | 1406.745 | −10.1 | UNIQUE | 738 |

Position 3 Cyclopentylglycine (Ceg): x, CycloHexylglycine (Chg): i, position 7 Cyclobutylalanine (Cba): x, 4-phenyl phenylalanine(Phf): j, 6-C1 tryptophan (CIW): m, QuinolylAlanine (Qua): n, Napthylalanine(Naf): o, Aminoadipic acid (Aad): b, Homocysteic acid (Hoc): z, Hydroxyproline (Hyp): p, Amino isobutyric (Aib): d

TABLE 38
Condition II-4

| | Peptide | ALC | | | | | | Unique- | SEQ ID |
|---|---|---|---|---|---|---|---|---|---|
| Scan | 12-mer | (%) | m/z | Z | RT | Mass | ppm | ness | Nos: |
| 7856 | bjiLdExLCKbp | 98 | 788.428 | 2 | 40.15 | 1574.83 | 7.2 | UNIQUE | 739 |
| 7296 | boiLdExLCbKp | 97 | 775.4211 | 2 | 39.14 | 1548.812 | 10.2 | UNIQUE | 740 |
| 7698 | boxLdExLCKGV | 96 | 718.3984 | 2 | 39.91 | 1434.777 | 4 | UNIQUE | 741 |
| 8060 | bjiLdExLCVER | 95 | 788.4294 | 2 | 40.65 | 1574.842 | 1.6 | UNIQUE | 742 |
| 8356 | bjxLdExLCVGK | 95 | 731.4125 | 2 | 41.27 | 1460.794 | 10.9 | UNIQUE | 743 |
| 8018 | bjxLdExLCYLQ | 94 | 791.4242 | 2 | 40.51 | 1580.816 | 11.5 | UNIQUE | 744 |
| 8005 | bjiLdExLCbpK | 93 | 788.431 | 2 | 40.55 | 1574.83 | 11 | UNIQUE | 745 |
| 8490 | boiLdExLCFKp | 93 | 777.425 | 2 | 41.34 | 1552.818 | 11.3 | UNIQUE | 746 |
| 7418 | boxLdExLCVRQ | 92 | 767.926 | 2 | 39.34 | 1533.82 | 11.5 | UNIQUE | 747 |
| 7359 | boiLdExLCQbR | 91 | 796.9303 | 2 | 39.17 | 1591.834 | 7.6 | UNIQUE | 748 |
| 8295 | bjxLdExLCGKd | 91 | 724.4051 | 2 | 41.12 | 1446.773 | 15.5 | UNIQUE | 749 |
| 8507 | bjxLdExLCVAK | 91 | 738.4205 | 2 | 41.57 | 1474.81 | 11.2 | UNIQUE | 750 |
| 8118 | bjiLdExLCbHp | 90 | 792.9119 | 2 | 40.87 | 1583.794 | 9.6 | UNIQUE | 751 |
| 6664 | bWxLdExLCYRd | 89 | 772.9131 | 2 | 37.39 | 1543.801 | 6.8 | UNIQUE | 753 |
| 7869 | bjxLdExLCGRd | 89 | 738.4037 | 2 | 40.26 | 1474.779 | 9.3 | UNIQUE | 754 |
| 8093 | bjxLdExLCGKA | 89 | 717.3977 | 2 | 40.82 | 1432.763 | 12.3 | UNIQUE | 755 |
| 7671 | bWiLdExLCKVG | 88 | 719.9104 | 2 | 39.87 | 1437.794 | 8.2 | UNIQUE | 756 |
| 7859 | bjiLdExLCFLH | 88 | 794.9324 | 2 | 40.25 | 1587.841 | 5.8 | UNIQUE | 757 |
| 8003 | bjxLdExLCGKG | 88 | 710.39 | 2 | 40.69 | 1418.748 | 12.6 | UNIQUE | 758 |
| 7053 | boiLdExLCKFH | 87 | 789.4293 | 2 | 38.59 | 1576.834 | 6.4 | UNIQUE | 759 |
| 7455 | boiLdExLCKQd | 87 | 753.9211 | 2 | 39.43 | 1505.813 | 10.1 | UNIQUE | 760 |
| 9076 | boxLdExLCVVK | 87 | 739.4266 | 2 | 42.74 | 1476.824 | 10.2 | UNIQUE | 761 |
| 6671 | boxLdExLCKEM | 86 | 770.402 | 2 | 37.67 | 1538.77 | 12.8 | UNIQUE | 762 |
| 8952 | boiLdExLCLAK | 86 | 739.4092 | 2 | 42.47 | 1476.828 | −16.3 | UNIQUE | 763 |
| 7184 | boxLdExLCQHb | 85 | 780.4032 | 2 | 38.83 | 1558.772 | 12.7 | UNIQUE | 764 |
| 8210 | bjiLdExLCdRG | 83 | 745.4109 | 2 | 40.97 | 1488.799 | 5.4 | UNIQUE | 765 |
| 8310 | boxLdExLCKFp | 82 | 770.4178 | 2 | 41.08 | 1538.798 | 15.1 | UNIQUE | 766 |
| 8373 | bjiLdExLCKVp | 82 | 766.4321 | 2 | 41.3 | 1530.836 | 9.2 | UNIQUE | 767 |
| 6934 | boxLdExLCdRG | 81 | 725.3948 | 2 | 38.28 | 1448.762 | 9.4 | UNIQUE | 706 |
| 7868 | bjiLdExLCEKp | 81 | 781.4221 | 2 | 40.22 | 1560.81 | 12.6 | UNIQUE | 768 |
| 8006 | bjiLdExLCYLH | 80 | 802.9313 | 2 | 40.55 | 1603.836 | 7.5 | UNIQUE | 769 |
| | Peptide | ALC | | | | | | Unique- | SEQ ID |
| Scan | 11-mer | (%) | m/z | Z | RT | Mass | ppm | ness | Nos: |
| 8469 | bjxLdExLCKQ | 88 | 717.3971 | 2 | 41.49 | 1432.763 | 11.5 | UNIQUE | 770 |
| 8477 | boiLdExL CHR | 87 | 729.8939 | 2 | 41.45 | 1457.772 | 1.1 | UNIQUE | 771 |
| 7104 | boxLdExLCLb | 81 | 704.3885 | 2 | 38.88 | 1406.739 | 17.1 | UNIQUE | 772 |

Position 3 Cyclopentylglycine (Ceg): x, CycloHexylglycine (Chg): i, position 7 Cyclobutylalanine (Cba): x, 4-phenyl phenylalanine(Phf): j, 6-Cl tryptophan (CIW): m, QuinolylAlanine (Qua): n, Napthylalanine(Naf): o, Aminoadipic acid (Aad): b, Homocysteic acid (Hoc): z, Hydroxyproline (Hyp): p, Amino isobutyric (Aib): d, C-term amide: U.

TABLE 39
Condition II-5

| | Peptide | ALC | | | | | | Unique- | SEQ ID |
|---|---|---|---|---|---|---|---|---|---|
| Scan | 12-mer | (%) | m/z | Z | RT | Mass | ppm | ness | Nos: |
| 7552 | bWiLdExLCEQH | 97 | 774.9003 | 2 | 36.89 | 1547.77 | 10.6 | UNIQUE | 773 |
| 7721 | bWiLdExLCbHp | 97 | 774.3949 | 2 | 37.57 | 1546.774 | 0.9 | UNIQUE | 691 |
| 9102 | boiLdExLCVAQ | 97 | 732.4012 | 2 | 38.39 | 1462.776 | 8.2 | UNIQUE | 774 |
| 10317 | bjiLdExLCVAK | 97 | 745.4128 | 2 | 41.84 | 1488.83 | −12.8 | UNIQUE | 775 |
| 10038 | bjxLdExLCVAK | 96 | 738.4169 | 2 | 41.66 | 1474.81 | 6.2 | UNIQUE | 750 |
| 8410 | boiLdExLCKEp | 95 | 768.4165 | 2 | 38.8 | 1534.792 | 17.2 | UNIQUE | 710 |
| 9291 | boiLdExLCAEK | 95 | 747.4048 | 2 | 40.38 | 1492.786 | 5.8 | UNIQUE | 776 |
| 9862 | bjiLdExLCKVp | 95 | 766.4261 | 2 | 40.5 | 1530.836 | 1.3 | UNIQUE | 767 |
| 8213 | boiLdExLCQQL | 94 | 767.9173 | 2 | 38.47 | 1533.813 | 4.6 | UNIQUE | 778 |
| 5866 | boiLdExLCKRd | 94 | 767.9344 | 2 | 33.77 | 1533.855 | −0.5 | UNIQUE | 779 |
| 9039 | bjxLdExLCEdR | 94 | 774.4143 | 2 | 39.96 | 1546.801 | 8.7 | UNIQUE | 780 |
| 7011 | bWxLdExLCQKb | 93 | 770.4219 | 2 | 36.33 | 1538.806 | 15.1 | UNIQUE | 781 |
| 9313 | boiLdExLCRVp | 93 | 767.4294 | 2 | 40.43 | 1532.824 | 13.3 | UNIQUE | 782 |
| 8341 | boiLdExLCKQA | 93 | 746.9169 | 2 | 39.17 | 1491.803 | 11.3 | UNIQUE | 783 |
| 8950 | bWxLdExLCbVK | 92 | 755.9186 | 2 | 39.76 | 1509.816 | 4.6 | UNIQUE | 784 |
| 9992 | bWiLdExLCVVK | 92 | 740.9333 | 2 | 41.63 | 1479.842 | 7.2 | UNIQUE | 785 |
| 8009 | bWiLdExLCVKG | 92 | 719.9115 | 2 | 38.05 | 1437.794 | 9.7 | UNIQUE | 786 |
| 9382 | bjxLdExLCKAQ | 92 | 752.9213 | 2 | 40.18 | 1503.8 | 18.4 | UNIQUE | 787 |
| 6910 | bWxLdExLCFKH | 91 | 776.9159 | 2 | 36.06 | 1551.812 | 3.4 | UNIQUE | 788 |
| 9428 | boiLdExLCAKd | 91 | 725.3933 | 2 | 39.72 | 1448.791 | −13.1 | UNIQUE | 789 |
| 8006 | bWiLdExLCVKp | 90 | 747.9232 | 2 | 37.89 | 1493.816 | 10.7 | UNIQUE | 790 |
| 6984 | bWxLdExLCLFH | 89 | 769.415 | 2 | 36.16 | 1536.801 | 9.4 | UNIQUE | 791 |
| 9523 | bjxLdExLCVtp | 88 | 738.4008 | 2 | 40.83 | 1474.786 | 0.7 | UNIQUE | 792 |
| 7615 | bWiLdExLCtHF | 87 | 762.9116 | 2 | 37.35 | 1523.802 | 4.4 | UNIQUE | 793 |
| 8536 | boiLdExLCbKp | 86 | 775.4216 | 2 | 39.13 | 1548.812 | 10.8 | UNIQUE | 740 |
| 8837 | bjxLdExLCdRG | 86 | 738.4034 | 2 | 39.52 | 1474.779 | 8.8 | UNIQUE | 794 |
| 7965 | bWiLdExLCKYp | 83 | 779.9204 | 2 | 38 | 1557.811 | 9.9 | UNIQUE | 795 |
| 9017 | boiLdExLCKdA | 83 | 725.3933 | 2 | 39.72 | 1448.791 | −13.1 | UNIQUE | 796 |
| 8255 | boxLdExLCbLd | 82 | 746.9005 | 2 | 38.39 | 1491.786 | 0.6 | UNIQUE | 797 |
| 12319 | boiLdExLCpYK | 82 | 785.4205 | 2 | 45.95 | 1568.813 | 8.7 | UNIQUE | 798 |
| 6720 | bjiLdExLCdRH | 82 | 785.4293 | 2 | 35.68 | 1568.837 | 4.6 | UNIQUE | 799 |
| 7513 | bWiLdExLCFQb | 81 | 786.9171 | 2 | 37 | 1571.799 | 13 | UNIQUE | 800 |
| 7899 | boxLdExLCKQG | 81 | 732.8989 | 2 | 37.97 | 1463.767 | 11.2 | UNIQUE | 801 |
| 8703 | boxLdExLCAKG | 81 | 704.3903 | 2 | 39.34 | 1406.745 | 14.7 | UNIQUE | 802 |
| 9784 | boiLdExLCKLp | 81 | 760.4313 | 2 | 41.26 | 1518.834 | 9.6 | UNIQUE | 803 |
| 5981 | boxLdExLCRKA | 80 | 753.9285 | 2 | 34.26 | 1505.825 | 11.7 | UNIQUE | 804 |
| | Peptide | ALC | | | | | | Unique- | SEQ ID |
| Scan | 11-mer | (%) | m/z | Z | RT | Mass | ppm | ness | Nos: |
| 8244 | bWiLdExLCRQ | 94 | 719.8934 | 2 | 37.47 | 1437.769 | 2 | UNIQUE | 47 |
| 9431 | bjxLdExLCQK | 91 | 717.3807 | 2 | 40.62 | 1432.763 | −11.3 | UNIQUE | 101 |
| 8862 | bWiLdExLCHR | 88 | 724.3903 | 2 | 38.58 | 1446.77 | − 2.5 | UNIQUE | 102 |

Position 3 Cyclopentylglycine (Ceg): x, CycloHexylglycine (Chg): i, position 7 Cyclobutylalanine (Cba): x, 4-phenyl phenylalanine(Phf): j, 6-Cl tryptophan (C1W): m, QuinolylAlanine (Qua): n, Napthylalanine(Naf): o, Aminoadipic acid (Aad): b, Homocysteic acid (Hoc): z, Hydroxyproline (Hyp): p, Amino isobutyric (Aib): d

### Example 15—Cell Toxicity Assays with Reactide 13

Cell toxicity of Reactide 13 was determined in a cell toxicity assay using the HPV16 E6 positive cell lines SiHa and CaSki.

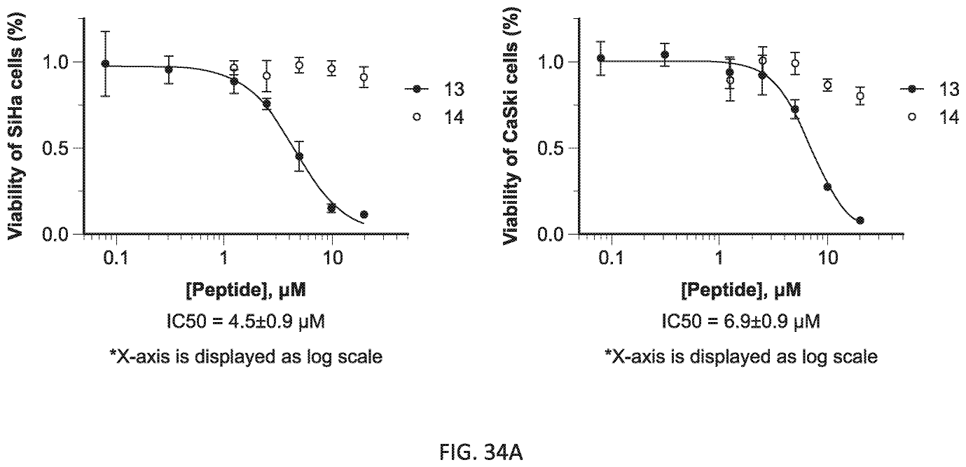

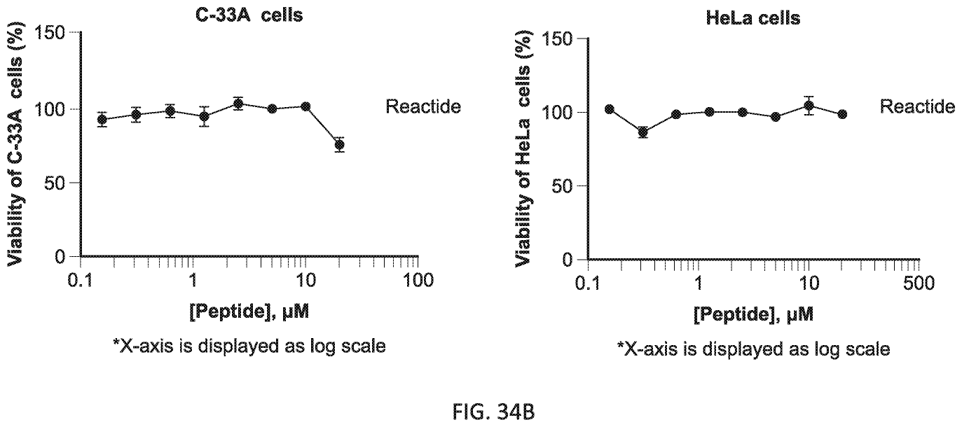

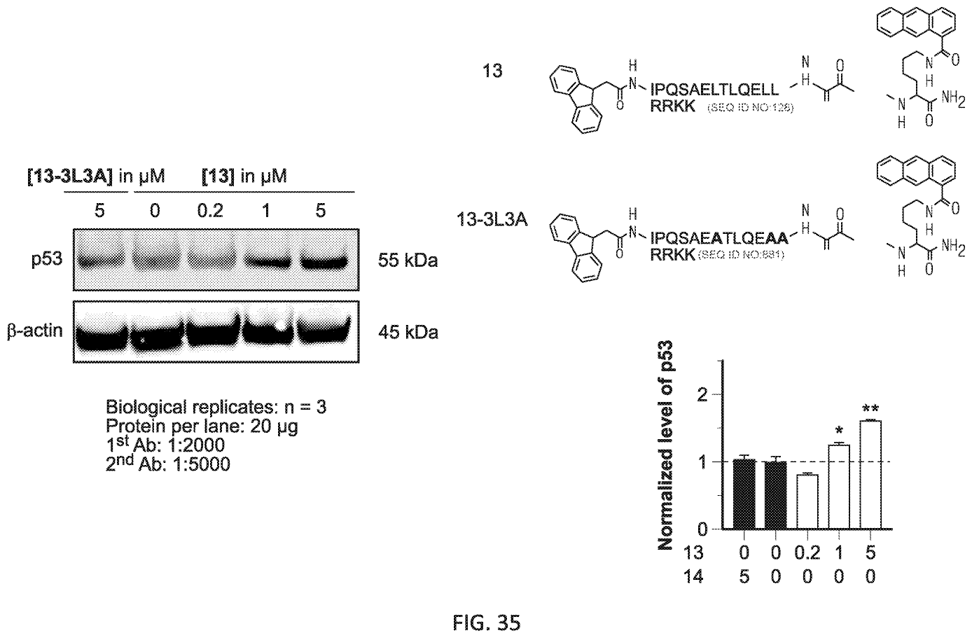

Briefly, HPV16 E6 positive cell lines SiHa and CaSki cells and control cell lines lacking 16E6 expression (HeLa and c-33a) were incubated with reactide 13 and reactide 13-3L3A as a control. At 96 hr, cell viability was estimated using MTT assay. Concentration of the compounds was 20 μM, 10 μM, 5 μM, 2.5 μM, 1.25 μM, 0.4 μM, and 0.08 μM. P53 levels were determined with western Blot. The results are shown in FIG. 34A-34B and FIG. 35.

### REFERENCES

(1) Lombard, I.; Vincent-Salomon, A.; Validire, P.; Zafrani, B.; de la Rochefordibre, A.; Clough, K.; Favre, M.; Pouillart, P.; Sastre-Garau, X. Human Papillomavirus Genotype as a Major Determinant of the Course of Cervical Cancer. J. Clin. Oncol. 1998, 16 (8), 2613-2619. https://doi.org/10.1200/JCO.1998.16.8.2613.
(2) Pal, A.; Kundu, R. Human Papillomavirus E6 and E7: The Cervical Cancer Hallmarks and Targets for Therapy. Front. Microbiol. 2020, 10, 3116. https://doi.org/10.3389/fmnicb.2019.03116.
(3) Graham, S. V. Human Papillomavirus: Gene Expression, Regulation and Prospects for Novel Diagnostic Methods and Antiviral Therapies. Future Microbiol. 2010, 5 (10), 1493-1506. https://doi.org/10.2217/fmnb.10.107.
(4) Schiffman, M.; Doorbar, J.; Wentzensen, N.; De Sanjosé, S.; Fakhry, C.; Monk, B. J.; Stanley, M. A.; Franceschi, S. Carcinogenic Human Papillomavirus Infection. Nat. Rev. Dis. Prim. 2016, 2 (1), 16086. https://doi.org/10.1038/nrdp.2016.86.
(5) Pai, S. I.; Westra, W. H. Molecular Pathology of Head and Neck Cancer: Implications for Diagnosis, Prognosis, and Treatment. Annu. Rev. Pathol. 2009, 4, 49-70. https://doi.org/10.1146/annurev.pathol.4.110807.092158.
(6) Landy, R.; Chaturvedi, A. K. HPV16 E6 Seropositivity and Oropharyngeal Cancer: Marker of Exposure, Risk, or Disease?EBioMedicine 2021, 63, 103190. https://doi.org/10.1016/j.ebiom.2020.103190.
(7) Brenner, N.; Mentzer, A. J.; Hill, M.; Almond, R.; Allen, N.; Pawlita, M.; Waterboer, T. Characterization of Human Papillomavirus (HPV) 16 E6 Seropositive Individuals without HPV-Associated Malignancies after 10 Years of Follow-up in the UK Biobank. EBioMedicine 2020, 62, 103123. https://doi.org/10.1016/j.ebiom.2020.103123.
(8) Martinez-Zapien, D.; Ruiz, F. X.; Poirson, J.; Mitschler, A.; Ramirez, J.; Forster, A.; Cousido-Siah, A.; Masson, M.; Pol, S. Vande; Podjamy, A.; et al. Structure of the E6/E6AP/P53 Complex Required for HPV-Mediated Degradation of P53. Nature 2016, 529 (7587), 541-545. https://doi.org/10.1038/nature16481.
(9) Bosch, F. X.; Broker, T. R.; Forman, D.; Moscicki, A.-B.; Gillison, M. L.; Doorbar, J.; Stem, P. L.; Stanley, M.; Arbyn, M.; Poljak, M.; et al. Comprehensive Control of Human Papillomavirus Infections and Related Diseases. Vaccine 2013, 31, H1-H31. https://doi.org/10.1016/j.vaccine.2013.10.003.
(10) McBride, A. A. Human Papillomaviruses: Diversity, Infection and Host Interactions. Nat. Rev. Microbiol. 2022, 20 (2), 95-108. https://doi.org/10.1038/s41579-021-00617-5.
(11) Muñoz, N.; Bosch, F. X.; de Sanjosé, S.; Herrero, R.; Castellsagud, X.; Shah, K. V.; Snijders, P. J. F.; Meijer, C. J. L. M. Epidemiologic Classification of Human Papillomavirus Types Associated with Cervical Cancer. N. Engl. J. Med. 2003, 348 (6), 518-527. https://doi.org/10.1056/nejmoa021641.
(12) Cohen, P. A.; Jhingran, A.; Oaknin, A.; Denny, L. Cervical Cancer. Lancet 2019, 393 (10167), 169-182. https://doi.org/10.1016/S0140-6736(18)32470-X.
(13) Ghebre, R. G.; Grover, S.; Xu, M. J.; Chuang, L. T.; Simonds, H. Cervical Cancer Control in HIV-Infected Women: Past, Present and Future. Gynecol. Oncol. reports 2017, 21, 101-108. https://doi.org/10.1016/j.gore.2017.07.009.
(14) Wu, Q.; Qian, W.; Sun, X.; Jiang, S. Small-Molecule Inhibitors, Immune Checkpoint Inhibitors, and More: FDA-Approved Novel Therapeutic Drugs for Solid Tumors from 1991 to 2021. J. Hematol. Oncol. 2022, 15 (1), 143. https://doi.org/10.1186/s13045-022-01362-9.
(15) Hollebecque, A.; Meyer, T.; Moore, K. N.; Machiels, J.-P. H.; De Greve, J.; López-Picazo, J. M.; Oaknin, A.; Kerger, J. N.; Boni, V.; Evans, T. R. J.; et al. An Open-Label, Multicohort, Phase I/II Study of Nivolumab in Patients with Virus-Associated Tumors (CheckMate 358): Efficacy and Safety in Recurrent or Metastatic (R/M) Cervical, Vaginal, and Vulvar Cancers. J. Clin. Oncol. 2017, 35 (15_suppl), 5504-5504. https://doi.org/10.1200/JCO.2017.35.15_suppl.5504.
(16) FDA Approves Pembrolizumab for Advanced Cervical Cancer with Disease Progression during or after Chemotherapy. 2018.
(17) Chung, H. C.; Schellens, J. H. M.; Delord, J.-P.; Perets, R.; Italiano, A.; Shapira-Frommer, R.; Manzuk, L.; Piha-Paul, S. A.; Wang, J.; Zeigenfuss, S.; et al. Pembrolizumab Treatment of Advanced Cervical Cancer: Updated Results from the Phase 2 KEYNOTE-158 Study. J. Clin. Oncol. 2018, 36 (15_suppl), 5522-5522. https://doi.org/10.1200/JCO.2018.36.15_suppl.5522.
(18) Roden, R. B. S.; Stem, P. L. Opportunities and Challenges for Human Papillomavirus Vaccination in Cancer. Nat. Rev. Cancer 2018, 18 (4), 240-254. https://doi.org/10.1038/nrc.2018.13.
(19) White, E. A.; Sowa, M. E.; Tan, M. J. A.; Jeudy, S.; Hayes, S. D.; Santha, S.; Muriger, K.; Harper, J. W.; Howley, P. M. Systematic Identification of Interactions between Host Cell Proteins and E7 Oncoproteins from Diverse Human Papillomaviruses. Proc. Natl. Acad. Sci. U.S.A. 2012, 109 (5), E260-E267. https://doi.org/10.1073/pnas.1116776109.
(20) Chen, J. J.; Hong, Y.; Rustamzadeh, E.; Baleja, J. D.; Androphy, E. J. Identification of an a Helical Motif Sufficient for Association with Papillomavirus E6. J. Biol. Chem. 1998, 273
(22), 13537-13544. https://doi.org/10.1074/JBC.273.22.13537.
(21) Ronco, L. V.; Karpova, A. Y.; Vidal, M.; Howley, P. M. Human Papillomavirus 16 E6 Oncoprotein Binds to Interferon Regulatory Factor-3 and Inhibits Its Transcriptional Activity. Genes Dev. 1998, 12 (13), 2061-2072. https://doi.org/10.1101/gad.12.13.2061.
(22) Tong, X.; Howley, P. M. The Bovine Papillomavirus E6 Oncoprotein Interacts with Paxillin and Disrupts the Actin Cytoskeleton. Proc. Natl. Acad. Sci. 1997, 94 (9), 4412-4417. https://doi.org/10.1073/pnas.94.9.4412.
(23) Araujo-Arcos, L. E.; Montano, S.; Bello-Rios, C.; Garibay-Cerdenares, 0. L.; Leyva-Vizquez, M. A.; Illades-Aguiar, B. Molecular Insights into the Interaction of HPV-16 E6 Variants against MAGI-1 PDZ1 Domain. Sci. Rep. 2022, 12 (1), 1898. https://doi.org/10.1038/s41598-022-05995-1.
(24) Howie, H. L.; Katzenellenbogen, R. A.; Galloway, D. A. Papillomavirus E6 Proteins. Virology 2009, 384 (2), 324-334. https://doi.org/10.1016/j.virol.2008.11.017.
(25) Moody, C. A.; Laimins, L. A. Human Papillomavirus Oncoproteins: Pathways to Transformation. Nat. Rev. Cancer 2010, 10 (8), 550-560. https://doi.org/10.1038/nrc2886.
(26) Hoppe-Seyler, K.; Bossler, F.; Braun, J. A.; Herrmann, A. L.; Hoppe-Seyler, F. The HPV E6/E7 Oncogenes: Key Factors for Viral Carcinogenesis and Therapeutic Targets. Trends in Microbiology. 2018, pp 158-168. https://doi.org/10.1016/j.tim.2017.07.007.
(27) Kumar, A.; Rathi, E.; Hariharapura, R. C.; Kini, S. G. Is Viral E6 Oncoprotein a Viable Target?A Critical Analysis in the Context of Cervical Cancer. Med. Res. Rev. 2020, 40 (5), 2019-2048. https://doi.org/10.1002/med.21697.
(28) Hassin, 0.; Oren, M. Drugging P53 in Cancer: One Protein, Many Targets. Nat. Rev. Drug Discov. 2022, 1-18. https://doi.org/10.1038/s41573-022-00571-8.
(29) Jones, D. L.; Thompson, D. A.; Münger, K. Destabilization of the RB Tumor Suppressor Protein and Stabilization of P53 Contribute to HPV Type 16 E7-Induced Apoptosis. Virology 1997, 239 (1), 97-107. https://doi.org/10.1006/VIRO.1997.8851.
(30) Zerfass, K.; Schulze, A.; Spitkovsky, D.; Friedman, V.; Henglein, B.; Jansen-Dürr, P. Sequential Activation of Cyclin E and Cyclin A Gene Expression by Human Papillomavirus Type 16 E7 through Sequences Necessary for Transformation. J. Virol. 1995, 69 (10), 6389-6399. https://doi.org/10.1 128/JVI.69.10.6389-6399.1995.
(31) Lee, J.-H.; Kang, Y.-S.; Koh, J.-W.; Park, S.-Y.; Kim, B.-G.; Lee, E.-D.; Lee, K.-H.; Park, K.-B.; Seo, Y.-L. P53 Gene Mutation Is Rare in Human Cervical Carcinomas with Positive HPV Sequences. Int. J. Gynecol. Cancer 1994, 4 (6), 371-378. https://doi.org/10.1046/j.1525-1438.1994.04060371.x.
(32) Niu, X. Y.; Peng, Z. L.; Duan, W. Q.; Wang, H.; Wang, P. Inhibition of HPV 16 E6 Oncogene Expression by RNA Interference in Vitro and in Vivo. Int. J. Gynecol. Cancer 2006, 16 (2). https://doi.org/10.1136/ijgc-00009577-200603000-00044.
(33) Chang, J. T.-C.; Kuo, T.-F.; Chen, Y.-J.; Chiu, C.-C.; Lu, Y.-C.; Li, H.-F.; Shen, C.-R.; Cheng, A.-J. Highly Potent and Specific SiRNAs against E6 or E7 Genes of HPV16- or HPV18-Infected Cervical Cancers. Cancer Gene Ther. 2010, 17 (12), 827-836. https://doi.org/10.1038/cgt.2010.38.
(34) Scheffner, M.; Werness, B. A.; Huibregtse, J. M.; Levine, A. J.; Howley, P. M. The E6 Oncoprotein Encoded by Human Papillomavirus Types 16 and 18 Promotes the Degradation of P53. Cell 1990, 63 (6), 1129-1136. https://doi.org/10.1016/0092-8674(90)90409-8.
(35) Scheffner, M.; Huibregtse, J. M.; Vierstra, R. D.; Howley, P. M. The HPV-16 E6 and E6-AP Complex Functions as a Ubiquitin-Protein Ligase in the Ubiquitination of P53. Cell 1993, 75 (3), 495-505. https://doi.org/10.1016/0092-8674(93)90384-3.
(36) Ansari, T.; Brimer, N.; Vande Pol, S. B. Peptide Interactions Stabilize and Restructure Human Papillomavirus Type 16 E6 To Interact with P53. J. Virol. 2012, 86 (20), 11386-11391. https://doi.org/10.1 128/jvi.01236-12.
(37) Hafner, A.; Bulyk, M. L.; Jambhekar, A.; Lahav, G. The Multiple Mechanisms That Regulate P53 Activity and Cell Fate. Nat. Rev. Mol. Cell Biol. 2019, 20 (4), 199-210. https://doi.org/10.1038/s41580-019-0110-x.
(38) Chao, C. C.-K. Mechanisms of P53 Degradation. Clin. Chim. Acta. 2015, 438, 139-147. https://doi.org/10.1016/j.cca.2014.08.015.
(39) Yamagishi, Y.; Shoji, I.; Miyagawa, S.; Kawakami, T.; Katoh, T.; Goto, Y.; Suga, H. Natural Product-like Macrocyclic N-Methyl-Peptide Inhibitors against a Ubiquitin Ligase Uncovered from a Ribosome-Expressed de Novo Library. Chem. Biol. 2011, 18 (12), 1562-1570. https://doi.org/10.1016/j.chembiol.2011.09.013.
(40) Bandilovska, I.; Keam, S. P.; Gamell, C.; Machicado, C.; Haupt, S.; Haupt, Y. E6AP Goes Viral: The Role of E6AP in Viral- and Non-Viral-Related Cancers. Carcinogenesis 2019, 40 (6), 707-714. https://doi.org/10.1093/carcin/bgz072.
(41) Avagliano Trezza, R.; Sonzogni, M.; Bossuyt, S. N. V; Zampeta, F. I.; Punt, A. M.; van den Berg, M.; Rotaru, D. C.; Koene, L. M. C.; Munshi, S. T.; Stedehouder, J.; et al. Loss of Nuclear UBE3A Causes Electrophysiological and Behavioral Deficits in Mice and Is Associated with Angelman Syndrome. Nat. Neurosci. 2019, 22 (8), 1235-1247. https://doi.org/10.1038/s41593-019-0425-0.
(42) Sutcliffe, J. S.; Jiang, Y. H.; Galijaard, R. J.; Matsuura, T.; Fang, P.; Kubota, T.; Christian, S. L.; Bressler, J.; Cattanach, B.; Ledbetter, D. H.; et al. The E6-Ap Ubiquitin-Protein Ligase (UBE3A) Gene Is Localized within a Narrowed Angelman Syndrome Critical Region. Genome Res. 1997, 7 (4), 368-377. https://doi.org/10.1101/gr.7.4.368.
(43) Offensperger, F.; Müller, F.; Jansen, J.; Hammler, D.; Götz, K. H.; Marx, A.; Sirois, C. L.; Chamberlain, S. J.; Stengel, F.; Scheffner, M. Identification of Small-Molecule Activators of the Ubiquitin Ligase E6AP/UBE3A and Angelman Syndrome-Derived E6AP/UBE3A Variants. Cell Chem. Biol. 2020, 27 (12), 1510-1520.e6. https://doi.org/10.1016/j.chembiol.2020.08.017.
(44) da Silva, S. P.; Santos, J. M. O.; Mestre, V. F.; Medeiros-Fonseca, B.; Oliveira, P. A.; Bastos, M. M. S. M.; Gil da Costa, R. M.; Medeiros, R. Human Papillomavirus 16-Transgenic Mice as a Model to Study Cancer-Associated Cachexia. Int. J. Mol. Sci. 2020, 21 (14), 1-17. https://doi.org/10.3390/ijms21145020.
(45) Yamato, K.; Yamada, T.; Kizaki, M.; Ui-Tei, K.; Natori, Y.; Fujino, M.; Nishihara, T.; Ikeda, Y.; Nasu, Y.; Saigo, K.; et al. New Highly Potent and Specific E6 and E7 SiRNAs for Treatment of HPV16 Positive Cervical Cancer. Cancer Gene Ther. 2008, 15 (3), 140-153. https://doi.org/10.1038/sj.cgt.7701118.
(46) Sugo, T.; Terada, M.; Oikawa, T.; Miyata, K.; Nishimura, S.; Kenjo, E.; Ogasawara-Shimizu, M.; Makita, Y.; Imaichi, S.; Murata, S.; et al. Development of Antibody-SiRNA Conjugate Targeted to Cardiac and Skeletal Muscles. J. Control. Release 2016, 237, 1-13. https://doi.org/10.1016/J.JCONREL.2016.06.036.
(47) Alvarez-Salas, L. M.; Cullinan, A. E.; Siwkowski, A.; Hampel, A.; Dipaolo, J. A. Inhibition of HPV-16 E6/E7 Immortalization of Normal Keratinocytes by Hairpin Ribozymes. Proc. Natl. Acad. Sci. U.S.A 1998, 95 (3), 1189-1194. https://doi.org/10.1073/pnas.95.3.1189.
(48) Cherry, J. J.; Rietz, A.; Malinkevich, A.; Liu, Y.; Xie, M.; Bartolowits, M.; Davisson, V. J.; Baleja, J. D.; Androphy, E. J. Structure Based Identification and Characterization of Flavonoids That Disrupt Human Papillomavirus-16 E6 Function. PLoS One 2013, 8 (12), e84506. https://doi.org/10.1371/joumal.pone.0084506.
(49) Yuan, C. H.; Filippova, M.; Krstenansky, J. L.; Duerksen-Hughes, P. J. Flavonol and Imidazole Derivatives Block HPV16 E6 Activities and Reactivate Apoptotic Pathways in HPV+ Cells. Cell Death Dis. 2016, 7 (1), e2060-e2060. https://doi.org/10.1038/cddis.2015.391.
(50) Malecka, K. A.; Fera, D.; Schultz, D. C.; Hodawadekar, S.; Reichman, M.; Donover, P. S.; Murphy, M. E.; Marmorstein, R. Identification and Characterization of Small Molecule Human Papillomavirus E6 Inhibitors. ACS Chem. Biol. 2014, 9 (7), 1603-1612. https://doi.org/10.1021/cb500229d.
(51) Zhu, J.; Kamara, S.; Wang, Q.; Guo, Y.; Li, Q.; Wang, L.; Chen, J.; Du, Q.; Du, W.; Chen, S.; et al. Novel Affibody Molecules Targeting the HPV16 E6 Oncoprotein Inhibited the Proliferation of Cervical Cancer Cells. Front. Cell Dev. Biol. 2021, 9, 1150. https://doi.org/10.3389/fcell.2021.677867.
(52) Zhang, W.; Shan, H.; Jiang, K.; Huang, W.; Li, S. A Novel Intracellular Nanobody against HPV16 E6 Oncoprotein. Clin. Immunol. 2021, 225, 1521-6616. https://doi.org/10.1016/j.clim.2021.108684.
(53) Ramirez, J.; Poirson, J.; Foltz, C.; Chebaro, Y.; Schrapp, M.; Meyer, A.; Bonetta, A.; Forster, A.; Jacob, Y.; Masson, M.; et al. Targeting the Two Oncogenic Functional Sites of the HPV E6 Oncoprotein with a High-Affinity Bivalent Ligand. Angew. Chemie 2015, 127 (27), 8069-8073. https://doi.org/10.1002/anie.201502646.
(54) Zanier, K.; Charbonnier, S.; Sidi, A. O. M. hamed O.; McEwen, A. G.; Ferrario, M. G.; Poussin-Courmontagne, P.; Cura, V.; Brimer, N.; Babah, K. O.; Ansari, T.; et al. Structural Basis for Hijacking of Cellular LxxLL Motifs by Papillomavirus E6 Oncoproteins. Science (80-.). 2013, 339 (6120), 694-698. https://doi.org/10.1126/science.1229934.
(55) Huibregtse, J. M.; Scheffner, M.; Howley, P. M. Localization of the E6-AP Regions That Direct Human Papillomavirus E6 Binding, Association with P53, and Ubiquitination of Associated Proteins. Mol. Cell. Biol. 1993, 13 (8), 4918-4927. https://doi.org/10.1128/mcb.13.8.4918-4927.1993.
(56) Elston, R. C.; Napthine, S.; Doorbar, J. The Identification of a Conserved Binding Motif within Human Papillomavirus Type 16 E6 Binding Peptides, E6AP and E6BP. J. Gen. Virol. 1998, 79 (2), 371-374. https://doi.org/10.1099/0022-1317-79-2-371.
(57) Muttenthaler, M.; King, G. F.; Adams, D. J.; Alewood, P. F. Trends in Peptide Drug Discovery. Nat. Rev. Drug Discov. 2021, 20 (4), 309-325. https://doi.org/10.1038/s41573-020-00135-8.
(58) Saw, P. E.; Xu, X.; Kim, S.; Jon, S. Biomedical Applications of a Novel Class of High-Affinity Peptides. Acc. Chem. Res. 2021, 54 (18), 3576-3592. https://doi.org/10.1021/acs.accounts.1c00239.
(59) Zanier, K.; Stutz, C.; Kintscher, S.; Reinz, E.; Sehr, P.; Bulkescher, J.; Hoppe-Seyler, K.; Travé, G.; Hoppe-Seyler, F. The E6AP Binding Pocket of the HPV16 E6 Oncoprotein Provides a Docking Site for a Small Inhibitory Peptide Unrelated to E6AP, Indicating Druggability of E6. PLoS One 2014, 9 (11), e112514. https://doi.org/10.1371/joumal.pone.0112514.
(60) Dymalla, S.; Scheffner, M.; Weber, E.; Sehr, P.; Lohrey, C.; Hoppe-Seyler, F.; Hoppe-Seyler, K. A Novel Peptide Motif Binding to and Blocking the Intracellular Activity of the Human Papillomavirus E6 Oncoprotein. J. Mol. Med. 2009, 87 (3), 321-331. https://doi.org/10.1007/s00109-008-0432-1.
(61) Stutz, C.; Reinz, E.; Honegger, A.; Bulkescher, J.; Schweizer, J.; Zanier, K.; Travé, G.; Lohrey, C.; Hoppe-Seyler, K.; Hoppe-Seyler, F. Intracellular Analysis of the Interaction between the Human Papillomavirus Type 16 E6 Oncoprotein and Inhibitory Peptides. PLoS One 2015, 10 (7), e0132339. https://doi.org/10.1371/joumal.pone.0132339.
(62) Liu, Y.; Liu, Z.; Androphy, E.; Chen, J.; Baleja, J. D. Design and Characterization of Helical Peptides That Inhibit the E6 Protein of Papillomavirus. Biochemistry 2004, 43 (23), 7421-7431. https://doi.org/10.1021/bi049552a.
(63) Shuman, C. F.; Markgren, P. O.; Hämäläinen, M.; Danielson, U. H. Elucidation of HIV-1 Protease Resistance by Characterization of Interaction Kinetics between Inhibitors and Enzyme Variants. Antiviral Res. 2003, 58 (3), 235-242. https://doi.org/10.1016/S0166-3542(03)00002-0.
(64) Mah, R.; Thomas, J. R.; Shafer, C. M. Drug Discovery Considerations in the Development of Covalent Inhibitors. Bioorganic Med. Chem. Lett. 2014, 24 (1), 33-39. https://doi.org/10.1016/j.bmcl.2013.10.003.
(65) Bauer, R. A. Covalent Inhibitors in Drug Discovery: From Accidental Discoveries to Avoided Liabilities and Designed Therapies. Drug Discov. Today 2015, 20 (9), 1061-1073. https://doi.org/10.1016/j.drudis.2015.05.005.
(66) Abdeldayem, A.; Raouf, Y. S.; Constantinescu, S. N.; Moriggl, R.; Gunning, P. T. Advances in Covalent Kinase Inhibitors. Chem. Soc. Rev. 2020, 49 (9), 2617-2687. https://doi.org/10.1039/c9cs00720b.
(67) Berdan, V. Y.; Klauser, P. C.; Wang, L. Covalent Peptides and Proteins for Therapeutics. Bioorganic Med. Chem. 2021, 29, 115896. https://doi.org/10.1016/j.bmc.2020.115896.
(68) Boike, L.; Henning, N. J.; Nomura, D. K. Advances in Covalent Drug Discovery. Nat. Rev. Drug Discov. 2022, 1-18. https://doi.org/10.1038/s41573-022-00542-z.
(69) De Araujo, A. D.; Lim, J.; Wu, K. C.; Xiang, Y.; Good, A. C.; Skerlj, R.; Fairlie, D. P. Bicyclic Helical Peptides as Dual Inhibitors Selective for Bcl2A1 and Mcl-1 Proteins. J. Med. Chem. 2018, 61 (7), 2962-2972. https://doi.org/10.1021/acs.jmedchem.8b00010.
(70) Nominé, Y.; Charbonnier, S.; Ristriani, T.; Stier, G.; Masson, M.; Cavusoglu, N.; Van Dorsselaer, A.; Weiss, E.; Kieffer, B.; Travé, G. Domain Substructure of HPV E6 Oncoprotein: Biophysical Characterization of the E6 C-Terminal DNA-Binding Domain. Biochemistry 2003, 42 (17), 4909-4917. https://doi.org/10.1021/bi026980c.
(71) Ye, X.; Lee, Y.-C.; Gates, Z. P.; Ling, Y.; Mortensen, J. C.; Yang, F.-S.; Lin, Y.-S.; Pentelute, B. L. Binary Combinatorial Scanning Reveals Potent Poly-Alanine-Substituted Inhibitors of Protein-Protein Interactions. Commun. Chem. 2022, 5 (1), 128. https://doi.org/10.1038/s42004-022-00737-w.
(72) Be, X.; Hong, Y.; Wei, J.; Androphy, E. J.; Chen, J. J.; Baleja, J. D. Solution Structure Determination and Mutational Analysis of the Papillomavirus E6 Interacting Peptide of E6AP. Biochemistry 2001, 40 (5), 1293-1299. https://doi.org/10.1021/bi0019592.
(73) Ko, L.; Cardona, G. R.; Chin, W. W. Thyroid Hormone Receptor-Binding Protein, an LXXLL Motif-Containing Protein, Functions as a General Coactivator. Proc. Natl. Acad. Sci. U.S.A. 2000, 97 (11), 6212-6217. https://doi.org/10.1073/pnas.97.11.6212.
(74) Lonsdale, R.; Ward, R. A. Structure-Based Design of Targeted Covalent Inhibitors. Chem. Soc. Rev. 2018, 47 (11), 3816-3830. https://doi.org/10.1039/c7cs00220c.
(75) Chalker, J. M.; Gunnoo, S. B.; Boutureira, O.; Gerstberger, S. C.; Femindez-Gonzilez, M.; Bernardes, G. J. L.; Griffin, L.; Hailu, H.; Schofield, C. J.; Davis, B. G. Methods for Converting Cysteine to Dehydroalanine on Peptides and Proteins. Chem. Sci. 2011, 2 (9), 1666-1676. https://doi.org/10.1039/clsc00185j.
(76) Eng, H.; Dantonio, A. L.; Kadar, E. P.; Scott Obach, R.; Di, L.; Lin, J.; Patel, N. C.; Boras, B.; Walker, G. S.; Novak, J. J.; et al. Disposition of Nirmatrelvir, an Orally Bioavailable Inhibitor of SARS-CoV-2 3C-Like Protease, across Animals and HumansS. Drug Metab. Dispos. 2022, 50 (5), 576-590. https://doi.org/10.1124/dmd.121.000801.
(77) de Jong, H.; Bonger, K. M.; Löwik, D. W. P. M. Activatable Cell-Penetrating Peptides: 15 Years of Research. RSC Chem. Biol. 2020, 1 (4), 192-203. https://doi.org/10.1039/D0CB00114G.
(78) Wolfe, J. M.; Fadzen, C. M.; Choo, Z. N.; Holden, R. L.; Yao, M.; Hanson, G. J.; Pentelute, B. L. Machine Learning to Predict Cell-Penetrating Peptides for Antisense Delivery. ACS Cent. Sci. 2018, 4 (4), 512-520. https://doi.org/10.1021/acscentsci.8b00098.
(79) Conrady, M. C.; Suarez, I.; Gogl, G.; Frecot, D. I.; Bonhoure, A.; Kostmann, C.; Cousido-Siah, A.; Mitschler, A.; Lim, J.; Masson, M.; et al. Structure of High-Risk Papillomavirus 31 E6 Oncogenic Protein and Characterization of E6/E6AP/P53 Complex Formation. J. Virol. 2020, 95 (2). https://doi.org/10.1128/jvi.00730-20.
(80) Frank, A. O.; Vangamudi, B.; Feldkamp, M. D.; Souza-Fagundes, E. M.; Luzwick, J. W.; Cortez, D.; Olejniczak, E. T.; Waterson, A. G.; Rossanese, O. W.; Chazin, W. J.; et al. Discovery of a Potent Stapled Helix Peptide That Binds to the 70N Domain of Replication Protein A. J. Med. Chem. 2014, 57 (6), 2455-2461. https://doi.org/10.1021/jm401730y.
(81) Torner, J. M.; Yang, Y.; Rooklin, D.; Zhang, Y.; Arora, P. S. Identification of Secondary Binding Sites on Protein Surfaces for Rational Elaboration of Synthetic Protein Mimics. ACS Chem. Biol. 2021, 16 (7), 1179-1183. https://doi.org/10.1021/acschembio.1c00418.
(82) Paulussen, F. M.; Grossmann, T. N. Peptide-Based Covalent Inhibitors of Protein-Protein Interactions. J. Pept. Sci. 2023, 29 (1), 1-12. https://doi.org/10.1002/psc.3457.
(83) Jones, L. H. Dehydroamino Acid Chemical Biology: An Example of Functional Group Interconversion on Proteins. RSC Chemical Biology. Royal Society of Chemistry Dec. 8, 2020, pp 298-304. https://doi.org/10.1039/dOcb00174k.
(84) Dadová, J.; Galan, S. R.; Davis, B. G. Synthesis of Modified Proteins via Functionalization of Dehydroalanine. Curr. Opin. Chem. Biol. 2018, 46, 71-81. https://doi.org/10.1016/j.cbpa.2018.05.022.
(85) MacKintosh, R. W.; Dalby, K. N.; Campbell, D. G.; Cohen, P. P. T. W.; Cohen, P. P. T. W.; MacKintosh, C. The Cyanobacterial Toxin Microcystin Binds Covalently to Cysteine-273 on Protein Phosphatase 1. FEBS Lett. 1995, 371 (3), 236-240. https://doi.org/10.1016/0014-5793(95)00888-G.
(86) Runnegar, M.; Berndt, N.; Kong, S. M.; Lee, E. Y. C.; Zhang, L. F. In Vivo and in Vitro Binding of Microcystin to Protein Phosphatase 1 and 2A. Biochem. Biophys. Res. Commun. 1995, 216 (1), 162-169. https://doi.org/10.1006/bbrc.1995.2605.
(87) Downs, D. M.; Ernst, D. C. From Microbiology to Cancer Biology: The Rid Protein Family Prevents Cellular Damage Caused by Endogenously Generated Reactive Nitrogen Species. Mol. Microbiol. 2015, 96 (2), 211-219. https://doi.org/10.1111/mmi.12945.
(87) J. Rappsilber, M. Mann, Y. Ishihama, Nat. Protoc. 2007, 2, 1896-1906. (88) E. L. Huttlin, M. P. Jedrychowski, J. E. Elias, T. Goswami, R. Rad, S. A. Beausoleil, J. Villén, W. Haas, M. E. Sowa, S. P. Gygi, Cell. 2010, 143, 1174-1189.
(89) M. F. E. O'Brien, J. J, Raj, A., Gaun, A, Waite A, Li W., Hendrickson D., Olsson N., Nat. Methods. 2023, Accepted.
A. J. Mijalis, D. A. Thomas, M. D. Simon, A. Adamo, R. Beaumont, K. F. Jensen, B. L. Pentelute, Nat. Chem. Biol. 2017, 13, 464-466.
N. Hartrampf, A. Saebi, M. Poskus, Z. P. Gates, A. J. Callahan, A. E. Cowfer, S. Hanna, S. Antilla, C. K. Schissel, A. J. Quartararo, X. Ye, A. J. Mijalis, M. D. Simon, A. Loas, S. Liu, C. Jessen, T. E. Nielsen, B. L. Pentelute, Science (80-.). 2020, 368, 980-987.
X. Ye, Y.-C. Lee, Z. P. Gates, Y. Ling, J. C. Mortensen, F.-S. Yang, Y.-S. Lin, B. L. Pentelute, Commun. Chem. 2022, 5, 128.
(90) P. Labute, J. Chem. Inf Model. 2010, 50, 792-800.
(91) Y. Zhang, N. Wang, W. Wang, J. Wang, Z. Zhu, X. Li, Peptides 2016, 76, 45-50.
(92) M. Tahir ul Qamar, A. Bari, M. M. Adeel, A. Maryam, U. A. Ashfaq, X. Du, I. Muneer, H. I. Ahmad, J. Wang, J. Transl. Med. 2018, 16, 298.
(93) X. Daura, K. Gademann, B. Jaun, D. Seebach, W. F. van Gunsteren, A. E. Mark, A. Rigault, J. Siegel, J. Harrowfield, B. Chevrier, D. Moras, J. Lehn, M. Garrett, U. Koert, D. Meyer, J. Fischer, Peptide Folding: When Simulation Meets Experiment, 1998.
(94) M. J. Abraham, T. Murtola, R. Schulz, S. Pill, J. C. Smith, B. Hess, E. Lindahl, SoftwareX 2015, 1-2, 19-25.
(95) J. A. Maier, C. Martinez, K. Kasavajhala, L. Wickstrom, K. E. Hauser, C. Simmerling, J. Chem. Theory Comput. 2015, 11, 3696-713.
(96) J. Wang, W. Wang, P. A. Kollman, D. A. Case, J. Mol. Graph. Model. 2006, 25, 247-60.
(97) J. Wang, R. M. Wolf, J. W. Caldwell, P. A. Kollman, D. A. Case, J. Comput. Chem. 2004, 25, 1157-1174.
(98) G. Bussi, D. Donadio, M. Parrinello, J. Chem. Phys. 2007, 126, 014101.
(99) M. Parrinello, A. Rahman, J. Appl. Phys. 1981, 52, 7182-7190.
(100) U. Essmann, L. Perera, M. L. Berkowitz, T. Darden, H. Lee, L. G. Pedersen, J. Chem. Phys. 1995, 103, 8577-8593.
(101) S. Miyamoto, P. A. Kollman, J. Comput. Chem. 1992, 13, 952-962.
(102) B. Hess*, 2007, DOI 10.1021/CT700200B.
(103) G. A. Tribello, M. Bonomi, D. Branduardi, C. Camilloni, G. Bussi, Comput. Phys. Commun. 2014, 185, 604-613.

### INCORPORATION BY REFERENCE

The entire disclosure of each of the patent and scientific documents referred to herein is incorporated by reference for all purposes.

### EQUIVALENTS

An invention may be embodied in other specific forms without departing from the spirit or essential characteristics thereof. The foregoing embodiments are therefore to be considered in all respects illustrative rather than limiting on any invention disclosed herein. Scope of an invention is thus indicated by the appended claims rather than by the foregoing description, and all changes that come within the meaning and range of equivalency of the claims are intended to be embraced therein.

1. A synthetic peptide comprising an amino acid sequence IPESSELTLQELLGEER (SEQ ID NO: 1) or ELTLQELLGEER (SEQ ID NO: 5).

2. (canceled)

3. The synthetic peptide of claim 1, wherein the N-terminus of the synthetic peptide is modified with a chemical moiety, wherein the chemical moiety is selected from the group consisting of Fluorescein-5-Isothiocyanate, 9-fluorene acetamido, 1-fluorene acetamido, 1-Indane acetamido, 9-fluorenone-2-carboxamido, 9-fluorenone-1-carboxamido, 9-fluorenone-4-carboxamido, anthraquinone-2-carboxamido, xanthene-9-carboxamido, 1-anthracene carboxamido, 2-anthracene carboxamido, 1-adamantane carboxamido, Triphenyl acetamido, Diphenyl acetamido, 1-naphthyl carboxamido, 1,6-dihydrophenyl carboxamido, Pentafluorophenyl carboxamido, 6-hydroxy-2-naphthyl carboxamido, 1-pyrenebutyl carboxamido, 5-Acenaphthene carboxamide, 7-Methoxycoumarin-4-acetamido, 4-phenyl-phenylalanine, and Cyclohexyl-alanine and/or wherein the chemical moiety is selected from the group consisting of Biotin-PEG4, Fluorene, Anthracene, and Fluorene-(Biotin-PEG4-)K, and the second chemical moiety is selected from the group of 9-fluorene, 5-Acenaphthene, 1-naphthanlene, 2-anthraquinone, and 1-Anthracene.

4. The synthetic peptide of claim 1, wherein the C-terminus of the peptide is modified with a chemical moiety, wherein the chemical moiety is selected from the group consisting of Fluorescein-5-Isothiocyanate, fluorene-9-acetamido, fluorene-1-acetamido, 1-Indanecarboxamido, 9-fluorenone-2-carboxamido, 9-fluorenone-1-carboxamido, 9-fluorenone-4-carboxamido, anthraquinone-2-carboxamido, xanthene-9-carboxamido, 1-anthracene carboxamido, 2-anthracene carboxamido, 1-adamantane carboxamido, triphenylacetamido, diphenylacetamido, 1-naphthyl carboxamido, and Exo-norbornene-carboxamido.

5. The synthetic peptide of claim 1, wherein the peptide is substituted with a chemical moiety at the N-terminus or C-terminus, wherein the chemical moiety is selected from the group of 9-fluorenyl, 1-fluorenyl, 1-Indanyl, 9-oxofluoren-3-yl, 9-oxofluoren-4-yl, 9-oxofluoren-1-yl, 2-anthraquinonyl, 9-xanthyl, 1-anthracenyl, 2-anthracenyl, Adamantyl, Triphenylmethyl, Diphenylmethyl, 1-naphthyl, 2,6-dihydroxy-phenyl, Pentafluoro-phenyl, 6-Hydroxynaphthyl, 1-pyrenebutyl, 5-acenaphthyl, Coumarin, Biphenyl, Cyclohexyl, Norborenyl, and Fluorescein.

6-7. (canceled)

8. The synthetic peptide of claim 1, wherein the peptide comprises
(SEQ ID NO: 2)
Fluorene-IPESSELTLQELLGEERRAA-K(1-Anthracene)
or
(SEQ ID NO: 3)
Fluorene-IPESAELTLQELLGEERRAA-K(1-Anthracene).

9. A synthetic peptide modified with a warhead, wherein the peptide is selected from TABLE 2C, TABLE 2D, or TABLE 2E.

10. The synthetic peptide of claim 9, wherein the warhead is at position 9 with respect to SEQ ID NO: 3; wherein the warhead is selected from the group consisting of phenylacrylamide (Ph-acr), Dap-acrylamide (dap-acr), Dab-acrylamide (dab-acr), Dap-propiolamide (dap-ppa), and dehydroalanine Dha.

11. (canceled)

12. The synthetic peptide of claim 9, wherein the peptide comprises
(SEQ ID NO: 4)
Fluorene-IPQSAELTLQELL(DHA)RRKKK(Anthracene).

13. The synthetic peptide of claim 9, wherein the warhead is selected from the group of phenyl fluorosulfate (FS), phenyl sulfonyl fluoride (SF), phenyl Carbamate (p-PhC), phenyl Carbamate (m-PhC), and disulfide (DS).

14. A synthetic peptide comprising an amino acid sequence
(i) X₁X₂X₃X₂QX₁X₂X₂CEER (SEQ ID NO: 6), wherein X₁ is Nva, Leu, Aoc, Cpa, Cba, Cha, Phg, Hof, or Naf; X₂ is Aad, Glu, or Cya; X₃ is Thr, Asn, Hyp, Cpg, Cbg, or Ceg; and X₄ is d-Ala, Aib, Gly, or Gln; or
(ii) Aad-X₁-X₂-Leu-Aib-Aad-Cba-Leu-Cys-X₃-X₃-X₃ (SEQ ID NO: 20) wherein X₁ is PhF, Naf, Qua, Clw, or Trp; X₂ is Chg or Ceg; and X₃ is d-Ala, Aib, Gly, Ser, Aad, Glu, Gln, Hoc, Val, Leu, Met, Pro, Dap, Lys, Arg, His, Trp, Phe, Tyr, or Null.

15. The synthetic peptide of claim 14, wherein the peptide is selected from TABLES 3-31.

16-18. (canceled)

19. The synthetic peptide of claim 4, wherein the peptide is 11 or 12 amino acids long.

20. The synthetic peptide of claim 14, wherein the N-terminus of the peptide is further modified with a modifier selected from 2-Phenyl-4-quinolinecarboxylic acid, 1-(Phenylsulfonyl)-1H-indole-2-carboxylic acid, 6-Fluoro-2-naphthoic acid, 4-phthalimidobenzoic acid, Xanthene 9-carboxylic acid, 1-Pyrenebutyric acid, 5-Acenaphthenecarboxylic acid, 1-Phenyl-1H-indole-2-carboxylic acid, Indole-2-carboxlic acid, quinoline-4-carboxylic acid, and 10-(Carboxymethyl)-9(10H)acridone.

21. The synthetic peptide of claim 14, wherein the peptide is selected from TABLES 32-39.

22. A synthetic peptide comprising the formula

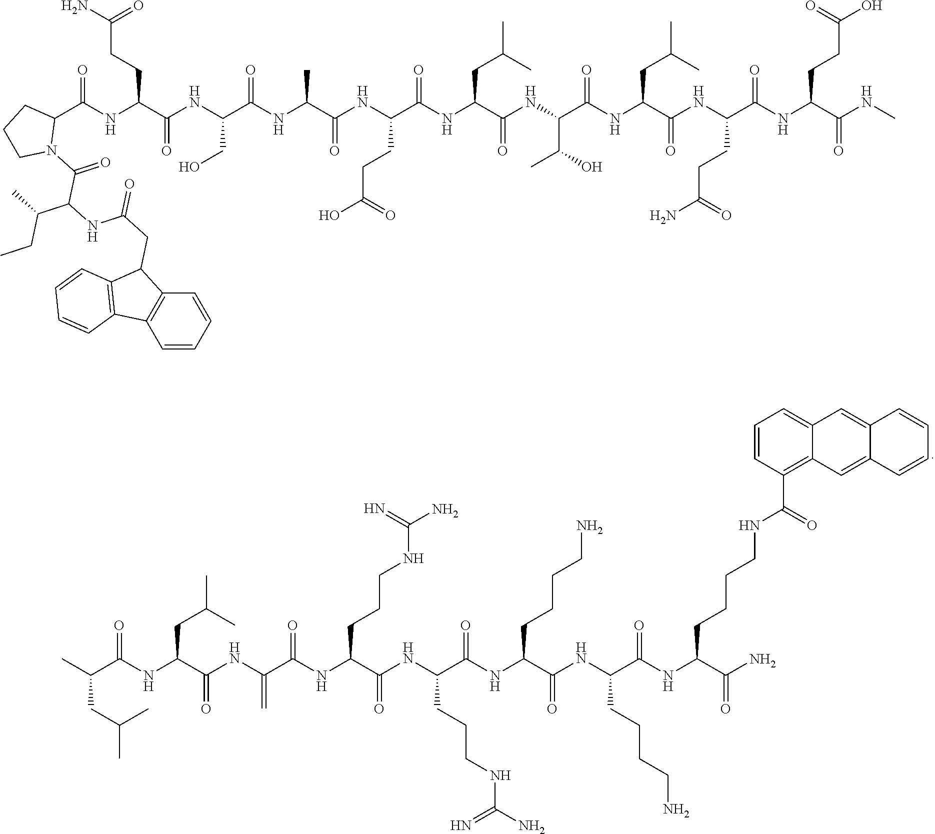

23. The synthetic peptide of claim 14, further comprising a warhead.

24. The synthetic peptide of claim 23, wherein the warhead is selected from the group of phenylacrylamide (Ph-acr), Dap-acrylamide (dap-acr), Dab-acrylamide (dab-acr), Dap-propiolamide (dap-ppa), and dehydroalanine Dha.

25. (canceled)

26. A synthetic peptide of claim 1 comprising one or more additional modifications selected from:
a) acetylated, formylated, propanoylated, hexanoylated, or myristoylated N-terminus;
b) amidated C-terminus;
c) substitution of one or more L-amino acid with a D-amino acid;
d) substitution of one or more amino acid with a methyl-amino acid; and
e) substitution of an α-amino acid with a β-amino acids.

27. A synthetic peptide/HPV16E6 complex, wherein the peptide is the synthetic peptides of claim 1; wherein the synthetic peptide and the HPV16E6 in the complex are covalently linked.

28. (canceled)

29. The synthetic peptide/HPV16E6 complex of claim 27, wherein the synthetic peptide is covalently linked to amino acid residue Cys-58, Arg-84, or Arg-13 of HPV16E6.

30-31. (canceled)
